SEQUENCE LISTING (182 sequences)

SEQ ID NO: 1 - DNA, 1860 bp, Artificial Sequence
  Synthesized menD
atgctggcgcaggcattgtccgtggttccgaccccgagcaagagtggcaggaaatcgacaacaaagcggcagggctgcgtactttattacaaatggaatagtaatgagtcgcatcattaccgattcatatcaataatctatttttgtagctccttatacttagccgcaatattgataccggacaaactcatgtcagtaagcgcatttaaccgacgctgggcggcggtcattctggaagcattaacgcgtcacggcgtcagacacatctgtatcgcccctggctcgcgttctacaccgttaacgttagcggcggcggagaattccgcatttattcatcatacccatttcgatgagcgtggactggggcatctggcactggggctggcgaaagtcagcaagcagccggtggcggtgattgtgacctccggcacggcggtagcaaatctctatccggcactgattgaagctgggttaaccggagaaaaactgatcctgttaaccgccgatcgcccgccggagctgattgactgcggcgcgaatcaggcgattcgtcagccgggaatgttcgcctctcaccccacgcacagtatttcactgccgcgcccgacccaggatatccccgcacgttggctggtttctaccatcgaccacgctctcggtacgcttcatgctggtggagtccatatcaactgcccgtttgctgaaccgctgtatggcgaaatggacgacaccgggattagctggcaacagcggctgggcgactggtggcaggacgacaaaccgtggctgcgtgaagcgcctcgtcgggaaagtgaaaaacagcgtgactggttcttctggcgacaaaagcgcggcgtggtggttgccgggcgcatgagtgcggaagagggcaaaaaagttgcactgtgggcgcaaactcttggctggccgctgattggcgacgtgctgtcgcaaaccggacagccgctgccgtgtgccgatctctggttaggcaatgccaaagcgaccagcgaactgcaacaggcacaaatagtggtgcaactgggaagcagcctgacggggaaacgactcctccaatggcaggcaagctgtgaaccagaagagtactggattgttgatgacattgaagggcgacttgatccggcacaccatcgcggacgtcgcttaattgccaatattgccgactggctggagctgcatccggcagaaaaacgccagccctggtgcgttgaaatcccgcgcctggcggaacaggcaatgcaggcggttattgcccgtcgcgatgcgtttggcgaagcgcaactggcgcatcgcatcagcgactacttgcctgaacaggggcaattgtttgtcggtaacagcctggtggtgcgtctgattgatgcgctttcgcaacttccggcaggttacccggtgtacagcaaccgtggtgccagcggtatcgacgggctgctctcgactgccgccggcgttcagcgggcaagcggcaaaccgacgctggcgattgtgggcgatctctccgcactttacgatctcaacgcgctggcgttattgcgccaggtttccgcaccgctggtattaattgtggtgaacaacaacggcgggcaaattttttcgctgttgccaacgccgaaaagcgagcgcgagcgtttctatctgatgccgcaaaacgtccattttgagcacgccgccgcgatgttcgagctgaaatatcatcgtccgcaaaactggcaggaacttgaaacgacactagtcgatgcctggcgtacgccgaccaccacggtgattgaaatggtggttaacgacaccgatggtgcgcaaacgctccagcaacttctggcgcaggtaagccatttatga

SEQ ID NO: 2 - DNA, 1572 bp, Artificial Sequence
  Synthesized leuA
atgagccagcaagtcattattttcgataccacattgcgcgacggtgaacaggcgttacaggcaagcttgagtgtgaaagaaaaactgcaaattgcgctggcccttgagcgtatgggtgttgacgtgatggaagtcggtttccccgtctcttcgccgggcgattttgaatcggtgcaaaccatcgcccgccaggttaaaaacagccgcgtatgtgcgttagctcgctgcgtggaaaaagatatcgacgtggcggccgaatccctgaaagtcgccgaagccttccgtattcatacctttattgccacttcgccaatgcacatcgccaccaagctgcgcagcacgctggacgaggtgatcgaacgcgctatctatatggtgaaacgcgcccgtaattacaccgatgatgttgaattttcttgcgaagatgccgggcgtacacccattgccgatctggcgcgagtggtcgaagcggcgattaatgccggtgccaccaccatcaacattccggacaccgtgggctacaccatgccgtttgagttcgccggaatcatcagcggcctgtatgaacgcgtgcctaacatcgacaaagccattatctccgtacatacccacgacgatttgggcctggcggtcggaaactcactggcggcggtacatgccggtgcacgccaggtggaaggcgcaatgaacgggatcggcgagcgtgccggaaactgttccctggaagaagtcatcatggcgatcaaagttcgtaaggatattctcaacgtccacaccgccattaatcaccaggagatatggcgcaccagccagttagttagccagatttgtaatatgccgatcccggcaaacaaagccattgttggcagcggcgcattcgcacactcctccggtatacaccaggatggcgtgctgaaaaaccgcgaaaactacgaaatcatgacaccagaatctattggtctgaaccaaatccagctgaatctgacctctcgttcggggcgtgcggcggtgaaacatcgcatggatgagatggggtataaagaaagtgaatataatttagacaatttgtacgatgctttcctgaagctggcggacaaaaaaggtcaggtgtttgattacgatctggaggcgctggccttcatcggtaagcagcaagaagagccggagcatttccgtctggattacttcagcgtgcagtctggctctaacgatatcgccaccgccgccgtcaaactggcctgtggcgaagaagtcaaagcagaagccgccaacggtaacggtccggtcgatgccgtctatcaggcaattaaccgcatcactgaatataacgtcgaactggtgaaatacagcctgaccgccaaaggccacggtaaagatgcgctgggtcaggtggatatcgtcgctaactacaacggtcgccgcttccacggcgtcggcctggctaccgatattgtcgagtcatctgccaaagccatggtgcacgttctgaacaatatctggcgtgccgcagaagtcgaaaaagagttgcaacgcaaagctcaacacaacgaaaacaacaaggaaaccgtgtga

SEQ ID NO: 3 - DNA, 1602 bp, Artificial Sequence
  Synthesized ECBD_4023
atgactgaacaggcaacaacaaccgatgaactggctttcacaaggccgtatggcgagcaggagaagcaaattcttactgccgaagcggtagaatttctgactgagctggtgacgcattttacgccacaacgcaataaacttctggcagcgcgcattcagcagcagcaagatattgataacggaacgttgcctgattttatttcggaaacagcttccattcgcaatgctgattggaaaattcgcgggattcctgcggacttagaagaccgccgcgtagagataactggcccggtagagcgcaagatggtgatcaacgcgctcaacgccaatgtgaaagtctttatggccgatttcgaagattcactggcaccagactggaacaaagtgatcgacgggcaaattaacctgcgtgatgcggttaacggcaccatcagttacactaatgaagcaggcaaaatttaccagctcaagcccaatccagcggttttgatttgtcgggtacgcggtctgcacttgccggaaaaacatgtcacctggcgtggtgaggcaatccccggcagcctgtttgattttgcgctctatttcttccacaactatcaggcactgttggcaaagggcagtggtccctatttctatctgccgaaaacccagtcctggcaggaagcggcctggtggagcgaagtcttcagctatgcagaagatcgctttaatctgccgcgcggcaccatcaaggcgacgttgctgattgaaacgctgcccgccgtgttccagatggatgaaatccttcacgcgctgcgtgaccatattgttggtctgaactgcggtcgttgggattacatcttcagctatatcaaaacgttgaaaaactatcccgatcgcgtcctgccagacagacaggcagtgacgatggataaaccattcctgaatgcttactcacgcctgttgattaaaacctgccataaacgcggtgcttttgcgatgggcggcatggcggcgtttattccgagcaaagatgaagagcacaataaccaggtgctcaacaaagtaaaagcggataaatcgctggaagccaataacggtcacgatggcacatggatcgctcacccaggccttgcggacacggcaatggcggtattcaacgacattctcggctcccgtaaaaatcagcttgaagtgatgcgcgaacaagacgcgccgattactgccgatcagctgctggcaccttgtgatggtgaacgcaccgaagaaggtatgcgcgccaacattcgcgtggctgtgcagtacatcgaagcgtggatctctggcaacggctgtgtgccgatttatggcctgatggaagatgcggcgacggctgaaatttcccgtacctcgatctggcagtggatccatcatcaaaaaacgttgagcaatggcaaaccggtgaccaaagccttgttccgccagatgctgggcgaagagatgaaagtcattgccagcgaactgggcgaagaacgtttctcccaggggcgttttgacgatgccgcacgcttgatggaacagatcaccacttccgatgagttaattgatttcctgaccctgccaggctaccgcctgttagcgtaa

SEQ ID NO: 4 - DNA, 1167 bp, Artificial Sequence
  Synthesized Lbuc_0961
atgacaattggaatcgacaaaatgggtttctttacttctgattactatttggatatggcagatttggcaaatgcccgcggggaagatcccaataaatatttgattgggattggacagaagaaccaggcggtaatcccgccaacacaagacgtggtcacgatggctgccaatgctgccgccaagattttaagcgatgacgataaacaagcaattgatatggtgatctttggaactgaaaccggtgttgacaactccaagtcagcggccatctatgttcagtcgttgcttggaatatccaactccgccagatcgtttgaaattaagcaagcctgttatggtggtacggccggcgttcagatggctttcgatcatattgcaatgcatcctgaaagtaaagtgttggtcttggcagcagacattgcccgatatgggattggaacagctggtgaggtcacccaaggcggtggggcaattgccatgctgatgagtcaggatccaaaaattttagctttggacaatcaaagtgcgttttactcagaaaatattatggatttttggcggccgttttaccgtcgtgatgcaattgtcgacgggcattactcaacggatatttatatcgatttcttcaaccaaacatttaaagattactctgcgaaatatcaacggacgctgagtgatttttccgcgatggcttttcatctgccattcaccaagatgggcctaaaagcgcttcggacggttatcgatgatggtgctcactcacaacagctgctcaaagaattcgatcattctaaaaagtataatgccctcgtcggtaatctctataccgggtcactctacttaagcttgttatcactactggccaactccaaaacgcttcagccaggtgatcgaattggtttgtttagttatggatcgggagctcaaggggaattttacagtggtacaattgcccctgaaatccaaccgcagagccttgccggccaggttgaacaagtgattgccaaccgaacgaggctgtctgttcctgagtatgaagcgatgtatgcaaatggattgccaaacgccggtggagatgttacatttgatgctgccgatgatgacagtcgattcgtgttgcttggcagacaggccgaccagctggtttatcgagatcaacaggcataa

SEQ ID NO: 5 - DNA, 1074 bp, Artificial Sequence
  Synthesized pmvk
ttgattacagaacaagcaccaggaaagttgtatattgcgggagagtatgcagttcttgagcaaaactgccctgccattttagtagcagtaaatgaatttgtacgtgtttcaattgcaaaaagtacaggtacaagtgggttaattcattctaaacagtattctcaagattcaattcactggatccgtaaaggtaaccaaatggttattgataatcgtgataatccgtttgaatacattttatctgctattaaatttacagaacgtttttgtcttgaacaaaaagttccaatgtctttatacgacctacatgttaattcagatcttgattcagccgatggtaagaaatatggtcttggctcttcagctgctgtaacagttgctacagtgaaggctatccttaatttctatggattacactgtacaaaagatcttatttttaaactttctgctatttctcactacagcgttcaaggtaatggatctgctggtgatattgcagcaagtgtttatggtggttggcttgcttatcaaacttttgataaagcatggcttaagacagaattagctactaaatctcttagcgaagttttaaatgaagcttggcctggtcttaagattcaactattaactcctcccgaaggattaaacttggtaattggttggagtcaaaagcctgcttcaacttctcaattagttgataagactaatgcaaagaaaaagttcattaggacgcaatacgacacctttttagatgaatcccgaaaatgtgttcttgatatgattaggggctttaatgaaaaaaatatttctttaattcaaaaacaaattcgtttaaatcgtcagttattaaaagactttgcttctcttaaccatattgctatcgaaatcccacgtttaactaaattaattaatattgctgaacaatttaatggtgctgctaagacttctggtgcaggaaatggcgattgtggtattgtgattgcagatgaaaaaactgatatcgaagaaatgaaaaataattggcgtaaaaatggaattatgccattgaactttctagttcactcaattgcttag

SEQ ID NO: 6 - DNA, 1008 bp, Artificial Sequence
  Synthesized mvaK2
atgattgctgttaaaacttgcggaaaactctattgggcaggtgaatatgctattttagagccagggcagttagctttgataaaggatattcccatctatatgagggctgagattgctttttctgacagctaccgtatctattcagatatgtttgatttcgcagtggacttaaggcctaatcctgactacagcttgattcaagaaacgattgctttgatgggagacttcctcgctgttcgtggtcagaatttaagacctttttctctagaaatctgtggcaaaatggaacgagaagggaaaaagtttggtctaggttctagtggcagcgtcgttgtcttggttgtcaaggctttactggctctgtatgatgtttctgttgatcaggagctcttgttcaagctgactagcgctgtcttgctcaagcgaggagacaatggttccatgggcgaccttgcctgtattgtggcagaggatttggttctctaccagtcatttgatcgccagaaggtggctgcttggttagaagaagaaaacttggcgacagttctggagcgtgattggggcttttcaatttcacaagtgaaaccaactttagaatgtgatttcttagtgggatggaccaaggaagtggctgtatcgagtcacatggtccagcaaatcaagcaaaatatcaatcaaaattttttaagttcctcaaaagaaacggtggtttctttggtcgaagccttggaacaggggaaatcagaaaagattatcgagcaagtagaagtagccagcaagcttttagaaggcttgagtacagatatttacacgcctttgcttagacagttgaaagaagccagtcaagatttgcaggccgttgccaagagtagtggtgctggtggtggtgactgtggcatcgccctgagttttgatgcgcaatcaaccaaaaccttaaaaaatcgttgggccgatctggggattgagctcttatatcaagaaaggataggacatgacgacaaatcgtaa

SEQ ID NO: 7 - DNA, 1080 bp, Artificial Sequence
  Synthesized mvaK2 (2)
atgaaagtgcaagtaaagataccaggaaaactctttctggcaggggaatatgcagttgttgaagctggctatcctgcggtgattgcggcggttggccagtacctaaccgtcaccattgaaacaagtgaccaaggcagtcttcattctagtcaaaaggcagacctttatctgacctgggagcgtaaggaaggtgccgttcgtattcaaggtagccatccctatgccttgattgaatcagctatgcaggtgacagaagcctatctgacggccaagggctatgcttgtcatggaacctatagcctatctgtacagtccgacctagatgaccaagcctctggtgccaagtacggtctggggtcttcaggagcagtaacggttgcgacggtcaaggccctgttgacctattacggtcatcaagcggatgcccttttgacctacaagctagcagctctgacccagtccaagctaggcatgacaggttcctttggagatttggcggcttccagttttggcggcttgatagcctatcattccctggaccgttcttggcttttagggaaaatggcagagctgtctctgctggatgtggtggaaagtgattggcaaagtttgtctatcagccctatccagttaccccaaggtctagacctcttggttggttggacagggtcggcggcttcaacggatagtctagtttcccagatggaaagccaaaaaagtcagacagaaaaagagcaaattcatagccagtttttagccgactccaagacttgtgtagagcagttgatagtggcctgtcagacaaatgattcagtatcagctagacaggccttgtcagctaatcgcaagctcttacaggactttgcgagaggaatgggcttggtcattgaaacgcctcaactgagtcaactctgtgatctggcccaaacttatggagcggtggccaaatcatctggtgctggcggaggtgactgcggtatttgcttggtagacagtaaggaacaaaaagcagcgatagagacggcttggcaagaagccggtatttttccactcaccctaaacatcgcaagtagagaataa

SEQ ID NO: 8 - DNA, 1128 bp, Artificial Sequence
  Synthesized mvaK2 (3)
ttgattactgaaaaagctcccggtaaattgtatattgccggcgaatatgctgttgttgaaaatggttatcctgcagttttagttgcgttagatcaatttgttacctgtacaatcgaggaaagcactcaagaagttggtcaaattattagtcgccaatataataacgatcaattatcctggcgacgtttaggagaccaaatggtagttgacaaacgtgacaatcctttttcttacattctttctgctattagagttacagaagaatatgctcgtacttttagtcgcgacttacgaatatttaatatccatattgacagtcaattagacagtgctaatggtcgaaaatatggtcttggttcctctgccgctgttactgtagcaactgttaaggctctttgtcggttctataatttgcccgttaacaaggaccaaatctttaaattagctgcaattgcccattttgaagttcaaggaaatggttcgttgggtgatgttgccgcttccgtatatggtggttggatttcctatcattcattcgatcggcagtggcttgcccaacaacgtaagtatcttgacctaaagacgatcattgatttaccttggcctgatctgaatattgagtctctaactgctcccaaaaacttagaacttttaattggctggactggtaaaccagcctcaacttcccaacttgttgataaaatttctctttttaaagctcgtcgtcaaactgaatatcatcattttcttgaaaaaagtaaatcatgtatccaacgaatggttgatggttttcaccgtcaagatcttgaagcgattaaaaatgaaattcgttataaccgtgacctcttaaaacaactcggcactaattcgggggtaaacatcgaaaccgaatcattatctaagctttgccaaatcgctgaatcttttggtggggctgctaagacatctggtgctggtggcggtgattgtgggatcgtcgctattgatagcaactctaactttgggaacgttctaaagagctgggccaaaaaccgcatcgaacaacttcctttatccgtacactttatcgataacgcaaaaagcttcaagaaatctgttaagcaccatctttcttacattcacaagtag

SEQ ID NO: 9 - DNA, 1332 bp, Artificial Sequence
  Synthesized Erg12
atgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataa

SEQ ID NO: 10 - DNA, 1528 bp, Artificial Sequence
  Synthesized Erg12
attttaactgtgttttgaagttgaacgtaaaaaaaatttctgaaatgtcaaaatcgcttattgtttcgtcgccaggaaaaacgattttgtttggggaacatgccgttgtatatggagctgtaagtttacctaaggtttaaagcagattacattagcgagagtttttaacaggctagtactggatacagctctgttatgcatatatgatttctttgtaacatagtatttgcaaagcgaacaccacttttaactttgacagtcgtcgcaaatgtcattctgctatagccatgcttatttttgattctaatcttaagccattttaaagcgaaaatatggagatcatttaaatttttatgctaactttctagactgcgttagctgcagctgtatcgttacggagttactgtaaattacagacgactaataacaatgaaatagtaattgtgatgagtgatatagggaccgaacgccgatggaatcttcaatcgctaccttggcagcatgtaacagtggaaaacgttcagcacccggcatcatctcccaatctggaccttttacaaggattaggagagctattaaaaaatgaagaaaacggacttattcactcagcaatgctttgtaccctttacttgttcacgtctttgtcttctccttctcagggttgtactttaactattagctcccaagtacctttgggtgctggattaggtagtagtgctactatatcagttgttgtcgctacaagtttactactagcttttggtaatattgaacctcctagctcaaattctcttcaaaacaacaaagcacttgcgttgatagaggcttggtcttttctaggtgaatgctgtattcatggaacaccaagtggtattgataatgcagtagcaacaaatggaggacttatcgcttttcgtaaagctacagctcatcagagtgccatgaaagaattcttaaagcctaaagataccttatctgttatgatcactgataccaaacaaccaaaaagtactaaaaaacttgtacaaggagtttttgaactgaaggaaagactaccaactgtgattgactcaataatagatgcaatcgatggcatatcaaagtctgccgtcctcgcattgacttcggagagcgataaaaactcctccgctaaaaagttaggagagtttattgttcttaatcaaaaactcttagaatgcttgggtgtatcccattattccattgatcgcgttttacaagccactaagtcaattggatggacgaagcttacaggtgccggtggtggaggttgtacgattactttattaacacctgagtgcaaagaagaggaatttaagttatgtaaagaatcactattagcccataaaaattctatttacgatgttcaattaggtggacctggtgtttcagtggtaaccgactcagattcatttttccctcaatatgagtctgactttgattttaaaaaattgaatttactcagcaaatttaataaatattatatttaaagtttttatgtataatcaaa

SEQ ID NO: 11 - DNA, 1482 bp, Artificial Sequence
  Synthesized SFUL_6422
atgtcgtcccgtaccgtcgtcatcggcgtggacagctccacccagtccaccaaggccgccgtgatcgacgccgtgaccggcgaacagctcgccgtggggcgcgccccgcacgtcgtcaccggggagggcggcgcccgggagagcgacccggaggtctggtggcgggcgctgggcgacgcggtggcggccggactgaaggagtcggggctgcccgcccgctcggtcaccgggatcgcggtggccggacagcagcacgggctcgtcgtgctggaccgtacgggccgtccgctgcggcccgccctgctgtggaacgacacccgatccgccccgcaggccgccgccctcacagccgggctcggcgggcccgacgcctggaccgcgcggaccggttcggtgccggtcgcctcgatcaccgcctccaagtggcagtggctgcgcgagaacgacccggacgcggcgaaggccgccgtgggggtacggctgccgcacgacttcctcaccgagcggctcgcgggcgtcgccgcgaccgaccccggggacgcctccggcaccggctggtacgccaccgccaccggcgcctacgaccccgtactgctggacctgctcggcctggacccggcactcctccccgaggtcgcgcccaccggggcggccaggatcggctcgctcaccgacgcggcggccgaggcgctgggactgccgcccggcatcgcggtcgcggcggggaccggggacaacatgagcgctgccgtgggcctcggtctcggcggcgccgggctgctggaccacccggtcctctccctcggcacctcgggcgtcgtcttcgccgcctccctcacccgctccaccgaccccgcgctctccgggttcgccgccgccgacggtacgtacctcccgctggcctgcaccctcaactgcacgctcgccgtggacaaggtcgcctccctgctcggcctgcaccgcgaggacacggcccccggcggcgaagccgtactcctcccgtacctcgacggcgaacgcacccccgacctgcccaccgcgtcggggctcctcaccggcctgcgccacgacaccaccccgcagcaactgctcggcgccgcctacgagggcgcggccgtcaccgtcctgcgggccctggacacgcttctacgggcctgcggcctggaccccgacgcccccgaggccgcctcccgcccgctgcggctgatcggcggcggcgcgcaggggcgcggctgggtggagacggtccgccggctctccggacgcccgctggtcatcccggcgggcggcgagctggtcgccctcggggcggccgcgctcgccgcgtccgcagcgggcggcggggacccggtggccctggcgacctcctggggtgcgggcacaacgggcagtcaactcgacgcccaggagcgggacatggagacctggcagcgggtcgcctcggtgctggaccgcgcctcggaaccgctgctcggaggctga

SEQ ID NO: 12 - DNA, 822 bp, Artificial Sequence
  Synthesized Nmlp_1448
atgtcagattcggcgcgcgcgttcgtccccggacacgtgacggggtttttcagcgcccatccggacgacgacccgacgaaggccggctcccgcggtggcggcctcgccatcggcgacggcgtcacggtcgaactcgagcccggcgagggactcgaactcaacggcgaggcggcggcgatcgaggccgtcgaccgggtgctcgacgccctccgtgccaccgcccgcgtcgtcgtcgagaccgacctgccgttgggggccggcttcggcgtctcgggggccgcggcgctcgggactgcgctgacgacgaacgccgtcttcggccgcgggctctcggagaacgaactcgtcaccatcgcccacggcgcggaggtccaggcgggaaccggcctcggtgacgtggtcgcgcaggcccgcggcggcgtcccgatccggctcgaacccggtgcgcctgcccacggcgcgatggacggcatcccacagcgtcggccactcgagtaccgtagcttcggcgaactccccaccgaggagatcatcgccggcgacaccgacgagttgaccgcggccggcgaccgggcgctgtcggcgctcgtcgccgagccgacgctcgagacgttcatggagtcctccaggcgcttcgcccgcgaagcggggctgttgacgccgcgggtagcggaggtcgtccgcgacgtctccgaggccggcggggaggcgtcgatggcgatgctcggcgagacggtgttcgccctcggcagcggcctctcggatgcaggctacgacgccgaacagtgttcgctctcgctggcgggcgcacacgtcctgtga

SEQ ID NO: 13 - DNA, 522 bp, Artificial Sequence
  Synthesized aroK
atggcagagaaacgcaatatctttctggttgggcctatgggtgccggaaaaagcactattgggcgccagttagctcaacaactcaatatggaattttacgattccgatcaagagattgagaaacgaaccggagctgatgtgggctgggttttcgatttagaaggcgaagaaggcttccgcgatcgcgaagaaaaggtcatcaatgagttgaccgagaaacagggtattgtgctggctactggcggcggctctgtgaaatcccgtgaaacgcgtaaccgtctttccgctcgtggcgttgtcgtttatcttgaaacgaccatcgaaaagcaacttgcacgcacgcagcgtgataaaaaacgcccgttgctgcacgttgaaacaccgccgcgtgaagttctggaagcgttggccaatgaacgcaatccgctgtatgaagagattgccgacgtgaccattcgtactgatgatcaaagcgctaaagtggttgcaaaccagattattcacatgctggaaagcaactaa

SEQ ID NO: 14 - DNA, 1116 bp, Artificial Sequence
  Synthesized pdhA
atggcaaaggctaagaaacaaaaacctattgactttaaagagctaatggctaaagtcgacgctgatttcccaactttccaaatcttggatcaagatggaaaaattgtgaatgaagatttagtacctgatttatcggatgaggaattagttgaattaatgacacgcatggtttggtctcgtgtgttagaccaacgttctactgcattaaaccgtcaaggacgcttaggattcttcgcgccaacagctggacaagaagcaagccaattggcaagtcaatttgcaatggaaaaagaagactacttactaccaggttaccgtgatgtacctcaattagtacaacatggtttaccattaagagaagctttcttatggtctcgtggtcacgtagcagggaactactacgcggaagatttaaatgcattaccaccacaaattatcattggtgctcaatacatccaagcagctggtgttgctttaggattgaaaaaacgtggaaaagaaaatgttgtcttcacttatactggtgacggcggttcttcacaaggggacttctatgaagcaattaactttgctggtgcttaccaagcaaacggtgtcttcattatccaaaacaatggttttgcgatttctacacctcgtgaaaaacaaacagcggctaaaactttagctcaaaaagctgttgcagcaggaattcctggtattcaagttgatggtatggatccattagcagtttacgcaattgcaaaagaagcacgcgattggtcagctgcaggaaacggtccagttttaattgaaacattaacttatcgttatggtccacatactttatctggagacgatccaacacgttaccgttcaaaagaaatggatgacgaatgggtacaaaaagatccattgactcgtttccgtaaatatctaacagataaaggcttatggtctgaagcaaaagaagaagaaattattgaaaaaacaaaagaagaaatcaaagtagcgattgcagaagcggataaagcgccaaaacaaaaagtttctgatttcttgaaaaatatgtttgaagttcaacctcaaacaattaaagaacaaattgcattttatgaagcgaaggagtcgaaataa

SEQ ID NO: 15 - DNA, 978 bp, Artificial Sequence
  Synthesized pdhB
atggcacaaaaaactatgatccaagcaattacagatgccttagctcttgaattagagaaagacgaaaatgtcttaatcttcggtgaagacgttggtaacaacggtggggttttccgtgcaactgaaggtttacaagaaaaatttggtgaagaccgcgtcttcgatacacctttagctgaatctggtatcggtggattggctttcggtcttgccttgcaaggttaccgtccagttcctgaaatccaattctttggtttcgtttttgaagtatttgacgaaatcgttggtcaaatggctcgtacgcgttaccgtatgggtggaactcgtaatatgccaattactgttcgtgccccatttggtggtggtgttcatacaccagaacttcactcagataacttagaaggattaatcgcacaatcaccaggtgttcgtgttgttattccatcaaacccttacgatgcaaaaggactattaatttcatctattcgtagcaacgatccagttgtttacttagagcacatgaaattataccgttcattccgtgaggaagtgccagacgaagcttatgaagtgcctttagataaagcggctgtaactcgtgaaggaacagacgtatcaatcatcacttacggtgctatggttcgtgaagcgattaaagcagctgatagcttagcgaaagacaatatttcagcagaaatcattgacttacgtacagtggctcctttagatgtggaaacaattattaactctgttgaaaaaactggccgtgtggttgtcgttcaagaagcacaaaaacaagctggcgttggcgctatggttgtttctgaaatttctgaacgtgccgtattatcattagaagcaccaatcggacgtgtatctgctccagatacaatcttcccattcggacaagcagaaaatatctggttaccaaatgcgaaagatatcgaagcaaaagctagagaaatcgtcgaattttaa

SEQ ID NO: 16 - DNA, 1620 bp, Artificial Sequence
  Synthesized aceF
atggcttatcagtttaaattaccggatatcggtgaagggattgccgaaggcgaaatcgttaaatggtttgtaaaacctggcgatacaatcaacgaagacgatacgttattagaagtacaaaatgacaaatcagtggaagaaattccatcaccagtaacaggtactgtaaaaaatatcgttgtaccagaaggaacagttgcaaacgttggtgacgtgttaatcgaaatcgacgcacctggtcacgaagataacgatgcagcaccagcagctcctgcacaagaacaaacaccagcacaacctgctgctgtaccaacaaccgaagcagctggcggatttttccaattcaaattaccagacatcggtgaaggaattgccgaaggcgaaatcgttaaatggttcgttaaagcgggcgacacaattaatgaagatgattcattattagaagtacaaaatgacaaatcagtagaagaaattccatcaccagtaacaggtactgtaaaaaatatcgttgtaccagaaggaacagttgccaatgtgggtgacgtgttagttgaaattgacgcacctggtcataattcagcagcaccgtcagtcgcagcaccagctactgacgctcctaaagcggaagcatcagctccagccgcttcaacaggcgtagttgcagccgctgatccaaacaaacgcgttttagcaatgccatctgttcgtcagtatgcgcgtgaaaaagacgttgatattacacaagtaactgcaactggtaaaggtggccgtgtcattaaagcggatattgatgcctttgtctctggtggttctcaagcagccccagctactgaagctgccgcaacagaagcagcacctaaagcggaagcagctgcacctaaagcagcgccaaaagcctttacttctgatttaggcgaaatggaaacacgtgaaaaaatgacaccaacacgtaaagcaattgctaaagcaatggttaacagcaaacacactgctcctcacgtaacattacatgatgaagtagaagtttctaaattatgggatcaccgtaagaaatttaaagatgttgctgctgcaaatggtacaaaattaacattcttaccatacgttgtaaaagcattgacttcaactgttcaaaaattcccaatcttgaatgcatcaatcgatgacgcagcacaagaaattgtttacaaaaattactttaacattggtatcgctactgatacagatcatggcttatatgtaccaaatgttaaaaatgctaatacgaagagcatgtttgctatcgctgatgaaatcaacgaaaaagcagcattggctatcgaaggtaaattaactgcacaagatatgcgtgatggtacaatcacaattagtaacattggttcagtcggtggcggctggtttacaccagtaatcaactaccctgaagttgctattttaggcgttggtacaattgcacaagaaccagttgttaatgcagacggcgaaatcgttgtgggacgcatgatgaaattatcattaagctttgaccaccgtatcgttgacggcgcaactgctcaaaaagcaatgaacaacattaaacgcttattagctgatccagaattactattaatggaaggatga

SEQ ID NO: 17 - DNA, 1263 bp, Artificial Sequence
  Synthesized PDA1
atgcttgctgcttcattcaaacgccaaccatcacaattggtccgcgggttaggagctgttcttcgcactcccaccaggataggtcatgttcgtaccatggcaactttaaaaacaactgataagaaggcccctgaggacatcgagggctcggacacagtgcaaattgagttgcctgaatcttccttcgagtcgtatatgctagagcctccagacttgtcttatgagacttcgaaagccaccttgttacagatgtataaagatatggtcatcatcagaagaatggagatggcttgtgacgccttgtacaaggccaagaaaatcagaggtttttgccatctatctgttggtcaggaggccattgctgtcggtatcgagaatgccatcacaaaattggattccatcatcacatcttacagatgtcacggtttcacttttatgagaggtgcctcagtgaaagccgttctggctgaattgatgggtagaagagccggtgtctcttatggtaagggtggttccatgcacctttacgctccaggcttctatggtggtaatggtatcgtgggtgcccaggttcctttaggtgcaggtttagcttttgctcaccaatacaagaacgaggacgcctgctctttcactttgtatggtgatggtgcctctaatcaaggtcaagtttttgaatctttcaacatggccaaattatggaatttgcccgtcgtgttttgctgtgagaacaacaagtacggtatgggtaccgccgcttcaagatcctccgcgatgactgaatatttcaagcgtggtcaatatattccaggtttaaaagttaacggtatggatattctagctgtctaccaagcatccaagtttgctaaggactggtgtctatccggcaaaggtcctctcgttctagaatatgaaacctataggtacggtggccattctatgtctgatcccggtactacctacagaactagagacgagattcagcatatgagatccaagaacgatccaattgctggtcttaagatgcatttgattgatctaggtattgccactgaagctgaagtcaaagcttacgacaagtccgctagaaaatacgttgacgaacaagttgaattagctgatgctgctcctcctccagaagccaaattatccatcttgtttgaagacgtctacgtgaaaggtacagaaactccaaccctaagaggtaggatccctgaagatacttgggacttcaaaaagcaaggttttgcctctagggattaa

SEQ ID NO: 18 - DNA, 1101 bp, Artificial Sequence
  Synthesized PDB1
atgttttccagactgccaacatcattggccagaaatgttgcacgtcgtgccccaacttcttttgtaagaccctctgcagcagcagcagcattgagattctcatcaacaaagacgatgaccgtcagagaggccttgaatagtgccatggcggaagaattggaccgtgatgatgatgtcttccttattggtgaagaagttgcacaatataacggggcttataaggtgtcaaagggtttattggacaggttcggtgaacgtcgtgtggttgacacacctattaccgaatacgggttcacaggtttggccgttggtgccgctttgaagggtttgaagccaattgtagagtttatgtcgttcaatttctctatgcaagctatcgatcatgttgtcaattccgctgcaaagactcactacatgtctggtggtactcaaaaatgtcaaatggtcttcagaggtcctaatggtgctgcagtgggtgttggtgctcaacattcacaggacttttctccttggtacggttccattccagggttaaaggtccttgtcccttattctgctgaagatgctaggggtttgttaaaggccgccatcagagatccaaaccctgttgtatttttagagaacgaattgttgtacggtgaatcttttgaaatctcagaagaagctttatcccctgagttcaccttgccatacaaggctaagatcgaaagagaaggtaccgatatttccattgttacgtacacaagaaacgttcagttttctttggaagccgctgaaattctacaaaagaaatatggtgtctctgcagaagttatcaacttgcgttctattagacctttagatactgaagctatcatcaaaactgtcaagaagacaaaccacttgattactgttgaatccactttcccatcatttggtgttggtgctgaaattgtcgcccaagttatggagtctgaagcctttgattacttggatgctccaatccaaagagttactggtgccgatgttccaacaccttacgctaaagaattagaagatttcgctttccctgatactccaaccatcgttaaagctgtcaaagaagtcttgtcaattgaataa

SEQ ID NO: 19 - DNA, 1233 bp, Artificial Sequence
  Synthesized PDX1
atgctaagtgcaatttccaaagtctccactttaaaatcatgtacaagatatttaaccaaatgcaactatcatgcatcagctaaattacttgctgtaaagacattttcaatgcctgcaatgtctcctactatggagaaaggggggattgtgtcttggaaatataaagttggcgaaccattcagcgcgggcgatgtgatattagaagtggaaacagataaatctcaaattgatgtggaagcactggacgatggtaaactagctaagatcctgaaagatgaaggctctaaagatgttgatgttggtgaacctattgcttatattgctgatgttgatgatgatttagctactataaagttaccccaagaggccaacaccgcaaatgcgaaatctattgaaattaagaagccatccgcagatagtactgaagcaacacaacaacatttaaaaaaagccacagttacaccaataaaaaccgttgacggcagccaagccaatcttgaacagacgctattaccatccgtgtcattactactggctgagaacaatatatccaaacaaaaggctttgaaggaaattgcgccatctggttccaacggtagactattaaagggtgatgtgctagcatacctagggaaaataccacaagattcggttaacaaggtaacagaatttatcaagaagaacgaacgtctcgatttatcgaacattaaacctatacagctcaaaccaaaaatagccgagcaagctcaaacaaaagctgccgacaagccaaagattactcctgtagaatttgaagagcaattagtgttccatgctcccgcctctattccgtttgacaaactgagtgaatcattgaactctttcatgaaagaagcttaccagttctcacacggaacaccactaatggacacaaattcgaaatactttgaccctattttcgaggaccttgtcaccttgagcccaagagagccaagatttaaattttcctatgacttgatgcaaattcccaaagctaataacatgcaagacacgtacggtcaagaagacatatttgacctcttaacaggttcagacgcgactgcctcatcagtaagacccgttgaaaagaacttacctgaaaaaaacgaatatatactagcgttgaatgttagcgtcaacaacaagaagtttaatgacgcggaggccaaggcaaaaagattccttgattacgtaagggagttagaatcattttga

SEQ ID NO: 20 - DNA, 1503 bp, Artificial Sequence
  Synthesized Ald6p
atgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgacgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacagaagtcggtaagagtgttgctgtcgactcttctgaatctaacttgaagaaaatcactttggaactaggtggtaagtccgcccatttggtctttgacgatgctaacattaagaagactttaccaaatctagtaaacggtattttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaa

SEQ ID NO: 21 - DNA, 1000 bp, Artificial Sequence
  Synthesized adhE
atggctgataaaaaagtagtatcaccagaagaaaagttggtagaagcgcgcaagcacgtcgatgagcttgttcaaaaaggtttggttgctcttgatgagttccgtaaacttggtcaagaagaagtagactatattgtagcgaaagcttcggttgcggcccttgataaacatggcgaattggcaatgcacgcctttgaagaaaccaaacgtggtgtattcgaagacaaggctactaagaacttgtttgcctgtgagcacgttgtcaataacatgcgccatacaaaaacagttggtgtcatcgaggaagatgatgtaacaggtttgactttgattgctgagccggtcggtgttgtttgtggtatcacaccaacaacaaacccaacttctacagcaatctttaaatcattgattgccttgaagacacgtaatccaattgtctttgccttccacccatctgcgcaagaatcttctgctcatgcagctcgtgttgtctatgaagcagctgtggcagctggtgcccctgaaaactgtatccagtgggtaacaaaaccatctatggaagcaacgtctgagttgatgaaacatgatggtattgcaacaatccttgcaactggtggtaacgcaatggtgcgtgcagcttactcttgtggtaaacctgcccttggtgtaggtgcaggtaacgttccagcctatgttgaaaaatcagcaaacatccgtcaggcagcacatgacatcgttatgtctaagtcatttgacaacggtatggtctgtgcgtcagaacaagcggttatcattgataaagaagtttacgatgagtttgtagcagaattcaaatcttacaaaacttacttcgttaataagaaagaaaaagctcttcttgaagaatactgcttcggtgtgaaagctaacagcaaaaactgtgctgaaggtaaattgaacgcagacatcgttggtcgcccagctgcatggatcgctgagcaggccggcttctcagtaccag

SEQ ID NO: 22 - DNA, 1000 bp, Artificial Sequence
  Synthesized FdsA
atgaatgacatgagccaggatggccttcgtggtgggtgcgggtccggatcgtgcgcttgccgcgcgaacaatgtaccgctggacgacgtcgatttcggtacgccggcccgccacggcccaagcgtgtcgctgcagatcgacggcgtgcaggtggatgtgccggcaggcacatcggtcatgcgcgcggcgatccacgccggcgttcgcgtgccgaaactatgcgcaaccgattctctagaggcgttcggctcgtgccggctgtgcctggtcgagatcgaaggccgccggggttatccggcctcctgcacgacgccggtcgagccgggcatgaaggtgcgcacgcaaagcgacaagctgcaggcgctgcgtcgcaacgtgatggagttgtacatttccgatcacccgttagactgtctgacgtgcccgtccaacgggcgttgcgagttgcaggacatggcgggcgtcgtggggctgcgcgaggtccgctacggctacgatggcgccaatcatctgcgcgacgcgaaggacgaatcgaacccgtacttcacctatgacgccagcaagtgcatcgtgtgcaaccgatgcgtgcgcgcgtgcgagcaaacgcaaggcacctttgcgctgaccatcgcgggacgcggcttcgagtcgcgcgttgcggcaagcgaaggcgtctcgttcatggcatcggagtgtgtctcgtgtggagcgtgcgtggccgcttgcccgaccgcgacgttgcacgaaaaatcgattgccgaactgggccaaccggagcacgccgtcgtcacgacctgtgcttattgcggtgtcggctgttcgctaaaggccgagatgaagggcaatcaggtggtacggatggtgccgcacaaggacggccgcgccaacgagggccatgcttgcgtgaaggggcgctttgcatggggctatgcgacgcaccgcgaccgcatcacgcggccgatgatccgcgcgtcgatcgacgcgccgtggcgcgaagtca

SEQ ID NO: 23 - DNA, 1000 bp, Artificial Sequence
  Synthesized FdsB
gtgctaatggcgattcgcatttacgtgccgtccgatacgacagcgatcgcgctcggagcagacgagatcgcgcaggccatcgcgggacaagccgcgcgacgcgcgctcgacatcgagctggtacgcaacggctcgcgaggattgtcctggttggagccactcgtggaggtcggcacggccgccggccgcatcggctacgctaacgttgcgttgcaggacatcgacgcgttgttcgactcgggtttccacaccgacgacgctgcgcaagcacgggcagcggcccatccgaaatgcgtcggcctcgtcgaagccctcccctacctaagcaagcaacagcgtttgaccttttcccggatcggcctcatcgatccgctatcgatcgacgactatgttgcgcacggtggcctgcaaggcctgcaccgcgcgttgcagatggcgccagccgccgcctgccagttactgattgacgctggactgcgcggacgcggcggcgccgcgtttccggcaggtattaagtggcgcacggtactgcaagcccaggcagaccaaaaatacgtagtctgcaacgcggacgaaggtgacagcggcacgttctcggaccgcttgtcgatggaatgtgacccatttggcctgatcgaaggcatgatcatcgccggcgtgtcgaccggtgcgacgcaaggctacctctacgtgcgcagcgaatacccgttggcgatcgcgcgattgaacgcggcgatcgcccgtgcgcgcgccgccggctggctcggcccagatgtgctgggctccggtcgggctttcgatctgttcgtcgcgaaaggcgccggcgcgtatgtctgcggcgaggagacggcgctactcgaatcgctcgaaggcaagcgaggcatcgtgcgcgccaagccaccgctgcccgcgttggccggctaccgaggcaagccgacggtgatcaacaacgtgatcacgctggcgtccgtgccgatcattttcgcgcgcggcgatgcgt

SEQ ID NO: 24 - DNA, 579 bp, Artificial Sequence
  Synthesized FdsG
ttggcagaaccctctaccggccgtgcaacgggcgccgcattggcgccggacgcggcagctcggcttgtcgcgcaccacacgagtgcggcagcgcgcgccgtaccgggacattcgctattaagcgttttgcacgcaatccaggacgatgctggttacatccccgacagtgtcatcgccccgcttgcccaggcaatgaacttgtcgcgcgccgaagtccatggcgtgatcacgtattaccatcactttcgcacatcgccaccggccgccgtgacagtccagctgtgccgcgccgaatcctgtcgcgcgatgggcagcgaagcgctggcgcggcatgccgagacccgcacgggtcaccgcttcgatgcgtgtcgccacggcgacgaccatcgcgcggcgccaccggccgcaccgctcgccggccacccggcagttgagttgcaatccgcgtactgcctgggcttgtgcagcacgtccccggcgatgatggtcaatggcaagccctacgcccgcgtcacgccggagaagctcgatacagtgctcgccgcggcgatcggcgacgcgcaacggggagcgtgctaa

SEQ ID NO: 25 - DNA, 258 bp, Artificial Sequence
  Synthesized FdsD
atggcaatgccgctgcacaccctggtcgacatggccaaccgcattggagagttcttcgaatcgatgcccgatcggcacgccgcgctcgaaggtatcgccgatcatctaaagcgcttttgggagccgcgcatgcggcgcacactgctcgacgcgctcgatcgcggcatggatccggcgttggacgagttgatgccgatcgcgcgcgaggcccttgttcgctatcgtgacaggttaatgccggccgccgcgccggcataa

SEQ ID NO: 26 - DNA, 414 bp, Artificial Sequence
  Synthesized mgsA
atgaaaattgctttgatcgcgcatgacaagaaaaaacaggatatggttcaatttacgactgcctatcgggatattttaaagaatcatgatctatacgcaaccggaaccacagggttgaaaattcatgaggcgacaggtcttcaaattgaacgttttcaatccggccctttagggggagaccagcaaatcggtgcactgatcgctgccaatgcactcgatcttgtcatttttttgcgcgacccgctgaccgcgcagccgcatgaaccggatgtctcggcattaatccgtttatgtgatgtgtattccattccgctcgccacaaatatgggtactgcggaaattcttgtgcgcacacttgatgaaggtgttttcgaattccgtgaccttcttcggggagaagagccgaatgtataa

SEQ ID NO: 27 - DNA, 893 bp, Artificial Sequence
  Synthesized ispS
gtcttgcaacaaagttgcattttgctaaagacaggttaattgaaagcttttactgggcagttggagttgcgttcgaacctcaatacagtgattgccgtaattcagtagcaaaaatgttttcatttgtaacaatcattgatgatatctatgatgtttatggtactctggatgagctggagctatttacagatgctgttgagagatgggatgttaacgccatcaatgatcttccggattatatgaagctctgcttcctagctctctacaacactatcaatgagatagcttatgacaatctgaaggacaagggggaaaacattcttccatacctaacaaaagcgtgggcagatttatgcaatgcattcctacaagaagcaaaatggctgtacaataagtccacaccaacatttgatgactatttcggaaatgcatggaaatcatcctcagggcctcttcaactaatttttgcctactttgccgtggttcaaaacatcaagaaagaggaaattgaaaacttacaaaagtatcatgatatcatcagtaggccttcccacatctttcgtctttgcaacgacctggcttcagcatcggctgagatagcgagaggtgaaactgcgaattccgtatcctgctacatgcgtacaaaaggcatttctgaggaacttgctactgaatccgtaatgaatttgatcgacgaaacctgtaaaaagatgaacaaagaaaagcttggtggctctttgtttgcaaaaccttttgtcgaaacagctattaaccttgcacggcaatcccattgcacttatcataacggagatgcgcatacttcaccagacgagctaactaggaaacgtgtcctgtcagtaatcacagagcctattctaccctttgagagataa

SEQ ID NO: 28 - DNA, 892 bp, Artificial Sequence
  Synthesized IspS
tcttgcaacaaagttgcattttgctagagacaggttaattgaaagcttttactgggcagttggagttgcgtttgaacctcaatacagtgattgccgtaattccgtagcaaaaatgttttcgtttgtaacaatcattgatgatatctatgatgtttatggtactctggatgagttggagctatttacagatgctgttgagagatgggatgttaatgccatcgatgatcttccggattatatgaagctctgcttcctagctctctataacactatcaatgagatagcttatgataatctgaaggacaagggggaaaacattcttccatacctaacaaaagcgtgggcagatttatgcaatgcattcctacaagaagcaaaatggttgtacaataagtccacaccaacatttgatgaatatttcggaaatgcatggaaatcatcctcagggcctcttcaactagtttttgcctactttgccgttgttcaaaacatcaagaaagaggaaattgataacttacaaaagtatcatgatatcatcagtaggccttcccacatctttcgtctttgcaacgacttggcttcagcatcggctgagatagcgagaggtgaaaccgcgaattctgtatcatgctacatgcgtacaaaaggcatttctgaggaacttgctactgaatccgtaatgaatttgatcgacgaaacctggaaaaagatgaacaaagaaaagcttggtggctctctgtttgcaaaaccttttgtcgaaacagctattaaccttgcacgacaatcccattgcacttatcacaacggagatgcgcatacttcaccagatgagctcactaggaaacgtgtcctgtcagtaatcacagagcctattctaccctttgagagataa

SEQ ID NO: 29 - DNA, 651 bp, Artificial Sequence
  Synthesized atoA
atggatgcgaaacaacgtattgcgcgccgtgtggcgcaagagcttcgtgatggtgacatcgttaacttagggatcggtttacccacaatggtcgccaattatttaccggagggtattcatatcactctgcaatcggaaaacggcttcctcggtttaggcccggtcacgacagcgcatccagatctggtgaacgctggcgggcaaccgtgcggtgttttacccggtgcagccatgtttgatagcgccatgtcatttgcgctaatccgtggcggtcatattgatgcctgcgtgctcggcggtttgcaagtagacgaagaagcaaacctcgcgaactgggtagtgcctgggaaaatggtgcccggtatgggtggcgcgatggatctggtgaccgggtcgcgcaaagtgatcatcgccatggaacattgcgccaaagatggttcagcaaaaattttgcgccgctgcaccatgccactcactgcgcaacatgcggtgcatatgctggttactgaactggctgtctttcgttttattgacggcaaaatgtggctcaccgaaattgccgacgggtgtgatttagccaccgtgcgtgccaaaacagaagctcggtttgaagtcgccgccgatctgaatacgcaacggggtgatttatga

SEQ ID NO: 30 - DNA, 663 bp, Artificial Sequence
  Synthesized atoD
atgaaaacaaaattgatgacattacaagacgccaccggcttctttcgtgacggcatgaccatcatggtgggcggatttatggggattggcactccatcccgcctggttgaagcattactggaatctggtgttcgcgacctgacattgatagccaatgataccgcgtttgttgataccggcatcggtccgctcatcgtcaatggtcgagtccgcaaagtgattgcttcacatatcggcaccaacccggaaacaggtcggcgcatgatatctggtgagatggacgtcgttctggtgccgcaaggtacgctaatcgagcaaattcgctgtggtggagctggacttggtggttttctcaccccaacgggtgtcggcaccgtcgtagaggaaggcaaacagacactgacactcgacggtaaaacctggctgctcgaacgcccactgcgcgccgacctggcgctaattcgcgctcatcgttgcgacacacttggcaacctgacctatcaacttagcgcccgcaactttaaccccctgatagcccttgcggctgatatcacgctggtagagccagatgaactggtcgaaaccggcgagctgcaacctgaccatattgtcacccctggtgccgttatcgaccacatcatcgtttcacaggagagcaaataa

SEQ ID NO: 31 - DNA, 1509 bp, Artificial Sequence
  SynthesizeD actA
atgtctgatcgcattgcttcagaaaagctgcgctccaagctcatgtccgccgacgaggcggcacagtttgttaaccacggtgacaaggttggtttctccggcttcaccggcgctggctacccaaaggcactgcctacggcaatcgctaaccgggctaaagaagcacacggtgcaggcaacgactacgcaatcgacctgttcactggcgcatcgaccgcccctgactgcgatggcgtacttgcagaagctgacgctatccgctggcgcatgccatacgcatctgatccaatcatgcgtaacaagatcaactccggctccatgggatactccgatatccacctgtcccactccggccagcaggttgaagagggcttcttcggccagctcaacgtagctgtcattgaaatcacccgcatcactgaagagggctacatcatcccttcttcctccgtgggtaacaacgttgagtggctcaacgctgcagagaaggtcatcctcgaggttaactcttggcagtctgaagacctcgaaggtatgcacgacatctggtctgttcctgccctgccaaaccgcattgccgtgccaatcaacaagccaggcgaccgcatcggtaagacctacatcgagttcgacaccgacaaggttgttgctgttgttgagaccaacaccgcagaccgcaacgcaccattcaagcctgtcgacgacatctctaagaagatcgctggcaacttcctcgacttcctggaaagcgaagttgctgcaggtcgcctgtcctacgacggctacatcatgcagtccggcgtgggcaacgtgccaaacgcggtgatggcaggcctgctggaatccaagtttgagaacatccaggcctacaccgaagttatccaggacggcatggtggacctcatcgacgccggcaagatgaccgttgcatccgcaacttccttctccctgtctcctgagtacgcagagaagatgaacaacgaggctaagcgttaccgcgagtccattatcctgcgcccacagcagatctctaaccacccagaggtcatccgccgcgttggcctgatcgccaccaacggtctcatcgaggctgacatttacggcaacgtcaactccaccaacgtttctggctcccgcgtcatgaacggcatcggcggctccggcgacttcacccgtaacggctacatctccagcttcatcaccccttcagaggcaaagggcggcgcaatctctgcgatcgttcctttcgcatcccacatcgaccacaccgagcacgatgtcatggttgttatctctgagtacggttacgcagaccttcgtggtctggctccacgtgagcgcgttgccaagatgatcggcctggctcaccctgattaccgcccactgctcgaggagtactacgctcgcgcaacctccggtgacaacaagtacatgcagacccctcatgatcttgcaaccgcgtttgatttccacatcaacctggctaagaacggctccatgaaggcataa

SEQ ID NO: 32 - DNA, 378 bp, Artificial Sequence
  Synthesized Cg0592
atgaatggtatcggcggctcgggcgatttcacgcgtaacgcctttgcttccacatttatctctccctcggcagccaaagttgatgcgatttccgcgattgtgcctttcgcgtcccatatcgatcacacggaacatgatgcgatggttgtcattactgaatatggctacgcagacctgcgcgggctatcgccaaaacaacgagtccccaaaatgattgccatcgcccacccggactatcgaccactgctggaagcatactttgaccgggcgctgaacagtgctgattcctatcagcacaccctgcatgatctgcgcaccgccttcgatttccataatcgcttgaactcacaaggaaccatgaaaatcgaaaaagcatag

SEQ ID NO: 33 - DNA, 657 bp, Artificial Sequence
  Synthesized ctfA
atgaactctaaaataattagatttgaaaatttaaggtcattctttaaagatgggatgacaattatgattggaggttttttaaactgtggcactccaaccaaattaattgattttttagttaatttaaatataaagaatttaacgattataagtaatgatacatgttatcctaatacaggtattggtaagttaatatcaaataatcaagtaaaaaagcttattgcttcatatataggcagcaacccagatactggcaaaaaactttttaataatgaacttgaagtagagctctctccccaaggaactctagtggaaagaatacgtgcaggcggatctggcttaggtggtgtactaactaaaacaggtttaggaactttgattgaaaaaggaaagaaaaaaatatctataaatggaacggaatatttgttagagctacctcttacagccgatgtagcattaattaaaggtagtattgtagatgaggccggaaacaccttctataaaggtactactaaaaactttaatccctatatggcaatggcagctaaaaccgtaatagttgaagctgaaaatttagttagctgtgaaaaactagaaaaggaaaaagcaatgacccccggagttcttataaattatatagtaaaggagcctgcataa

SEQ ID NO: 34 - DNA, 666 bp, Artificial Sequence
  Synthesized ctfB
atgattaatgataaaaacctagcgaaagaaataatagccaaaagagttgcaagagaattaaaaaatggtcaacttgtaaacttaggtgtaggtcttcctaccatggttgcagattatataccaaaaaatttcaaaattactttccaatcagaaaacggaatagttggaatgggcgctagtcctaaaataaatgaggcagataaagatgtagtaaatgcaggaggagactatacaacagtacttcctgacggcacatttttcgatagctcagtttcgttttcactaatccgtggtggtcacgtagatgttactgttttaggggctctccaggtagatgaaaagggtaatatagccaattggattgttcctggaaaaatgctctctggtatgggtggagctatggatttagtaaatggagctaagaaagtaataattgcaatgagacatacaaataaaggtcaacctaaaattttaaaaaaatgtacacttcccctcacggcaaagtctcaagcaaatctaattgtaacagaacttggagtaattgaggttattaatgatggtttacttctcactgaaattaataaaaacacaaccattgatgaaataaggtctttaactgctgcagatttactcatatccaatgaacttagacccatggctgtttag

SEQ ID NO: 35 - DNA, 1753 bp, Artificial Sequence
  Synthesized Ach1
agaaatctgctacgaactgggaacctattttgtgggacagcgcgactacgcggaagcggttctctggttctacaatgccgcctatgagacggaaagcatcctggacgttcacacaagcggggatcttccgctgctcggtcttgtcgaatgttacgagacgctcctcgccggggaggaagccaaaattccttccgacacagcgcttaccatccagtacgaaatgatgctcgacaaataccgggaggcttccagagactggcggatgccggaggagacctgatcttacaaatctccggaaatacgctccggcagggcttgtaaaatacgacataaagtgataggatgaaactatggtaaaattttaacaatcttttgtgtgggaggtatttgagatggattttcgtgaagaatacaaacagaagcttgtctccgcagatgaggcggtaaagctcatcaaatccggagactgggtagattacggctggtgcaccaacaccgttgacgcactggatcaggctctcgcaaagcgcaccgacgaactgacagacgtcaagctgcgcggcggtatcctgatgaagccgctggctgtttttgcacgtgaggatgcaggtgagcatttctgctggaactcctggcatatgtccggtatcgagcgcaagatgataaacagaggcgtggcttactactgtccgatccgctactccgagctgccgcgctactaccgcgagcttgactgcccggatgacgttgccatgttccaggttgctccgatggatgcgcacggctactttaacttcggtccgagtgcctcacatctgggtgcaatgtgcgagcgcgcaaagcacatcatcgtagaagtcaatgaaaatatgccacgctgcctcggcggtaccgagtgtggcatccacatttccgatgtcacctacatcgtggaaggctccaacccgccaatcggtgaactgggtgcaggcggtcctgctacagatgtggataaggctgtcgcaaagctgatcgtcgatgagattccgaacggtgcctgcttacagctcggtatcggcggcatgccaaacgctgtcggttccctgattgcagagtccgacttgaaggatctcggcgttcacactgagatgtacgtggatgcatttgtcgatattgcaaaggcaggtaagatcaacggttccaaaaagaatatcgaccgttaccgccagacctacgctttcggcgccggcaccaagaaaatgtacgattatctggacgacaacccggaactgatgagcgctccggtcgactacacgaacgacatccgctcgatctccgcactggataactttatttccatcaacaatgccgtggatattgatctctatggtcaggtaaatgcagagtctgcaggcatcaagcagatcagcggcgcaggcggacagcttgacttcgtgctcggagcttatctgtccaagggcggcaagagctttatctgcttatcctctaccttcaagaccaaggacggtcaggtgcagtcccgtatccgcccgacgctggcaaacggttccatcgttaccgacgcaagaccgaatacacactatgttgtaaccgaatacggcaaggtgaacttaaagggtctgtctacctggcagagagccgaggctctgatctcgatcgcgcatcccgatttccgcgacgacctcatcaaagaggcggagcagatgcacatctggagaagaagcaaccgctagtaccggaggacgactgacgg

SEQ ID NO: 36 - DNA, 1000 bp, Artificial Sequence
  Synthesized Pct
gaattcaaaattgctatcgttgatgacgatttggctcaggaatccagacagattcgtgttgacgttctggatggcgaaggtggccctctttatagaatggcaaaagcttggcagcaaatgtacggttgctctcttgcaactgatacaaagaaaggccgcggcagaatgctgatcaacaagacaattcagacaggtgcagatgctatcgttgttgcgatgatgaaattctgtgatcctgaagaatgggattaccctgtaatgtacagagaatttgaagaaaaaggcgttaagagtctgatgatcgaagttgatcaggaagtttcttccttcgaacagatcaagacaagactgcagtctttcgtagaaatgctgtaatttgaacaatcgtttgctgaaaaactgtacactggggtgggtgactgctccagtgtattgtaataagcaaataagcaaaaatcgataagatttaggaggattttcgacaatgagaaaggttcccattattaccgcagatgaggctgcaaagcttattaaagacggtgatacagttacaacaagtggtttcgttggaaatgcaatccctgaggctcttgatagagctgtagaaaaaagattcttagaaacaggcgaacccaaaaacattacatatgtttattgtggttctcaaggtaacagagacggaagaggtgctgagcactttgctcatgaaggccttttaaaacgttacatcgctggtcactgggctacagttcctgctttgggtaaaatggctatggaaaataaaatggaagcatataatgtatctcagggtgcattgtgtcatttgttccgtgatatagcttctcataagccaggcgtatttacaaaggtaggtatcggtactttcattgaccccagaaatggcggcggtaaagtaaatgatattaccaaagaagatattgttgaattggtagagattaagggtcaggaatatttattctaccctgcttttcctattcat

SEQ ID NO: 37 - DNA, 1554 bp, Artificial Sequence
  Synthesized Cbei_4543
gtgagaaaagtaaaagttttaacaagtcgcgaagcagtacaaatagtgaaggatggagatgtgttagtaactggcggatttgttggtagttgtgcacctgaaactcttagttgtgctttagaaaaacgtttcattgaaacaaatcatccgcaaaatataactttatttcatgcagcaggacaaggcgatagtaaggggaaaggttcagatcattatgcccacgaaggcttacttaagagagtggttgcaggtcattataatttagcaccgaaaattggaaagttaattaatgaaaataaaatagaagcttataatctaccacaagggacaatttctcaattatttagagatattgcgggaaaaagaattgggacaataactcacgttggattgaatacatttgtggatccaagaattagtggtggaaaattaaatgaaaaaacaaaagaagatctagtaaagctaataaatatagaaggtgaagaaaaattattatacaaatcaattccagttaatgtctgcttcttaagaggatcttttgcagatgaatacggtaatgtatcattagaaaaagaaatagctacacttgaggatacgtcaatagcccaagcttgtaagaataatggcggaaaagtaatagttcaagtagaaaaagtagttgaagcaggatctttagacccacgtcttataaaaattccaggtatatatgtagatgcggttgtaatctcaactcccgaagagcatgaacaatccttcgaatgcccatttaatccagcagtaacaggtgaaatgagaattccattaaacagtgtagaaaaagctccattaaatgagagaaagataattgcgagaagagcagctatggaattaaagaaagatacggtagtaaatttaggtatagggataccagaagttatttctttagttgcgaatgaagaaggaattggtgaatatatgacattaactgtagaagccggtccaataggaggtataccacaaggatgcacagcttttggagcgagtataaatccagaagctattatagatcagccatatcaatttgatttttatgatggtggaggcgtcgatatagcatttttaggactagctcaggttgatgaacatggaaatttgaatgtaagtaagtttgggcctagaattgctggatgtggtggattcataaatataactcaaaatgctaagaaagtgttattttgtggaacattcactgcaggaggcttaaaagtagtaacaggagatggcaaattagaaattaaacaagaaggaaaagctaaaaaattcattaaggatgtagagcaaattacatttagtggagattatgcaagaaggatggatcaacaagttatgtatataactgagagagcagtatttgagttaaggaaagatggattataccttacagaaatagcgcctgggatagatctaaaaaaggatgtattggatttaatggatttcaaacctaaaatggatggagtacctagactaatgaatggaagaatattttatgataagttgatgggattaagggagtaa

SEQ ID NO: 38 - DNA, 687 bp, Artificial Sequence
  Synthesized pcaI
atgatagataaaagtgcagcgaccctaacggaagcgctctcccagatccacgacggtgccaccatcctgattggtggttttggaacagccggccaacccgccgagctgattgacggactgattgaactaggtcgcaagaacctaaccatcgtcagcaacaacgccggcaatggagactatggactggccaagctgctaaaaactggcgccgtcaaaaagatcatctgttccttcccacgtcaggccgactcctacgtatttgacgagctataccgtgcgggcaaaattgaacttgaaatcgtgccgcagggcaatctggcctgtcgtatacaggccgccggcatggggctggggccgatctacaccccaaccggttttggcactttactcgcagaaggtaaacctaccctgaactttgatggcaaagactacgtactggaaaacccgatcaaggccgactttgccctgatcaaagcctacaagggcgaccgctggggcaatctggtctatcgcaaatcagcacgaaacttcggcccgatcatggccatggccgccaacgtgaccatcgcacaagtgagcgaagtggtggcactaggagaactcgacccggaaaacgtggtgaccccaggcatctttgttcaacacgttgtaccagtccaatctaccccagcaagcgctgcaccataa

SEQ ID NO: 39 - DNA, 654 bp, Artificial Sequence
  Synthesized PcaJ
atgagttatcacaaactgacccgtgaccagatcgcccagcgcgttgcccaagacattccggaaggctcctatgtcaatcttggcattggcctgccgaccaagattgccagctatctgcctgccgacaaagacgtatttctacattcagaaaacggactgctggcctttggcccaccaccagcggccggcgaagaagatccggaactgatcaacgcaggcaaagaatacgtaaccatgctcgaaggcggttgcttctttcaccatggcgactccttcgccatgatgcgcggtggacatctggatatctgcgtattaggcgcattccagatcgccgccaatggagacctggccaactggcacaccggtgcaccggatgccataccgtcggtcggtggagccatggatcttgcggttggggcaaaaaaagtttttgtaaccaccgatcatgtcaccaaaaaaggtgagccgaagattgtagctgaactgacgtatccagccacgggtcagaaatgtgtcgaccggatctacaccgacctgtgcatcatcgatgtggtgccagaaggactgaaagtgatcgagaaagtcgaaggcttaagctttgaagaactacaacgcctgaccggtgcaacactgatcgatgcgacacaaggctaa

SEQ ID NO: 40 - DNA, 696 bp, Artificial Sequence
  Synthesized pcaI (1)
ttgatcaataaaacgtacgagtccatcgccagcgcggtggaagggattaccgacggttcgaccatcatggtcggtggcttcggcacggctggcatgccgtccgagctgatcgatggcctcattgccaccggtgcccgcgacctgaccatcatcagcaacaacgccggcaacggcgagatcggcctggccgccctgctcatggcaggcagcgtgcgcaaggtggtctgctcgttcccgcgccagtccgactcctacgtgttcgacgaactgtaccgcgccggcaagatcgagctggaagtggtcccgcagggcaacctggccgagcgtatccgcgccgcaggctccggcattggtgcgttcttctcgccaaccggctacggcaccctgctggccgagggcaaggaaacccgtgagatcgatggccgcatgtacgtgctggaaatgccgctgcacgccgacttcgcactgatcaaggcgcacaagggtgaccgttggggcaacctgacctaccgcaaggccgcccgcaacttcggcccgatcatggccatggctgccaagaccgccatcgcccaggtcgaccaggtcgtcgaactcggtgaactggacccggaacacatcatcaccccgggtatcttcgtccagcgcgtggtcgccgtcaccggtgctgccgcttcttcgattgccaaagctgtctga

SEQ ID NO: 41 - DNA, 642 bp, Artificial Sequence
  Synthesized pcaJ
atgaccatcaccaaaaagctctcccgcaccgagatggcccaacgcgtggccgcagacatccaggaaggcgcgtatgtaaacctgggtatcggcgcaccaaccctggtggccaactacctgggcgacaaggaagtgttcctgcacagcgaaaacggcctgctgggcatgggcccaagccctgcgccgggcgaggaagacgatgacctgatcaacgccggcaagcagcacgtcaccctgctgaccggtggtgccttcttccaccatgccgattcgttctcgatgatgcgtggcggccacctggacatcgccgtactgggtgccttccaggtgtcggtcaagggcgacctggccaactggcacacgggtgccgaaggttcgatcccggccgtaggcggcgcaatggacctggccaccggcgcccgccaggtgttcgtgatgatggaccacctgaccaagaccggcgaaagcaagctggtgcccgagtgcacctacccgctgaccggtatcgcgtgcgtcagccgcatctacaccgacctggccgtgctggaagtgacaccggaagggctgaaagtggtcgaaatctgcgcggacatcgactttgacgaactgcagaaactcagtggcgtgccgctgatcaagtaa

SEQ ID NO: 42 - DNA, 717 bp, Artificial Sequence
  Synthesized ScoA
atgggaaaagtgctgtcatcaagcaaggaagctgcgaaactgattcatgatggggatacgctgatcgcgggagggtttgggctgtgcggcatccctgaacagctcattttgtctataagagatcagggagtaaaggatttaaccgttgtcagcaataactgcggagtcgatgactgggggcttggtttgcttctggctaacaagcaaatcaagaaaatgatcgcttcctatgtcggtgaaaataaaatttttgagcggcagtttttaagcggagagcttgaggtagagcttgttccccaaggaacgctcgctgagagaattcgtgcaggcggtgcaggcataccgggattttatacggcgacaggcgtcggcacctccatagccgagggaaaagaacataaaacattcggcggccggacttatgtgctggagcgaggcattaccggcgatgtggcgatcgtcaaagcgtggaaagcggacaccatgggcaatttgatttttaggaaaacggcgagaaatttcaatcccattgccgccatggcaggcaagatcacgattgccgaggcggaagaaatcgtggaagcaggagagctcgatccagatcacatccatacgccgggaatttacgtacagcatgtcgtgcttggcgcgagccaagaaaaacggattgaaaaacgaacagttcagcaagcatcgggaaagggtgaggccaagtga

SEQ ID NO: 43 - DNA, 651 bp, Artificial Sequence
  Synthesized ScoB
gtgaaggaagcgagaaaacgaatggtcaaacgggctgtacaagaaatcaaggacggcatgaatgtgaatctcgggattggaatgccgacgcttgtcgcaaatgagatacccgatggcgttcacgtcatgcttcagtcggaaaacggcttgctcggaattggcccctatcctctggaaggaacggaagacgcggatttgatcaatgcgggaaaggaaacgatcactgaagtgacaggcgcctcttattttgacagcgctgagtcattcgcgatgataagaggcgggcatatcgatttagctattctcggcggaatggaggtttcggagcagggggatttggccaattggatgatcccgggcaaaatggtaaaagggatgggcggcgccatggatctcgtcaacggggcgaaacgaatcgttgtcatcatggagcacgtcaataagcatggtgaatcaaaggtgaaaaaaacatgctcccttccgctgacaggccagaaagtcgtacacaggctgattacggatttggctgtatttgattttgtgaacggccgcatgacactgacggagcttcaggatggtgtcacaattgaagaggtttatgaaaaaacagaagctgatttcgctgtaagccagtctgtactcaattcttaa

SEQ ID NO: 44 - DNA, 1617 bp, Artificial Sequence
  Synthesized Cat1
atgagtaaagggataaagaattcacaattgaaaaaaaagaatgtaaaggctagtaatgtggcagaaaagattgaagagaaagttgaaaaaacagataaggttgttgaaaaggcagctgaggttacagaaaaacgaattagaaacttgaagcttcaggaaaaagttgtaacagcagatgtggcagctgatatgatagaaaacggtatgattgttgcaattagcggatttactccttccgggtatcctaaagaagtacctaaagcattgactaaaaaagttaatgccttagaggaagaattcaaggtaacactttatacaggttcatctacaggagccgatatagacggagaatgggcaaaagcaggaataatagaaagaagaattccatatcagacaaattctgatatgaggaaaaaaataaatgatggttctattaagtatgctgatatgcatttaagccatatggctcaatatattaattattctgtaattcctaaagtagatatagctataatagaggcagtagctattacagaagaaggggatattattccttcaacaggaattggaaatacagctacttttgtggaaaatgcagataaggtaatagtggaaattaatgaggctcaaccgcttgaattggaaggtatggcagatatatatacattaaaaaaccctccaagaagagagcccatacctatagttaatgcaggcaataggatagggaccacatatgtgacctgtggttctgaaaaaatatgcgctatagtgatgacaaatacccaggataaaacaagacctcttacagaagtgtctcctgtatctcaggctatatccgataatcttataggatttttaaataaagaggttgaagagggaaaattacctaagaacctgcttcctatacagtcaggagttggaagtgtagcaaatgcagttttggccggactttgtgaatcaaattttaaaaatttgagttgttatacagaagttatacaggattctatgctgaagcttataaaatgtggtaaagcagatgtggtgtcaggcacttccataagtccttcaccggagatgttgcctgagttcataaaggacataaatttctttagagaaaagatagtattaagaccacaggaaataagtaataatccagagatagcaagaagaataggagttatatccataaacactgctttggaagtagatatatatggtaatgtaaactccactcatgttatgggaagcaaaatgatgaatggtataggcggttctggagactttgccagaaatgcatatttgactatattcactacagagtctatcgccaaaaaaggagatatatcatctatagttcctatggtatcccatgtggatcatacagaacatgatgtaatggtaattgttacagaacagggagtagcagatttaagaggtctttctcctagggaaaaggccgtggctataatagaaaattgtgttcatcctgattacaaggatatgcttatggaatattttgaagaggcttgtaagtcatcaggtggaaatacaccacataatcttgaaaaagctctttcctggcatacaaaatttataaaaactggtagtatgaaataa

SEQ ID NO: 45 - DNA, 1290 bp, Artificial Sequence
  Synthesized Cat2
atggagtgggaagagatatataaagagaaactggtaactgcagaaaaagctgtttcaaaaatagaaaaccatagcagggtagtttttgcacatgcagtaggagaacccgtagatttagtaaatgcactagttaaaaataaggataattatataggactagaaatagttcacatggtagctatgggcaaaggtgaatatacaaaagagggtatgcaaagacattttagacataatgctttatttgtaggcggatgtactagagatgcagtaaattcaggaagagcagattatacaccttgttttttctatgaagtgccaagtttgtttaaagaaaaacgtttgcctgtagatgtagcacttattcaggtaagtgagccagataaatatggctactgcagttttggagtttccaatgactataccaagccagcagcagaaagtgctaagcttgtaattgcagaagtgaataaaaacatgccaagaactcttggagattcttttatacatgtatcagatattgattatatagtggaagcttcacacccattgttagaattgcagcctcctaaattgggagatgtagaaaaagccataggagaaaactgtgcatctttaattgaagatggagctactcttcagcttggaataggtgctataccagatgcggtacttttattcttaaagaacaaaaagaatttaggaatacattctgagatgatatcagatggtgtgatggaactggtgaaggcaggggttatcaataacaagaaaaagaccctccatccaggcaaaatagttgtaacatttttaatgggaacaaaaaaattatatgattttgtaaacaataatccaatggtagaaacttattctgtagattatgtaaataatccactggtaattatgaaaaatgacaatatggtttcaataaattcttgtgttcaagtagacttaatgggacaagtatgttctgaaagtataggattgaaacagataagtggagtgggaggccaggtagattttattagaggagctaatctatcaaagggtggaaaggctattatagctataccttccacagctggaaaaggaaaagtttcaagaataactccacttctagatactggtgctgcagttacaacttctagaaatgaagtagattatgtagttactgaatatggtgttgctcatcttaagggcaaaactttaagaaatagggcaagagctctaataaatatcgctcatccaaaattcagagaatcattaatgaatgaatttaaaaagagattttag

SEQ ID NO: 46 - DNA, 1314 bp, Artificial Sequence
  Synthesized Cat3
atggtttttaaaaattggcaggatctttataaaagtaaaattgttagtgcagacgaagctgtatctaaagtaagctgtggagatagcataattttaggcaatgcttgtggagcatctcttacacttttagatgccttggctgcaaataaggaaaagtataagagtgtaaagatacacaatcttatacttaattataaaaatgatatatatactgatccggaatcagaaaagtatattcatggaaatactttctttgtaagtggaggtacaaaggaagcagttaattgtaatagaacagattatactccatgctttttttatgaaataccaaaattattaaaacaaaagtatataaatgcagatgtagcttttattcaagtaagtaagcctgatagccatggatactgtagctttggagtatcaaccgattattcacaggcaatggtacagtctgcaaagcttataattgcagaagtaaacgatcagatgccaagagttttaggagacaattttatacacatttctgatatggattacatagtagaaagttcacgtccaattctagaattgactcctcctaaaataggagaagtagagaagacaataggaaaatactgtgcatctcttgtagaagatggttctacacttcagcttggaataggagctattccagatgcagtacttttattcttgaaggataaaaaggatttgggtatacattcagaaatgatatccgatggtgttgttgaattagttgaagcaggggtaattacaaataagaaaaagtcccttcatccaggaaaaataattattacattcttaatgggaactaagaaattatatgatttcataaatgataatcctatggtagaaggataccctgtagattatgtaaatgatcctaaggttattatgcaaaattctaagatggtatgtataaactcctgtgtagaagtggatttcacaggacaagtgtgtgctgaaagtgtaggatttaaacaaataagcggtgtaggtggacaagttgattacatgagaggagctagcatggctgatggaggaaaatcaattcttgctataccatctactgcagctggcggcaaaatttcaagaatagttcctattttaactgaaggagcgggggttactacttcaagatatgatgttcaatatgttgttacagaatatggtattgcacttctcaagggcaaatccataagagaaagagctaaggagcttataaaaattgcacatcctaaatttagggaagaattaacagctcaatttgaaaaaagattcagttgtaagctttaa

SEQ ID NO: 47 - DNA, 963 bp, Artificial Sequence
  Synthesized gctA
ttgagtaaagtaatgacgttaaaagacgcaatcgccaagtatgtgcacagtggtgatcacattgctctgggtggttttacgacggaccgtaaaccctatgcggctgtgttcgaaatcctgagacagggtatcacggatctgaccggtctgggcggcgctgccggcggcgactgggatatgctgatcggcaacggccgtgtgaaagcctacatcaactgctacaccgccaactccggtgtgaccaacgtttccagacggttcagaaaatggttcgaagccggcaaactgaccatggaagactattcccaggatgttatctacatgatgtggcatgccgccgctctgggcctgcccttcctgcctgtaaccctgatgcagggctccggcctgaccgatgaatggggcatcagcaaggaagtccgtaaaaccctggacaaagttcctgatgacaaattcaaatacatcgacaaccccttcaaaccgggtgaaaaagtcgtggctgttcctgttccgcaggttgatgtggccatcatccatgcccagcaggcttctcccgatggcaccgttcgcatctggggcggcaaattccaggatgtggatattgctgaagcagccaaatacaccatcgttacctgcgaagaaatcatttctgatgaagaaatcagaagagatcccaccaagaacgatatccccggcatgtgcgtagatgctgttgtcctggctccttacggtgcacatccttctcagtgctatggcctgtacgactacgacaatccgttcctgaaagtctatgacaaggtctccaagacccaggaagacttcgatgccttctgcaaggaatgggtgttcgacctgaaggatcatgacgaatacctgaacaaactgggtgccactcgtctgatcaacctgaaggttgttcctggtctgggctaccacatcgacatgacgaaggaggacaaataa

SEQ ID NO: 48 - DNA, 963 bp, Artificial Sequence; identical to SEQ ID NO: 47
  Synthesized gctB

SEQ ID NO: 49 - DNA, 1251 bp, Artificial Sequecne
  Synthesized frc
atgtcaactccacttcaaggaattaaagttctcgatttcaccggtgtgcaatctggcccatcttgtactcaaatgctggcctggtttggcgctgacgttattaaaattgaacgtcccggcgttggtgacgtaacgcgtcaccagctgcgagatattcctgatatcgatgcgctttacttcaccatgcttaacagtaacaaacgttctattgagttaaataccaaaacagcggaaggcaaagaggtaatggaaaagctgatccgcgaagctgatatcttagtcgagaactttcatccaggggccattgatcacatgggcttcacctgggagcatattcaagaaatcaatccacgtctgatttttggttcgatcaaagggtttgatgagtgttcgccttatgtgaatgtaaaagcctatgaaaacgttgctcaggcagcgggtggcgcggcatccactacgggtttttgggatggtccgccgctggtaagcgctgcagcgttgggtgacagcaacaccggaatgcatttgctgatcggtttacttgctgctttgctgcatcgcgaaaaaacggggcgtgggcaacgagtcaccatgtcaatgcaggatgccgtattgaacctttgccgcgtgaaattacgtgaccagcagcgtctcgataaattgggttatctggaagaatacccgcagtatccgaatggtacatttggtgatgcagttccccgcggtggtaatgcaggtggtggcggtcagcctggctggatcctgaaatgtaaaggctgggaaaccgatcctaacgcctatatttatttcactattcaggagcaaaactgggaaaacacctgtaaagccatcggcaaaccagaatggattaccgatccggcatacagtacagcccatgcacgacagccacatattttcgatatttttgctgaaatcgaaaaatacactgtcactattgataaacatgaagcggtggcctatttgactcagtttgatattccttgtgcaccggttttaagtatgaaagaaatttcacttgatccctctttgcgccaaagtggcagtgttgttgaagtggaacaaccgttgcgtggaaaatatctgaccgttggttgtccaatgaaattctctgcctttacgccggatattaaagctgcgccgctattaggtgaacataccgctgctgtattgcaggagctgggttatagcgacgatgaaattgctgcaatgaagcaaaaccacgccatctga

SEQ ID NO: 50 - DNA, 1278 bp, Artificial Sequence
  Synthesized BBta_3113
atgaccaaggcgctcgagggcgttcgcattctcgacttcacccacgtccagtccggaccgacctgcacccagctgctggcctggttcggcgccgacgtgatcaaggtcgagcggccgggcgtgggtgacatcacccgcggccagctgcaggacattcccaacgtggacagcctgtatttcacgatgctgaaccacaacaagcggtcgatcacgctcgacaccaagaaccccaagggcaaggaggttctgaccgagctgatcaagaagtgcgacgtgctggtcgagaatttcggccccggcgtgcttgaccgcatgggcttcccctgggagaagatccaggccatcaacccgaagatgatcgtcgcctcgatcaagggtttcggccctggcccttacgaggactgcaaggtctacgagaacgtcgcgcagtgcaccggcggcgccgcctcgaccaccggcttccgtgacggcctgccgctggtcaccggcgcgcagatcggcgattccggcaccggcctgcacctcgcgctcggcatcgtcaccgcgctctatcagcgcacccataccggcaagggccagcgcgtcacggctgcgatgcaggacggcgtgctcaacctctgccgtgtcaagctgcgcgaccagcagcgcctggagcgcggcccgctcaaggaatacagccagttcggtgagggcgttccgttcggcgacgccgtgccgcgcgccggcaacgattccggcggtggccagccgggccgcatcctgaagtgcaagggctgggagaccgacccgaacgcctacatctacttcatcacccaggccccggtctgggagaagatctgcgacgtgatcggcgagcccacctggaagaccgatccgaactacgccaagccggccgcccgcctgccgcgcctgaacgagatcttcggccgcatcgagcagtggaccatgaccaagaccaagttcgaggccatggacatcctcaacgagttcgacatcccctgcggcccgatcctgtcgatgaaggagatcgccgaggacgagtcgctgcgcaagaccggcaccctggtcgaggtcgaccacccgacccgcggcaaatatctctcggtcggcaacccgatcaagctgtcggacagcccggccgaggtgacccgctcgcctttgctcggcgagcacaccgatgagatcctgcgccaggtgcttggcttcagcgaccaccaggtcgccgagatccacgactccggcgcgctcgatccaccgcgtaaggaagctgcggagtaa

SEQ ID NO: 51 - DNA, 1326 bp, Artificial Sequence
  Synthesized RPA1945
atgggagagatgccgcttcggcgcgcaagagacaacaggagcacgaccatgaccaaggcgctcgacggcgttcgcgttctcgacttcacccacgtccaatccggcccgacctgcacgcagctcttggcgtggttcggtgccgacgtgatcaaggtggagcgccccggcagcggcgacatcacccgcggtcagctgcaggacatcccgaaggtggacagcctgtatttcaccatgctgaaccacaacaagcggtcgatcacgctcgacaccaagaacccgaagggcaaggaggtgctgaccgcgctgatccgcacctgcgacgtgctggtagagaatttcggccccggtgtgctcgaccggatgggcttcacctgggagaagatccaggagatcaacccgcggatgatcgtcgcctcgatcaagggcttcggtcccggcccgtatgaagactgcaaggtgtacgagaacgttgcgcagtgcaccggcggcgccgcctcgaccaccggattccgcgaaggcctgccgctggtcaccggcgcgcagatcggcgatagcggcaccggcctgcatctcgcgctcggcatcgtcaccgcgctgtatcagcgccaccacaccggccgcggccagcgcgtcaccgcggcgatgcaggacggcgtgctgaacctctgccgcgtcaagctgcgcgatcagcagcgcctcgaccatggtccgctgaaggaatacagccagttcggcgaaggcatcccgttcggcgatgcggtgccgcgtgccggcaacgattccggtggcggccagcccggccgcatcctgaagtgcaagggctgggagcaggatccgaacgcctacatctacgtcatcacccaggcgccggtgtgggagaagatctgcgacgtgatcggcgagaccggctggaagacgcaccccgactacgccacgccgccggcgcggctgtcgcggctcaacgagatcttcgcgcgcattgagcaatggaccatgaccaagaccaagttcgaggccatggagatcctcaacgccgacgacatcccctgcggcccgatcctgtcgatgaaggaactcgccgaagatcagtcgctgcgcgccaccggcaccatcgtcgaggtcgatcacccgacccgcggcaagtatctgtcggtcggcaacccgatcaagctgtcggactccccgaccgaggtgaagcgctcgccgctactcggtgaacacaccgacgaaatcctgcgcgacgtcctcggctacagcgacgcgcacgtcgcagagatccacgactccggcgcgaccgctccgccgcgcaagcaagcggcggagtaa

SEQ ID NO: 52 - DNA, 1251 bp, Artificial Sequence
  Synthesized SDY_2572
atgtcaactccacttcaaggaattaaagttctcgatttcaccggtgtgcaatctggcccatcttgtactcaaatgctggcctggtttggcgctgacgtcattaaaattgaacgccccggcgttggtgacgtaacgcgtcaccagctgcgagatattcctgatatcgatgcgctttacttcaccatgcttaacagtaacaaacgttctattgagttaaataccaaaacagcggaaggcaaagaggtaatggaaaagctgatccgcgaagctgatatcttagtcgagaactttcatccaggggccattgatcacatgggcttcacctgggagcatattcaagaaatcaatccacgtctgatttttggttcgatcaaagggtttgacgagtgttcgccttatgtgaatgtaaaagcctatgaaaacgttgctcaggcagcgggtggcgcggcatccactacgggtttttgggacggtccgccgctggtaagcgctgcagcgttaggagacagcaacaccggaatgcatttgctgatcggtttacttgctgctttgctgcatcgcgaaaaaacggggcgtgggcaacgagtcaccatgtcaatgcaggatgccgtattgaacctttgccgcgtgaaattacgcgaccagcagcgtctcgataaattgggttatctggaagaatacccgcagtatccgaatggtacatttggtgatgcagttccccgcggaggtaatgcgggtggtggcggtcaacctggatggatcctgaaatgtaaaggctgggaaacagatcctaacgcctatatttatttcactattcaggagcaaaactgggaaaacacctgtaaagccatcggcaaaccagattggattaccgatccggcatacagtacagcccatgcccgacagccacatattttcgatatttttgctgaaatcgaaaaatacactgtcactattgataaacatgaagcggtggcctatttgactcagtttgatattccttgtgcaccggttttaagtatgaaagaaatttcacttgatccctctttacgccaaagtggcagtgttgtcgaagtggaacaaccgttgcgtggaaaatatctgacagttggttgtccaatgaaattctctgcctttacgccagatattaaagctgcgccgctattaggtgaacataccgctgctgtattacaggagctgggttatagcgacgatgaaattgctgcaatgaagcaaaaccacgccatctga

SEQ ID NO: 53 - DNA, 1278 bp, Artificial Sequence
  Synthesized RPB_3427
atgaccaaggcgctcgacggcgttcgcattctcgatttcacccacgtccagtccggcccgacctgcacccagttgctggcgtggttcggcgccgacgtcatcaaggtcgagcgtcccggcaccggcgacatcacccgcgggcagttgcaggacatcccgaaggtggacagcctgtatttcaccatgctgaaccacaacaagcgctcgatcacgctcgacaccaagaaccccaagggcaaggaggtgctgaccgcgctgatccgctcctgcgacgtgctggtggagaatttcggccccggcgtgctcgatcgcatgggcttcacctgggacaagatccaggagatcaacccgcggatgatcgtcgcctcgatcaagggtttcggcccgggtccctatgaagactgcaaggtctacgagaacgtcgcgcaatgcaccggcggcgccgcctcgaccaccggcttccgcgacggcccgccgctggtcaccggcgcacagatcggcgactcgggcaccgggctgcatctcgcgctcggcatcgtcaccgcgctgtatcagcgccatcacaccggccgcggccagcgcgtcaccgccgcgatgcaggacggcgtgctcaatttgtcgcgcgtcaagctgcgcgatcagcagcgcctcgcccacggcccgctcaaggaatacagccagttcggcgaaggcattccgttcggcgacgcggtgccgcgcgccggcaatgattccggcggcggccagcccggccgcatcctgaaatgcaagggctgggagaccgatcccaacgcctacatctacttcatcgcgcaggccccggtgtgggagaagatctgcgacgtgatcggcgagaccggctggaagacccatccggactacgcgacgccgccggcgcggctgaagcacctcaacgacatcttcgcccgcatcgaacaatggaccatgaccaagaccaagttcgaggcgatggacatcctcaacagggacgacattccctgcgggccgatcctgtcgatgaaggaactcgccgaggacgcctcgctgcgcgccaccggcacgatcgtcgaggtcgatcatccgacccgcggcaaatatctgtcggtcggcaacccgatcaaactgtcggactcgccgacccatgtcgagcgctcgccgcttctcggcgagcacaccgacgaaattctgcgcgacgtcctcggcttcaacgatcatcaggtcgctgaaatccacgattccggcgcactcgctccgccgcgcaagcaggccgcagagtaa

SEQ ID NO: 54 - DNA, 1290 bp, Artificial Sequence
  Synthesized frc (1)
atgagcaaggcaccgggcaaggccctcgagggcgttcgcatcctcgatttcacccatgttcaatcggggccgacctgcacgcaattgctcgcgtggttcggggccgacgtcatcaaggtcgagcggccgggtgcgggcgacgcgacgcgccagcagcttcaggaccttcccggcgtggacagcctctatttcacgatgctgaaccacaacaagcgttcgatcacgctcgacggcaagaaccccaagggcaacgcgatcctctggcggctcattgccgagtgcgacgtgctggtcgagaacttcgcccccggtgcgctcgaccgcatggggctgacctgggagaagctgcaggccgccaatccgggcctgatcctggcctcggtgaagggcttcgggcccggccgctaccaggattgcaaggtctacgagaacgtcgcccaatgcgtcggcggcgcggcctccaccaccggctggcgcgacggcgtgccgatggtgtcgggggcgcagatcggcgattccggcaccggcctgcatctggcgctcggcatcgtcacggccctctaccagcgcacccagacggggcagggccagcgcgtcgattgtgccatgcaggacggggtgctcaacctctgccgggtgaagctgcgggaccagcagcgcctcgcccacggcccgctgatggaatacagccagtacggcgagggcgtccccttcggcgaggcggtgccgcgggccggcaacgattccggcggggggcagcccggccgcatcctcaagtgcaagggctgggagcaggatcccaacgcttacatctacttcatcacgcagggcgcggtctgggggccgatctgcgacatcatcggcgagccggactggaagaccgatccggcctacgcgacgccgaaagcccgcctgccgcatctcaacgagatcttcacgcgcatcgaagcgtggacgatgaagcacgacaagctcgaggcgatggagatcctcaacgcctacgagatcccgtgcggaccgatcctgtcgatgcgggagatcgccgaggatccgatgctgcgggcgaacggcacggtggtcgaggtcgagcacccgacccgcggggcctatctgacggtgggcaacccgatcaagctgtcggcgagccccaccgagatcacccgcgcgccgctgctcggcgagcataccgacgagatcctgcgcgaggtgctgggctgcaccgatacggaaatcagcgacatcctcggttcgggtgcggtgggcggcgtccaccgcatcgccgcggagtag

SEQ ID NO: 55 - DNA, 1251 bp, Artificial Sequence
  Synthesized H16_B1711
gtgaacctcccactcaacggcatcaagatcatcgacttcacgcacgtccaggccggtcccgcctgcacgcagcttctcgcgtggttcggtgcggacgtgatcaaggtcgagcgccccggttccggcgacgtgacgcgcacccagctgcgcgacatcccggatgtcgatgccctgtacttcaccatgctcaacagcaacaagcgcagcctgacgctggataccaagaagccggaaggcaagaagatcctggagcagctgatccgcgagtcggacgtgctggtcgagaacttcggcccgggcgcgctggaccgcatggggttctcgtgggaacgcatcaacgaactgaacccgaagatgatcgtggcttcggtcaagggcttcagcgacggccaccactatgaagacctgaaggtctacgagaacgtggcccagtgcgccggcggcgcggcctcgaccaccggcttctgggatggcccgccgacggtgtccgccgcggcgctgggcgattccaacaccggcatgcacctggccatcggcatcctcaccgcgctgatcggccgcgacaagaccggcaagggccagaaggtggctgtgtcgatgcaggatgcggtgctgaacctgtgccgggtcaagctgcgcgaccagcagcgcctggaccgcctgggctacctggaggagtacccgcagtatccgcacggcagcttcagcgacgtggtgccgcgcggcggcaacgcgggcggcggcggccagccgggctgggtgctgaagtgcaaggggtgggaaaccgaccccaacgcctatatctacttcaccatccagggccatgcctgggagccgatctgcaaggcgctgggcaagccggaatggatttccgatcccaactacgccaccgccaaggctcgccagccgcatatcttcgatatcttcaacaccatcgaggaatggctggccgacaagaccaagtacgaggccgtggacatcctgcgcaagttcgacatcccgtgctcgccggtgctgtcgatgaaggaaatcgccgccgatccgtcgctgcgcgccagcggcagcatcaccgaggtgccgcacaaggagcgcggtacctacctgacggtgggcagcccgatcaagttctccgacctcaagccggagatcaccgggtcgccactgctgggcgagcatagcgaagaggtgctggccggcctgggctacggcgcggacgacatcaagcgcctgcgcgagtcccaggtgatctga

SEQ ID NO: 56 - DNA, 1251 bp, Artificial Sequence
  Synthesized Bxe_B2760
atgaccaaacctctcgaaggcatccggatcatcgacttcacccatgttcaagccggccctgcatgcacccagttgctcgcctggttcggcgcggacgtcatcaaggttgaacggccgggttcgggcgacgtgacgcgcaaccagttgcgcgatattcccgacgccgacgcgttgtacttcacgatgctcaacagcaacaagaaatcgctgacgttggacacaaaaaaacccgaaggcaaggaagtactcgaaaagctgattcgcgaatccgacgtgctggtggagaatttcggcccgggcgcgttggaccgcatgggcttttcgtgggaacggctgaatgaactcaatccgaagatgatcgtcgcctcggtgaaaggcttcagcgacggccaccactacgacgacctgaaggtctacgaaaacgtggcgcaatgcgcgggcggtgcggcctccaccaccggcttctgggacggtccgcccaccatcagcgccgccgcgctcggcgacagcaataccggtatgcatctggccatcggcattctgaccgcgctgctcggtcgcgacaaaaccggcaaaggccagaaggtcgcagtgtccatgcaggacagcgtgctgaatctgtgccgcgtgaagcttcgtgaccagcagcggctggaacgcgttggctatctcgaggagtatccgcaatatccgcacggcgaattcagcgacgtggtaccgcgcggcggcaatgcaggcggcggcggccagccgggttgggtgctcaaatgcaaaggctgggaaacggatccgaacgcctacatctacttcacgattcagggccatgcgtgggagcccatctgcaaggcgctcggcaagcccgagtggatcgacgacccggcctacaagactgcggaagcgcgtcaaccgcatatcttcgatatcttccagaccatcgaaacctggctcgcggacaaaaccaagttcgaagcggtcgacatcttgcgcaagttcgacattccgtgcgcaccggtgctgaccatgaaggaactggccaacgatccgtcgttgcgcgcgagcggcacgatcgtcgaagtaccgcacaagaaacgcggcacgtatctgactgtcggcagcccgatcaagttttcggatctgaagccggaagtcaccgcgtcgccgctgctcggcgaacacaccgacgaggtgctggcgagccttggctacagccagcagcaaatcttcaacctgcgcgaagtcaaggcagtttaa

SEQ ID NO: 57 - DNA, 1524 bp, Artificial Sequence
  Synthesized cat
atgaacgaacgcatctccaacgagaagttgcgcggaaagatcatgaccgccgatgaggccgcagcccttatccccaatggagcccagatcggcttcggcggtttcaccggctcgggttacccgaaggaactgccccaggccctggccaacaggatcaaggcagcccacgagcgcggcgaggaattcaccgtcaacgcgttcaccggtgcgtccaccgctcccgagctcgatggtgccctggccggcgtcgatggcatgcactaccgcatgccgtaccagtcggatccgacgctgcgcaagaagatcaacgacggcaccacgctgtacaccgacatccacctgtcgcactcggcccagctggtggccgagggcttcatgggcaagctcgacttcgccgtggtcgaggccgtgcgcatcaccgaggacgggaacatcgttcccacctcgtcggtgggcaacaacaagacctacctcgacatggccgagaagatcatcatcgaggtgaactcctggcagtcgcccgacctcgagggaatgcacgatatctggaatggatatctgactcccccgaaccgtccgatcatcccgatcaacaatgtgggcgaccgcatcggcgacaccttcctgaccatcgattccgacaaggtcgtggcggtcatcgagaccaccgaccgtgaccgcaactcgccgttcaagccgattgacgacgattcgcgcgccattgccggctacctgctcgacttctatgccaacgaggtcaagcacggccgcctgccgaagaacctgctgccgctgcagtccggcgtgggcaatatccccaacgccgtgctcgacggcctgctgcattccgacctcgagaacctcaccagctacaccgaggtgatccaggacggcatggtcgacctgatcgacgccggcaagctggccgtcgcctcggcaacggccttctcgctgagccccgattacgcgcacaacatgaatgagaacgcgaagaactaccgcaagtcgatcgttcttcgtccccaggagatctcgaaccatcctgaggtcatccgtcgcctgggcgtgctgtcgtgcaatggcatgatcgaggccgacatctacggcaatgtgaactccacccacgtgatgggcacccgcatgatgaacggcatcggtggttccggtgacttcacgcgcaacgcctacatctcggcattcgtgtcgccgtcgaccgccaaggacggcgccatctcggcgatcgtgccgatggtgagccacgtcgaccacaccgagcatgacgtgatggtgatcatcaccgagcagggcatcgccgacctccgtggcctggcaccgcgcctgcgcgcccagaagatcatcgacaattgcgtgcatcccgatttccgtgcgcagctgcaggagtacttcgaccactcgctggtgacgtcgaaggccaagcacacgccgcacgacctgcgccattcctacgattggcacctgaacttcatcgagaacggcacgatgaagctttcgcactaccgttga

SEQ ID NO: 58 - DNA, 423 bp, Artificial Sequence
  Synthesized Orf1
tcagtccttcggcggttccagatagcgcccgaagcgctcgcgccattcgtcgtcgatcaaggtcgcgcgcggggcgccgccgaggtcggcccacacgaccgtctgcttcgcgcggaagcgcacctgctcgcccatcgacgcggtcgtgacgatgtccatcgagctgccgccgatgcgcgcgacgtagagcgtgaaggtgagctcatcgccgtgcatgctcggtgcgaaaaagtcgacttcgaggtggcgcatcggcacgccgcggcggatctccgcgtgcagcttgtagaagtccacgccgatgccgcggtcgaaccagtcctcgaccacctcattgcacagcaccaggcactgcgggtagaagacgatgccggccgggtcgcagtggtggaaacggatggatttcttgcattcgaagatcat

SEQ ID NO: 59 - DNA, 432 bp, Artificial Sequence
  Synthesized COG0824
tcattgggccgcaacctccaccagccgggtgcgataggcttccaggcgttcgcgcatgggaccgggcatgggaaccgccttcaccttttcctgatcggcgacgacacagacgaaactggtctcgaaggccaccacgccgtcaccccgcgcgccgatggtgcggaaatgaatggaagagccccccaccctgtccaccaggaccgagatatccacccggtcgccgggccgaagcggcgatttgatctccatgccgatcttgacgaagggcgtgccgaagccgtgttccttgttgatggtgtaccagtcatagccgatgacatcggccatgaagacctccagcgcctccatggcgtattccaggaagcggggcgtatagacgatgcgcgccgcgtcggaatcgccgaaatggacccggcggcggtgaatgaacac

SEQ ID NO: 60 - DNA, 411 bp, Artificial Sequence
  Synthesized Jann_0674
atgacccacctctggcccctgcgcgtctactatgaagacgtcgatctggcggggatcgtctactacgccaactacctgaaatacctggagcgggggcgctctgaaatggtgcgtgaggccggcatttcccagctcgacatgaaagctgcggggctggtctttgccgtgcggcgggtggaggcggaatacctcaaacccgccaaatacgatgatgagctggtcgtggagacgcagctggaccgcctgaaaggggccagtttcgacatgccccagcgggtcctgcgcggcgatgacgtgctgctggacgcgcggatcaaggttgtgatcctcaacgcggacggccgggcggcgcgacttccggcggatattcgcgcaaaagtcacagccgtcgcggcaagtgatggcccgtaa

SEQ ID NO: 61 - DNA, 432 bp, Artificial Sequence
  Synthesized SSE37_24444
atgtcgcaggaggaagccgtggggcagccgttcgagcatgagatccgggtgacctggggggactgcgatcccgcgcggatcgcctatacggcgcgcatcccctggttcgcgctggatgcgatcaacgcctggtgggaggagaagctgggcggcggctggttccagatggagctggaccgcggtgtcggcacgccgttcgtcaacatgaccatcgatttccgcagtccggtcacgccgcgccaccggctgctctgcgccgtgcgcccggtgcggctgggcgagacctcggtcagtttcgaagtgctgggacggcaggacggtgtgctgtgtttcgaggggcggttcacctgcgtgttcatcgccgtgccgcgttttcgcaaggcgccgccgccggaggatatccgggcggtggtggaggcgcatctgaactag

SEQ ID NO: 62 - DNA, 414 bp, Artificial Sequence
  Synthesized entH
atgatctggaaacgccatttaacgctcgacgaactgaacgccaccagcgataacacaatggtggcgcatctgggaattgtgtatacccgtctgggcgatgatgtgctggaagccgaaatgccggttgatacccgtactcatcagccgttcggtttactacatggcggcgcgtcggcggcgctggcggaaacgctgggatcgatggccggatttatgatgacccgcgacggacagtgtgtggtaggcacagaacttaatgcaacacaccatcgcccggtgtctgagggaaaggtacgcggcgtctgccagccgctgcatcttggtcggcaaaatcagagctgggaaatcgtcgttttcgatgaacaggggcggcgttgctgcacttgtcggctgggtacggcagttttgggatga

SEQ ID NO: 63 - DNA, 1989 bp, Artificial Sequence
  Synthesized acs
atgtcggaaaacatcactatcctgcctacacagtatgcagattacccggctctgatgccacctgcggaactggccgccatgcagcgctatgcacgccgagacccggatggtttttggctgcaacaggcccggcgtgtgcactggcaccgcaagcctaggcgaggctttacgggcagctttacgggtgatgtgtccataagctggtttgaagatggccttatcaacgcatccgtatgctgtattgataagcatctgacagacaaggctgatcagattgcccttatcagccaccgtgaaggccgggccgaggcagaaaaaattacatatgccatgctgcatgaacgggtttgccgcctgtctaacgcgctggtgcatttgggggtggaggaagggcaccgcgttgccatttgcctgcccatgatttcagaagccgtggtggccatgctggcctgtgcgcgtattggcgcggtgcatgtggtgctgtttggtggtttttcggcagaaggtattgcagaacgtattatagatagcggcgcagttgcggtaattaccgccagcgaaagcatgcgcggcaacaagatcgtgccctttaaagcgattatggatgaagccctgtgcaaggcaggtgcagaaagtggcgtgcgggctgttctagttgtgcgcacgtctgatgcacctgttcccatgctgcctggtagggattacgattttcatgattttgtagattcgtttgaggcagattttgtgcccgttgtcatgcgggcagaagcaccattatttatgctctacacatctggcagcacaggcaagcccaaagcagttgtgcatgccactggtggctatatggtgtgggcagcttacactatggacatggtgtaccatcatcaacctggtgatgtgctgtggtgcacggcagatgtggcatggataaccgggcatacatccgttgtgtatggcccgctggccaatggcggaaccaccatgatttccgatagcctgccttcataccccgctccgggcagatggttggatctgatagatgagcataaggtgaccatgctgtttaccgcccccacagccgtgcgcgccatgatggccgatggtgatgatgtggtgaacgcccgcaatctggagtctctgcgtttgctgggtgtggcgggggagcccataagcccggatgcgtggctatggtatcacgatgttgtgggtaaaaagcgttgccccgtggtggatacatggtggcagacagaaaccgccggcattgtgctggggccagtgccgggtgtgcaaccgcttaaacccggctctgccagcacgccgctgccggggttggaaatggtcatagccgatacgcagggcaggccggtgcaggggcctgcagaaggtagcctgtgcattgcgcgttcatggccggggcaggcccgcacaatctggaaagatcatgctcgcttctgccagacatattttggtatggttccggggcattatttcacgggtgatggcgcacggcgagatgccgatggctattactggattacggggcgcatggacgatgttatcaatattgcagggcaccgtttgggtacagcagaagtggaagatgcgttggcagcagatcatcgtattgtggaatctgctgcagtgggcatcccgcacccggtaaaggggcaggcgctggcggtatttgttatccagcgccagaacgtggctacggaactgacagaaaaaggcataagccgccttatctccggtatgttggggcgttatgccacgccagaggccgtttatctggtgccagatctgcctcgcacgcgctctggcaagattgtacgccgcctgctgcgcaaaattgccagtggggaaatggataatctgggagatctttcatcgctgaatgatccttccatcgtgcgtatgctgtgtgacagagtatggagccacatggcttttgatgaggaatctgcacctcgcacacaggcaagggcctga

SEQ ID NO: 64 - DNA, 1890 bp, Artificial Sequence
  Synthesized Avin_10660
atgaactatcagcactaccatgaacgctccatcgccgatcccgccggtttctgggccgaacaggcgcaggccgtgcgctggttccgccagccgacggaaattctccgcgccctggcggacggcacgcaccagtggttcgccgacggccggttgaacagttgctatctggccctggatcatcagatcgaacagggccgtggcgagcagacggccctgatccacgactcgccggtcaccggcggcaaggcccgctacagctaccgcgaactgcgcgacgaagtggcgcgcctggccggcgccctgcgcgagctgggcgtggaaaagggcgaccgggtcatcatctacatgccgatggtgccgcaagcggccatggccatgctcgcctgcgcgcggatcggcgcggtgcactcggtggtgttcggcggcttcgcccctcacgaactggcgctgcgcatcgacgacgcccggcccaaactgctgctcaccgcgtcctgcggcctggagttcgaccgggtcatcgaatacaaaccgctggtcgacaaggccctggaactggccagccaccagcccgggcacgtactggtgctgcaacggccacaagcgagcgccgcgctgctcccagggcgcgacctggactggcaggccagggtcccgctggccgcgccggtggagcccgtgcccctggacagcggcgatccgctgtacatcatgtacacctccggcaccaccggaaaacccaagggcgtcgtgcgcgacaacggcggcaacgcggtggccctgagcttcgccatgcgccatgtctacgccatgcgggccggcgacgtctggtggggcatctccgacgtcggctgggtggtcggccattcgctgatcgtctacggcccgctgatgaacggatgcaccagcatcctctacgaaggcaagccggtccgcacgcccgacgccggcgcctactggcgggtgatcgaggaatacggcgtcaacggcctgttctgcgcgccgacggcgatccgcgccatgcgcaaggaagatccttcgggcgaactgagcgggcgccacgacctgggctcgctgcggcacctgttcctggccggcgagaagctcgattcgagcacccaccggtggctggaggaactgaccgggaagccggtgcacgaccactggtggcagaccgagaccggctggccggtcaccgctccctgcgccgggctggagggccacaccgcacgccacggttcgagcaaccgcccggtgcccggctatcgcgtccaggtgatggacgaacagggtcacctgctcggagcgaaccggcagggctcgatcgtcatcgccctgcccctgccgccgggctgcgcgcagaccctgtggaacgaccacgagcgctatctgcgctcttatctgagctcctatcccggctactaccacaccggcgacggcggctacctggacgacgagggcttcgtctacatcatgggccgcaccgacgacgtgataaacgtggccggccaccgcctctccaccggagaaatggaagacctggtggcccggcatccggcggtggccgaatgcgcggtgatcggcatccccgacgcgatcaagggacaggtgccgctgggcctgatcgtcctcaaggacggcagccgaatccgcgaggagcaactgcagcgggagttgaccgcctcgatccgcgagcagatcggcgcgctggcctgcttccagcggatagcgacggtcaagcgcctgccgaagacccgttcgggcaaaatcctccgggcggtgctgcgcaagatcgccaacggcgaggaggtggccacgcccatgaccatcgacgatccggcgatactcggggaaatcggcgccgccctggcgttgtacacgcgcgccagttga

SEQ ID NO: 65 - DNA, 1959 bp, Artificial Sequence
  Synthesized acs (1)
atgagtaccgaagaaaagaagtttgacacgcaaaacctgcctaccaagacttatttctggccgctgaaaagataccaggacctttataacagctcactggctgacccggaggctttctgggccaaacactcagacgtgctttcatgggaaaagccttgggaaaaagtactggactggaatccgccttatgcccgctggtttgtaggcggcaagctgaatatgtcttaccaatgcgtagaccgccatgccaaaagctggcgtaagagcaaggtagctatctattgggaaggcgaaaacggggatacccagaccataagctattcagacctttacgaaaatgtaaaccgttatgcatccgtcctgaaaaagctgggcatatgcaagggtgacagggtaactgtctacctgcccatgatacctgaaatggtctatattctattagcctgcaaccgggttggagccgtccataacgtaatattctcaggtttctcttcccagtctatcgcagacagggtaaatgactccggttcaaaaatggttgttaccgccagcggcggacaccgccgcggtaagatactgcctcttaaagaaatcgtagatgaggctgtaaaatccaccccgactatagaacatgtactggttattaaatataccggccacgaagtagccatggaccccaccagagacgtatgggcacatgatctgctgaaagatgcagataaatacgtagcccctgaagctatggaatccaccgacccgctttttatcctgtacacctcaggcactaccggtaaaccgaagggtattctgcatggtaccggcggctacggcgtctgggcgtgcaatacccttaagtgggctttcaaacccacggacgaatcagtcttctggtgcacggcagacgtaggctggattaccgggcacacatatgttgtatatgccccgctggcgctgggacttacccaggttatttacgagggagctccggattatccttcagtagaccgctggtgggagattattgataaatacggggtaagcatattctatacctcgcctaccgccatacgcatgtttatgcgccacggcgaggagttgcctgccagacacgaccttggcactctggaaatgctgggaagcgtgggcgaacccattaaccctgaagcctgggaatggtattacaagaatataggccatgagaactgccccatttccgatacttggtggcagaccgaaacaggcggttttatgattaccccctgccccggcatacaatccttcccgctcaaaccgggctcagccactttgcctctaccgggagttgacccggtagtggtagatgctgaaggcaaggaactgccggctaatgaaaccgggtttattgccatccgcaaaccttggccgggcataatgctgggtatatataacggtgatgaactttataaaaagacctactggagccgtttccccggctggtattgtccgggagacttttcaatgaaagattctgacggatatctgtggctgctgggacgggctgacgaagttatcaaggtagccggtcaccgcataagcaccgccgaattggagcatgctctggtaggccatagttcagttgccgaagcggcagtagcctcccgccctgacgaagtaaagggtgaagctattgtggttttcgtcaccctgaaaaaaggtgtagaagcctctgcggaagtaaagagagagcttacccatcacctccgctctgctatcggcactatagccaccccggaagagatcattttcgtggagaaactgcccaaaacccgttcgggcaagattatgcgccgcctgctgaaggccgttgccaacgaagtacccattggtgataccactacacttgatgatgagacttcggtaaatgaggccagagcggcttttgatgaactgctggcagcacgcaaacaccacaaacactaa

SEQ ID NO: 66 - DNA, 2052 bp, Artificial Sequence
  Synthesized Acs2
atgacaatcaaggaacataaagtagtttatgaagctcacaacgtaaaggctcttaaggctcctcaacatttttacaacagccaacccggcaagggttacgttactgatatgcaacattatcaagaaatgtatcaacaatctatcaatgagccagaaaaattctttgataagatggctaaggaatacttgcattgggatgctccatacaccaaagttcaatctggttcattgaacaatggtgatgttgcatggtttttgaacggtaaattgaatgcatcatacaattgtgttgacagacatgcctttgctaatcccgacaagccagctttgatctatgaagctgatgacgaatccgacaacaaaatcatcacatttggtgaattactcagaaaagtttcccaaatcgctggtgtcttaaaaagctggggcgttaagaaaggtgacacagtggctatctatttgccaatgattccagaagcggtcattgctatgttggctgtggctcgtattggtgctattcactctgttgtctttgctgggttctccgctggttcgttgaaagatcgtgtcgttgacgctaattctaaagtggtcatcacttgtgatgaaggtaaaagaggtggtaagaccatcaacactaaaaaaattgttgacgaaggtttgaacggagtcgatttggtttcccgtatcttggttttccaaagaactggtactgaaggtattccaatgaaggccggtagagattactggtggcatgaggaggccgctaagcagagaacttacctacctcctgtttcatgtgacgctgaagatcctctatttttattatacacttccggttccactggttctccaaagggtgtcgttcacactacaggtggttatttattaggtgccgctttaacaactagatacgtttttgatattcacccagaagatgttctcttcactgccggtgacgtcggctggatcacgggtcacacctatgctctatatggtccattaaccttgggtaccgcctcaataattttcgaatccactcctgcctacccagattatggtagatattggagaattatccaacgtcacaaggctacccatttctatgtggctccaactgctttaagattaatcaaacgtgtaggtgaagccgaaattgccaaatatgacacttcctcattacgtgtcttgggttccgtcggtgaaccaatctctccagacttatgggaatggtatcatgaaaaagtgggtaacaaaaactgtgtcatttgtgacactatgtggcaaacagagtctggttctcatttaattgctcctttggcaggtgctgtcccaacaaaacctggttctgctaccgtgccattctttggtattaacgcttgtatcattgaccctgttacaggtgtggaattagaaggtaatgatgtcgaaggtgtccttgccgttaaatcaccatggccatcaatggctagatctgtttggaaccaccacgaccgttacatggatacttacttgaaaccttatcctggtcactatttcacaggtgatggtgctggtagagatcatgatggttactactggatcaggggtagagttgacgacgttgtaaatgtttccggtcatagattatccacatcagaaattgaagcatctatctcaaatcacgaaaacgtctcggaagctgctgttgtcggtattccagatgaattgaccggtcaaaccgtcgttgcatatgtttccctaaaagatggttatctacaaaacaacgctactgaaggtgatgcagaacacatcacaccagataatttacgtagagaattgatcttacaagttaggggtgagattggtcctttcgcctcaccaaaaaccattattctagttagagatctaccaagaacaaggtcaggaaagattatgagaagagttctaagaaaggttgcttctaacgaagccgaacagctaggtgacctaactactttggccaacccagaagttgtacctgccatcatttctgctgtagagaaccaatttttctctcaaaaaaagaaataa

SEQ ID NO: 67 - DNA, 2142 bp, Artificial Sequence
  Synthesized Acs1
atgtcgccctctgccgtacaatcatcaaaactagaagaacagtcaagtgaaattgacaagttgaaagcaaaaatgtcccagtctgccgccactgcgcagcagaagaaggaacatgagtatgaacatttgacttcggtcaagatcgtgccacaacggcccatctcagatagactgcagcccgcaattgctacccactattctccacacttggacgggttgcaggactatcagcgcttgcacaaggagtctattgaagaccctgctaagttcttcggttctaaagctacccaatttttaaactggtctaagccattcgataaggtgttcatcccagaccctaaaacgggcaggccctccttccagaacaatgcatggttcctcaacggccaattaaacgcctgttacaactgtgttgacagacatgccttgaagactcctaacaagaaagccattattttcgaaggtgacgagcctggccaaggctattccattacctacaaggaactacttgaagaagtttgtcaagtggcacaagtgctgacttactctatgggcgttcgcaagggcgatactgttgccgtgtacatgcctatggtcccagaagcaatcataaccttgttggccatttcccgtatcggtgccattcactccgtagtctttgccgggttttcttccaactccttgagagatcgtatcaacgatggggactctaaagttgtcatcactacagatgaatccaacagaggtggtaaagtcattgagactaaaagaattgttgatgacgcgctaagagagaccccaggcgtgagacacgtcttggtttatagaaagaccaacaatccatctgttgctttccatgcccccagagatttggattgggcaacagaaaagaagaaatacaagacctactatccatgcacacccgttgattctgaggatccattattcttgttgtatacgtctggttctactggtgcccccaagggtgttcaacattctaccgcaggttacttgctgggagctttgttgaccatgcgctacacttttgacactcaccaagaagacgttttcttcacagctggagacattggctggattacaggccacacttatgtggtttatggtcccttactatatggttgtgccactttggtctttgaagggactcctgcgtacccaaattactcccgttattgggatattattgatgaacacaaagtcacccaattttatgttgcgccaactgctttgcgtttgttgaaaagagctggtgattcctacatcgaaaatcattccttaaaatctttgcgttgcttgggttcggtcggtgagccaattgctgctgaagtttgggagtggtactctgaaaaaataggtaaaaatgaaatccccattgtagacacctactggcaaacagaatctggttcgcatctggtcaccccgctggctggtggtgttacaccaatgaaaccgggttctgcctcattccccttcttcggtattgatgcagttgttcttgaccctaacactggtgaagaacttaacaccagccacgcagagggtgtccttgccgtcaaagctgcatggccatcatttgcaagaactatttggaaaaatcatgataggtatctagacacttatttgaacccttaccctggctactatttcactggtgatggtgctgcaaaggataaggatggttatatctggattttgggtcgtgtagacgatgtggtgaacgtctctggtcaccgtctgtctaccgctgaaattgaggctgctattatcgaagatccaattgtggccgagtgtgctgttgtcggattcaacgatgacttgactggtcaagcagttgctgcatttgtggtgttgaaaaacaaatctagttggtccaccgcaacagatgatgaattacaagatatcaagaagcatttggtctttactgttagaaaagacatcgggccatttgccgcaccaaaattgatcattttagtggatgacttgcccaagacaagatccggcaaaattatgagacgtattttaagaaaaatcctagcaggagaaagtgaccaactaggcgacgtttctacattgtcaaaccctggcattgttagacatctaattgattcggtcaagttgtaa

SEQ ID NO: 68 - DNA, 810 bp, Artificial Sequence
  Synthesized mhpD
atgacgaagcatactcttgagcaactggcggcggatttacgccgcgccgcagagcagggcgaagcgattgcaccgctgcgcgatctgattggtatcgataacgctgaagcggcttacgccattcagcacataaatgtgcaacatgacgttgcgcaggggcgtcgcgtggtagggcgtaaagtgggcctgacacatccgaaagtgcaacaacaactgggcgttgatcaaccggattttgggacgttatttgccgacatgtgttatggcgataacgaaatcattcctttttcccgtgttctgcaaccccgcattgaagcggagatcgcactggtgttgaaccgcgatttgcccgcaaccgatatcaccttcgacgaattgtataacgccattgaatgggtacttccggcgctggaagtggtggggagccgcattcgcgactggtcgattcagtttgtcgataccgtggcagataacgcctcctgtggggtgtatgtcatcggcggtccggcgcaacgtccggcggggttagacctgaaaaactgcgccatgaagatgacgcgtaataacgaagaggtttctagcgggcgcggcagcgaatgcctgggacatccgcttaatgcggccgtctggctggcacgcaaaatggccagtctgggtgaaccgctgcgcaccggagatatcattcttaccggggcattaggtccgatggtggcggtgaatgcgggcgatcgttttgaagcccatattgaaggcataggttcagttgctgcgacattttcaagcgcagccccaaaaggaagtctgtcatga

SEQ ID NO: 69 - DNA, 789 bp, Artificial Sequence
  Synthesized ctmF
atgaatgaagccaacgtgattgcgaacctgttatgggatgcgcagcggcaaaagctgccctgtgcaccggtgcgggaatatttcgaggggaagagcgaggttgaccaggcgctattggcctatgccgtacagcaggtgaatgttcagcgccaggtggagggcggccgacgtatcgtcggtcgcaagatcggccttacctctccggcagtgcagaagcaattgggtgtagatcggccggacttcggcacgttgctggacgacatggcgatcgtcgatggcgagccgatcaacactgcgcgtcttctgcagcccaaggtcgaagctgagatcgccctggtactcgagcgtgacctcgatcgggagcgtcatacagtcgccgacctgatcgacgcgacagcgtatgcacttgctgcaatcgaggtggtggatagccgtatcaccggttggaacatccgctttgttgacaccgtggcagacaacgcctcatcgggcttgttcgtactcggtactcagcctgttggcctgtcgaagcttgatctggccggtatgtcgatgcgcatggcgcgtggcgaagagcttgtatcgcaaggggctggagctgcctgccttggcaacccgttgaacgcagcgcgttggcttgctgacacgttggtccaagtgggcacgccattgcgtgccggcgatgtggttctgaccggcgctctggggccaatggtcgcggtcgagtccggtcacacctatacggcatggatcgatggcttcgccccggtacgagcaattttctcctga

SEQ ID NO: 70 - DNA, 807 bp, Artificial Sequence
  Synthesized todG
atgagcgaactagataccgcgcggacaggtgccgtgcgtaaagctgccgacctgctgtacgaagccacccggtccggtgtggccgtggtgccggtgcgcaatctgatcggcgagacggatttggaggcagcctatgcagtacaggaggttaatacacagagagcattggttgccgggcggcgcctggttggacgcaagattgggctgacctctgtcgctgtacagaagcagctcggagtggaacagcccgactatggcatgttgttcgcagacatggcgcgtaccgagggggaggaaatcgcccttgatgacgtgctccaacctaaagtcgaagccgagatcgcctttgtcctgggacgtgacctcgatggcgatcaattgacggtggccgacctctttcgcgccatcgagttcgccgttccggcgatcgagatcgtgggttcgcggataaccaattgggatatccgtatcacggacaccattgctgacaatgcttcgtctggcctgtatgtgctgggctccacgccgaagcgcttgtgcgattttgactcgcgccaggcaggcatggtgatggagcggcaaggcataccggtgtcttccggggtaggggccgcctgccttggagcgcctctcaacgcagtcctttggttggccagggtcatggctcgagcgggccgtccgttgcgcactggcgacacggtgctttccggtgcgctgggccccatggtgccagtggcaggaggagatgtattcgatgtgcggatagccgggcttggatcggtgaccgccgcttttgcaaaggcataa

SEQ ID NO: 71 - DNA, 804 bp, Artificial Sequence
  Synthesized hpaH
atgctcgataaacagacccgtaccctgattgcccagcggctgaaccaggccgaaaagcagcgtgaacagatccgcgcgatctcgctggattatccgtcgatcaccattgaggacgcctacgccgtccagcgcgagtgggtcgagatgaagatcgccgaaggccgcgtgctcaaaggccacaagatcggcctgacctctaaagcgatgcaggccagttcgcagatcagcgagccggactacggcgcgctgctcgacgatatgttcttccacgacggcagcgatattcccaccgaccgctttatcgttccgcgtatcgaagtcgagctggccttcgtgctggccaaaccgctgcgcggcccgaactgtacgctgtttgatgtctacaacgccaccgactacgttatcccggcgctggagcttatcgacgcgcgctgccacaacatcgacccggaaacccagcgtccgcgcaaagtgttcgacaccatctccgacaacgccgccaacgccggggtgatcctcggcggccggccgattaaaccggacgagctcgacctgcgctggatctccgccctgctgtatcgcaacggcgtaattgaagagaccggcgtcgccgcgggcgtactcaatcatccggccaacggcgtggcctggctggccaacaagctggcgccgtacgatgtccagctcgaagccgggcagattatcctcggcggctccttcacccgcccggtcccggcgcgcaagggcgataccttccacgtcgactacggcaacatgggcgtcatcagctgccggtttgtctag

SEQ ID NO: 72 - DNA, 804 bp, Artificial Sequence
  Synthesized hpaH (1)
atgttcgacaaacacacccacaccctgatcgcccagcgtctggatcaggcagaaaaacagcgcgaacagatccgcgcgatctcgctggattacccggagatcaccatcgaagacgcttacgcggtgcagcgtgaatgggttcgactgaaaatcgccgaaggtcgcacgctgaaaggccacaaaatcggcctgacttcgaaagcgatgcaggccagctcgcagatcagcgaaccggattacggtgcactgctggacgacatgttcttccacgatggcagcgatatcccgaccgatcgctttatcgtgccgcgcattgaagtggagctggcttttgtgctggcaaaaccgctgcgtggaccaaactgcacgctgttcgacgtttacaacgccacggactatgtgatcccggcgctggagctgatcgacgctcgctgccacaacatcgatccggaaacccagcgcccgcgtaaagtgttcgacaccatttctgataacgccgccaatgccggggtgatcctcggtggtcgtcccattaagcccgatgagttggatctacgttggatctccgccctgatgtatcgcaatggcgtgattgaagaaaccggcgtcgccgctggcgtgctgaatcatccggcaaacggcgtggcctggctggcgaacaaactcgccccctatgacgtacaactggaagccgggcaaatcattctcggcggttcgttcacccgcccggttccggcgcgtaagggcgacaccttccacgtcgattacggcaacatgggctccattagctgccgctttgtttaa

SEQ ID NO: 73 - DNA, 840 bp, Artificial Sequence
  Synthesized cnbE
atgaaccgaacacaagccaaagtagtcgaaggcaaatttcccacacagaacaccatggacaactccaagatccagcactacggcgacgagctctaccagtcgctgctcgaccgccaacccgtcgctccgctgaccgaccgcgaagcggacatcaccatcgaggacgcctaccagatccagctgcgcatgatccagcgccggctggacgcgggcgagcgcgtggtgggcaagaaaataggcgtgacgagcaaggtcgtgatggacatgctcaaggtcaaccagcccgacttcggccacctgctctcgggcatggtctacaacgaaggccagcccatcccggtgagcagcatgatcgcgcccaaggccgaggcagaggtcgccttcatcctggcgcgcgacctcgaaggccccggcgtcaccgcggccgacgtgctgcgcgccaccgactgcgtgatgccgtgcttcgagatcgtcgactcgcgcatcaaggactggaagatcaagatccaggacaccgtggccgacaacgcctcctgcggcgtgctcacgctcggcggcctgcgcaagagcccgcgcgacctcgacctcgcgctggccggcatggtgctggaaaagaacggcgaaatcatcagcacgtcctgcggcgcatcggtgcagggctcgccggtcaacgcggtggcctggctggccaacacgctcggccgtctgggcatcggcctcaaggccggcgacatcatcctctctggctcgcagtcgccgctggtgccggtggtcgcgggcgacagcctgtattgcagcgtcggcggcctgggcggcacgtcggtgcgtttcgtcgcctga

SEQ ID NO: 74 - DNA, 507 bp, Artificial Sequence
  Synthesized leuD
atgagaagtataataaagggaagagtttggaagtttggaaataacgtagatacagatgctatattaccagcaaggtatttagtttatacaaaaccagaggaattagctcagtttgttatgactggggcagacccagattttccaaagaaggttaagccaggagatataatagttggaggaaagaactttggatgtggttcaagtagagagcatgccccattaggattaaaaggagctggaatcagctgtgttattgctgagagcttcgcaagaatattttatagaaatgccataaatgttggattaccattaattgaatgtaagggcatttcagagaaagtcaatgaaggggatgagttagaggttaatttagagactggagagattaaaaacttaaccactggagaggttttaaaaggtcaaaaattaccagaattcatgatggaaattttagaggctggaggattaatgccatacttaaagaaaaagatggctgaaagccaataa

SEQ ID NO: 75 - DNA, 1263 bp, Artificial Sequence
  Synthesized dmdA
ttgggtatgacaatgactcagaaaatattggcggcacatgctggtctggaatccgtaaaaccgggtgatttgatcatggcagacctggatctggtgttggggaatgatattacctcaccggtagccatcaatgtttttaaaaatattaataaggaaaccgtttttgacaaagacaaggttgcgctggtcccagaccattttgcgccgaacaaggatattaaggctgcggagcagtgcaaacaggtgcgctgttttgcctgtgagcaggatgtcaccaactattttgaaatcggcgaaatgggtgtagagcatgctctgctgccggaaaagggactggtcgttgccggcgatgtcgtgattggggcagattcgcacacctgtacctatggtgcgcttggggctttctcaaccggtgtgggttctaccgacatggccgttggtatggcaaccggtaaagcctggtttaaggtaccgtctgccattaaattcaatctgactggcgctttcaaagaaggtgtttcaggaaaagacctgattcttcacattatcggaatgattggtgtggatggtgcgctttataaatcaatggaatttgccggagagggtgtgtcaagcctgacgatggatgatcgcttcaccattgcgaatatggccattgaagctggcggtaaaaatggtatcttccctgtcgacgataagaccatcgaatatatgaaggagcattctaccaaggaatacaaggcctttgaagcagacgcagacgccgagtatgacgctgtgtacgatattaatctggcagatatcaagtctacggtagcattcccgcacttgcctgaaaacactaaaaccgttgatgaaattactgaaccggttaagattgaccaggttgttatcggctcatgcaccaatggacgtttctcagactttaaaaaggccgcagatctgatgcgcggtaagcatgttgccaaaggaatccgtgttttgattatcccagcaactcagcagatttacctggattgtatggaagcgggatatttaaaagactttattgaagcgggcgcaacggtgagcacaccgacctgcgggccatgcctgggcggacatatggggattctggcagcgggagaacgctgcgtttccacaacaaaccgtaactttgtcggacgcatgggccatgtggactcggaagtctatctggcgagccccgaggttgcggcggcatctgctatcctgggccgtattgccggaccagaagaattataa

SEQ ID NO: 76 - DNA, 492 bp, Artificial Sequence
  Synthesized dmdB
atgaaagcaaaaggaaaagtatttagatatggcaacaatgttgatacagacgttattattcccgcaagatacctgaacaccagcgatcctctggaattagcggagcattgtatggaggatattgacaaggattttataaaacgcgtggaggacggcgatatcatcgtcgctgatgataattttggctgcggctcttcaagagagcatgcgcccattgccatcaaagcctcaggtgtctcctgtgtaatcgccaatagctttgcgcgtattttttatcgcaattccatcaatatcgggctgccgattctggaatgtccggaagcggtggcagcgattgaagcaggcgacgaagtagaagtggattttgactctggcgttatcactgacgtgaccaagggacagagcttccagggacaggcattccctgaatttatgcagaagctgatcgcagcaggcggcctggtaaattacgtcaacgagaatctcatttag

SEQ ID NO: 77 - DNA, 1770 bp, Artificial Sequence
  Synthesized Olhyd_maccj
atgtactatagtaatggaaactatgaagcatttgcaagaccgaagaagccggaaggggtagataataagtctgcatatttagttggttctggtttagcgtcattagcagcggcaagttttttaatacgagatggtcaaatgaaaggtgaaaatattcatatattagaagaactcgatctccctggaggaagcttggatggaatattgaatcctgaacgtggctatataatgcgtggcggtcgtgagatggagaatcattttgaatgtttatgggatttatttcgttcagtaccatcattggaagtcgaagatgcttctgttctggatgaattttactggttaaataaagaagatccaaactattcgaagtgccgcgtaatagaaaatcgtggacaacgcctagaatcagatggaaaaatgactctaacaaaaaaagcaaataaagaaattatccagctgtgcttaatgaaagaagaacagctgaatgatgtgaagatctctgatgtcttcagtaaagacttcttagactcaaacttctggatctactggaaaacgatgtttgcatttgaaccttggcattctgctatggagatgcgtcgatatttaatgcgtttcatccatcatattggtggacttgcagacttttcagctctaaaatttacgaagttcaatcagttcgaatcacttgttatgcctctgattgagcatcttaaagcgaagaacgttacatttgaatatggtgtaactgttaagaatatacaagttgaatgttcaaaagagtcaaaagttgcaaaggcaatagacatcgtgcgcagaggtaacgaggaatcaattcctttaactgaaaatgatttagtatttgtaacaaatggcagtatcactgaaagtactacttatggagataatgacacacctgcaccgcctacatcaaaacctggtggcgcatggcaactatgggaaaacttaagtacgcaatgtgaggagtttggtaatccagctaaattctataaagatttaccagaaaaaagctggttcgtgtctgctacagcaacaacaaataacaaagaagttatagattatattcaaaaaatttgtaaacgcgatccattatcaggtcgtacagtaactggcggtatcgttactgtagatgattcaaattggcagttaagctttacgctaaatcgacaacagcagtttaaaaatcaacctgatgatcaagtgagtgtatggatttacgcactttattcagatgaacgtggagaacgtacaaataaaacaattgttgagtgttctggtaaagaaatttgtgaagaatggctttatcatatgggtgttcctgaagagaagatttcagcactagcagcagaatgtaatacaattccaagctatatgccgtacattaccgcttactttatgccgcgtaaagaaggagatcgtcctttagtagtaccacatggttcaaagaatattgcatttataggtaactttgcagaaacagaaagagataccgtatttacaacagaatattcagtaagaactgctatggaagcggtgtataaacttctagaagtagaccgtggagtgcctgaagtattcgcttcagtatacgatgtgagaattttattacatgcgttatctgtactgaatgatggcaagaaactagatgaaattgatatgccattctatgaaagattggtagaaaaacgcttgttgaagaaagcatctggtacgttcattgaagaactgttagaagaagcaaatttgatataa

SEQ ID NO: 78 - DNA, 831 bp, Artificial Sequence
  Synthesized ech
atgagcaaatacgaaggccgctggaccaccgtgaaggtcgaactggaagcgggcatcgcctgggtgaccctcaatcgcccggaaaaacgcaatgccatgagccccaccctgaaccgggaaatggtcgacgtgctggaaacccttgagcaggacgctgacgctggcgtgctggtattgaccggtgccggcgagtcctggaccgccggcatggacctgaaggagtacttccgcgaggtggacgccggcccggaaatcctccaggaaaagattcgtcgcgaagcctcgcaatggcaatggaagttgctgcgtctgtatgccaaaccgaccatcgccatggtcaacggctggtgcttcggcggcggcttcagcccactggtggcatgcgacctggcgatctgcgccaacgaagcgaccttcggcctgtcggaaatcaactggggcatcccgcctggtaacctggtcagcaaggccatggccgataccgttggccatcgtcagtcgctgtactacatcatgaccggcaagaccttcgatggtcgcaaggctgccgagatgggcctggtgaacgacagtgtgccgctggccgagctgcgtgaaaccacccgcgagttggcgctgaacctgctggaaaagaacccggtggtgctgcgtgccgcgaagaatggcttcaagcgttgccgcgagctgacctgggaacagaacgaggactacctctacgccaagctcgaccagtcgcgcctgctggacactaccggcggccgcgagcagggcatgaagcagttcctcgacgacaagagcatcaagccaggcctgcaggcctacaagcgctga

SEQ ID NO: 79 - DNA, 786 bp, Artificial Sequence
  Synthesized crt
atggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagatag

SEQ ID NO: 80 - DNA, 792 bp, Artificial Sequence
  Synthesized phaB
atgactttccagcacatcctgttttccatcgaggacggcgttgccttcctttcattgaaccgccccgagcagctgaacagcttcaatacggccatgcacctggaggtgcgcgaagcgctcagacaagtgcgccagagcagtgacgcgcgggtgctgctgctgacggctgaaggccgcggcttctgcgccggccaggacctgtccgaccgcaacgttgccccaggcgccgagatgccagacctgggccagtcgatcgacaagttctacaacccgctggtgcgcaccctgcgcgacctgcctttgccggtgatatgtgcggtcaacggcgtggcggccggtgccggtgccaacattcccttggcctgcgacctggtgctggccgcccgctcggccagtttcatccaggccttctgcaagatcggcctggtgccggactccggcggtacttggctgctgccgcgcttggtcggcatggcccgggccaaggcgctggccatgctgggcgagcgccttggcgccgaacaggccgagcaatgggggctgatctaccgcgtggtggatgatgcagcgctgcgtgatgaagccctcaccctcgcccgccacctcgccgcccagcccacctacggcctgacactgatcaagcgcagcctcaatgccagtttcgacaatggttttgaggcgcagctggagctggagcgcgacctgcagcgcctggcagggcgcagcgaggactaccgcgaaggcgtgaacgccttcatgaacaaacgcacgccagccttcaaggggcgctga

SEQ ID NO: 81 - DNA, 1164 bp, Artificial Sequence
  Synthesized fadA
atggaacaggttgtcattgtcgatgcaattcgcaccccgatgggccgttcgaagggcggtgcttttcgtaacgtgcgtgcagaagatctctccgctcatttaatgcgtagcctgctggcgcgtaacccggcgctggaagcggcggccctcgacgatatttactggggttgtgtgcagcagacgctggagcagggttttaatatcgcccgtaacgcggcgctgctggcagaagtaccacactctgtcccggcggttaccgttaatcgcttgtgtggttcatccatgcaggcactgcatgacgcagcacgaatgatcatgactggcgatgcgcaggcatgtctggttggcggcgtggagcatatgggccatgtgccgatgagtcacggcgtcgattttcaccccggcctgagccgcaatgtcgccaaagcggcgggcatgatgggcttaacggcagaaatgctggcgcgtatgcacggtatcagccgtgaaatgcaggatgcctttgccgcgcggtcacacgcccgcgcctgggccgccacgcagtcggccgcatttaaaaatgaaatcatcccgaccggtggtcacgatgccgacggcgtcctgaagcagtttaattacgacgaagtgattcgcccggaaaccaccgtggaagccctcgccacgctgcgtccggcgtttgatccagtaaacggtatggtaacggcgggcacatcttctgcactttccgatggcgcagctgccatgctggtgatgagtgaaagccgcgcccatgaattaggtcttaagccgcgcgctcgtgtgcgttcgatggcggtcgttggttgtgacccatcgattatgggttacggcccggttccggcctcgaaactggcgctgaaaaaagcggggctttctgccagcgatatcggcgtgtttgaaatgaacgaagcctttgccgcgcagatcctgccatgtattaaagatctgggactaattgagcagattgacgagaagatcaacctcaacggtggcgcgatcgcgctgggtcatccgctgggttgttccggtgcgcgtatcagcaccacgctgctgaatctgatggaacgcaaagacgttcagtttggtctggcgacgatgtgtatcggtctgggtcagggtattgcgacggtgtttgagcgggtttaa

SEQ ID NO: 82 - DNA, 2190 bp, Artificial Sequence
  Synthesized fadB
atgctttacaaaggcgacaccctgtaccttgactggctggaagatggcattgccgaactggtatttgatgccccaggttcagttaataaactcgacactgcgaccgtcgccagcctcggcgaggccatcggcgtgctggaacagcaatcagatctaaaagggctgctgctgcgttcgaacaaagcagcctttatcgtcggtgctgatatcaccgaatttttgtccctgttcctcgttcctgaagaacagttaagtcagtggctgcactttgccaatagcgtgtttaatcgcctggaagatctgccggtgccgaccattgctgccgtcaatggctatgcgctgggcggtggctgcgaatgcgtgctggcgaccgattatcgtctggcgacgccggatctgcgcatcggtctgccggaaaccaaactgggcatcatgcctggctttggcggttctgtacgtatgccacgtatgctgggcgctgacagtgcgctggaaatcattgccgccggtaaagatgtcggcgcggatcaggcgctgaaaatcggtctggtggatggcgtagtcaaagcagaaaaactggttgaaggcgcaaaggcggttttacgccaggccattaacggcgacctcgactggaaagcaaaacgtcagccgaagctggaaccactaaaactgagcaagattgaagccaccatgagcttcaccatcgctaaagggatggtcgcacaaacagcggggaaacattatccggcccccatcaccgcagtaaaaaccattgaagctgcggcccgttttggtcgtgaagaagccttaaacctggaaaacaaaagttttgtcccgctggcgcataccaacgaagcccgcgcactggtcggcattttccttaacgatcaatatgtaaaaggcaaagcgaagaaactcaccaaagacgttgaaaccccgaaacaggccgcggtgctgggtgcaggcattatgggcggcggcatcgcttaccagtctgcgtggaaaggcgtgccggttgtcatgaaagatatcaacgacaagtcgttaaccctcggcatgaccgaagccgcgaaactgctgaacaagcagcttgagcgcggcaagatcgatggtctgaaactggctggcgtgatctccacaatccacccaacgctcgactacgccggatttgaccgcgtggatattgtggtagaagcggttgttgaaaacccgaaagtgaaaaaagccgtactggcagaaaccgaacaaaaagtacgccaggataccgtgctggcgtctaacacttcaaccattcctatcagcgaactggccaacgcgctggaacgcccggaaaacttctgcgggatgcacttctttaacccggtccaccgaatgccgttggtagaaattattcgcggcgagaaaagctccgacgaaaccatcgcgaaagttgtcgcctgggcgagcaagatgggcaagacgccgattgtggttaacgactgccccggcttctttgttaaccgcgtgctgttcccgtatttcgccggtttcagccagctgctgcgcgacggcgcggatttccgcaagatcgacaaagtgatggaaaaacagtttggctggccgatgggcccggcatatctgctggacgttgtgggcattgataccgcgcatcacgctcaggctgtcatggcagcaggcttcccgcagcggatgcagaaagattaccgcgatgccatcgacgcgctgtttgatgccaaccgctttggtcagaagaacggcctcggtttctggcgttataaagaagacagcaaaggtaagccgaagaaagaagaagacgccgccgttgaagacctgctggcagaagtgagccagccgaagcgcgatttcagcgaagaagagattatcgcccgcatgatgatcccgatggtcaacgaagtggtgcgctgtctggaggaaggcattatcgccactccggcggaagcggatatggcgctggtctacggcctgggcttccctccgttccacggcggcgcgttccgctggctggacaccctcggtagcgcaaaatacctcgatatggcacagcaatatcagcacctcggcccgctgtatgaagtgccggaaggtctgcgtaataaagcgcgtcataacgaaccgtactatcctccggttgagccagcccgtccggttggcgacctgaaaacggcttaa

SEQ ID NO: 83 - DNA, 702 bp, Artificial Sequence
  Synthesized fadI
atgacaaccttaagctgtaaagtgacctcggtagaagctatcacggataccgtatatcgtgtccgcatcgtgccagacgcggccttttcttttcgtgctggtcagtatttgatggtagtgatggatgagcgcgacaaacgtccgttctcaatggcttcgacgccggatgaaaaagggtttatcgagctgcatattggcgcttctgaaatcaacctttacgcgaaagcagtcatggaccgcatcctcaaagatcatcaaatcgtggtcgacattccccacggagaagcgtggctgcgcgatgatgaagagcgtccgatgattttgattgcgggcggcaccgggttctcttatgcccgctcgattttgctgacagcgttggcgcgtaacccaaaccgtgatatcaccatttactggggcgggcgtgaagagcagcatctgtatgatctctgcgagcttgaggcgctttcgttgaagcatcctggtctgcaagtggtgccggtggttgaacaaccggaagcgggctggcgtgggcgtactggcaccgtgttaacggcggtattgcaggatcacggtacgctggcagagcatgatatctatattgccggacgttttgagatggcgaaaattgcccgcgatctgttttgcagtgagcgtaatgcgcgggaagatcgcctgtttggcgatgcgtttgcatttatctga

SEQ ID NO: 84 - DNA, 2145 bp, Artificial Sequence
  Synthesized fadJ
atggaaatgacatcagcgtttacccttaatgttcgtctggacaacattgccgttatcaccatcgacgtaccgggtgagaaaatgaataccctgaaggcggagtttgcctcgcaggtgcgcgccattattaagcaactccgtgaaaacaaagagttgcgaggcgtggtgtttgtctccgctaaaccggacaacttcattgctggcgcagacatcaacatgatcggcaactgcaaaacggcgcaagaagcggaagctctggcgcggcagggccaacagttgatggcggagattcatgctttgcccattcaggttatcgcggctattcatggcgcttgcctgggtggtgggctggagttggcgctggcgtgccacggtcgcgtttgtactgacgatcctaaaacggtgctcggtttgcctgaagtacaacttggattgttacccggttcaggcggcacccagcgtttaccgcgtctgataggcgtcagcacagcattagagatgatcctcaccggaaaacaacttcgggcgaaacaggcattaaagctggggctggtggatgacgttgttccgcactccattctgctggaagccgctgttgagctggcaaagaaggagcgcccatcttcccgccctctacctgtacgcgagcgtattctggcggggccgttaggtcgtgcgctgctgttcaaaatggtcggcaagaaaacagaacacaaaactcaaggcaattatccggcgacagaacgcatcctggaggttgttgaaacgggattagcgcagggcaccagcagcggttatgacgccgaagctcgggcgtttggcgaactggcgatgacgccacaatcgcaggcgctgcgtagtatcttttttgccagtacggacgtgaagaaagatcccggcagtgatgcgccgcctgcgccattaaacagcgtggggattttaggtggtggcttgatgggcggcggtattgcttatgtcactgcttgtaaagcggggattccggtcagaattaaagatatcaacccgcagggcataaatcatgcgctgaagtacagttgggatcagctggagggcaaagttcgccgtcgtcatctcaaagccagcgaacgtgacaaacagctggcattaatctccggaacgacggactatcgcggctttgcccatcgcgatctgattattgaagcggtgtttgaaaatctcgaattgaaacaacagatggtggcggaagttgagcaaaattgcgccgctcataccatctttgcttcgaatacgtcatctttaccgattggtgatatcgccgctcacgccacgcgacctgagcaagttatcggcctgcatttcttcagtccggtggaaaaaatgccgctggtggagattattcctcatgcggggacatcggcgcaaaccatcgctaccacagtaaaactggcgaaaaaacagggtaaaacgccaattgtcgtgcgtgacaaagccggtttttacgtcaatcgcatcttagcgccttacattaatgaagctatccgcatgttgacccaaggtgaacgggtagagcacattgatgccgcgctagtgaaatttggttttccggtaggcccaatccaacttttggatgaggtaggaatcgacaccgggactaaaattattcctgtactggaagccgcttatggagaacgttttagcgcgcctgcaaatgttgtttcttcaattttgaacgacgatcgcaaaggcagaaaaaatggccggggtttctatctttatggtcagaaagggcgtaaaagcaaaaaacaggtcgatcccgccatttacccgctgattggcacacaagggcaggggcgaatctccgcaccgcaggttgctgaacggtgtgtgatgttgatgctgaatgaagcagtacgttgtgttgatgagcaggttatccgtagcgtgcgtgacggggatattggcgcggtatttggcattggttttccgccatttctcggtggaccgttccgctatatcgattctctcggcgcgggcgaagtggttgcaataatgcaacgacttgccacgcagtatggttcccgttttaccccttgcgagcgtttggtcgagatgggcgcgcgtggggaaagtttttggaaaacaactgcaactgacctgcaataa

SEQ ID NO: 85 - DNA, 720 bp, Artificial Sequence
  Synthesized fadR
atggtcattaaggcgcaaagcccggcgggtttcgcggaagagtacattattgaaagtatctggaataaccgcttccctcccgggactattttgcccgcagaacgtgaactttcagaattaattggcgtaacgcgtactacgttacgtgaagtgttacagcgtctggcacgagatggctggttgaccattcaacatggcaagccgacgaaggtgaataatttctgggaaacttccggtttaaatatccttgaaacactggcgcgactggatcacgaaagtgtgccgcagcttattgataatttgctgtcggtgcgtaccaatatttccactatttttattcgcaccgcgtttcgtcagcatcccgataaagcgcaggaagtgctggctaccgctaatgaagtggccgatcacgccgatgcctttgccgagctggattacaacatattccgcggcctggcgtttgcttccggcaacccgatttacggtctgattcttaacgggatgaaagggctgtatacgcgtattggtcgtcactatttcgccaatccggaagcgcgcagtctggcgctgggcttctaccacaaactgtcggcgttgtgcagtgaaggcgcgcacgatcaggtgtacgaaacagtgcgtcgctatgggcatgagagtggcgagatttggcaccggatgcagaaaaatctgccgggtgatttagccattcaggggcgataa

SEQ ID NO: 86 - DNA, 1000 bp, Artificial Sequence
  Synthesized ldi
atgcggttcacattgaagacgacggcgattgtgtcggccgccgccctgctggccggtttcgggccgccgccccgcgcggcggaactgccgccggggcggctcgccaccaccgaggactatttcgcgcagcaggcgaagcaggccgtcacccccgacgtgatggcccagctggcctacatgaactacatcgatttcatctcgcccttctacagccggggctgctccttcgaggcctgggagctcaagcacacgccgcagcgggtcatcaagtattcgatcgccttctatgcgtatggcctggccagcgtggcgctcatcgacccgaagctgcgtgcgctcgccggccatgacctggacatcgcggtctccaagatgaagtgcaagcgggtctggggcgactgggaggaagacgggttcggcaccgacccgatcgagaaagagaacatcatgtacaagggccacctgaacctgatgtacggcctctatcagctggtgaccggcagccgccggtacgaagccgagcatgcccacctcacccgcatcatccatgacgagatcgcggccaacccctttgccggcatcgtctgcgagccggacaattattttgtccagtgcaattcggtcgcctacctgagcctgtgggtctatgaccggctgcatggcaccgactaccgggcggccaccagggcctggctggatttcatccagaaggacctgatcgatcccgagcggggcgccttctacctgtcctatcaccccgagtccggcgcggtgaagccgtggatctcggcgtatacgacagcctggacgctcgccatggtgcacggcatggaccccgccttttccgagcgctactacccccggttcaagcagaccttcgtcgaggtctacgacgagggccgcaaggcccgggtgcgcgagacggccggcacggacgacgcggatggcggggtgggcctggcttcggcgttcaccctgctgctggcccgcgagatgggcg

SEQ ID NO: 87 - DNA, 585 bp, Artificial Sequence
  Synthesized dhaB2
gtgcaacagacaactcaaattcagccctcttttaccctgaaaacccgcgagggcggggtagcttctgccgatgaacgtgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgccgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacgtcgacttagtaagggagtga

SEQ ID NO: 88 - DNA, 1668 bp, Artificial Sequence
  Synthesized dhaB1
atgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgagctggacggcaaacgccgggaccagtttgacatgatcgaccggtttatcgccgattacgcgatcaacgttgagcgcacagagcaggcaatgcgcctggaggcggtggaaatagcccgcatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctatgcgccgtttaacgccctggcgctgttggtcggctcgcagtgcggccgtcccggcgtgttgacgcagtgctcggtggaagaggccaccgagctggagctgggcatgcgtggcttaaccagctacgccgagacggtgtcggtctacggcactgaagcggtatttaccgacggcgatgatactccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattaccaaaggcgccggggttcaggggctgcaaaacggcgcagtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacattctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcgacagttcgaggtggtgagcgcggtcaacgacatcaatgactatcaggggccgggcaccggctatcgcatctctgccgaacgctgggcggagatcaaaaatattccgggcgtggttcagcctgacaccattgaataa

SEQ ID NO: 89 - DNA, 1000 bp, Artificial Sequence; identical to SEQ ID NO: 86
  Synthesized ldi (1)

SEQ ID NO: 90 - DNA, 1392 bp, Artificial Sequence
  Synthesized abdD
atgagatcaaaagaagatttcctaaagtccttaaaagatggaagaaatttgtattatagggggaagttagtagaagatataacaacacatcagatcttaaagacagccgcattgcacgcagctaagttatatgaatacgctgatagagtctatgaggataataaaatgggaaaaatgagcaagttctttaaggtaccttggacatctcaagatttgctagatagacataaactaatttacgatttaacgatgtattgtaatggggtatttaacatttcacaagcaataggaagtgatgcgatctttgcccttatgatcacggcaaaacaagttgatagaaaatacggaactgattactcaaaacgtgttgaaaaatattttgagagagttgctaaagaagatttaacgttagccactgcccagactgacgttaagggagatcgaagtaagaggccttctgaacaagttgatccagatatgtatgttagagtagttgatgtgaaaagcgatggaatagttgttagaggagcaaaggctcatacaactcaatctgcggtatctgatgagattattgtcataccaaccagagtaatgagggatagcgataaagattacgcagtagcctttgcggttccagctaatactaaaggtttgaagatgtatattagaccaattgatgaaattgagggcaattcctcctcagtactcagtagaaaagattatgagctagaaacattaaccgtcttcaacgacgttttcgttccttgggatagggtatttttatttaaggaatacgactacgctggaacattggctatgctatttgcaaccttccataggtttactgcattatcgtataggtcagcgaccatgaatctatatttgggagcatctaaagtggcatctcaagtaaatggcattgagaatgaaaagcatgtgagagatgatatagttgatataattctctacaaggaaattatgaggagtagcgcgatagctgcggctgtgtatccagtaaacatggagggtatagctgtgcccaacccgctttttactaatgttggtaaattatactccaatatgcatttccatgatgttgtaagagatttaattgacattgctggggggataatagctactatgccctctcaagaagatttggaaagtgatgaaggaaagaatattgttaaatatttaaggggctcagttgatggagaggaaagagcaaaagtgttaaaactagctaaggaattaggggctagtacgtttactggctatttgctaactggtatgatacatgcggaaggttctatggaagctagcaaaatagagctattcagaagttataattttaaggaggccgagaacttagttaaaagggtattaagctag

SEQ ID NO: 91 - DNA, 1206 bp, Artificial Sequence
  Synthesized paaJ
atgcgtgaagcctttatttgtgacggaattcgtacgccaattggtcgctacggcggggcattatcaagtgttcgggctgatgatctggctgctatccctttgcgggaactgctggtgcgaaacccgcgtctcgatgcggagtgtatcgatgatgtgatcctcggctgtgctaatcaggcgggagaagataaccgtaacgtagcccggatggcgactttactggcggggctgccgcagagtgtttccggcacaaccattaaccgcttgtgtggttccgggctggacgcactggggtttgccgcacgggcgattaaagcgggcgatggcgatttgctgatcgccggtggcgtggagtcaatgtcacgggcaccgtttgttatgggcaaggcagccagtgcattttctcgtcaggctgagatgttcgataccactattggctggcgatttgtgaacccgctcatggctcagcaatttggaactgacagcatgccggaaacggcagagaatgtagctgaactgttaaaaatctcacgagaagatcaagatagttttgcgctacgcagtcagcaacgtacggcaaaagcgcaatcctcaggcattctggctgaggagattgttccggttgtgttgaaaaacaagaaaggtgttgtaacagaaatacaacatgatgagcatctgcgcccggaaacgacgctggaacagttacgtgggttaaaagcaccatttcgtgccaatggggtgattaccgcaggcaatgcttccggggtgaatgacggagccgctgcgttgattattgccagtgaacagatggcagcagcgcaaggactgacaccgcgggcgcgtatcgtagccatggcaaccgccggggtggaaccgcgcctgatggggcttggtccggtgcctgcaactcgccgggtgctggaacgcgcagggctgagtattcacgatatggacgtgattgaactgaacgaagcgttcgcggcccaggcgttgggtgtactacgcgaattggggctgcctgatgatgccccacatgttaaccccaacggaggcgctatcgccttaggccatccgttgggaatgagtggtgcccgcctggcactggctgccagccatgagctgcatcggcgtaacggtcgttacgcattgtgcaccatgtgcatcggtgtcggtcagggcatcgccatgattctggagcgtgtttga

SEQ ID NO: 92 - DNA, 1221 bp, Artificial Sequence
  Synthesized phaD
atgaatgaaccgacccacgccgatgccttgatcatcgacgccgtgcgcacgcccattggccgctatgccggggccctgagcagcgtgcgcgccgacgacctggcggccatcccgctcaaagccttgatccagcgtcaccccgaactggactggaaagccattgatgacgttatcttcggctgtgccaaccaggctggcgaagacaaccgcaacgtggcccacatggcgagcctgctggccgggctgccactcgaagtaccagggaccacgatcaaccgcctgtgcggttccggtctggatgccatcggtaatgcggcacgtgccctgcgctgcggtgaagcggggctcatgctggccggtggtgtggagtccatgtcgcgtgcaccgtttgtgatgggtaagtcggagcaggcattcgggcgtgcggccgagctgttcgacaccaccatcggctggcgtttcgtcaacccgctgatgaaggccgcctacggcatcgattcgatgccggaaacggctgaaaacgtggccgaacagttcggcatctcgcgcgccgaccaggatgcctttgccctgcgcagccagcacaaagccgcagcagctcaggcccgcggccgcctggcgcgggaaatcgtgccggtcgaaatcccgcaacgcaaaggcccagccaaagtggtcgagcatgacgagcacccgcgcggcgacacgaccctggagcagctggctcggctcgggacgccgtttcgtgaaggcggcagcgtaacggcgggtaatgcctccggcgtgaatgacggcgcttgcgccctgctgctggccagcagcgccgcggcccgccgccatgggttgaaggcccgcggccgcatcgtcggcatggcggtggccggggttgagcccaggctgatgggcattggtccggtgcctgcgacccgcaaggtgctggcgctcaccggcctggcactggctgacctggatgtcatcgaactcaatgaggcctttgccgcccaagggctggccgtgttgcgcgagctgggcctggccgacgacgacccgcgagtcaaccgcaacggcggcgccatcgccctgggccatcccctgggcatgagcggtgcccggttggtgaccactgccttgcacgagcttgaagaaacggccggccgctacgccctgtgcaccatgtgcatcggcgtaggccaaggcattgccatgatcatcgagcgcctctga

SEQ ID NO: 93 - DNA, 1203 bp, Artificial Sequence
  Synthesized pcaF
atgcacgacgtattcatctgtgacgccatccgtaccccgatcggccgcttcggcggcgccctggccagcgtgcgggccgacgacctggccgccgtgccgctgaaggcgctgatcgagcgcaaccctggcgtgcagtgggaccaggtagacgaagtgttcttcggctgcgccaaccaggccggtgaagacaaccgcaacgtggcccgcatggcactgctgctggccggcctgccggaaagcatcccgggcgtcaccctgaaccgtctgtgcgcgtcgggcatggatgccgtcggcaccgcgttccgcgccatcgccagcggcgagatggagctggtgattgccggtggcgtcgagtcgatgtcgcgcgccccgttcgtcatgggcaaggctgaaagcgcctattcgcgcaacatgaagctggaagacaccaccattggctggcgtttcatcaacccgctgatgaagagccagtacggtgtggattccatgccggaaaccgccgacaacgtggccgacgactatcaggtttcgcgtgctgatcaggacgctttcgccctgcgcagccagcagaaggctgccgctgcgcaggctgccggcttctttgccgaagaaatcgtgccggtgcgtatcgctcacaagaagggcgaaatcatcgtcgaacgtgacgaacacctgcgcccggaaaccacgctggaggcgctgaccaagctcaaaccggtcaacggcccggacaagacggtcaccgccggcaacgcctcgggcgtgaacgacggtgctgcggcgatgatcctggcctcggccgcagcggtgaagaaacacggcctgactccgcgtgcccgcgttctgggcatggccagcggcggcgttgcgccacgtgtcatgggcattggcccggtgccggcggtgcgcaaactgaccgagcgtctggggatagcggtaagtgatttcgacgtgatcgagcttaacgaagcgtttgccagccaaggcctggcggtgctgcgtgagctgggtgtggctgacgatgcgccccaggtaaaccctaatggcggtgccattgccctgggccaccccctgggcatgagcggtgcacgcctggtactgactgcgttgcaccagctggagaagagtggcggtcgcaagggcctggcgaccatgtgtgtgggtgtcggccaaggtctggcgttggccatcgagcgggtttga

SEQ ID NO: 94 - DNA, 1212 bp, Artificial Sequence
  Synthesized pcaF (1)
atgacattaaaaaacgcttatatcatcgatgccatccgtactccattcggtcgttatgccggtggccttgcacctgtccgtgcagatgaccttggtgctgtgccgattaaagccctcatgcaacgtaacccaagtgtagattgggaacaggtcgatgatgtgatctatggctgtgccaaccaagccggtgaagataaccgtaatgtcggtcgtatgtcagcacttcttgcaggtttaccatatcaggtaccggcaaccactattaaccgtttatgcggttcttcactcgatgccattgccattgcagcccgtgctattaaagcaggtgaagcgaacttggtgattgcaggtggtgtagaaagcatgagccgtgcgccttatgtaatgggtaagtcagacagtgcttttggccgtagccagaagattgaagacaccaccatgggctggcgttttattaacccaaaacttaaagaattgtatggtgtagacaccatgccccagactgccgaaaacgtggctgaacagtttaacgtcaatcgtgcagatcaggaccagtttgccttggtgagccaacaacgcaccgcaagcgcgcaagccaaaggctttttttctaaagaaatcgtggcagttgaaatccctcagcgtaagggtgatgctgttgtgattgatactgatgaacatccacgtgcatcaaccacccttgaaggtttaagcaaacttaaatctgtggttaaagcagatggcacagtaacagcaggcaatgcttcaggtattaatgatggtgcagcagctctactgattgcttctgatgaagcagttcaggcatacaacctaaaaccccgcgccaagattattgcttcaacagcggtgggtgtagaaccacggattatgggctttgctccagcaccagccattaaaaaattacttaaacaagctaacctgactttagatcagatggatgtaattgagctcaatgaagcttttgctgctcaggctttggcagtgacccgtgatttaggtttgccagatgattctcacaaggtaaacccaaatggtggtgccattgctttgggtcatccacttggtgcttcaggtgcacgcatcgtgactacagccttgaaccagcttgaacaaacaggtggtcgctacgctttgtgttcaatgtgtattggggtgggccaaggcatcgcattgattattgagagagtctaa

SEQ ID NO: 95 - DNA, 1164 bp, Artificial Sequence
  Synthesized fadA (1)
atgaaagacgtagtcattgtcgactgtatccggaccccgatgggccggtccaagggcggcgccttccgcaacgtgcgtgcagaagacttgtccgcgcacctgatgaaatccatcctgctgcgcaaccccaacctcgacccgaacgagatcgaggatatctactggggctgcgtgcagcagaccctggagcagggcttcaacatcgcccgcaacgcagccttgctggccggcattcccaagcaggtgggggcggtcaccgtcaaccgcctgtgcggctccagcatgcaggcgctgcacgatgcctcccgcgccattcaggtaggtgatggggatatcttcatcatcggcggtgtcgagcacatgggccacgtgccgatgagccacggggtggacttccaccccggcatggccaagtcggtggcgaaagcctccggcatgatggggctgaccgccgagatgctcggcaagctgcacggcatcagtcgtcagcagcaggacgagtttgccgcccgctcccatcgtcgcgctcacgccgccaccgtggaaggacgtttcgccaaggagatcgtcgggctggaaggccatgacgccagcggcgcccgcttcttctacgactacgacgaggtgatccgccccgagaccacggtggaaaccctgagccagctgcgcccggtgttcgacccggtcaacggcaccgtcaccgccggcacctcgtcggccctgtccgatggcgccgccgccatgctggtgatgagtgcggaccgcgccaaggcgctcggcctcaccccgcgcgccaagatacgtgccatggccgtcgccggctgcgatgccgccatcatgggttacggcccggtaccggccacccagaaggcgctcaagcgggccggcctgaccatcggcgacatcgacctgttcgagctgaacgaggcgtttgccgcccagtccctgccttgcgtgaaggatctgggtctgcaagacgtggtggatgagaaggtgaacctgaacggcggcgccatcgccctgggtcacccgctcggctgctccggcgcccgcatctccaccaccctgctcaacctgatggaagagaaggacgccaccctgggggttgccaccatgtgcatcggcctgggtcagggcatcgccaccgtgttcgaacgagtgtaa

SEQ ID NO: 96 - DNA, 1005 bp, Artificial Sequence
  Synthesized AtoB
atggatattgtgattgttgcggccaagcgtacccccatgggggccttccagggagccttggccaacctgactgcccccgagcttggcgcttgcgccattgctgccgccatagcacaagccgggctcaagggggagcagatcgatgaagcctacatgggcaatgtgctcagtgccggggtggggcaggcacccgcccgtcaggctgtgttgaaggcaggtttgccggagagtgtgccatgcaccactgtcaacaaggtgtgtggttccggcatgaaggcggtgatgctggcggcagacagcttgcgtctgggtgacaccgacatagtgatcgccggtggcatggagagcatgagccgggcgccttacctgctcgacaaggcgcgcagcggttttcgcatggggcatcagagcgtgctggatcatatgttcctcgatggcttgcaggatgcttacgaaggccagttgatggggcattatgcccagttgagtgcggatcgcgccggtctggcccgctccgacatggacgcttttgccatcgcttccctgacgcgtgcgctggctgcccagcagagcggtgctttcaaggccgagctggcccaggttactgtcggtgacaccctgctgctcgccgaggatgagcagcctgccaaggccaggcccgacaagatccctcatctgaaaccggcattcagcaagcagggcaccataacggctgccaatgccagctccatctcggacggagcggcggcgctcatcctgatgcgagccgagacggcggcgcagctgggcctgcctgtgctggccatggcgggttgcaacctgcctcatgacaaggtgaacgtgaacggcggggcctgcgcactggggcatccactgggggcgagtggtgcccgtattctggttacgctcattcatgcactgcatgcgcgcagtctgaaacggggtgtggcaagcctgtgtatcggtggaggggaggcgactgccgtcgccatcgagttgagctaa

SEQ ID NO: 97 - DNA, 1206 bp, Artificial Sequence
  Synthesized pcaF (2)
atgagccgcgaggtattcatctgcgatgccgtgcgcacgccgatcggccgtttcggcggcagtctttccgcggtgcgcgccgacgacctcgcggcggtgccgctgaaggccctggtcgagcgcaacccgggggtcgactggtcggcgctggacgaggtgttcctcggctgcgccaaccaggccggcgaggacaaccgtaacgtggcgcgcatggcgctgctgctggccggtttgccggagagcgtgcccggcgtcaccctcaaccgcctctgcgcctcggggatggacgccatcggcacggcgttccgcgccatcgcctgcggcgagatggagctggccatcgccggcggcgtcgagtcgatgtcgcgcgcgccgtacgtgatgggcaaggccgatagcgccttcgggcgcggccagaagatcgaggacaccaccatcggctggcgcttcgtcaacccgctgatgaaggagcagtacggcatcgacccgatgccgcagaccgccgacaacgtcgccgacgactatcgcgtgtcgcgtgccgaccaggatgccttcgccctgcgcagccagcagcgcgccggcagggcgcaggcggccggtttcttcgccgaggaaatcgtcccggtgacgattcgcgggcgcaagggcgacaccctggtcgagtacgacgagcatccgcgtcccgacaccaccctggaggcgctggcccggctcaagccggtcaacgggccggagaagaccgtcaccgccggcaacgcgtccggggtcaacgacggcgccgccgcgctggtcctggcctccgccgaggcagtggagaagcacggcctgactccgcgcgcgcgggtgctgggcatggccagcgccggcgtcgccccacggatcatgggcatcggcccggtgccggcggtgcgcaagctgctgcggcgcctggacctggcgatcgacgccttcgacgtgatcgaactcaacgaagccttcgccagccagggcctggcctgcctgcgcgaactgggcgtggccgacgacagtgagaaggtcaacccgaacggcggtgccatcgccctcggccacccgctggggatgagcggtgcgcggctggtcctcaccgcgctccatcaacttgagaagagcggcggccggcgcggcctggcgaccatgtgcgtaggcgtcggccaaggcctggcgctggccatcgagcgggtctga

SEQ ID NO: 98 - DNA, 1185 bp, Artificial Sequence
  Synthesized bktB
atgacgcgtgaagtggtagtggtaagcggtgtccgtaccgcgatcgggacctttggcggcagcctgaaggatgtggcaccggcggagctgggcgcactggtggtgcgcgaggcgctggcgcgcgcgcaggtgtcgggcgacgatgtcggccacgtggtattcggcaacgtgatccagaccgagccgcgcgacatgtatctgggccgcgtcgcggccgtcaacggcggggtgacgatcaacgcccccgcgctgaccgtgaaccgcctgtgcggctcgggcctgcaggccattgtcagcgccgcgcagaccatcctgctgggcgataccgacgtcgccatcggcggcggcgcggaaagcatgagccgcgcaccgtacctggcgccggcagcgcgctggggcgcacgcatgggcgacgccggcctggtcgacatgatgctgggtgcgctgcacgatcccttccatcgcatccacatgggcgtgaccgccgagaatgtcgccaaggaatacgacatctcgcgcgcgcagcaggacgaggccgcgctggaatcgcaccgccgcgcttcggcagcgatcaaggccggctacttcaaggaccagatcgtcccggtggtgagcaagggccgcaagggcgacgtgaccttcgacaccgacgagcacgtgcgccatgacgccaccatcgacgacatgaccaagctcaggccggtcttcgtcaaggaaaacggcacggtcacggccggcaatgcctcgggcctgaacgacgccgccgccgcggtggtgatgatggagcgcgccgaagccgagcgccgcggcctgaagccgctggcccgcctggtgtcgtacggccatgccggcgtggacccgaaggccatgggcatcggcccggtgccggcgacgaagatcgcgctggagcgcgccggcctgcaggtgtcggacctggacgtgatcgaagccaacgaagcctttgccgcacaggcgtgcgccgtgaccaaggcgctcggtctggacccggccaaggttaacccgaacggctcgggcatctcgctgggccacccgatcggcgccaccggtgccctgatcacggtgaaggcgctgcatgagctgaaccgcgtgcagggccgctacgcgctggtgacgatgtgcatcggcggcgggcagggcattgccgccatcttcgagcgtatctga

SEQ ID NO: 99 - DNA, 1188 bp, Artificial Sequence
  Synthesized pimB
atgaccgaggccgttatcgtttcaaccgcgcgcacgccgatcggcaaggcgtatcgcggcgccctcaacgccaccgagggtgccacactgctcggccacgccatcgagcacgcggtgaagcgcgccggtatcgacccgaaggaggtcgaggacgtggtgatgggcgcggcgatgcagcagggcgccaccggcggcaacatcgcccgcaaggcgctgctgcgcgccggcctgccggtgactaccgccggcaccaccatcgatcggcagtgcgcctccggcctgcaggcgatcgcgctcgccgctcgctcggtgctgttcgacggcgtcgagatcgcggtcggcggtggcggcgagtcgatctcgctcgtccagaacgacaagatgaacaccttccacgccgtcgatccggcgctcgaggcgatcaagggcgacgtctacatggcgatgctcgacaccgccgaaaccgtggcgaagcgctacggcatctcgcgcgagcgccaggacgagtattcgctggaaagccagcgccgcaccgcggctgcgcagcagggcggcaagttcaacgacgagatcgcgccgatctcgaccaagatgggcgtcgtcgacaaggccaccggcgcggtgtcgttcaaggatatcacgctgtcgcaggacgaaggcccgcggccggaaaccaccgctgaaggtctcgccggtcttaaggccgtgcgtggtgaaggcttcaccatcactgccggcaatgccagccagctgtcggacggcgcctcggccacggtgatcatgagcgacaagacggcggccgcgaagggcctcaagccgctcggcatcttccgcggcatggtctcctacggctgcgagccggacgagatgggcatcggcccggtgttcgcggtgccgcgcctgttgaagcgccatggtctcagcgtcgacgacatcggtctgtgggagctgaacgaagccttcgccgtgcaggtgctgtactgccgcgacaagctcggcatcgatccggagaagctcaatgtcaacggcggcgcgatctcggtcggccacccctacggcatgtcgggtgcacgcctcgccggccacgcgctgatcgaaggccgtcgccgcaaggcgaagtacgcggtggtcacgatgtgcgtcggcggcggcatgggctccgccggcctgttcgagatcgtgcactga

SEQ ID NO: 100 - DNA, 1293 bp, Artificial Sequence
  Synthesized syn_02642
atgaaagatgtcgtcatcgtaagcggcgccagaaccgccgtgggtgcttttggcggatcgctgaaaggcgtgagagttacggatttgggagcgctggtcatcaaagaggccatcaagagagcggggctgcggccggccatcagtgaagaagtgaaaggctgccgttgcgataccttcggagaattcgacaagaccgaaatcaacaagaaatattatgattacgatgaatccctgacccccgtttatttcgacgagtgcatcatggggaactgcctgatcgccggcctgggacagaatcccggccgtcagtccagcatctatgccggtctgcccgaagaaacgaacaccatcacagtgaacaaggtctgcgcatccggcatgaaagccatcaccctggccgcccagatcatcaaagccggcgatgccgacatcatggtggccggcggcatggaaaacatgagcaatgtaccctacgccctgcccgacgcccgctggggataccggatgaacatgcctacgggttccatcatcgacctcatggttcatgatggtctctgggaaatcttcaacggctatcacatgggattcacggcggaaaatatcgcctcccgttatggaatcagccgtcaggcccaggacgagctggccctcatgagccatcagcgcgcccgtgcggccatcgccagcggcgccgtcgccgatgaaatcatccccgttccgctgcccgtgaagaaaggcgcggctccgcagtttttctccgtcgacgagcgtcccatggacaccagcctggaaaagatggcgaagctggccccagtcttcaagaaggacggaaccgtcacggcggccaacgcctcgggtatcaatgacggtgcggcggctgtcgtcgtgatgagcgccgacaaggcaaaggaactgggcctcaaaccgctggcgaagatcctcggctatgcctccggcggcgtcgatccggcatacatgggtctgggtccgattccggcaacccgcaaggtcttcaagaaactcggcctgaccatgaaggacatggacatcgtggaactgaacgaggcctttgcatcccaggccctgggctgcgtccaggaaatgggtgtggatctggacaaaaccaatctcaacggcagcgggatctccatcggtcaccccgtcggctgcaccggcgcccggatcacctacagcttggccatgcagctgcagaagaagaacgcgcacctcggactcgccacgctgtgtatcggtggcggacaggggatggccattgtcctggaaagagtgtaa

SEQ ID NO: 101 - DNA, 1209 bp, Artificial Sequence
  Synthesized phaA
atgcgcagagctgcaatcgtcactcccctccgcacgcccgtcggcaccttcggcggcagcctgcgcccggtgcccgtggaggagctggccgccaccgccgtgcgcgccgtggtggaacgcagcggcatcgatcccgcgcgtatcgatgacgtggtctttgcccagtcctacgccaacagcgaagtgccctgcgtcggccgctgggccgcgctgcaggccggcctgccggtcgaagtgccgggcatgcagctggaccgccgctgcggcggcggcctgcaggccatcgtcacggcctcgatgatggtgcaaagcggcgccgccgacgtggtgatcgcgggcggcgtcgagagcatgagcaatatcgagtactacaccaccgacatgcgctggggcgcgcgctcgggcaatgtgcgcttcttcgaccgcctcgaccgcggccgtgaacgctcccagccggtcgagcgcttcggcaagatctccgggatgatcgagacggccgagaacctggcgcgcgactacggcatcagccgcgaagcggccgatgtcttcgccgcccgcagccacgcacgcgccgcggcagcctgggaggccggccgcttcgatgccgaggtcgtccccgtgcaggtgccccagcgcaagggcgatccggtgcggttcgcgcgcgacgaaggtttccgcccggaaaccacgcgtgaaagcctgggcaagctgcgcacgctgatgccgaacggtaccgtcaccgccggcaacgccagccagcagaacgacgcctcggccgcgtgcctgatcgtggccgaagacaagctggccgaattgggcctcacccccatggcctcgctggtgggctgggcggcggctggctgcgagccctcgcacatgggcatcggcccggtgcccgcggtgaagaagctgctggcgcgcctgaacctgacgctggaccggatggacctggtcgagctgaacgaagccttcgcctgccaggtgctggccgtgctcaagggctgggaatggcatgaccaggacgcgatcgagcagaagctcaacgtgaacggctcgggcatctcgcttggccatccgatcggcgccaccggcgtgcgcatcctggccacgctgctgcacgaactgcagcgccgcggcggccgctatggcctggaaaccatgtgcatcggcggcggccagggtattgccgcggtcttcgaacgctactga

SEQ ID NO: 102 - DNA, 1185 bp, Artificial Sequence
  Synthesized atoB
atgaaaaattgtgtcatcgtcagtgcggtacgtactgctatcggtagttttaacggttcactcgcttccaccagcgccatcgacctgggggcgacagtaattaaagccgccattgaacgtgcaaaaatcgattcacaacacgttgatgaagtgattatgggtaacgtgttacaagccgggctggggcaaaatccggcgcgtcaggcactgttaaaaagcgggctggcagaaacggtgtgcggattcacggtcaataaagtatgtggttcgggtcttaaaagtgtggcgcttgccgcccaggccattcaggcaggtcaggcgcagagcattgtggcggggggtatggaaaatatgagtttagccccctacttactcgatgcaaaagcacgctctggttatcgtcttggagacggacaggtttatgacgtaatcctgcgcgatggcctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcgaaagaaaaccttcgtcttcagtcaagacgaattcccgaaagcgaattcaacggctgaagcgttaggtgcattgcgcccggccttcgataaagcaggaacagtcaccgctgggaacgcgtctggtattaacgacggtgctgccgctctggtgattatggaagaatctgcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaacctgggctttgattctgagaaagtgaatgtcaacggcggggccatcgcgctcgggcatcctatcggtgccagtggtgctcgtattctggtcacactattacatgccatgcaggcacgcgataaaacgctggggctggcaacactgtgcattggcggcggtcagggaattgcgatggtgattgaacggttgaattaa

SEQ ID NO: 103 - DNA, 1179 bp, Artificial Sequence
  Synthesized thlA
atgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctag

SEQ ID NO: 104 - DNA, 1179 bp, Artificial Sequence
  Synthesized thlB
atgagagatgtagtaatagtaagtgctgtaagaactgcaataggagcatatggaaaaacattaaaggatgtacctgcaacagagttaggagctatagtaataaaggaagctgtaagaagagctaatataaatccaaatgagattaatgaagttatttttggaaatgtacttcaagctggattaggccaaaacccagcaagacaagcagcagtaaaagcaggattacctttagaaacacctgcgtttacaatcaataaggtttgtggttcaggtttaagatctataagtttagcagctcaaattataaaagctggagatgctgataccattgtagtaggtggtatggaaaatatgtctagatcaccatatttgattaacaatcagagatggggtcaaagaatgggagatagtgaattagttgatgaaatgataaaggatggtttgtgggatgcatttaatggatatcatatgggagtaactgcagaaaatattgcagaacaatggaatataacaagagaagagcaagatgaattttcacttatgtcacaacaaaaagctgaaaaagccattaaaaatggagaatttaaggatgaaatagttcctgtattaataaagactaaaaaaggtgaaatagtctttgatcaagatgaatttcctagattcggaaacactattgaagcattaagaaaacttaaacctattttcaaggaaaatggtactgttacagcaggtaatgcatccggattaaatgatggagctgcagcactagtaataatgagcgctgataaagctaacgctctcggaataaaaccacttgctaagattacttcttacggatcatatggggtagatccatcaataatgggatatggagctttttatgcaactaaagctgccttagataaaattaatttaaaacctgaagacttagatttaattgaagctaacgaggcatatgcttctcaaagtatagcagtaactagagatttaaatttagatatgagtaaagttaatgttaatggtggagctatagcacttggacatccaataggtgcatctggtgcacgtattttagtaacattactatacgctatgcaaaaaagagattcaaaaaaaggtcttgctactctatgtattggtggaggtcagggaacagctctcgtagttgaaagagactaa

SEQ ID NO: 105 - DNA, 1197 bp, Artificial Sequence
  Synthesized ERG10
atgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatga

SEQ ID NO: 106 - DNA, 2283 bp, Artificial Sequence
  Synthesized pflB
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 107 - DNA, 741 bp, Artificial Sequence
  Synthesized pflA
atgtcagttattggtcgcattcactcctttgaatcctgtggaaccgtagacggcccaggtattcgctttatcacctttttccagggctgcctgatgcgctgcctgtattgtcataaccgcgacacctgggacacgcatggcggtaaagaagttaccgttgaagatttgatgaaggaagtggtgacctatcgccactttatgaacgcttccggcggcggcgttaccgcatccggcggtgaagcaatcctgcaagctgagtttgttcgtgactggttccgcgcctgcaaaaaagaaggcattcatacctgtctggacaccaacggttttgttcgtcgttacgatccggtgattgatgaactgctggaagtaaccgacctggtaatgctcgatctcaaacagatgaacgacgagatccaccaaaatctggttggagtttccaaccaccgcacgctggagttcgctaaatatctggcgaacaaaaatgtgaaggtgtggatccgctacgttgttgtcccaggctggtctgacgatgacgattcagcgcatcgcctcggtgaatttacccgtgatatgggcaacgttgagaaaatcgagcttctcccctaccacgagctgggcaaacacaaatgggtggcaatgggtgaagagtacaaactcgacggtgttaaaccaccgaagaaagagaccatggaacgcgtgaaaggcattcttgagcagtacggtcataaggtaatgttctaa

SEQ ID NO: 108 - DNA, 2364 bp, Artificial Sequence
  Synthesized pfl
atgaaaaccgaagttacggaaaatatctttgaacaagcttgggatggttttaaaggaaccaactggcgcgataaagcaagcgttactcgctttgtacaagaaaactacaaaccatatgatggtgatgaaagctttcttgctgggccaacagaacgtacacttaaagtaaagaaaattattgaagatacaaaaaatcactacgaagaagtaggatttcccttcgatactgaccgcgtaacctctattgataaaatccctgctggatatatcgatgctaatgataaagaacttgaactcatctatgggatgcaaaatagcgaacttttccgcttgaatttcatgccaagaggtggacttcgtgttgctgaaaagattttgacagaacacggtctctcagttgacccaggcttgcatgatgttttgtcacaaacaatgacttctgtaaatgatggaatctttcgtgcttatacttcagcaattcgtaaagcacgtcatgctcatactgtaacaggtttgccagatgcttactctcgtggacgtatcattggtgtctatgcacgtcttgccctttacggtgctgattaccttatgaaggaaaaagcaaaagaatgggatgcaatcactgaaattaacgaagaaaacattcgtcttaaagaagaaattaatatgcaataccaagctttgcaagaagttgtaaactttggtgctttatatggtcttgatgtttcacgtccagctatgaacgtaaaagaagcaatccaatgggttaacatcgcttatatggcagtatgtcgtgtcattaatggagctgcaacttcacttggacgtgttccaatcgttcttgatatctttgcagaacgtgaccttgctcgtggaacatttactgaacaagaaattcaagaatttgttgatgatttcgttttgaagcttcgtacaatgaaatttgcgcgtgcagctgcttatgatgaactttattctggtgacccaacattcatcacaacatctatggctggtatgggtaatgacggacgtcaccgtgtcactaaaatggactaccgtttcttgaacacacttgatacaatcggaaatgctccagaaccaaacttgacagtcctttgggattctaaacttccttactcattcaaacgttattcaatgtctatgagccacaagcattcttctattcaatatgaaggtgttgaaacaatggctaaagatggatatggcgaaatgtcatgtatctcttgttgtgtctcaccacttgatccagaaaatgaagaaggacgtcataacctccaatactttggtgcgcgtgtaaacgtcttgaaagcaatgttgactggtttgaacggtggttatgatgacgttcataaagattataaagtattcgacatcgaacctgttcgtgacgaaattcttgactatgatacagttatggaaaactttgacaaatctctcgactggttgactgatacttatgttgatgcaatgaatatcattcattacatgactgataaatataactatgaagcagttcaaatggccttcttgcctactaaagttcgtgctaacatgggatttggtatctgtggattcgcaaatacagttgattcactttcagcaattaaatatgctaaagttaaaacattgcgtgatgaaaatggctatatctacgattacgaagtagaaggtgatttccctcgttatggtgaagatgatgatcgtgctgatgatattgctaaacttgtcatgaaaatgtaccatgaaaaattagcttcacacaaactttacaaaaatgctgaagctactgtttcacttttgacaattacatctaacgttgcttactctaaacaaactggtaattctccagtacataaaggagtattcctcaatgaagatggtacagtaaataaatctaaacttgaattcttctcaccaggtgctaacccatctaataaagctaagggtggttggttgcaaaatcttcgctcattggctaagttggaattcaaagatgcaaatgatggtatttcattgactactcaagtttcacctcgtgcacttggtaaaactcgtgatgaacaagtggataacttggttcaaattcttgatggatacttcacaccaggtgctttgattaatggtactgaatttgcaggtcaacacgttaacttgaacgtaatggaccttaaagatgtttacgataaaatcatgcgtggtgaagatgttatcgttcgtatctctggttactgtgtcaatactaaatacctcacaccagaacaaaaacaagaattaactgaacgtgtcttccatgaagttctttcaaacgatgatgaagaagtaatgcatacttcaaacatctaa

SEQ ID NO: 109 - DNA, 2325 bp, Artificial Sequence
  Synthesized pfl (1)
atggcgactgttaaaacaaatgcagatgtttttgaaaaagcctgggaaggctttaaaggtactgactggaaagaaaaagccagcgtttctcgcttcgttcaagctaactacacaccatatgatggtgatgaaagcttcttagcaccagctactgaacgctctcttaaaatcaagaaaatcattgaagacactaaagctgaatacgaagcaactcgtttcccaatggacactcgtccaacatcaatcgcagatattcctgccggctatattcaaaaagacgatgaattaatctacggtattcaaaatgatgagttgttcaaattgaatttcatgccaaaaggtggtatccgtatggcagaaacagcacttaaagaacatggttatgaaccagatcctgctgttcatgaaattttcacaaaatacactactacagtaaatgacggaattttccgcgcttatacatctaatatccgccgtgcccgtcacgctcacacagtaactggtcttccagatgcttactcacgcggacgtatcatcggtgtttatgctcgtcttgctctttatggtgcagactacttgatgcaagaaaaagttaacgactggaacgctatcacagaaatcgacgaagaatctattcgtcttcgcgaagaagttaacatgcaataccaagctcttggtgaagttgttaaacttggtgacctttacggacttgatgtccgtaaaccagccatgaacgttaaagaagctatccaatgggtaaacatcgccttcatggccgtatgtcgtgttatcaacggtgctgctacttctcttggacgtgtgccaatcgttcttgatatctttgctgaacgtgaccttgctcgtggtactttcacagaatcagaaatccaagaatttgtcgatgactttgtcttgaaacttcgtactgtaaaatttgctcgtactaaagcttacgacgaactttactctggtgacccaacattcatcactacatctatggctggtatgggtgctgacggtcgtcaccgtgttactaaaatggactaccgtttcttgcacacacttgataatatcggtaacgctccagaaccaaacttgacagttctttggactgataaattgccatattcattccgtcgctactgtatgaaaatgtcacacaaacactcgtcaatccaatacgaaggtgtgacaacaatggctaaagatggttacggtgaaatgtcatgtatctcatgttgtgtatcaccacttgacccagaaaacgaagaacaacgtcacaacatccaatactttggtgctcgtgtaaacgtccttaaagctcttcttactggtttgaacggtggttatgacgacgtccacaaagactacaaagtatttgatatcgaaccagttcgtgatgaaatccttgatttcgaaacggttaaagctaatttcgaaaaatctcttgattggttgacttcaacttacgtagatgcccttaacatcattcactacatgactgataaatacaactacgaagctgttcaaatggcattcttgccaactaaacaacgtgccaacatgggattcggtatctgtggtttcgctaataccgttgatactttatcagcaatcaaatacgctactgttaaaccaatccgtgacgaagatggttacatctacgactacgaaacaactggtgacttccctcgttggggtgaagatgaccctcgttctaacgaacttgctgaatggttggtagaagcttacactactcgtcttcgtagccacaaactttacaagaacgctgaagctactgtatcacttcttacaatcacttcaaacgttgcttattctaaacaaactggtaactctccagttcacaaaggtgtttaccttaacgaagatggtactgtaaacctttctaaacttgaattcttctcaccaggtgccaacccatctaacaaagctcgtggtggttggttgcaaaacttgaactctcttgcaagccttgacttctcatatgctgcagatggtatctcacttacaactcaagtttctccacgcgctcttggtaagacatttgatgaacaagttgataacttggtaactatccttgatggttacttcgaaaacggtggacaacacgttaacttgaacgtcatggaccttaaagatgtttatgacaagattatgaatggtgaagatgttatcgttcgtatatcaggttactgtgtcaacactaaataccttactaaagaacaaaagacagaattgacacaacgcgtcttccacgaagttctttcaatggatgatgttgctgaaactgttgctgctaaataa

SEQ ID NO: 110 - DNA, 789 bp, Artificial Sequence
  Synthesized act
atgactgaaatagattacggaaaagtgacaggaatgattcattcaacagaaagttttggttctgtggatgggcctggtgttcgctttgtcatttttatgcaaggctgcaagatgcgttgccaatattgtcacaatccagatacttgggcattagagacaaataattctcgtgaacgcactgttgatgatgttttagcagaagctttgcgttatcgacatttctggggtgaaaatggtgggattaccgtttcaggtggtgaagccatgttgcaaattgagtttgtaacagccctttttaccaaggctaaagaattaggaattcattgcacgcttgatacgtgtggttttacgttccgagatacgcctgaatatcacgaaattgtggataagttactagctgtgacggatttagttcttttagatttaaaagaaatcaatcctaaacaacacattgttgtaacacgtcaacccaatactaatattctagcttttgctcgttatttgtctgataagggtgttccagtctggattcgtcatgtcttggttccaggattgaccgattttgatgaagacttaattgagctagggaaatttgttgaaacgttaaaaaacgtggataaatttgaaattttgccttatcataccttgggtgaattcaagtggcgtgaattgggaattccttatacccttgaaggggttaaaccaccgactagagaacgtgtccaaaatgctaaaaagcttatgcatacagagtcttacacagactacatgaaacgcattcatcactag

SEQ ID NO: 111 - DNA, 717 bp, Artificial Sequence
  Synthesized Clo1313_1716
atgacattaaagggcaggatacactcatttgaatcttttgggacactggacggaccgggtataagatttgtggttttcatgcagggctgtcccttgcgttgtatatattgccacaacagggatacctgggatgttaatgcggggagtgagtacactccccggcaagtaattgatgaaatgatgaaatacatagactatataaaggtctccggaggcggaataactgttaccggcggggagcctgttctccaggccgattttgtggccgaggtgttcagacttgcaaaagagcagggagtgcatacggcgctggataccaatggatttgctgacatagagaaggttgaaaggcttataaaatacaccgatcttgtattgctggatataaagcatgcccgggaggataaacataagataattaccggtgtgtccaacgaaaaaatcaagcgttttgcgctgtatctttcggaccagggagtgcctatctggataagatatgtccttgtccccggatataccgacgatgaagatgaccttaaaatggcggctgatttcataaaaaagcttaaaacggtggaaaaaatcgaagttcttccttatcacaacatgggagcatacaaatgggaaaaacttggtcagaaatacatgcttgaaggagtaaaggggccgagtgcgcaagaggtggaaaaagcaaagaggattctgtcaggcaaataa

SEQ ID NO: 112 - DNA, 1269 bp, Artificial Sequence
  Synthesized OleTJE
atggcaacacttaagagggataagggcttagataatactttgaaagtattaaagcaaggttatctttacacaacaaatcagagaaatcgtctaaacacatcagttttccaaactaaagcactcggtggtaaaccattcgtagttgtgactggtaaggaaggcgctgaaatgttctacaacaatgatgttgttcaacgtgaaggcatgttaccaaaacgtatcgttaatacgctttttggtaaaggtgcaatccatacggtagatggtaaaaaacacgtagacagaaaagcattgttcatgagcttgatgactgaaggtaacttgaattatgtacgagaattaacgcgtacattatggcatgcgaacacacaacgtatggaaagtatggatgaggtaaatatttaccgtgaatctatcgtactacttacaaaagtaggaacacgttgggcaggcgttcaagcaccacctgaagatatcgaaagaatcgcaacagacatggacatcatgatcgattcatttagagcacttggtggtgcctttaaaggttacaaggcatcaaaagaagcacgtcgtcgtgttgaagattggttagaagaacaaattattgagactcgtaaagggaatattcatccaccagaaggtacagcactttacgaatttgcacattgggaagactacttaggtaacccaatggactcaagaacttgtgcgattgacttaatgaacacattccgcccattaatcgcaatcaacagattcgtttcattcggtttacacgcgatgaacgaaaacccaatcacacgtgaaaaaattaaatcagaacctgactatgcatataaattcgctcaagaagttcgtcgttactatccattcgttccattccttccaggtaaagcgaaagtagacatcgacttccaaggcgttacaattcctgcaggtgtaggtcttgcattagatgtttatggtacaacgcatgatgaatcactttgggacgatccaaatgaattccgcccagaaagattcgaaacttgggacggatcaccatttgaccttattccacaaggtggtggagattactggacaaatcaccgttgtgcaggtgaatggatcacagtaatcatcatggaagaaacaatgaaatactttgcagaaaaaataacttatgatgttccagaacaagatttagaagtggacttaaacagtatcccaggatacgttaagagtggctttgtaatcaaaaatgttcgcgaagttgtagacagaacataa

SEQ ID NO: 113 - DNA, 684 bp, Artificial Sequence
  Synthesized PadA1
atgttcaactcacttctatccggtactactacaccaaactccggccgtgcatctcctcccgccagcgaaatgcccatcgataatgatcacgtggccgttgcccgtccagctccccgccgccgccgcattgtagtagccatgacgggtgccactggagccatgctcggcatcaaagtcctaattgctctgcgccgtctaaatgtggagacacacctggtgatgagtaaatgggcggaggctacgatcaaatacgagactgactaccatccctcaaacgtgcgagcgctggccgactacgtgcacaacatcaatgacatggccgccccagtatccagcggctcattccgcgcggacggaatgattgtggtaccgtgcagcatgaaaacattggctgctatccactcgggcttttgcgacgatctcatttcaaggacagcagatgtgatgctcaaggagcgcaggcggttggtgctagtagcgcgggagacgccattgagcgagatccatctgcgaaacatgttggaggttacacgcgctggggcagtcatcttccccccagtaccggcgttctacatcaaggccggaagtatcgaggacctcatcgaccagagtgttggacgaatgttggatttatttgacctcgacacgggggattttgaacgttggaatggatgggaaaaataa

SEQ ID NO: 114 - DNA, 1555 bp, Artificial Sequence
  Synthesized ohbA1
atgtctgcgcaacctgctcacctgtgtttccgctccttcgtcgaagccctcaaggtcgacaacgaccttgttgaaatcaataccccaattgaccccaatctcgaagctgctgctattacccgccgagtatgtgagaccaacgacaaggctcctttattcaacaacctcatcggcatgaaaaatggcctcttccgtatacttggggctcctggctctctcaggaagtcgtctgctgatcgctacggccgccttgctcgtcacctagccctcccacctacggcctcaatgcgtgagattctcgataagatgctctccgccagcgatatgcctcccatccctccgaccattgttcccaccgggccatgcaaggagaacagcttagatgactctgaattcgaccttaccgaactccccgttcctcttattcacaaatcggatggtggtaaatacatccaaacctatggcatgcacattgtgcagtctccggatggaacctggaccaactggtctattgcccgtgcgatggtccatgacaagaaccatctgaccggcctggttattccccctcagcacatctggcagattcaccagatgtggaagaaggaaggccgcagtgacgttccctgggctttggcctttggtgtcccacccgctgccattatggcctctagcatgcctattcccgatggtgtcaccgaagctgggtacgtgggagctatgacgggatcctccctggagcttgttaaatgtgatacgaacgatctatatgtccccgctacctcagaaatcgttctcgagggcacactctctatcagcgagacaggcccagagggacctttcggtgagatgcatggttacatcttccccggggatactcacctcggcgccaaatacaaggttaaccggatcacctaccgcaacaacgccatcatgcccatgtcttcttgtggccgcttgacggatgaaacggtaagtttagtccctgtcctgccatttatagccaaggactaacacggtctagcacaccatgatcggctctctggctgcggcggagatccgtaagctctgccagcagaatgacctccctatcactgatgccttcgctcctttcgagtctcaagttacctgggttgctctgcgggtcgatactgagaagctacgtgccatgaagacaacgtctgagggattccgcaagagagtgggagacgtcgtcttcaaccacaaggccggatacaccattcatcgtctggtgttggtcggtgacgacattgatgtctatgaaggaaaggatgtgctctgggcgttctccacccgttgccgtcctggtatggacgagactttgtttgaggatgttcgtgggttccccttgattccgtatatgggacacgggaatgggcccgcccaccgcggcggaaaggttgtgtccgacgctcttatgccgactgagtacaccactggtcgcaactgggaggctgctgacttcaaccaatcttatcccgaggatctgaagcagaaggtgttggacaactggacgaagatgggtttcagcaactaa

SEQ ID NO: 115 - DNA, 2224 bp, Artificial Sequence
  Synthesized sdrA
atgggcaccccgataaatcgtgaagagattgaccgcgtgctgcgaatgaaacgcaatcagcgcgaggctcgagcgtgttatccttgccgccagcgcaaggtgaaatgcgacagcactcagccgtgtcgaacatgtcgccgacgaggccatccccaaatatgtgtgtatgaccaagattcgtctgggtctaaaaaggctcgtagcaccggccaaagacgttcctctgctgcttctcgtggaacaaatcagacaccaaccgccgagcaggcattcgatgccgaaccacaatctctgccctcagcgcgcagtttaccagaagtccagccaaaaacaagacagtactatagtactcgaatcccgtcttccgatggccccgataatgatcttatctactcgggcgacaactcggtattgtcttatttgcgcaaccggacgcaagataccaatggctccatgacccgtgaggtgggctctgttctaggcctgcaaaatacctacggcagttatccatttatggactttcggacaccccaggaccggtggaaggagcttctacgtattattccgcagcgagcggaactgttgaagtaagcacatcttattgttgtttttgataacctctaacggatagcaggttcttccatttctacagaatatcagcttaccctttcaatccgatcatacttgacattgagagatttgagcaagatgtgtgttcatacctcaatgatcttgcagcaggagagctgcagaacacttcaaagatttgcgaacgttgggccactgatcggtctgtcgggctgatcagcctgctacttgcggccttggcttccggtgcgcattattctgacctggattacatgcaaagaacagagctatgccaggattttggtacgtaaccagtatctttacctatgcatgtttgactaaacaggagaagcaaaacgatcctttcaagctcttcgactagccaattttcttttccgtccgacgatggatataatacaagcacttctaatcataggaaacactctgcaaaacaatggccagtctgatgcagcatgggttttgttagggacaacagtccgtctcgcgcagacattaggtcttcacacagaaaagagtgtagcacgcctaccggatcatgtcaaatacaaagcacgaaagctatggtacataaaccatgctacaggtaacgacacaagctgacgcggctacaggtacactgtcgtttggcaagattgcctgctctgtttatgttacgaccggcctcgcgtagtctctatgaccgggtgggctccagattattcaatcctctcgagcagcgaactatctttcacagaagctatgtattttctatgccaaactgccttaaatatgatcacaacagacggaccggagatatcggaaaatgcgcgacagcttgacattttggccacgattgatagcctcaaccaacgcactcagccatatctgcgtgaccgccaggaatgcaaaaccctccaacacaatctggagcacctggcgttacgaatgcacatgtctctagttatttccgtcctgacacgtccagcactgaagcgcactgtaatgcaagacgcgtcctatgacatcttgcgcacccgcgccaaattgagcctgatcgacgcctctagggcctttttggattttcaggctctgagtgtggtacccctccgaagctggtcaatggtgcacacggtgcttagttccactttacttctctgcatttgggaggagacccgaaacgatcccgagtgtcgtgatttacagcaaaaggtgattgaggtcttttctgccgctggcacagtgggcacagtggagaacacagcatcggagaatgggcaatggctatcggaacggcatatacgagcgctaatcacactgcgcaattcggtccgaacggcagtcgaacgtgaaaagggggaggcaagcgttgggacagaacgcgcggagcagccccagcctttttttcctgtctatgggtatgtgcacccgctattgtctgataagtggagctgtgcgatggatgctgattttgcagtatgccgaacgggatcccggatgacttcggtcaagacttctcaccagcaagctatcttgactccattatgaacggtatgctgaggctcccgactatttatcgatcgaactaaccgtcgtagtacccatgtttgacttatcccaagagctgggttttctttga

SEQ ID NO: 116 - DNA, 723 bp, Artificial Sequence
  Synthesized padA1
atgctctcctccttccttccttccggcaccaacacatcaaactccggtcatcacagccccgacaatgcatccgaaacacaatcaaccacacagtccgcaccactcgagcacatatccaccgcaatgccaccagtcccaaccaaaggtcgacgcaaacgaatcgtcgtagccatgaccggagccaccggctcaatcctgggaatcaaagtcctcatcgcccttcgccgcctcaacatcgaaacccacctcgtaatcagcaaatgggccgaagcaaccataaaatacgaaacagactatcacccgcggaatgttcgtgccctagccgactacgtccacaacataaacgacatggcggcacccatatccagcgggtccttcaagaccgacggcatgatcgtcgtcccatgttccatgaaaacactcgccgctatcaactccgggttctgtgaagatctcatctcccggactgcagacgtcatgctcaaggagcgcaggaagctggttcttgttgctagggaaacgcctcttagtgatattcatcttcgcaatatgctttctgtgtctcaggctggggctattatcttcccgcctgtgccggcgtactatatcaaggcggcgtctgtggatgaacttgtggatcagagtgttgggcgcatgttggatctgtttgatctggatacggctgattttgctagatgggagggttggaagaaggataactga

SEQ ID NO: 117 - DNA, 1512 bp, Artificial Sequence
  Synthesized ohbA1 (1)
atggccgcgattaacgaagtcgatcattccttccgcgcctttgtcgaagccctcaaggccgacgatgacttggtcgagatcaacaccgagatcgactctaacctggaagccgccgcgatcactcgtcttgtctgcgagaccgatgacaaagcccccctcttcaataacctcaaaggcatgggaaagaatggcctcttccgtatcctgggcgctccgggctctctcagaaagtccaaacgtgaccgctacggccggctcgcccgccacctggcgctgcctcctactgccagcatgaaggaaatccttgacaagatgctctccgcctctcagctacctcccattgaccctaagattgtagagactggtcctgtgaaggacaattcccttgaaggcgacgaaatcgacctcactgcgctcccagtgcccatggtgcacaagtctgacggcggcaaatatctacaaacatacggaatgcatgtcgtgcagtctcctgatggaaagtggacgaactggtctatcgcccgtgcgatggtcaaggacaagaaccatttgacaggcctggttattgagccccagcatatttggcaaatccaccagatgtggaaaaaggagggaaaggatgtcccgtgggctctatgcttcggagttcctcctgccgctatcatggcatcatcgatgcccatcccggatggtgtaactgaggctggctacgttggtgccatgactggtcgcgccttggagctcgtcaagtgcgacaccaaccatctctacgtccctgccaatgcggagattgtcctcgagggtaccctctccatcactgaaaccgccgatgaaggccccttcggtgagatgcacggctacgtcttccccggcgatagccacaagtgtcccgtttacaaagttaacaagatcacctaccgcaccgatgctatcctgcccatgtccgcctgcggtcgtcttaccgacgagacccatactatgattggctcgttggctgccgctgagattcgtaaaatttgccaactggccggcctccccatcaccgacaccttttctcccttcgaggcacaggttacctgggtggctctcaaagttgacaccgcaaagcttcgtcaaatgaagctagcccctaaagagcttcagaaatgggtcggagacgtggtctttaaccacaaggctgggtacactatccaccgcctggtcctggttggcgatgatattgacccgtatgagtggaaggatgtcatgtgggctttcgcaacacggtgtcgacccaatgctgatgaaatgttctttgaagacgtccgtggtttcccccttatcccgtatatgggtcacggcacggggtcgcccaccaagggtggtaaggtggtttccgacgctctgatgcccacagagtataccacaggtgctgattgggaagctgctgactttgagcactcctatccggaggagatcaaggccaaggtgagggccaactgggaggctttgggattcagaaaacaggattaa

SEQ ID NO: 118 - DNA, 723 bp, Artificial Sequence; identical to SEQ ID NO: 116
  Synthesized sdrA (1)

SEQ ID NO: 119 - DNA, 1050 bp, Artificial Sequence
  Synthesized Mvd
ttgaatgatcttaatgtttatggtgaaaaaataagaaatatgcttcttgaacttggcatttataataaatcagatgattattcacctgatattaaatacaataaaacgttccacgcaaatggatacccaataacaggtctttataaattccttggatactatgatagggataataacatagccaactttccatcgatatcgttcacaacgaacttttcatcatgtgatgttacatgcagggtattaagatcaggcaatgacaggatcatattcaacgggaaaaacaatgaaaagtattacaaaagggctgaaaaggccctgtcatttctcaggaaaaaatatagaatagatgcagcatttgagtttaacatcaggataaatagaagatacagggatgccaaaggccttggagaatcggcagccgtggcatcggcaaccgccagggccgttgccgcagcagtctttggcatggatgctgcaaaagacaggggttttgtatcatacctggccaggcatgtctctggctccggtaccagatctgcggcaggaaacctttcaatgtggctttcatatcctggaatagacgatttatcttcaattggcttcgaaataagaaaagacgatttattccatttctatgccataccaatgagatcaagaatagagacattaaatgcacatgattatgcatcctcatcaattttttataatgcatgggtcaaatcaaaattttttgatataatagacatcattgaaaacaaattcaatacaaggatgatgcttgaatactccatgaaggatatgtacaggctgcaggcgcttttaatatcctctggatatatcatatatgaaaagcattatttagacattataagaaaattaagatcatcattaaataactacaaaaacgtttatttcacatctgatacaggaacaagcattgttgttatgtcaacatcaatgaatgagctttcaaggttcgttaacgatcttgatcttgatggtataagcggcaattttccagagaagatcattatagaggaactatga

SEQ ID NO: 120 - DNA, 975 bp, Artificial Sequence
  Synthesized mvd
atggaaaattacaatgttaagacaagggcgttcccaacaataggcataatactgcttggtgggatctcggataaaaagaacaggataccgctgcatacaacggcaggcatagcatatactggtataaacaatgatgtttacactgagacaaagctttatgtatcaaaagatgaaaaatgctatattgatggaaaggaaattgatttaaattcagatagatcaccatcgaaggttattgataaattcaagcatgaaatacttatgagagtaaatcttgatgatgaaaataacctttcaattgattcaaggaactttaatatattaagtggcagctcagattctggggccgctgcactgggagagtgcatagaatcaatttttgaatacaatataaatatatttacatttgaaaacgatcttcagaggatatcagaaagtgttggaagaagcctttacggtggtttaacagtaaactatgccaatggcagggaatcattaacagagccattacttgagcctgaggcatttaataactttacaataattggtgcacattttaacattgatagaaaaccatcaaatgagattcatgaaaatatcataaaacatgaaaattacagggaaagaataaaaagtgctgagagaaaggcgaaaaaacttgaggagctatcaaggaatgcaaacataaagggtatctttgaacttgcagaatccgatacagtggaataccataaaatgctccatgatgttggcgttgacataataaatgatagaatggagaacctcattgaaagggtaaaagaaatgaaaaataacttctggaattcatacatagttaccggcggcccgaacgtttttgtaataacagagaaaaaggacgttgataaggcaatggaaggattaaatgatctgtgcgatgatataagattattaaaagttgcaggaaagccacaggtcatttcaaaaaacttttaa

SEQ ID NO: 121 - DNA, 1191 bp, Artificial Sequence
  Synthesized mvd (1)
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 122 - DNA, 1182 bp, Artificial Sequence
  Synthesized mvd (2)
atggacaaaaaggtttatcaatgcaccgttagtgcgcctgttaatattgcagtaattaaatactggggaaagagagatgtggcattgaacttgcctaccaatagctcgatcagtgtgaccctttctcaagatgacttacgtactgttactacagctagttgtagcgagaagtttgagaatgatacactgtggttaaatggaaacgctgaggaaatctttgccaataaacgacttcgtgtctgtgtagaggaactgcgtaaagctagattagatctcgaagaggaaaatgatgatcttgacaagattggtgcattgaagcttcatgtcgtttcagaaaacaacttccctactgctgctggtttggcatcttcagctgctggttatgctgctttttgtgaagcaatcgctagattgtacgatttaccatggacacccactcaattatctcgcattgctagacaggggtctggaagtgcttgtcgtagcttgtttgggggctatgtagcctgggagatgggcgagcttcatagcggtgctgatagtgtagcagttcaagttgaacctgttgaaaattggcccgaaatacgtgttgctgttttagtagcgtccgctgccaaaaaaggggtttcctcaacagctggcatgcaagctacagttgcatcttctaccttgttccaacatcgtattcaaaacatcgttccacaacgtatccaagaaatgaagaccgccattcgtgagcgtgattttgagacttttgcgaagcttaccatgactgattccaatcaattccatgcgtgctgccttgatacttttccccctatcttttacttgaacgatacttcacgtgcggttatccgagttgttgagaatataaatgctactgctggaaagaccattgctgcctatacatttgatgctggcccaaatgctgttatttacttcttggaagaaaactccgagattgtattaaatacactttatgctgttactaaaaatgctgaaggatggagcaagcagtatggctcttcccccgttactgttgattctgctgcagccaatattgtatcatctggtataagccgagttatcttaactcgagtgggtaatgggcctcgagttttgacgattgacgaatctttgatcgatgcatctggcaaccctaaatttataggaagtcattaa

SEQ ID NO: 123 - DNA, 972 bp, Artificial Sequence
  Synthesized mvd (3)
atgaaagcgacagcgacggcccacccgatccaggggctggtgaagtaccacgggatacgcgaccccgaactccggacgccgtatcacgattcgatcagcctctgcactgcgccgagtaactccacgacgacggtcgccttcgaacccgagcgtcccgaggacgagtacgtcatcgacggcgaacacatcgacgggcgcggggccgagcgcatccggaccgtcgtcgataacgttcgcgaacgggccgatctcgacgagcgcgtccgcgtcgcaagtgagaacaacttcccgtcgaacgtcggctttggctcctcggcgtcgggattcgcggcgctggcgactgctctcgttgaggccgctggcctggacctctcacgcccggagatctcgacgattgcccgccgcggctcgacctcggcggcgcgggcggtcacgggtggcttttcggatctgcgggcgggcagtaacgacgccgactgccgttcgaagcgactcgacgtccccttggaggatgacgttcgcatcgtcggcgcagtgattcctgcatacaaagagaccgaggcggcccacgaggaggccgccgagagccacatgttcgagggccgactcgcccacgtccacgagcaactcgcggacatgcgcgacgcgctcggtcgcggggacttcgagcggtccttcgagatcgccgaacacgacacactctcgctggcggcgacgacgatgaccggaccgagcggctgggtctactggcaacccgagagcctcgaagtcttcgagacggttcgggaccttcgcgacgacggggttcccgtctacttctccggggataccggcgcaagcatctacgtcaacaccacggccgagtacgtcgaccgcgtcgaatcggcgatcgaaaccctcgggatcgagacgctcacctggcgcgtcggtggccccgcgcgcgtccgtgatcccgagaaggcactgttctga

SEQ ID NO: 124 - DNA, 984 bp, Artificial Sequence
  Synthesized mvd (4)
atgaaagcgaccgccatggcccacccgattcaggggctggtcaagtatcacgggatgcgagacgagatcgagcgcctgccgtatcacgacagtatcagtctctgtacggccccgagccacactcgcacgaccgtggagttctcgatggactacgaggaggacacgttcgtcgtcgacggcgaggaactcgacggccgggcctacgagcgcgtcgaagccgtcgtcgagaaggctcgttcgaagtccgacgcggcccacaccgtctatccggttcgcctcgagagcgagaacagtttcccgtcgaacgtcgggctgggctcctcttcctcgggcttcgccgccgccgcgatggcgctggccgaggccgccgaactcgacgcctcccgccaggagatttcgacgatcgctcgcgtcggctcggcgtcggccgcccgcgcggtcaccggcgcgttttcgcaactgcacacgggtctgaacgacgaggattgtcgctcgcggcgcatcccgagtgaccttcacgaggacctgaagatcgtcgtcggcctcgtcccctaccacaaggagaccgaggacgcccaccgcgaggccgccgacagccacatgttccaggcccgcaacgcccacatccacggccagatcgccgagatgcgcgacgccctgcggaacaacgagttcgaccgcgccttcgaactcgccgagcaggactccctctcgctggccgcgacgacgatgaccggcccctccgggtgggtctactggcagcccgctaccctgaagatcttcaatacggtgcgggaactccgcgaggaggaggacatccccgtctacttctcgacggacaccggcgccagcgtctacgtcaacaccaccgaggaacacgtcgacgaggtcgaggaggcggtctcggattgcggcgtctccaccaccgtctgggacgtcggcggccctgcgaagctgctagacgaggaaaagcacctgttctag

SEQ ID NO: 125 - DNA, 951 bp, Artificial Sequence
  Synthesized mvd (4)
atgcctacaacagccacagcacatactaatattgcatttattaaatattggggtaaaaaagatgcgcgcttaaatttaccgacaaccagttctttatccctaacactctcacaattttatacaacaacaacagtcacacaaaacaccgacaaagatcaacttgttttaaacggtgagctagccgaccctactagaatacatcattttttaaatacaatacgtgatatccttggtgattttcctgctgtgacagtcacttcagaaaaccatgtgccaaccagtgcaggtctagcctcttcggcttcatctttcgctgcgctaacaggtgcagtaacaagagaaatgggatttgatttgtctaatcaatccttatctcggttagcacgccgtggatctggttccgcctcacgatcgttttacagtcactttgctatctggcatgctggtatggatgatgcctcatcttttgctgaaagtttaaatgcccctgacatgccgattgcccttgtcgttgccgaagtgtccacttcagcaaagaaagtgagctcaagtgatggcatgcaacgtgcaatcacttcaccaaactacgatgattggctcaaccgcagcgcgacacaatttatggatatgcagtctgccattcaacaatcagacatcgaaaaaattggtacgcttgctgaagaaaacgctttagctatgcatgcgcttaatctcactgcacgccataaaccattcacctatttcacgcaagaaacccaacaaatacttgccctagtatcagatttacgacgacaagggatcctagccttcgcaacaatggatgctggtccaaacgtcaaaattataacgactttaaatgatgcaccaaaaattgttacagcactacattctgctttaccatatatccatctcgaaactgctacaagcggatcaggtattacctatgactaa

SEQ ID NO: 126 - DNA, 1005 bp, Artificial Sequence
  Synthesized dvd
atgcgcgcgacacccccgcatcgacgtatgaaagcaaccgcgcgcgcacaccccatccagggcctcgtgaaataccacgggatgcgcgacgagtcgcttcgcatgccgtaccacgactccatcagcgtctgcaccgcgcccagcaacaccacgacgaccgtcgagttcgatcccgaccgcgacgccgaccagtacgtcgtcgacggcgacacggtcaccggtcacggcgcggaccgcatccgcagtgtggtcgatgcggtccgcgaccgcgccgggttcgaccaccgcgtgcgcctggagagccagaacagcttccccacgaacatcggcctggggtcgtcgtcgtcggggttcgcggcggccgcgctggcgtgcgtccgcgccgccggcctggatctggacctcccgacggtgtcgacggtcgcgcgccgcggatcggcgtcggcggcccgcgccgtcacgggcgggttctcggatctgcacgcgggattgaacgacgccgactgccgcagcgaacgcctcgacgcccccgcggagttcgcgtccgatctgcgcatcgtcgtgggcgaagtgcccgcgtacaaggagacggagtctgcccacgccgaggccgccgacagccacatgttcgacgcgcggctggcacacgtccagggccaactcgcggagatgcgtgacgccgtccgcgcgggcgacttccagcgcgtcttcgagaccgccgaacacgactcgctgtcgctcgcggcgacgacgatgacggggccgtccgggtgggtgtactggaagcccgagacgctctcgatattcgagaccgtgcgggagctccgggcggacggcgtgccgacgtacttctcgacggataccggcgcgacagtgtacgtgaacaccactgcgagtcacgccgacgaggtcgaggctgcggtcgccgactgcggcgtcgacaccgccgtctgggaggtcggcgggcctgcccacgaactcgacgagcgcgacgcgatcttctga

SEQ ID NO: 127 - DNA, 1300 bp, Artificial Sequence
  Synthesized dfd
atggcggctgcggactcttcggtctatagggccaccactactgcccctgtcaatattgctgtcatcaagtaagttgactgccccccccccctaaataaaccaaccgcctccttttcttctatcattaaatttgtactaacgctgggacttctctagatactggggaaaacgggacgcaactctgaacctgcccaccaattcttccctctctgtgaccctttcccagcgttcgctccgcaccctcaccaccgcctcctgttctgctatctaccccaccgcagatgagcttatcctcaatggcaagcctcaagatatccaatcctccaagcgtacgctcgcctgtctctccagcctgcgctctcttcgccaggcgctggaatctacagactcatcgttgccgaaattatctacacttcccttgcggattgtttccgagaacaatttccccacggccgctggtcttgctagctcagctgctgggtttgcagccctcgttcgtgctgtagcgaacctctaccaacttccgcaatcacctcgggagctcagccgtatcgctcgtcagggatctggctctgcttgccggtctctgatgggcggctacgtggcttggcgcgctggagagttggaggacggcagcgatagtcttgctgaggaggttgcacctgcctcacactggcctgagatgcgtgccattgtcctggtggtcagcgccgagaagaaggatgtccccagtaccgagggcatgcagacgacggtcgctacctcgagtctcttcgctaccagagcgacatctgttgttcccgagcggatggctgccattgagacagcaatcctgaacaaggactttcctgccttcgccgaactcaccatgcgcgactctaacggcttccacgccacctgccttgactcctggcccccaattttctatatgaacgacgtttcccgggctgctgtcagaattgtccacgatatcaaccgtgctattggccgaactgtgtgtgcgtacacctttgatgctggaccgaatgctgttatctattatctggaaaaggattcggagctggtcgcaggaactgtcaaggcaatcttgaccaccaacactgacggctggaatggtcctttctacgatattctgaaggacgtcactgccccgggtgtttctttggataagattgactctagagccgttgaagttctcaaggagggagtcagccgcgtgattctgaccggtgttggtgagggtcctgtcagtgtagaagaccacctggtcagcgcaactggagatgttctttcgcactaa

SEQ ID NO: 128 - DNA, 1283 bp, Artificial Sequence
  Synthesized dfd (1)
atggcggctacttctgatcataccgtctatcgtgctaccactaccgccccggtcaatattgctgttattaagtgagttgactatcgccccctaatccgtcctgtggtgattcttgtttcctcctaacagggtcctctaggtattggggtaaaagagatgcgtctctgaatctgccaaccaattcctccctctctgttaccctctctcagcgctccctccgaaccctcactaccgcctcctgctcagctatctaccccgccgcagacgagctcatcttgaacggcaagccacaggatattcagtcctccaaacgcacactcgcttgtctctccaacctacgttccctccgtcaggctctcgaaaatgccgacccctcattgcctaaactgtctgctctcccattgcgaattgtttccgagaacaacttccccaccgctgctggtctcgcgagctcagctgctggtttcgcagcccttgtccgtgctatagcagatctttatcagcttccacaatctcctctggagctcagccgtattgcccgtcagggttccggctctgcttgtcggtctctgatgggcggttatgttgcctggcgtgctggcgagcgggaagatggtagcgacagtctggctgaggaagtcgctcccgcatctcattggcctgagatgcgtgcaattatcctggtggttagtgccgagaagaaagacgtccccagtacagagggtatgcagactacagttgctacctcgagtctctttgctacccgggccgcatctgttgtccctgagcggatggccgccattgagacggcaatccagaacaaggacttcgctacctttgcggaaatcaccatgcgtgactctaacagtttccacgcaacttgcctcgactcctggcctccgatcttctacatgaacgacgtctccagagctgccgtgagactcgtccacgacatcaaccgtgctgttggccggactgtgtgtgcttacacattcgacgctggcccgaatgccgttatctactaccttgagaaagactcggaggtggtcgcaggaaccgtcaaggctattttgggccccaacaccgaagggttcgacggcccattctatgatatcttgaagaatgtcactgcttcagtcgtgcctctggagaatgttgactctagagctgtagaagtcttgaagaacggcatcagccgcgtcattctgactggtgtcggggagggtcctatcagcgtggaggatcaccttgtgagcgcgacgggtgatatcctcgcttcttga

SEQ ID NO: 129 - DNA, 954 bp, Artificial Sequence
  SynthesizeD mvaD
atggatagagagcctgtaacagtacgttcctacgcaaatattgctattatcaaatattggggaaagaaaaaagaaaaagagatagtgcctgctactagcagtatttctctaactttggaaaatatgtatacagagacgaccttgtcgcctttaccagccaatgtaacagctgacgaattttacatcaatgctcagctacaaaatgaggtcgagcatgccaagatgagtaagattattgaccgttatcgtccagctggtgagggctttgtccgtatcgatactcaaaataatatgcctacggcagcgggcctgtcctcaagttctagtggtttgtccgccctggtcaaggcttgtaatgcttatttccagcttggtttgtctcggagtcagttggcacaggaggctaagtttgcctcaggttcttcttctcggagtttttatggaccactaggtgcctgggacaaggatagtgggggaatttaccctgtagagacaaacttgaaactagctatgatcatgttggtgctagaggacaagaaaaaaccaatctctagccgtgacgggatgaaactttgtgtggagacttcgacgacttttgacgactgggttcgtcagtctgagaaggactatcaggatatgctgatttatctcaaggaaaatgactttgccaagattggagaattaacggagaaaaatgctcttgctatgcacgctacgacaaaaacagcatcaccagccttttcttatctgaccgattcatcttatgaagcgatggactttgttcgtcaacttcgcgagcaaggagaggcctgctactttactatggatgccggtcctaatgtcaaagttctttgtcaagagaaagacttggagcatttatcaaaaatcttcggtcaacgttaccgcttgattgtgtcaaaaacaaaggatttgagtcaagatgattgctgttaa

SEQ ID NO: 130 - DNA, 1005 bp, Artificial Sequence
  Synthesized mvaD (1)
atgacaacttatgcacgtgcgcacactaacattgcattgatcaaatattggggcaaagcaaataagcaactgatgctgccggcaaccagcagtatttcgcttaccttgaatgacttttacacggacacggcggtaacttttgaccctgcactcgatcaggatcaattcacgttaaatcaccaaatgcagtcgcctactgctgtcagccgctttttggatcatgttcggcacctggcccaaattgatacacgcgctcgggtcaactcgttgaatcatgtaccgactgctgccggtttggccagttcggcttctgcgtttgcggcactggcactggctacaagtcgcgcggctggcctaaatttaacccctaccgctttgtcacggttggcacgtcgcggctcagggtcggccacccgttcaatctttggcggagcggtaatttggcaccgtggcagcgatgatcaatcctcgtttgccgaacccttaaccattcagccaactctgccgctgcggatgttggtcgtcacggtttccgatcagaaaaaggcagtcagctcccgcaccggcatggccaacacggttgcgaccagcccttattaccaggcatgggtacaatcgaatgaagcgttaatttcacctatgatcacggcattggccgaaaatgatctgacgacgattggtgcactcaccgaattatcgagtatgcgcatgcacgctgccattatggctgaggagccgccgttcacctactttttgccggaaactttacgcgcctggcaattggtgcaagaacaacgggcactcggcattccggcgtttgccacgatggatgccggacccaacgtcaagatcctcacaaccgcaccgtacgtggatgttctcatgaccgccttgcagcctgtttttggcgaccggattttgagcacccgcctcggcccggacgcgcaagtgattacaaaggagcaatttaatgacacagagtcagcaatcacatcgcaaggatga

SEQ ID NO: 131 - DNA, 939 bp, Artificial Sequence
  Synthesized mvaD (2)
atgaaagtaaagtgtaaagccaatgcaagcttggctttaattaaatattggggaaagaaggatgtttttttaaacattccagcgacttctagtcttgctgttagtgttgataaattttattcaataagtgagcttgaactttcagatcgagatgaaataattttaaattcaaagccagttatattgcaaaatagagaaaaggtgttttttgattatgcaagaaaaattcttagtgaaccgaatgttagatttaaaattaaaagtgaaaacaattttccaacagcagcaggccttgcaagttcaagttcaggatttgcttctattgctgcttgtattttgaaatattttgataaatattcttttaatagtgcatctaatcttgcaagagtaggatcagcttctgcagcaagggctatttacggagggtttactattttgaaagaaggttcaaaagaatcttttcaattaagagatgaatcttattttaatgatttgcgcataatatttgccataattgatagtagtgaaaaagaattgtcctcaagagccgcaatgaatatttgcaaacaccatggattttattatgatgcttggattgcttctagtaaaaagatttttaaagatgctttatatttttttttaaaaaaagattttgtgcattttggagcaactattgtaaaaagttatcagaatatgtttgctttaatgtttgcatcttctattttttattttaaaaatagcacaatagatttaattaaatatgccgcttatttaagaaataaaggaattttggtatttgagacaatggatgcgggcccccaagtgaagtttctttgtttggagaaaaatttaaatactattttaaaaggacttaagcagaattttactgacattgagtttattgtttcaaaggttggatgtgacttagaatggatttga

SEQ ID NO: 132 - DNA, 2778 bp, Artificial Sequence
  Synthesized Pdc2
atgctttccattcagcaaagatataatatttgtctaatggcggagaggcacccaaagtggacgcaacttgaattggcaaaatgggcttatgagacgttccagctgccaaaaattccatcccaaggcacaatatcgcgtttgttggcaaggaaatcaacttatatgaattgtaaagagcatgaaaaagatgcgaatagattaaggaagccaaataatcttttggttcggaaaattttacaagaatggatttctcaaagtttgtggaatggaatccctataacgtcacctattattcaagacactgcacaagctgtttggcacagaattcctgcggagcatcgcgagggaaatggttcttttagttataaatggatttcgaattttttatcaaaaatggatgtcaatatttctgttttagacgaagagttacccaaaaccccaaaagtctggacatttgaagagagggatgtattgaaggcttatttctccaaaattcctccaaaggatttattcactttagatgaagcgtttctctcctacaacctaccgttggattatgctcaatatgaagcaagtagcattcaaaggcgtatagaggtggcaactgtcatgctgtgctccaatttagatggctctgaaaagttaaaacctgttgtcgtgggcaaatatgatagttacaaatcattcaggaattatttccccaatgaaccgaatgatcctgtgtcacaatcaatgttgggtactaagatggctaagaaatttgatatctcataccatagtaacaggaaagcatggctaacgagcaatcttttccacaactggttagtcaggtgggataagaggttggttgctgtgaataggaagatttggattgttttggatgattcttgctgtcatcgaataattaatttgcgccttcaaaatataaaacttgtatacacttcctcaaattcaaagtttttgccatttaactggggtgtctgggatgaattcaaaacacgatacagaatacaacagtatcaggcgctcattgacttgcaaaatagaatttcgaagaatatccaaaataaaaataaatcagaacggaacgaatgcatacccaatggtaaaaaatgtttgattagctttgagcagagtcaactcacaatgtcaaatgcattcaaatttattaaaaaagcttgggatgatatacccgttgatgctatcaaagcaaattggaaaagttccggtctgcttcctcctgaaatgatacatttgaatgagaatgttagtatggcatttaagaaaaacgaagtcttagagagcgttttgaatagattatgtgatgaatactactgtgttaaaaaatgggaatatgaaatgttgttagatttaaacattgaaaacaaaaacacaaacttcttgagtacagaagaattagtggaaagtgctattgtggagccttgtgaacctgattttgatactgcgccaaaaggtaatgaggtccatgatgataattttgatgtatcagtttttgccaatgaagatgataataatcaaaatcatttaagcatgtcacaagctagccacaaccccgattacaacagtaatcacagcaacaatgctattgaaaatactaataatagaggcagtaataataataacaataataatggtagtagtaataatattaatgataatgatagtagcgtaaagtatttgcaacagaatactgttgataatagtaccaaaacaggtaaccctggacaaccaaatatttctagtatggaatcgcaaaggaactcttcgactacagatttagttgttgacggtaattatgacgtcaattttaacggccttttgaatgatccatataatacaatgaaacagccgggcccattagattataatgtcagtacattaatcgataaacctaatttattcttaagtcctgatttggatttatctactgttggcgttgatatgcaactaccatcatcagaatattttagcgaagtattttcttcagctatcagaaacaacgaaaaagctgcctcagatcagaacaaatcaactgatgaacttccttcaagcacggccatggcaaattcaaactcgataacgactgcccttctagagtcaagaaatcaagcacagccgtttgatgtcccacatatgaatgggttgctgagcgacacatcaaaaagcggacattctgttaattcctcaaatgctatatctcaaaattctctgaataactttcaacataattcggcgtccgtcgcggaagcttcgtctccttcaattacaccatctcctgtggcaataaactcaacaggcgctccagcgagatctattatatctgcacccatagactcaaattcctctgcgtcatcgccatcagctttagaacatcttgaaggtgctgtttccggtatgtcaccctcttccaccacaatattaagtaacttacaaacaaatataaatatcgccaaatcattgagtaccattatgaaacatgcagaatcaaacgaaatatcactgacgaaagaaacaataaatgaacttaatttcaattatttgacacttttaaaaaggattaaaaagactagaaaacaattaaatagcgaaagcattaaaataaacagtaagaatgcacaagaccatttagaaacccttctatctggggctgcagctgcagctgcaacttccgccaataacttggaccttccgactggtggttcaaacctcccagactctaataacttacacttacctggtaacacaggctttttttag

SEQ ID NO: 133 - DNA, 1707 bp, Artificial Sequence
  SynthesizeD pdc-1
atgagttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctag

SEQ ID NO: 134 - DNA, 939 bp, Artificial Sequence
  Synthesized mdh
atgaaagtcgcagtcctcggcgctgctggcggtattggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcgcgtaaaccgggtatggatcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 135 - DNA, 990 bp, Artificial Sequence
  Synthesized idhA
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgacgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactaccatctgttgaacgaagccgccttcgatcagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctgttctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 136 - DNA, 1050 bp, Artificial Sequence
  Synthesized idh
atgaagatctccctcaccagcgcccgccagcttgcccgcgacatcctcgccgcgcagcaggtgcccgccgacatcgctgacgacgtggccgagcacctggtcgaatccgaccgctgcggctatatcagccacggcctgtcgatcctgcccaactaccgcaccgccctcgacggccacagcgtcaacccgcaaggccgcgccaaatgcgtgctggaccagggcacgctgatggtgttcgacggcgacggcggcttcggccagcacgtgggcaagtccgtgatgcaagcagcgatcgagcgcgtgcgccagcatggccactgcatcgtcactctgcgccgctcgcaccatctcggccgcatgggccactacggcgagatggcggccgccgccggctttgtgctgctgagcttcaccaacgtgatcaaccgcgcgccggtggtggcgccgttcggcggccgcgtggcgcggctcaccaccaacccgctgtgtttcgccggcccgatgcccaacgggcggccgcctctggtggtggacatcgccaccagcgcgattgccatcaacaaggcccgtgtgctggccgagaaaggcgagccggcgcccgaaggcagcatcatcggcgccgacggcaaccccaccaccgacgcgtcaaccatgttcggcgaacaccccggcgcgctgctgccctttggcggccacaagggctacgcactgggcgttgtggccgagctgctggcgggcgtgctgtccggcggcggtaccatccagccagacaatccgcgcggcggcgtggccaccaacaacctgttcgcggtgctgctcaatcccgcgctggacctgggcctggactggcagagcgccgaggtcgaggcgttcgtgcgctacctgcacgacacaccgccggcgccgggcgtcgaccgcgtgcagtaccccggcgagtacgaggccgccaaccgggcgcaggccagcgacacgctaaacatcaacccggccatctggcgcaatcttgagcgcctggcgcagtcgctcaacgtggccgtccccacggcctga

SEQ ID NO: 137 - DNA, 1056 bp, Artificial Sequence
  Synthesized adh
atgaaaggttttgcaatgctaggtattaataagttaggatggatcgaaaaagaaaggccagttgcgggttcatatgatgctattgtacgcccattagcagtatctccgtgtacatcagatatacatactgtttttgagggagctcttggagataggaagaatatgattttagggcatgaagctgtaggtgaagttgttgaagtaggaagtgaagtgaaggattttaaacctggtgacagagttatagttccttgtacaactccagattggagatctttggaagttcaagctggttttcaacagcactcaaacggtatgctcgcaggatggaaattttcaaatttcaaggatggagtttttggtgaatattttcatgtaaatgatgcggatatgaatcttgcgattctacctaaagacatgccattagaaaatgctgttatgataacagatatgatgactactggatttcatggagcagaacttgcagatattcaaatgggttcaagtgttgtggtaattggcattggagctgttggcttaatgggaatagcaggtgctaaattacgtggagcaggtagaataattggagtggggagcaggccgatttgtgttgaggctgcaaaattttatggagcaacagatattctaaattataaaaatggtcatatagttgatcaagttatgaaattaacgaatggaaaaggcgttgaccgcgtaattatggcaggcggtggttctgaaacattatcccaagcagtatctatggttaaaccaggaggaataatttctaatataaattatcatggaagtggagatgctttactaataccacgtgtagaatggggatgtggaatggctcacaagactataaaaggaggtctttgtcctgggggacgtttgagagcagaaatgttaagagatatggtagtatataatcgtgttgatctaagtaaattagttacacatgtatatcatggatttgatcacatagaagaagcactgttattaatgaaagacaagccaaaagacttaattaaagcagtagttatattataa

SEQ ID NO: 138 - DNA, 1059 bp, Artificial Sequence
  Synthesized Adh
atgaaaggttttgcaatgctcagtatcggtaaagttggctggattgagaaggaaaagcctgctcctggcccatttgatgctattgtaagacctctagctgtggccccttgcacttcggacattcataccgtttttgaaggcgccattggcgaaagacataacatgatactcggtcacgaagctgtaggtgaagtagttgaagtaggtagtgaggtaaaagattttaaacctggtgatcgcgttgttgtgccagctattacccctgattggcggacctctgaagtacaaagaggatatcaccagcactccggtggaatgctggcaggctggaaattttcgaatgtaaaagatggtgtttttggtgaattttttcatgtgaatgatgctgatatgaatttagcacatctgcctaaagaaattccattggaagctgcagttatgattcccgatatgatgaccactggttttcacggagctgaactggcagatatagaattaggtgcgacggtagcagttttgggtattggcccagtaggtcttatggcagtcgctggtgccaaattgcgtggagccggaagaattattgccgtaggcagtagaccagtttgtgtagatgctgcaaaatactatggagctactgatattgtaaactataaagatggtcctatcgaaagtcagattatgaatctaactgaaggcaaaggtgtcgatgctgccatcatcgctggaggaaatgctgacattatggctacagcagttaagattgttaaacctggtggcaccatcgctaatgtaaattattttggcgaaggagaggttttgcctgttcctcgtcttgaatggggttgcggcatggctcataaaactataaaaggcgggctatgccccggtggacgtctaagaatggaaagactgattgaccttgttttttataagcgtgtcgatccttctaagctcgtcactcacgttttccggggatttgacaatattgaaaaagcctttatgttgatgaaagacaaaccaaaagacctaatcaaacctgttgtaatattagcataa

SEQ ID NO: 139 - DNA, 2537 bp, Artificial Sequence
  Synthesized Sadh
ctgcagggcttcaccctcggccactacacccacgtcttccccgagttcgcggcgaagatggggccgtggctcgcggccggcgacgtggtgttcgacgagacgatcgtcgacggcatcggcaactcggtcgatgccttcctcgacctcatgcgcgggcgcaacgtcggcaagatgctcgtccgaaccgcctgacgtccggagccggaacggccggcgtcgtgcagcggaagattcgctccagtgccgggcgggcgcaccttcccggccgtagagtcgggcgcatgaaagccctccagtacaccgagatcggctccgagccggtcgtcgtcgacgtccccaccccggcgcccgggccgggtgagatcctgctgaaggtcaccgcggccggcttgtgccactcggacatcttcgtgatggacatgccggcagagcagtacatctacggtcttcccctcaccctcggccacgagggcgtcggcaccgtcgccgaactcggcgccggcgtcaccggattcgagacgggggacgccgtcgccgtgtacgggccgtgggggtgcggtgcgtgccacgcgtgcgcgcgcggccgggagaactactgcacccgcgccgccgagctgggcatcaccccgcccggtctcggctcgcccgggtcgatggccgagtacatgatcgtcgactcggcgcgccacctcgtcccgatcggggacctcgaccccgtcgcggcggttccgctcaccgacgcgggcctgacgccgtaccacgcgatctcgcgggtcctgcccctgctgggacccggctcgaccgcggtcgtcatcggggtcggcggactcgggcacgtcggcatccagatcctgcgcgccgtcagcgcggcccgcgtgatcgccgtcgatctcgacgacgaccgactcgcgctcgcccgcgaggtcggcgccgacgcggcggtgaagtcgggcgccggggcggcggacgcgatccgggagctgaccggcggtgagggcgcgacggcggtgttcgacttcgtcggcgcccagtcgacgatcgacacggcgcagcaggtggtcgcgatcgacgggcacatctcggtggtcggcatccatgccggcgcccacgccaaggtcggcttcttcatgatcccgttcggcgcgtccgtcgtgacgccgtactggggcacgcggtccgagctgatggacgtcgtggacctggcccgtgccggccggctcgacatccacaccgagacgttcaccctcgacgagggacccacggcctaccggcggctacgcgagggcagcatccgcggccgcggggtggtcgtcccgggctgacacgacgacgaaggctccgcactcggatcgagtgcggagccttcgtcgggtacggggatcagcgagcgaacagcagcgcgcgcttgacctcctggatcgccttcgtcacctggatgccgcgcgggcacgcgtcggtgcagttgaaggtggtgcggcagcgccacacgccctcgacgtcgttgaggatgtcgagacgctcggcggcgccctcgtcacggctgtcgaagatgaaccggtgcgcgttgacgatggcggcgggaccgaagtagctgccgtcgttccagtacaccgggcacgaggtggtgcagcacgcgcacaggatgcacttggtggtgtcgtcgaaccgggcacggtcggcctgcgactggatccgctcgcgggtgggctcgttgcccgtggcgatgaggaacggcttcacggcgcggaacgcgtcgaagaagggctccatgtcgacgacgaggtccttctcgaccggcaggccgcggatcggctcgacggtgatggtcaccggcttgccgtccttgggcagcatgtccttcatcaggatcttgcaggccaggcggttgacgccgttgatccgcatggcgtccgagccgcacaccccgtgcgcgcagctgcggcggaacgtgagggtgccgtcgaggtagcccttcacgtagagcagcaggttgagcatgcggtccgacggcagcgccggaacctggaagctgtcccagtgctgacccttgccgtcctcggggttgaaccgcgcgatcttgagggtgaccatcgtggcgccctcgggcacgggtggcaggttcgagacgtcggcttcgttcttctcgagggttgtcatcaagtacttccgctccatcggctcgtagcgggtctgcaccaccggcttgtagtccaggcggatgggggagatcagctccgtcccctccttgtaggccatggtgtgcttgaggaacttctcgtcgtcgcgcttcgggaagtcctcgcgggcgtgaccgccgcgcgattccttccggttgagcgcaccggcgacggtgacctcggccatctcgagcaggaagcccagctcgacggcctcgagcaggtcgctgttgtagcgcttgcccttgtcctggacggtgatgttcttgtaccgctccttcagcgcgtggatgtcctcgagcgccttggtgagcgtctcctcggtgcggaacaccgaggcgttgttgtccatggactgca

SEQ ID NO: 140 - DNA, 1173 bp, Artificial Sequence
  Synthesized adhA
atgtttgagatatcaatttatcttcccacagaaatagtttttggtcctgggaagcttgaaatgcttcctaaactagtgaagaagcatgggctttctgggaaggccctaatagtaactggaaggagaagcacaaaggaaactggagttctttatagagttcaagaactacttaagcaagctggggtagagagcatagtttttgacaaaattattccaaatccaatatctactcatgtggatgaaggggcagagatagcgagaaaagaaaatgttagctttgttgttggcttgggtggtggaagtgcgatagatagtgcaaaagctatagcaatgactgccgccagtggaggtaaatattgggactatgttccagctgtgggaggaggaaagaagcctactggagcgcttccaatagttgcaattccaacaacccacgggactggaacggaggctgatccttatgctgttataactaatcctgaaacaaaggagaagcagggaattggatatgatgttctcttccccaaattctctatagttgatccagaacttatgcttactcttccaaaagatcaaacagtgtacacttcaatggatgctttctaccactccattgaggcctttcttaatgttagagcaaatccatattcggatgttctggctctcgactcaatgaggcgcattgttacataccttccattggcctacgaaaacttgagaaatcttgaagcaagaacgcaacttgcctgggcaagtactgaggctggaataacggaaacggtaacgggagttgtggcaaatcatgcacttgagcatggtctaagtggattctatcctgaagtgcctcatggtctgggcctctgcattctaggaccctacctctttgaatacattctcgactatgcctatgaaaagttggcgatagtcggaagagaggtatttggagtttacgagccaaatgacagaaaggcagcagagctagctattaagaagctacgtgacttccagagcctctttggagtaaacaagaagctcagagaattaggggttaaagaggaagacattccagagatggctaggactgcttatagaatgatgaaacctgttatagaggcaacaccgggagatttgaaagttgaagacttggaagagatctatagaagagcatactaa

SEQ ID NO: 141 - DNA, 2676 bp, Artificial Sequence
  Synthesized adhE
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggtgaaactgcagcgaagaaagaagctgctccggctaaagctgagaaaaaagcgaaaaaatccgcttaa

SEQ ID NO: 142 - DNA, 3015 bp, Artificial Sequence
  Synthesized adhE2
attttactttattctaataatacgtaatacacccacttataactagtatttggcaataaaaatagttataatcattaattattgttaaatgtttgacaatctttaattactgttatataataatattatagaaaataaaatgactgcataattttactatagaaatacaagcgttaaatatgtacatatcaacggtttatcacattagaagtaaataatgtaaggaaaccacactctataatttataaggcatcaaagtgtgttatataatacaataagttttatttgcaatagtttgttaaatatcaaactaataataaattttataaaggagtgtatataaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaaaaaataaagaatgtaaaatagtctttgcttcattatattagcttcatgaagcacatagactattttacattttactcttgttttttatctttcaa

SEQ ID NO: 143 - DNA, 2685 bp, Artificial Sequence
  Synthesized adhE
atgagcaagtaaaggagcaaagattatggcagaagcaattgcaaagaaacccgcaaaaaaggttttgacccctgaagaaaaagcggaattacaaacacaagctgagaagatgactgttgtattgattgaaaaatcacaaaaggcattgtctgaattttcaacattttcgcaagaacaagttgataaaattgttgcagctatggccttggcaggttctgagaattcacttctgttagcccatgctgctcacgacgagactggacgtggggttgtggaagataaggatacgaaaaatcgtttcgcctcagaatcagtttataacgctattaagtttgataagactgtgggtgttattagtgaagacaagattcaaggtaaggtagaattagcagccccacttggtattttggctggaatcgctccaacgacaaatccaacgtcgacaactattttcaaatcaatgttgacagcaaagacacgtaacacaattatctttgctttccatccccagcctcaaaaagcatcggttcttgctgcaaaaattgtttatgatgctgctgttaaagcaggcgcaccggaaaactttatccaatggattgaaaagccttcactttatgcaacaagtgcgctgatacaaaatcctcacattgcttcaattctagctactggtgggccatcaatggttaatgcagctttgaagtcaggaaatccatccatgggtgtcggtgctggaaacggtgcagtttatattgatgcaactgttgacacagatcgtgccgtgtctgatttgttgttatcaaagcgtttcgataatggcatgatttgtgccacagaaaactcagccgttattcaagcaccaatctatgacgaaattttaactaagttacaagaacaaggtgcataccttgttcctaagaaagactacaagaaaattgctgattatgtctttaagcctaacgcagagggatttggtattgctggtcctgttgctggtatgtcaggacgttggattgctgagcaagcaggcgtaaagattcctgatggtaaagatgtacttttgttcgaattagatcagaagaacataggtgaagcgttatcttctgaaaagttatcgccattactttcaatttataaagttgagaagcgtgaagaagctattgagactgttcaatccttgttaaactatcaaggcgcagggcacaacgcagcaattcaaattggttcacaagatgatccattcattaaagagtatgctgacgctattggtgcatcacgtattttggttaaccaacctgactcaatcggtggcgttggggatatttatacagatgctatgcgtccatcgttgacacttggtaccggatcatgggggaagaattcattgtctcataacttatcaacatacgacttacttaatattaagaccgtggctcgccgccgtaatcgtcctcaatgggttcgtttacctaaggaagtttactacgaaaccaatgccattacttacttacaagacttgcctactataaaccgtgcatttattgtcgctgatcctggtatggttcagttcggatttgttggcagagtactaggtcaacttaagttacgtcaagaacaggttgaaacaaatatctatggttcagttaagcctgacccaactttgtcacaagctgttgaaattgctcgccaaatggcagacttcaaaccagatacagttattttacttggcggtggttcggcacttgacgctggtaaaattggtcggttcttgtacgaatactcgacacgccatgaaggaattttagaagatgacgaggcgattaaagagctattcttagaactacaacaaaagtttatggatattcgtaagcgaatcgttaagttttaccacgcacgtttgacacaaatggttgcgattccaacaacttcaggtactggatcagaagtcacaccatttgccgttattacagatgatgaaacacatgtaaagtatccactagccgattatgaattgacaccggaagttgctattgttgatccagaatttgttatgaccgtaccacaacacacggtatcttggtcaggattagatgctttgtcacatgctttggaatcgtatgtctcagtgatggcttctgaattctcacgtccttgggcattacaagctattaagttgatttttgataacttaacaaattcatacaattatgatcctaaacacccaactaaggaaggtcagaatgcacgcacaaagatgcactatgcgtcaacattggctggtatgtcatttgcgaatgccttcttgggacttaaccactcactagcacacaaaactggtggagaattcggactacctcacggtatggcaatcgctattgcaatgccacatgtgattaagtttaatgcggtaacaggaaatgtaaagcgcacaccatacccacgatacgaaacctatacagcacaaaaagattatgctgatattgcacgttacttaggtttgaaaggtgaaacagatgctgaattggtcgatgtattgattgcagaaatcaagaagttggctgcatcagtgggtgtcaatcaaacactatctggcaacggtgtttcaaagcatgactttgatacaaagttagaaaagatgattgacttagtttacaatgaccaatgcacgccgggaaaccctcgccaacc

SEQ ID NO: 144 - DNA, 3164 bp, Artificial Sequence
  Synthesized HMG1
atgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagactccaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagtgtttccaaagaaatttcttctactgatggaacgaaatggaggttaagaagtgacagaaaaagtcttttcgacgtaaagacgttagcatattctctctacgatgtattttcagaaaatgtaacccaagcagacccgtttgacgtccttattatggttactgcctacctaatgatgttctacaccatattcggcctcttcaatgacatgaggaagaccgggtcaaatttttggttgagcgcctctacagtggtcaattctgcatcatcacttttcttagcattgtatgtcacccaatgtattctaggcaaagaagtttccgcattaactctttttgaaggtttgcctttcattgtagttgttgttggtttcaagcacaaaatcaagattgcccagtatgccctggagaaatttgaaagagtcggtttatctaaaaggattactaccgatgaaatcgtttttgaatccgtgagcgaagagggtggtcgtttgattcaagaccatttgctttgtatttttgcctttatcggatgctctatgtatgctcaccaattgaagactttgacaaacttctgcatattatcagcatttatcctaatttttgaattgattttaactcctacattttattctgctatcttagcgcttagactggaaatgaatgttatccacagatctactattatcaagcaaacattagaagaagacggtgttgttccatctacagcaagaatcattttaaagcagaaaagaaatccgtatcttctttcttaaatctcagtgtggttgtcattatcatgaaactctctgtcatactgttgtttgtcttcatcaacttttataactttggtgcaaattgggtcaatgatgccttcaattcattgtacttcgataaggaacgtgtttctctaccagattttattacctcgaatgcctctgaaaactttaaagagcaagctattgttagtgtcaccccattattatattacaaacccattaagtcctaccaacgcattgaggatatggttcttctattgcttcgtaatgtcagtgttgccattcgtgataggttcgtcagtaaattagttctttccgccttagtatgcagtgctgtcatcaatgtgtatttattgaatgctgctagaattcataccagttatactgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 145 - DNA, 1290 bp, Artificial Sequence
  Synthesized CtCNB1_3119
atggccgtcgattcgcgtcttcccaatttccgagctctcacccccgcacagcgctgggagcatgtcgccaccgcatgcaatctcagcgccgaagaacgcaatctactgacccaggcgggcgccctgcccgccaccttggctgacggcatgatcgaaaatgtggtgggcacgttcgagctacccatgggcatcgcaggcaacttccgcatcaacggtcgcgatgtgctgattccgctcgcagtggaagagccctccatcatcgctgctgcttcgtatatggccaagctggcccgtgaagacggaggctttgaaacgtcgagcaccttgccgctgatgcgtgcgcaggtgcaaatcgtcggcatcagcgacccctatggtgcaagactggcgttgttcaaggcccgcgatgagatcctcgcgcaagccaatagccgagacaaggtgctgatcagcctgggcggtggctgcaaggacatcgaaatccacgtcttcccagattctccgcgcggccctatggtcgtgatgcacttgatcgtggacgtgcgcgatgccatgggtgccaacaccgtgaacaccatggccgaatcagtctcgccactggtggaaaagattaccggtggttcggtgcgcctgcgcattctctcgaacctggcagacctgcgcctggcccgtgctcgtgtacgcctgacaccgcaaaccttggccaccaaagagcgcagcggcgaagcaattattgaaggcgtgctcgacgcctacactttcgccgccattgacccctaccgcgccgctacccacaacaagggcatcatgaacggtatcgaccccgtcatcgtcgctacaggcaacgattggcgcgcggtcgaagccggtgcccatgcctatgccagccgcaacggccaatacacctcgctgacgcactgggaaaaagacaatgccggcgccttggtgggaacgatcgagctacccatgcccgtgggcttggtgggcggtgccaccaagacccatccgctggcgcgcctggcgctcaagatcatggaggtgaagtctgcccaggaactgggcgagattgccgccgcagtgggtctggcccagaacctgggtgctttgcgcgcgctggccaccgaaggcattcagcgcggccatatggcacttcatgctcgcaatattgcgcaggtcgcaggagccgtgggtgaagaagtagagatcgtcgccaagcgcctggctaccgagcatgacgtgcgcaccgatcgcgcactggaagtgctgcaagaaattcgcgcccagcgctaa

SEQ ID NO: 146 - DNA, 1284 bp, Artificial Sequence
  Synthesized DKAM_0720
atggagaagacaagccgtatacagggcttctacaagcttccccttgaagaaagacggaggatagtctgcgagtgggctgggctaacagaggaagagtgcaggacactgagcgaattcggtaatctaccagttaagataggggacagcatgattgagaacgttataggcgcgatgagctatcccttcgcagtagcgacaaacttcctgatcaatgggagggattaccttgtcccaatggttatagaggagacaagcgtcgtagcggctgcaagcaatgcggccaggatgcttaggcatgggaaagggatacttgcaaatgctgagagacaggagatgatcagccaaatacacctggttaaagtaaactccccacgctttaaagccatgaagattatcgaggccaagaaggagctactggactacgcggcacagcaggatccaaccctgctaaagtacggcgggggtcccagggacctcgaggtaagagcaatggagcaccctgctttaggcggggtcataatagtccacctagtagtagacgtcagagacgccatgggtgctaacactgttaacacgatggctgaagcgatagccccgcttctagagaagataacgggtggggaagcaaggctcagaatagtttcaaaccacgcagtatacagggttacacgggcatgggctgcgacacctgtcgaagaagtgggaggccttgaagtagccaggaggataatggaggcatctatactcgccgagatagatccctatagggcggtaacccataacaagggcataatgaatggagtaatagcagtagccctcgcgacgggacaggatcaccgcgccatagaggctggagcccatgcatacgcctctagaacgggggtctacaagcccctcagctactgggaggtaacaagcgataactatcttgcgggaagccttgagatacctctccaaataggcgttgttggaggagcagtcaaggtacaccctgtggcaaagatagcattgaagatcctaggggtaaacacggctagggagctcgccgaggtaatggctgcggtagggctagcccagaacctagccgctctaagagccctcgtgacagagggtattcagaaaggccatatgaggctccacgccagaaacctcgctataatggctggtgcatcaggagatctaatagataagatagccgagaaaatgatcagggacggtagaataagatacgactacgctaaacaactagtagagaaagcactacagggcgagccattagactag

SEQ ID NO: 147 - DNA, 1281 bp, Artificial Sequence
  Synthesized mvaA
atgcaaaatttagataagaattttcgacatttatctcgtaaagaaaagttacaacaattggttgataagcaatggttatcagaagaacaattcgacattttactgaatcatccattaatcgatgaagaagtagccaatagtttaattgaaaatgtcatcgcgcaaggtgcattacccgttggattattaccgaatatcattgtggacgataaggcatatgttgtacctatgatggtggaagagccttcagttgtcgctgcagctagttatggtgcaaagctagtgaatcagactggcggatttaaaacggtatcttctgaacgtattatgataggtcaaatcgtctttgatggcgttgacgatactgaaaaattatcagcagacattaaagctttagaaaagcaaattcataaaattgcggatgaggcatatccttctattaaagcgcgtggtggtggttaccaacgtatagcgattgatacatttcctgagcaacagttactatctttaaaagtatttgttgatacgaaagatgctatgggcgctaatatgcttaatacgattttagaggccataactgcatttttaaaaaatgaatttccgcaaagcgacattttaatgagtattttatccaatcatgcaacagcgtccgttgttaaagttcaaggcgaaattgatgttaaagatttagcaaggggcgagagaactggagaagaggttgccaaacgaatggaacgtgcttctgtattggcacaagtagatattcatcgtgcagcaacacataataaaggtgttatgaatggcatacatgctgttgttttagcaacaggaaatgatacgcgtggtgcagaagcaagtgcgcatgcatacgcaagtcgtgacggacagtatcgtggtattgctacatggcgttacgatcaagatcgtcaacgattgattggtacaattgaagtgcctatgacattggcaattgttgggggtggtacgaaagtattaccaatagctaaagcttcattagagctactaaatgtagagtcagcacaagaattaggtcatgtagttgctgccgttggtttagcgcaaaactttgcagcatgtcgcgcgcttgtgtcagaaggtattcaacaaggtcatatgagtttacaatataaatcattagctattgttgtaggagcaaaaggtgatgaaattgctaaagtagctgaagctttgaaaaaagaaccccgtgcaaatacacaagcagcggaacgtattttacaagatttaagaagccaacaatag

SEQ ID NO: 148 - DNA, 1236 bp, Artificial Sequence
  Synthesized LJ1608
atgaaattagaagaatcatctaaaaagaaattttatcaatggttaccagaggaaagaagagtctttttaactgaaaaaggaattaaactaagtgagattgagtctgaaactttggaaagactagataaacttagtgaaaatgtaattggtcaagtccgtcttcctcttggtgtgcttcctaagttaatagttaacgggaaagattatcaagtaccaatggccgtagaagaaccatcggttgttgcagcagcaaaccatgcagctaaaatttttaatcaaaatggtggagcagtagctgatagtagacgaaatggaatatatggtcaaattgttttagaggtaactgataattttgatttaactaagtttactactgaatttcctcaattaattagcttagctaataaaaaattcgttagcttagtcaagcatggtggaggagttcgtaaaattgaagcttctcaaaaagaaaatttagtttttcttagagttttggttgacccagcagaagctatgggagctaataaaacaaatgctattttagaatttttaggaaatgaattagagaagcagccagatattgaacaaactctgtatgcaattttgtctaattatcctacgcaattgactagtgctaaagtaagtctttcaattgacagtgtaggaggattaaaagttgctaaaaagatagctttattgagtaaaataggacaaactgatatttaccgggcagtgactaataataaaggaattatgaatggtattgatagtgtattggttgcaactggtaatgattatcgtggagttgaagcagcaactgctgtttgggctaataaaaatggtgcctatacatctttgagtaagtggaaaattgaagaagatagactagtggggactgtaacagttcccttagcaatcggtgtagtaggtggctcaattaaggctcgtcgagacgttcaacaaagctttagtttattaggtaatatatctgccaagcaactagcagaagttattgcgacaactggcttagcaaataacttttcagctcttttagcaatttctactaagggaattcaagctgggcatatgaaattgcaggcgagaaatttagtagcaaccttaaaagctagtgaaggtgaaaaagcaatagttttaaaaaaattgcaggaaagtaaaaaatatactcaagaagcagcttttgaatttttaagcgaaataagaaaggatcaaaaataa

SEQ ID NO: 149 - DNA, 912 bp, Artificial Sequence
  Synthesized acr1
ttgatatcaatcagggaaaaacgcgtgaacaaaaaacttgaagctctcttccgagagaatgtaaaaggtaaagtggctttgatcactggtgcatctagtggaatcggtttgacgattgcaaaaagaattgctgcggcaggtgctcatgtattattggttgcccgaacccaagaaacactggaagaagtgaaagctgcaattgaacagcaagggggacaggcctctatttttccttgtgacctgactgacatgaatgcgattgaccagttatcacaacaaattatggccagtgtcgatcatgtcgatttcctgatcaataatgcagggcgttcgattcgccgtgccgtacacgagtcgtttgatcgcttccatgattttgaacgcaccatgcagctgaattactttggtgcggtacgtttagtgttaaatttactgccacatatgattaagcgtaaaaatggccagatcatcaatatcagctctattggtgtattggccaatgcgacccgtttttctgcttatgtcgcgtctaaagctgcgctggatgccttcagtcgctgtctttcagccgaggtactcaagcataaaatctcaattacctcgatttatatgccattggtgcgtaccccaatgatcgcacccaccaaaatttataaatacgtgcccacgctttccccagaagaagccgcagatctcattgtctacgccattgtgaaacgtccaaaacgtattgcgacgcacttgggtcgtctggcgtcaattacctatgccatcgcaccagacatcaataatattctgatgtcgattggatttaacctattcccaagctcaacggctgcactgggtgaacaggaaaaattgaatctgctacaacgtgcctatgcccgcttgttcccaggcgaacactggtaa

SEQ ID NO: 150 - DNA, 1670 bp, Artificial Sequence
  Synthesized acr1 (1)
cagaagatatggttcggttatcggttgggattgaacatattgatgatttgattgcagatctggaacaagcattggccacagtttgagcgtaaattttataaaaaacctctgcaatttcagaggtttttttatatttgctttattatcgtatgatgttcataattgatctagcaaataataaaaattagagcaattactctaaaaacatttgtaatttcagatacttaacactagattttttaaccaaatcactttagattaactttagttctggaaattttatttccctttaaccgtcttcaatccaaatacaataatgacagcctttacagtttgatatcaatcagggaaaaacgcgtgaacaaaaaacttgaagctctcttccgagagaatgtaaaaggtaaagtggctttgatcactggtgcatctagtggaatcggtttgacgattgcaaaaagaattgctgcggcaggtgctcatgtattattggttgcccgaacccaagaaacactggaagaagtgaaagctgcaattgaacagcaagggggacaggcctctatttttccttgtgacctgactgacatgaatgcgattgaccagttatcacaacaaattatggccagtgtcgatcatgtcgatttcctgatcaataatgcagggcgttcgattcgccgtgccgtacacgagtcgtttgatcgcttccatgattttgaacgcaccatgcagctgaattactttggtgcggtacgtttagtgttaaatttactgccacatatgattaagcgtaaaaatggccagatcatcaatatcagctctattggtgtattggccaatgcgacccgtttttctgcttatgtcgcgtctaaagctgcgctggatgccttcagtcgctgtctttcagccgaggtactcaagcataaaatctcaattacctcgatttatatgccattggtgcgtaccccaatgatcgcacccaccaaaatttataaatacgtgcccacgctttccccagaagaagccgcagatctcattgtctacgccattgtgaaacgtccaacacgtattgcgacgcacttgggtcgtctggcgtcaattacctatgccatcgcaccagacatcaataatattctgatgtcgattggatttaacctattcccaagctcaacggctgcactgggtgaacaggaaaaattgaatctgctacaacgtgcctatgcccgcttgttcccaggcgaacactggtaaaatttataaaagaagcctctcataccgagaggcttttttatggttacgaccatcagccagatttagaggaaattgacttttcctgtttttacatcataaatcgcaccaacaatatcaatttctttgcgatccagcatatctttaagtacagaactatgctgaataatgtattgaatattatagtgaacattcatagcagtcacctgatcaataaatgctttgcttaattcacgcggttgcataatatcaaatacactgccaaccgaatgcatgagtggcccaagcacgtattggatgtgtggcatttcctgaatatcggaaatctgcttatgttgcaatcttaactggcatgcgctggtgaccgcaccacagtcggtatgtcccaaaaccagaatcactttggaacctttggcttgacaggcaaa

SEQ ID NO: 151 - DNA, 1362 bp, Artificial Sequence
  Synthesized sucD
atgagtaatgaagtatctataaaagaattaattgaaaaggcaaaggtggcacaaaaaaaattggaagcctatagtcaagaacaagttgatgtactagtaaaagcactaggaaaagtggtttatgataatgcagaaatgtttgcaaaagaagcagttgaagaaacagaaatgggtgtttatgaagataaagtagctaaatgtcatttgaaatcaggagctatttggaatcatataaaagacaagaaaactgtaggcataataaaagaagaacctgaaagggcacttgtttatgttgctaagccaaagggagttgtggcagctactacgcctataactaatccagtggtaactcctatgtgtaatgcaatggctgctataaagggcagaaatacaataatagtagcaccacatcctaaagcaaagaaagtttcagctcatactgtagaacttatgaatgctgagcttaaaaaattgggagcaccagaaaatatcatacagatagtagaagcaccatcaagagaagctgctaaggaacttatggaaagtgctgatgtagttattgctacaggcggtgctggaagagttaaagctgcttactccagtggaagaccagcttatggcgttggacctggaaattcacaggtaatagttgataagggatacgattataacaaagctgcacaggatataataacaggaagaaaatatgacaatggaattatatgttcttcagagcaatcagttatagctcctgctgaagattatgataaggtaatagcagcttttgtagaaaatggggcattctatgtagaagatgaggaaacagtagaaaagtttagatcaactttatttaaagatggaaaaataaacagcaagattataggtaaatccgtccaaattattgcggatcttgcaggagtaaaagtaccagaaggtactaaggttatagtacttaagggtaaaggtgcaggagaaaaagatgtactttgtaaagaaaaaatgtgtccagttttagtagcattgaaatatgatacttttgaagaagcagttgaaatagctatggctaattatatgtatgaaggagctggtcatacagcaggcatacattctgacaatgacgagaacataagatatgcaggaactgtattacctataagcagattagttgtaaatcagcctgcaactactgctggaggaagtttcaataatggatttaaccctactactacactaggctgcggatcatggggcagaaacagtatttcagaaaatcttacttacgagcatcttataaatgtttcaagaatagggtatttcaataaagaagcaaaagttcctagctatgaggaaatatggggataa

SEQ ID NO: 152 - DNA, 1356 bp, Artificial Sequence
  SynthesizeD sucD (1)
atggaaatcaaagaaatggtgagccttgcacgcaaggctcagaaggagtatcaagctacccataaccaagaagcagttgacaacatttgccgagctgcagcaaaagttatttatgaaaatgcagctattctggctcgcgaagcagtagacgaaaccggcatgggcgtttacgaacacaaagtggccaagaatcaaggcaaatccaaaggtgtttggtacaacctccacaataaaaaatcgattggtatcctcaatatagacgagcgtaccggtatgatcgagattgcaaagcctatcggagttgtaggagccgtaacgccgacgaccaacccgatcgttactccgatgagcaatatcatctttgctcttaagacctgcaatgccatcattattgccccccaccccagatccaaaaaatgctctgcacacgcagttcgtctgatcaaagaagctatcgctccgttcaacgtaccggaaggtatggttcagatcatcgaagaacccagcatcgagaagacgcaggaactcatgggcgccgtagacgtagtagttgctacgggtggtatgggcatggtgaagtctgcatattcttcaggaaagccttctttcggtgttggagccggtaacgttcaggtgatcgtggatagcaacatcgatttcgaagctgctgcagaaaaaatcatcaccggtcgtgctttcgacaacggtatcatctgctcaggcgaacagagcatcatctacaacgaggctgacaaggaagcagttttcacagcattccgcaaccacggtgcatatttctgtgacgaagccgaaggagatcgggctcgtgcagctatcttcgaaaatggagccatcgcgaaagatgtagtaggtcagagcgttgccttcattgccaagaaagcaaacatcaatatccccgagggtacccgtattctcgttgttgaagctcgcggcgtaggagcagaagacgttatctgtaaggaaaagatgtgtcccgtaatgtgcgccctcagctacaagcacttcgaagaaggtgtagaaatcgcacgtacgaacctcgccaacgaaggtaacggccacacctgtgctatccactccaacaatcaggcacacatcatcctcgcaggatcagagctgacggtatctcgtatcgtagtgaatgctccgagtgccactacagcaggcggtcacatccaaaacggtcttgccgtaaccaatacgctcggatgcggatcatggggtaataactctatctccgagaacttcacttacaagcacctcctcaacatttcacgcatcgcaccgttgaattcaagcattcacatccccgatgacaaagaaatctgggaactctaa

SEQ ID NO: 153 - DNA, 1406 bp, Artificial Sequence
  Synthesized bld
atgattaaagacacgctagtttctataacaaaagatttaaaattaaaaacaaatgttgaaaatgccaatctaaagaactacaaggatgattcttcatgtttcggagttttcgaaaatgttgaaaatgctataagcaatgccgtacacgcacaaaagatattatcccttcattatacaaaagaacaaagagaaaaaatcataactgagataagaaaggccgcatagaaaataaagagattctagctacaatgattcttgaagaaacacatatgggaagatatgaagataaaatattaaagcatgaattagtagctaaatacactcctgggacagaagatttaactactactgcttggtcaggagataacgggcttacagttgtagaaatgtctccatatggcgttataggtgcaataactccttctacgaatccaactgaaactgtaatatgtaatagtataggcatgatagctgctggaaatactgtggtatttaacggacatccaggcgctaaaaaatgtgttgcttttgctgtcgaaatgataaataaagctattatttcatgtggtggtcctgagaatttagtaacaactataaaaaatccaactatggactctctagatgcaattattaagcacccttcaataaaactactttgcggaactggagggccaggaatggtaaaaaccctcttaaattctggtaagaaagctataggtgctggtgctggaaatccaccagttattgtagatgatactgctgatatagaaaaggctggtaagagtatcattgaaggctgttcttttgataataatttaccttgtattgcagaaaaagaagtatttgtttttgagaacgttgcagatgatttaatatctaacatgctaaaaaataatgctgtaattataaatgaagatcaagtatcaaagttaatagatttagtattacaaaaaaataatgaaactcaagaatactctataaataagaaatgggtcggaaaagatgcaaaattattcttagatgaaatagatgttgagtctccttcaagtgttaaatgcataatctgcgaagtaagtgcaaggcatccatttgttatgacagaactcatgatgccaatattaccaattgtaagagttaaagatatagatgaagctattgaatatgcaaaaatagcagaacaaaatagaaaacatagtgcctatatttattcaaaaaatatagacaacctaaataggtttgaaagagaaatcgatactactatctttgtaaagaatgctaaatcttttgccggtgttggttatgaagcagaaggctttacaactttcactattgctggatccactggtgaaggaataacttctgcaagaaattttacaagacaaagaagatgtgtactcgccggttaa

SEQ ID NO: 154 - DNA, 1407 bp, Artificial Sequence
  Synthesized Cbei_3832
atgaataaagacacactaatacctacaactaaagatttaaaagtaaaaacaaatggtgaaaacattaatttaaagaactacaaggataattcttcatgtttcggagtattcgaaaatgttgaaaatgctataagcagcgctgtacacgcacaaaagatattatcccttcattatacaaaagagcaaagagaaaaaatcataactgagataagaaaggccgcattacaaaataaagaggtcttggctacaatgattctagaagaaacacatatgggaagatatgaggataaaatattaaaacatgaattggtagctaaatatactcctggtacagaagatttaactactactgcttggtcaggtgataatggtcttacagttgtagaaatgtctccatatggtgttataggtgcaataactccttctacgaatccaactgaaactgtaatatgtaatagcataggcatgatagctgctggaaatgctgtagtatttaacggacacccatgcgctaaaaaatgtgttgcctttgctgttgaaatgataaataaggcaattatttcatgtggcggtcctgaaaatctagtaacaactataaaaaatccaactatggagtctctagatgcaattattaagcatccttcaataaaacttctttgcggaactgggggtccaggaatggtaaaaaccctcttaaattctggtaagaaagctataggtgctggtgctggaaatccaccagttattgtagatgatactgctgatatagaaaaggctggtaggagcatcattgaaggctgttcttttgataataatttaccttgtattgcagaaaaagaagtatttgtttttgagaatgttgcagatgatttaatatctaacatgctaaaaaataatgctgtaattataaatgaagatcaagtatcaaaattaatagatttagtattacaaaaaaataatgaaactcaagaatactttataaacaaaaaatgggtaggaaaagatgcaaaattattcttagatgaaatagatgttgagtctccttcaaatgttaaatgcataatctgcgaagtaaatgcaaatcatccatttgttatgacagaactcatgatgccaatattgccaattgtaagagttaaagatatagatgaagctattaaatatgcaaagatagcagaacaaaatagaaaacatagtgcctatatttattctaaaaatatagacaacctaaatagatttgaaagagaaatagatactactatttttgtaaagaatgctaaatcttttgctggtgttggttatgaagcagaaggatttacaactttcactattgctggatctactggtgagggaataacctctgcaaggaattttacaagacaaagaagatgtgtacttgccggctaa

SEQ ID NO: 155 - DNA, 855 bp, Artificial Sequence
  Synthesized yeaE
atgcaacaaaaaatgattcaatttagtggcgatgtctcactgccagccgtagggcagggaacatggtatatgggcgaagatgccagtcagcgcaaaacagaagttgctgcactacgcgcgggcattgaactcggtttaaccctcattgataccgccgaaatgtatgccgatggcggtgccgaaaaggtggttggggaagcattaaccggtctgcgagagaaggtctttctcgtctctaaagtctatccgtggaatgctggcgggcaaaaagcgataaatgcatgcgaagccagtttacgccgtctcaatactgattatctcgatctttacttattacactggtctggcagtttcgcttttgaagagactgtcgcagcgatggaaaaattgatcgcccagggaaaaatccgccgctggggcgtttctaaccttgattatgctgatatgcaggaactctggcagctgccggggggaaatcagtgtgccactaatcaggtgctttaccatctcggttcacgaggaattgagtacgatctactcccctggtgccagcaacagcagatgccggtgatggcttacagtccgttagcccaggccgggcggttgcgcaatggactgttaaaaaacgcggtagtcaacgaaattgcacatgctcacaatatcagcgcggcacaagtattgttggcgtgggtgatcagtcatcagggtgtgatggcgattccaaaagcggccacgattgcccatgtccaacaaaatgcggctgtgcttgaggtcgaactttcttcagcggaattagctatgctggataaggcatatccggcaccaaaaggaaaaactgcgctggatatggtgtga

SEQ ID NO: 156 - DNA, 1164 bp, Artificial Sequence
  Synthesized dhaT
atgagctatcgtatgtttgattatctggtgccaaacgttaacttttttggccccaacgccatttccgtagtcggcgaacgctgccagctgctgggggggaaaaaagccctgctggtcaccgacaaaggcctgcgggcaattaaagatggcgcagtggacaaaaccctgcattatctgcgggaggccgggatcgaggtggcgatctttgacggcgtcgagccgaacccgaaagacaccaacgtgcgcgacggcctcgccgtgtttcgccgcgaacagtgcgacatcatcgtcaccgtgggcggcggcagcccgcacgattgcggcaaaggcatcggcatcgccgccacccatgagggcgatctgtaccagtatgccggaatcgagaccctgaccaacccgctgccgcctatcgtcgcggtcaataccaccgccggcaccgccagcgaggtcacccgccactgcgtcctgaccaacaccgaaaccaaagtgaagtttgtgatcgtcagctggcgcaacctgccgtcggtctctatcaacgatccgctgctgatgatcggtaaaccggccgccctgaccgcggcgaccgggatggatgccctgacccacgccgtagaggcctatatctccaaagacgctaacccggtgacggacgccgccgccatgcaggcgatccgcctcatcgcccgcaacctgcgccaggccgtggccctcggcagcaatctgcaggcgcgggaaaacatggcctatgcctctctgctggccgggatggctttcaataacgccaacctcggctacgtgcacgccatggcgcaccagctgggcggcctgtacgacatgccgcacggcgtggccaacgctgtcctgctgccgcatgtggcccgctacaacctgatcgccaacccggagaaattcgccgatatcgctgaactgatgggcgaaaatatcaccggactgtccactctcgacgcggcggaaaaagccatcgccgctatcacgcgtctgtcgatggatatcggtattccgcagcatctgcgcgatctgggagtaaaagaggccgacttcccctacatggcggagatggctctgaaagacggcaatgcgttctcgaacccgcgtaaaggcaacgagcaggagattgccgcgattttccgccaggcattctga

SEQ ID NO: 157 - DNA, 1152 bp, Artificial Sequence
  Synthesized fucO
atgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaaggactttccccgaccaataaacccagtgtaccgattctggcaatccccaccacagcaggcactgcggcagaagtgaccattaactacgtgatcactgacgaagaaaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgtggggttagggttggtgcatggtatggcgcatccactgggcgcgttttataacactccacacggtgttgcaaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaa

SEQ ID NO: 158 - DNA, 1164 bp, Artificial Sequence
  Synthesized Cbei_2421
atggaaaattttaattattcaatacctactaaagtttattttggaaaagggcaaatcaaaaatcttgctgccataattaaagaatctggtaataaaatacttatagcatatggcggaggaagtattaagaaaattgggttatacgatgaaatgattaaaatattaaatgataattcaatatcatatgttgaattgtcaggaatagaaccaaatccaagaattgaaacagtaagaaaaggaattaaaatttgtaaggaaaataatgttgaagtagttcttgctgtaggaggcggaagtacaatagattgtgcgaaagttattgcggcaggagtaaaatacgaaggagatccatgggatttagtaactagtccacaaaaaattaatgaagtattgcctatagtaacaatattaacactatcagcgactggttctgagatggatccacatgctgtgatttctgatatgacaactaatcaaaaattgggtacaggtcatgaaaatatgaaaccaaaagcttcaattttagatcctgaatatacttattcagttcctaaaaatcaaactgcagctggaactgctgatattatgagtcatatatttgaaacttattttaatcatacaaagggtgtggatatccaagatagtacagccgaaggattacttagagcttgcataaaatatggtaagattgcaatagagaatccaaaggattacgatgcaagagcaaatttaatgtgggcttcaagctgggctattaatggcttgatttcatatggaacaaattcaccttgggtggtacatccaatggagcatgaattgagtgctttttatgatataacacatggggttggattggctatattaacacctcattggatgaaatattctttagacgatactactgtttttaagtttgctcaatatggaataaatgtttggggaatagataaaaacttagataagtttgaaatagcaaataaagcaatagaaaagacatctgaattctttaaggaattgggtataccaagtactttaagggaagttggcattgaagaagataagttagaattaatggcaaaaaaggctatgaatccatattttaagtatgcttttaagccattagatgaaaatgatatattaaagatttttaaagcagcactttag

SEQ ID NO: 159 - DNA, 933 bp, Artificial Sequence
  Synthesized Lreu_0194
atgacacgaaaagttggagttatcggaatgggtaatgtggggtcaacagttgcccactatattgtggcaatgggttttgcagatgacctagttctgatcgacaaaaacgaagcaaaggttaaggcagatgcacttgattttgaagatgcgatggctaatctgcctttccataccaatattactgttagtgattacagtgcattgaaggatgcggatgtaattgtatccgcgttgggaaatatcaaacttcaagataatcctaatgctgatcgttttgcggaacttccatttacccgccgagcagtaaaagaggttgcacaaaagattaaggaaagtggctttaacggtaagattgtggctattactaacccggttgatgttattacctcgctctaccaaaaaataactggtcttccgaagaaccatgtattaggaacagggaccctgcttgactctgcacgaatgaagcgggcagtggctgaacggcttaacttagatccgcgttctgtagatggttataatcttggcgagcatggtaattcacaatttacagcatggtccactgttcgcgttcttggccgtccattaactgaattagcggataagcgtggattagacttagaagagcttgataaggaagctaagatgggtggctggactgtctttcaagggaaaaagtatactaattatggggttgcaacggcagctgttaagcttgtcaatgcaattttatccgattcattgactgaattaccggtatcaaatttccgtgaagaatatggagtttacttgtcttacccagcggttgttggtcgtgatggagttgtagagcaagcccaacttgacttgacagaagaagaactgcaaaagctgcaaacatcagccgatttcattaaggaaaagtatcaagaaagtttgcaagcaaaagattaa

SEQ ID NO: 160 - DNA, 3525 bp, Artificial Sequence
  Synthesized car
atggcagtggattcaccggatgagcggctacagcgccgcattgcacagttgtttgcagaagatgagcaggtcaaggccgcacgtccgctcgaagcggtgagcgcggcggtgagcgcgcccggtatgcggctggcgcagatcgccgccactgttatggcgggttacgccgaccgcccggccgccgggcagcgtgcgttcgaactgaacaccgacgacgcgacgggccgcacctcgctgcggttacttccccgattcgagaccatcacctatcgcgaactgtggcagcgagtcggcgaggttgccgcggcctggcatcatgatcccgagaaccccttgcgcgcaggtgatttcgtcgccctgctcggcttcaccagcatcgactacgccaccctcgacctggccgatatccacctcggcgcggttaccgtgccgttgcaggccagcgcggcggtgtcccagctgatcgctatcctcaccgagacttcgccgcggctgctcgcctcgaccccggagcacctcgatgcggcggtcgagtgcctactcgcgggcaccacaccggaacgactggtggtcttcgactaccaccccgaggacgacgaccagcgtgcggccttcgaatccgcccgccgccgccttgccgacgcgggcagcttggtgatcgtcgaaacgctcgatgccgtgcgtgcccggggccgcgacttaccggccgcgccactgttcgttcccgacaccgacgacgacccgctggccctgctgatctacacctccggcagcaccggaacgccgaagggcgcgatgtacaccaatcggttggccgccacgatgtggcaggggaactcgatgctgcaggggaactcgcaacgggtcgggatcaatctcaactacatgccgatgagccacatcgccggtcgcatatcgctgttcggcgtgctcgctcgcggtggcaccgcatacttcgcggccaagagcgacatgtcgacactgttcgaagacatcggcttggtacgtcccaccgagatcttcttcgtcccgcgcgtgtgcgacatggtcttccagcgctatcagagcgagctggaccggcgctcggtggcgggcgccgacctggacacgctcgatcgggaagtgaaagccgacctccggcagaactacctcggtgggcgcttcctggtggcggtcgtcggcagcgcgccgctggccgcggagatgaagacgttcatggagtccgtcctcgatctgccactgcacgacgggtacgggtcgaccgaggcgggcgcaagcgtgctgctcgacaaccagatccagcggccgccggtgctcgattacaagctcgtcgacgtgcccgaactgggttacttccgcaccgaccggccgcatccgcgcggtgagctgttgttgaaggcggagaccacgattccgggctactacaagcggcccgaggtcaccgcggagatcttcgacgaggacggcttctacaagaccggcgatatcgtggccgagctcgagcacgatcggctggtctatgtcgaccgtcgcaacaatgtgctcaaactgtcgcagggcgagttcgtgaccgtcgcccatctcgaggccgtgttcgccagcagcccgctgatccggcagatcttcatctacggcagcagcgaacgttcctatctgctcgcggtgatcgtccccaccgacgacgcgctgcgcggccgcgacaccgccaccttgaaatcggcactggccgaatcgattcagcgcatcgccaaggacgcgaacctgcagccctacgagattccgcgcgatttcctgatcgagaccgagccgttcaccatcgccaacggactgctctccggcatcgcgaagctgctgcgccccaatctgaaggaacgctacggcgctcagctggagcagatgtacaccgatctcgcgacaggccaggccgatgagctgctcgccctgcgccgcgaagccgccgacctgccggtgctcgaaaccgtcagccgggcagcgaaagcgatgctcggcgtcgcctccgccgatatgcgtcccgacgcgcacttcaccgacctgggcggcgattccctttccgcgctgtcgttctcgaacctgctgcacgagatcttcggggtcgaggtgccggtgggtgtcgtcgtcagcccggcgaacgagctgcgcgatctggcgaattacattgaggcggaacgcaactcgggcgcgaagcgtcccaccttcacctcggtgcacggcggcggttccgagatccgcgccgccgatctgaccctcgacaagttcatcgatgcccgcaccctggccgccgccgacagcattccgcacgcgccggtgccagcgcagacggtgctgctgaccggcgcgaacggctacctcggccggttcctgtgcctggaatggctggagcggctggacaagacgggtggcacgctgatctgcgtcgtgcgcggtagtgacgcggccgcggcccgtaaacggctggactcggcgttcgacagcggcgatcccggcctgctcgagcactaccagcaactggccgcacggaccctggaagtcctcgccggtgatatcggcgacccgaatctcggtctggacgacgcgacttggcagcggttggccgaaaccgtcgacctgatcgtccatcccgccgcgttggtcaaccacgtccttccctacacccagctgttcggccccaatgtcgtcggcaccgccgaaatcgtccggttggcgatcacggcgcggcgcaagccggtcacctacctgtcgaccgtcggagtggccgaccaggtcgacccggcggagtatcaggaggacagcgacgtccgcgagatgagcgcggtgcgcgtcgtgcgcgagagttacgccaacggctacggcaacagcaagtgggcgggggaggtcctgctgcgcgaagcacacgatctgtgtggcttgccggtcgcggtgttccgttcggacatgatcctggcgcacagccggtacgcgggtcagctcaacgtccaggacgtgttcacccggctgatcctcagcctggtcgccaccggcatcgcgccgtactcgttctaccgaaccgacgcggacggcaaccggcagcgggcccactatgacggcttgccggcggacttcacggcggcggcgatcaccgcgctcggcatccaagccaccgaaggcttccggacctacgacgtgctcaatccgtacgacgatggcatctccctcgatgaattcgtcgactggctcgtcgaatccggccacccgatccagcgcatcaccgactacagcgactggttccaccgtttcgagacggcgatccgcgcgctgccggaaaagcaacgccaggcctcggtgctgccgttgctggacgcctaccgcaacccctgcccggcggtccgcggcgcgatactcccggccaaggagttccaagcggcggtgcaaacagccaaaatcggtccggaacaggacatcccgcatttgtccgcgccactgatcgataagtacgtcagcgatctggaactgcttcagctgctctga

SEQ ID NO: 161 - DNA, 438 bp, Artificial Sequence
  Synthesized acpS
atgacgatcctcggtatcggcttggacttggtgactgtctccgagttcgccgaacagctcgaacgtgccggaaccaccatgctccgggaaagtttcaccgcgggcgagcgacgctactgtcagagcaaggggaccgatccggcacgcagctatgcggcgcggtgggcggcgaaggaggcggtgctcaaggcctgggcatcgtctcggttcgcccgccgcccgcagatcggtgacaacccctacccgctgatcgaagtggtcaacgacgcatggggcaggccgagcatcaagctgcacggcctggctgccgaattcctgccgcgcgcacgggttcacctctcgctcacgcacgacggcgacatggccgccgcgatggtggtgctggaggacccgggcgaactggccgatctgatcgcaggccgcgaaggccagggctga

SEQ ID NO: 162 - DNA, 840 bp, Artificial Sequence
  Synthesized crt (1)
atgaaagttggtgttataggtgctggagctatgggctctggtattgctcaagcttttgctcaaactgaaggttatgaagtttgtttatgtgatattaaagaagaatttgctgctagcggtaaggctagaattgctaaaagttttgacggaagaattgctaagggtaaaatggaaaaagctgatgctgataaaattttatctaaaatatctattggtttaaaagatatttgtaaagactgcgatttaatcgttgaggctgcttttgaaaatatggaagttaaaaaaactactttcaaagaattacatgaaatttgtaagcctgattgtattttctcttctaacacttcttcactttctattactgaaataggtgttggtattggaagacctgttgttggtatgcacttctttaaccctgctcctgttatgaaattggttgaagttatttctggtatcaatactcctagagatgttgttgaaaaaataatcaaaatatctgaagaaattggaaaaactcctgttgaagttaatgagactgctggtttcgttgttaaccgtatattagtacctatgattaatgaagctatagagttatattctatgggagttgcttctgttgagggtatagataatgctatgaaacttggtgctaatcaccctatgggtcctttagctttaggtgatttaattggtcttgatattgtacttgctattatggaagtacttcaaagagaaactggagaccctaaatatagacctagtgctttacttagaaaaatggttcgcggcggtttactcggacaaaaaactggtaaaggtttctatgaatattctaaataa

SEQ ID NO: 163 - DNA, 2190 bp, Artificial Sequence
  Synthesized fadb
atgctttataaaggcgacaccctgtacctcgactggctggaagatggcatcgccgaactggtgttcgatgctccaggctcggtcaacaaactcgacaccacaaccgtcgccagcctcggccaggcgcttgaggtgctggaaaagcaacacgatttaaaagggctgctgctgcgctcgaataaagcggcctttattgtcggcgcggacatcaccgaatttctttcactgttcctcgtccccgaagagcagttaagccaatggctgcatttcgctaatagcgtctttaaccgtctggaagatttacccgtaccgacccttgccgccgtaaacggctatgcgctgggcggcgggtgcgaatgcgtgctggccaccgattaccgtctggcgacgccagacctccgtatcggtctgccggaaactaaactgggcatcatgccgggtttcggcggctccgttcgtctgccgcgtatgctgggcgcagacagcgctctggagattatcgcggcaggtaaagatgtcggtgctgagcacgcgctgaaaatcggccttgtggacggcgtggtcaaacaagagaagctaattgaaggcgcgatagcggtgttacgccaggccattaccggcgatctcgactggagagctaaacgccagccgaaactggaaccgttgaaactcagcaaaattgaagcggcgatgagctttaccatcgctaaaggcatggtcgcacaaacggcgggtaaacattatccggcgccgatgaccgcagtgaaaaccattgaagccgccgcgcgttttggtcgtgaagaagcgctgaatctggaaaataaaagctttgttccgctggcgcataccaatgaggctcgtgcgctggtcggtatcttcctcaacgatcaatatgtaaaaggtaaagccaaaaagctgaccaaagatattgagacgccgaaacaggcagccgtactgggcgcgggcattatgggcggcggtatcgcttaccagtcggcctggaaaggcgtaccggtcatcatgaaagatatcaacgataaatcgttgaacctcggcatgaccgaagcggccaagctgctgaacaaacagctggagcgcggcaagatcgacggtctgaagctggcgggcgtcatttcgaccatccacccgactctcgattatgccggtttcgatcgcgtagatgttgttgtcgaagcggtagtcgaaaatccgaaagtcaaaaaagcggtactggcggaaaccgagcagaaagtgcgcccggaaaccgtgctggcctcaaatacctccaccatcccaattggcgaactggcaagcgccctggaacgtccggaaaacttttgcggaatgcacttcttcaacccggtacaccggatgccgctggttgaaatcattcgcggcgagaagagttcagacgaaaccattgccaaagtcgtcgcctgggccagcaaaatgggcaagacaccgattgtagtcaacgactgccccggcttctttgttaaccgcgtgctgttcccctatttcgccggtttcagtcaactgctgcgcgacggcgcggatttccgcaaagtcgataaagtgatggaaaaacagttcggctggccgatgggcccggcctatttgctggatgttgtcggcattgataccgcgcatcacgcgcaggcggtaatggcggcaggcttcccgcaacggatgcagaaagagtatcgcgacgcgatcgacgcgctgtttgacgccagccgttttgggcaaaaaaacggtctgggcttctggcgctataaagaagacagcaagggtaagccgaagaaagaagaagatgccgcggtggatgacctgctggcaagcgtcagtcagccgaaacgcgacttcagcgacgacgagataatcgcccgtatgatgatcccgatgattaacgaagtggttcgctgtctggaagaaggcattatcgcaagcccggcggaagccgatatggcgctggtctacggcctgggcttccctccgttccacggcggcgcattccgctggctggatacacagggcagcgcgaaatatcttgatatggcgcagcagtatcaacacctcggcccgctgtatgaagtgccggaagggctgcgtgataaagcgcgccataacgaaccgtattatcccccggttgaaccagcccgtccggttggttctctgaaaacggcttaa

SEQ ID NO: 164 - DNA, 2324 bp, Artificial Sequence
  Synthesized tpa
gtagagaggcctgaacaagatggcagctgcttgtcggagcgtgaaggtaagtgggttttcgcccttctgcacccactgggacggcgcagccatagaaccagcggggagagatcggtgctgggagaaagcgtcactcttatctaggcctgttattgcgacggtggggctcgggaggtgaaggtcaactctccggggcacgtgcatgtgccctggatgtggatgtgcactggctggctgtctgaggatgagacacccagagggtcccatcgttaagatgaggaggcacatctgggtcaagtcgcggcggtgaccgggagcctccctcgcagaagcctggagaatgggtgggggtggtccgcacagtgtagttgttgaccgagctcctcttcacagggtctggttgccttaataaccggcggagcctcaggcctaggcttggccacagcggagcgactggtggggcagggggccactgctgtacttttggacctgcccaactcagatggggagacccaggccaagaagttagggaagagctgcgcctttgccccagccgacgtaagtggggttacctcacctctcccatggggttccttttcataaaggtgcctgtctttgtgggcaatggactcatctccttttcccttttggtctgagctgttttcatacgcctccggataaagaatttctaaagctgtgattcacaacagggaggagggcggggagggaggttggagattagccatatcagaatctcctcaaggagatatgtagagttattgtgccctgtaattttaatatttacaaagtcaaaaaacttatcctgaaacaaactacagaaagtgaagtttgacaaaatcatattagcaagtaggggtaccaaaaagttagtttatattcatatcaataattctcacaacttatctgatccacagcagttgagaatctccaggggtatgtaagtttatcaaaagagggtatagatttcaaagatgttttgaaacactgctctgtaatacctaacattttcacctttgtctcactctagagttttctttccttggtctctcccttgcttacacagtcactcgaagactggatttgccctgcgcccccaaccccctgcatgacttagtggtatgtgtgtgatggtagatgaataccttctccacttttccctttgaaggtgacctcagagaaggatgtgcaagcagccctgactctagcaagagaaaagtttggccgtgtggatgtggcagtcaactgtgcaggcattgcagtggccagcaagacatataacttaaagaaaagccaggcccataccttggaggactttcagcgagttatcaatgtaaggccttagaggttctccaggggtagtagtaagaagtatctgactgtgagtgtccccacacccacaaaaggctacctctgcttcaagtcctatatcatgtctcttctggtcctccaggtgaatctcataggcaccttcaatgtgatccgcctggtggctggtgagatgggccagaacgaaccagaccagggaggccaacgtggggtcatcatcaatacagccagcgtagctgcctttgagggccaggtgtgtgagtagggacaaggcttgtaccttggtgacttgttgggggccctgccccctatgacctccactccctctccaggttggacaagctgcatactctgcttccaaggggggcatagtgggcatgacattgcccattgctcgggatctggctcccatgggtatccgggtgatgaccattgctccaggtagacatatcccctccctgccacacctgggattgggtgggatctgtggacaattgagcagggaagttatctaccatctaaacagcagcagccttctatctctggacatagggaagcaccacaagtagagttacggtgaggagggaccagggagatataaaagtgagggtgggtctctgattctggctgtcttatctaggcctatttggcaccccgctgttgaccaccctcccagataaagtgcgcaacttcctggccagccaggtgcccttccccagccgacttggtgaccctgccgagtatgctcatctggtacaggccatcatcgagaactcattcctcaatggagaagtcatccggctggatggggccatccgcatgcagccttgaagggggaaggcagagagaacaaaggttcctctgtccttccccctcctggagtgctctactccagtttggaaggaagcccagtagccattttgtaacctctgcctatcagtcaccctgtgtgcctaataaagtctctgtttctcacg

SEQ ID NO: 165 - DNA, 2178 bp, Artificial Sequence
  Synthesized fadb (1)
atgctttataaaggcgacaccctgtacctcgactggctggaagatggcatcgccgaactggtgttcgatgctccaggctcggtcaataaactcgacaccgcaaccgtcgccagcctcggccaggcgcttgaggtgctggaaaagcaacacgatttaaaagggctgctgctgcgctctaataaagcggcctttattgtcggcgcggacatcaccgaatttctttcactgttcctcgtccccgaagagcagttaagccaatggctgcatttcgctaatagcgtctttaaccgtctggaagatttacccgtaccgacccttgccgccgtaaacggctatgcgctgggcggcgggtgtgaatgcgtgctggcgaccgattaccgtctggcgacgccagacctccgtatcggcctgccggaaactaaactgggcatcatgccgggtttcggcggctccgttcgtctgccgcgtatgctgggcgcggacagcgcgctggagattatcgcggcaggtaaagatgtcggtgctgagcacgcgctgaaaatcggccttgtggacggcgtggtcaaacaagagaagctaattgaaggcgcgatagcggtgttacgccaggccattaccggcgatctcgactggagagctaaacgccagccgaaactggaaccgttgaaactcagcaaaattgaagcagcgatgagctttactatcgctaaaggcatggtcgcacaaacggcgggtaaacattatccggcgccgatgaccgcagtgaaaaccattgaagccgccgcgcgttttggtcgtgaagaagcgctgaatctggaaaataaaagctttgttccgctggcgcataccaatgaggctcgtgcgctggtcggtatcttcctcaacgatcaatatgtaaaaggtaaagccaaaaagctgaccaaagatattgagacgccgaaacaggcagccgtactgggcgcgggcattatgggcggcggtatcgcttaccagtcggcctggaaaggcgtaccggtcatcatgaaagatatcaacgataaatcgttgaacctcggcatgaccgaagcggccaagctgctgaacaaacagctggaacgcggcaagatcgacggtctgaagctggcgggcgtcatttcgaccatccacccgactctcgattatgccggtttcgatcgtgtagatgttgttgtcgaagcggtggtcgaaaatcccaaagtcaaaaaagcggtactggcggaaaccgagcagaaagtgcgcccggaaaccgtgctggcctcaaatacctctaccatcccgattggcgaactggcaagcgccctggaacgtccggaaaacttttgcggaatgcacttcttcaacccggtacaccggatgccgctggttgaaatcattcgcggcgagaagagttcagacgaaaccattgccaaagtcatcgcctgggcaagcaaaatgggcaagacgccgattgtagtcaacgactgccccggcttctttgttaaccgcgtgctgttcccctatttcgccggtttcagtcaactgctgcgcgacggcgcggatttccgcaaagtcgataaagtgatggaaaaacagttcggctggccgatgggcccggcctatctgctggatgttgtcggcattgataccgcgcatcacgcgcaggcggtaatggcggcaggcttcccgcaacggatgcagaaagagtatcgcgatgcgatcgacgctctgtttgatgctaaccgtttcggacagaaaaacggtctgggcttctggcgctataaagaagacagcaaaggcaagccgaagaaagaagaagatgccgcggtggatgacctgctggcaagcgtcagccagccgaaacgcgacttcagcgacgacgagataatcgcccgtatgatgatcccgatgattaacgaagtggttcgctgtctggaagaaggcattatcgcaagcccggcggaagccgatatggcgctggtctacggcctgggcttccctccgttccacggcggcgcattccgctggctggatacacagggcagcgcgaaatatcttgatatggcgcagcagtatcaacacctcggcccgctgtatgaagtgccggaagggctgcgtaataaagcgcgccataacgaacctattatcccccggttgaaccagcccgtccggttggttctctga

SEQ ID NO: 166 - DNA, 945 bp, Artificial Sequence
  Synthesized fadb (2)
atgagtactgccacgatacggcgcgtagcggtagtgggaacgggggtgattggcgcgagctgggccgcctgttttctgtcgcgcggtctgcaggttgccgcgaccgatccttcgagcggcgcacgccagcgtctggagcacgccgtgatgcagcattggccgacgctcgtcacggccgggctcgcatccggcgcgtcgctgggcaatctcacgttccacgattcgctcgatgctgcgctggaaggtgcgcagttcgttcaggaaaatggcccggagcgcatcgacgcgaagcgtgagctgttcgccgcaatcgacgccgctacggcaagcgatgtggtaattgcatcgagctcatcgggattgctgatcagcgatgtgcaaaccgcctgccggcacccggagcgggtcgtgctcggtcatccgttcaatccaccgcatctgattccgctcgtcgaggtgatcggcggacgcttgacgtcggagaccgcgattcgcgccgcgatcgatttctatcgtgcgatcggcaagcgccccattaacccgcaaaaagaaatttccggccatatcgcgaaccggttgcaggctgcgctgtggcgggaggcgttccatctggtgtcgatcggcgctgccaccgtgcaggacatcgacgatgcgatcgcgtatgggccaggtttgcgttgggccgtgatgggaccatttgcgaatctgcatctgtcaggcggagccgagggaatgcggcacctgctcgagcatctgggcgccccgatcgagaactggtgggcgcaactgggcacgccggccatgacgcccgacttgaagcggatggtgatcgacggtgtcgatgccgcgcttgccggccgcaaggacagcgacctggctgcgcaacgcgacctgatcattctcgacgtgctacgtgcgaaggctgccgcgtcggccattccgtga

SEQ ID NO: 167 - DNA, 999 bp, Artificial Sequence
  Synthesized Msed_1426
atgaaagctgtcgtagtgaaaggacataaacagggttatgaggtcagggaagttcaggacccgaaacctgcttcaggagaagtaatcatcaaggtcaggagagcagccctgtgttatagggaccttctccagctacaggggttctaccctagaatgaagtaccctgtggttctaggacatgaggttgttggggagatactggaggtaggtgagggagtgaccggtttctctccaggagacagagtaatttcactcctctatgcgcctgacggaacctgccactactgcagacagggtgaagaggcctactgccactctaggttaggatactctgaggaactagatggtttcttctctgagatggccaaggtgaaggtaaccagtctcgtaaaggttccaacgagagcttcagatgagggagccgttatggttccctgcgtcacaggcatggtgtacagagggttgagaagggccaatctaagagagggtgaaactgtgttagttacgggagcaagcggtggagttggaatacatgccctgcaagtggcaaaggccatgggagccagggtagtgggtgtcacgacgtcggaggagaaggcatccatcgttggaaagtatgctgatagggtcatagttggatcgaagttctcggaggaggcaaagaaagaggacattaacgtggtaatagacaccgtgggaacgccaaccttcgatgaaagcctaaagtcgctctggatgggaggtaggatagtccaaataggaaacgtggacccaacccaatcctatcagctgaggttaggttacaccattctaaaggatatagccataattgggcacgcgtcagccacaaggagggatgcagagggagcactaaagctgactgctgaggggaagataagaccagtggttgcgggaactgttcacctggaggagatagacaagggatatgaaatgcttaaggataagcacaaagtggggaaagtactccttaccacgtaa

SEQ ID NO: 168 - DNA, 1005 bp, Artificial Sequence
  Synthesized ST0480
atgaaagcaattgtagttccaggacctaagcaagggtataaacttgaagaggtacctgatcctaagccgggaaaagatgaagtaataattagggtagatagagctgctctttgttatagagatttgcttcaactacaaggatattatccaagaatgaaatacccagttatactagggcatgaagttgtaggaaccatagaagaagtcggagaaaatataaagggatttgaagtaggtgataaagtaatttctttattatatgcaccagatggtacatgcgaatattgccaaataggtgaggaagcatattgtcatcataggttaggctactcagaagagctagacggattttttgcagagaaagctaaaattaaagtaactagcttagtaaaggttccaaaaggtaccccagatgagggagcagtacttgtaccttgtgtaaccggaatgatatatagaggtattagaagggctggtggtatacgtaaaggggagctagtgttagttactggtgccagtggtggagtaggaatacatgcaattcaagttgctaaggccttaggtgctaaagttataggggtaacaacatcagaagaaaaagcaaagataattaagcagtatgcggattatgtcatcgttggtacaaagttttctgaagaagcaaagaagataggtgatgttactttagttattgatactgtgggtactcctactttcgatgaaagcttaaagtcattgtggatgggcggaaggattgttcaaatagggaatgtcgacccttctcaaatctataatttaagattgggctacataatattaaaagatttaaagatagttggtcatgcctcagctaccaaaaaagatgctgaagatacactaaaattaacacaagagggaaaaattaaaccagttattgcaggaacagtcagtcttgaaaatattgatgaaggttataaaatgataaaggataagaataaagtaggcaaagtcttagtaaaaccataa

SEQ ID NO: 169 - DNA, 999 bp, Artificial Sequence
  Synthesized Mcup_0809
atgaaagctgttatcgttaagggagccaaacaaggttatgaagtcagagacgttcaagatccgaaacctcaacctgatgaggtagtaataaaggttaacagagctgccctatgttacagagatctccttcaacttcaggggttttaccccaggatgaaatacccagtggttctgggacacgaagtgataggcgaaatcgttgacgtaggtagagacgtgaaggggttcgccataggggatagagtcatatccttactttacgctcctgacggtagctgtcactactgtaaaaggggagaggaggcatactgtcactctagactgggctattctgaggagcttgatggattcttcgcggagatggcaagggttaaagtaagtagcctcgttaaggtacctcctggagtttccgatgaggggggagtcatggtaccttgcgtaaccgggatgatatatagaggtttaagaagagctaacttaagcgaaggggagaccgttttagtgacaggggccagtggaggagtcggaatacacgccctgcaagtcgcgaaaggaatgggggccagagtgattggggtgacgacttcagaggagaagagttcgattatagcgaagtactctgacagggtaatagtaggttccaagttctcggaagaggccaagaaagaggacgtcaacgtgatcattgataccgttggaactcctacgtttgaggaaagcctcagatcgttatggatgggaggtagaatagtccagattggtaacgtagatcctacacaggcttaccaattgagattaggctacacgattctcaaagatattgccataattgggcatgcctcagctaccaaacgcgatgctgaagccgctttaaaactaacttcagaaggcaaggtaaggccgatagtagctggaaccgtcagcttagaggagatagataagggttacgaaatcctcaaggacaaacacaaagtagggaaggtattgctaaagccttag

SEQ ID NO: 170 - DNA, 1788 bp, Artificial Sequence
  Synthesized RBRH_02090
atgggacagtacgctgcaccgttgcgcgacatgcaattcgtcttgcacgaattgctgaacgtcgaagccgaactgaagcaactgcctaagcacgcggatctggatgccgatacgatcaatgcggtgctgcaggaggcgggcaagttctgctccgaggtcctgtttccgttgaaccaggttggcgaccagcagggttgtacgtatgtcggcgacggcgtggtgaccacgcccgagggcttcaagcaagcgtaccagcagtatatcgaggccggctggccggcgttgggctgcgatccggcctatggcggccagggcttgcccgcgttcgtgaacaacgcgctgtacgagatgctcaattcggcgaaccaggcatggaccatgtatcctggcctgtcgcacggcgcgtacgaatgcctgcacgcgcacggcacgccggagcttcaacagcggtatctaccgaagctggtatccggtcagtggaccggcacgatgtgcttgaccgagccgcattgcggcaccgaccttgggatcttgcgcacgcgggccgagcccaacggcgacggctcgtactcgattaccggcacgaagatctttatttcgagcggcgagcacgacctcgccgacaacatcgtccacctggtgctcgcgcggttgccggacgcgccggcggggaccaagggcatttcattgttcatcgtgcccaagttcatcccggacgacaacggcgagcctgggcagcgcaacggcgtcaagtgtggctcgatcgagcacaagatgggcatccatggcaatgcgacgtgcgtaatcaatctggatgatgccaggggctggctggtcggcgagccgaacaagggcttgaatgcgatgttcgtgatgatgaatgcggcgcggctcggcgtgggcatgcaaggcctggggctgaccgaagtcgcgtaccagaactcgctcgcctacgcgaggcagcggctgcagatgcgctcgcttagcggtcctaaggcgccggacaaggcggccgacccgatcatcgtgcacccggatgtgcgacgcatgttgttgacgcagaaggcctacgtcgaggcggggcgcgcgttcacgtactgggcggctctgcagatcgacaaggaactgtcgcacgaggacgaggcggtgcgccgggatgcggccgacctggttgcgttgctcacaccggtcatcaaggcgttcctgaccgacaacgcgttcgaggcgaccaacaacgccatgcaggtgttgggcggccatggctatatcgctgagtggggcatcgagcaatatgtgcgtgatgcgcgcatcaacatgatttacgaaggcactaacacgattcagtcgctggacctgctggggcgcaaggtgctcggcgacatgggcgcgaagctgaagaagtttggcaagctcgtgcaggattttgtccaggccgagggcatcaaccccgacatgcaggagttcgtcaatccgctggcggacatcggcgaaaaggtacagaagctgacgatggaaatcggcatgaaggcgatgcagagcccggacgaagttggcgccgcggcggtaccgtacctgcgcacggtcgggcatttagtgttctcgtacttttgggcgcgcatggcccgtctggcgctggacaagcaaggtagcggcgacccattctaccggtccaagctcgcgaccgcgcggttctactttgcgaagctgttacccgagacggccttcacgatccgcgccgcgcgtgccggagccaagccgctgaccgagatcgacgaagcgctgttttaa

SEQ ID NO: 171 - DNA, 981 bp, Artificial Sequence
  Synthesized RSP_1434
gtgagagccgttctgatagagaaatccgacgatacgcagtccgtttcggtgacggagcttgccgaggaccagctgcccgagggcgacgttctggtcgacgtcgcctattcgaccttgaactacaaggacgcgctggcgatcaccggcaaggcgccggtcgtgcggcgcttccccatggtgccgggcatcgacttcacgggcacggtggcacaaagcagccatgccgatttcaagcccggcgaccgggtcatcctgaatggctggggcgtgggggaaaaacactggggcgggctggccgaacgggcacgggtccgcggcgactggctggttccgctgccggcgcccctcgacttgcggcaggcggcgatgatcggcacggcgggctatacggccatgctctgcgttctggcgctcgagcggcacggggtcgtgcccggcaatggcgagatcgtcgtgagcggcgccgctggcggtgtcggcagcgttgcgacgacacttcttgccgcgaagggctacgaagttgctgcggtcaccggccgtgcctccgaggcggagtatctgcgcggtctgggcgccgcgtcggtgatcgaccgcaacgaactgaccggcaaggtccgtccgctggggcaggagcgttgggccggcggcatcgatgttgcgggcagcacggtgctggcgaacatgctctcgatgatgaaataccggggcgtcgtcgcggcctgcggtcttgccgcgggaatggatctgcccgcgtcggtggcgcccttcatcctgcgcggtatgaccctggccggggtcgacagcgtcatgtgcccgaaaaccgaccgccttgcggcctgggctcggctcgccagcgatctcgatccggcaaagctcgaggagatgacgaccgaactgcccttctccgaggtcatcgagaccgccccgaagttccttgacgggaccgtccgaggacgcatcgtcattccggtcaccccctga

SEQ ID NO: 172 - DNA, 1104 bp, Artificial Sequence
  Synthesized acrA
atggcttttaacagtgctgatataaattcgtttcgagatatttgggtattttgcgaacagagagaaggcaagcttattaatacagattttgagctgatttcagaaggaagaaagctcgctgatgagcggggctcaaaattggttggtattttattaggacatgaggtagaagagattgcaaaagagctaggtggatatggtgcagataaagtgattgtttgcgaccatcccgaattaaaattttataccacagatgcttatgccaaggtactttgtgatgtggttatggaggagaaacccgaggttattttgattggtgcaacaaatattggccgtgatttaggccccagatgtgcagcacgcttacatacgggtttaacagcagattgtacccatttggatattgatatgaacaaatatgtggactttctttccacctcctcaacattggatatatcatccatgacctttcctatggaggacacaaatttaaaaatgacccgtcctgcctttggcggacatttgatggcaactatcatttgccctagattccgcccttgtatgtctactgtaagacccggggttatgaaaaaagcagagtttagccaggaaatggctcaggcttgtcaggttgttacacgccatgtaaatttatctgatgaggacttaaagacgaaagtaatcaatattgtgaaagaaactaaaaaaattgttgatttaatcggcgccgaaattattgtttctgttggacgtggaatcagcaaggatgtgcaagggggcattgccctagcagaaaagcttgccgatgcgtttgggaatggtgttgttggcggttctcgtgcggttattgattccggttggctccctgcggatcatcaggttgggcagacgggaaaaaccgtgcatcctaaggtatatgttgcccttggtatttccggcgccattcagcataaggcaggtatgcaggattcagagttgattattgcggtaaataaagacgagactgctcctattttcgattgtgctgattatggtataacaggggatttgtttaaaattgtaccaatgatgattgatgcaattaaggaaggtaaaaatgcttga

SEQ ID NO: 173 - DNA, 804 bp, Artificial Sequence
  Synthesized acrB
atgagaatttatgtttgtgtaaaacaagttccggatacatcgggaaaggttgctgtaaaccccgatggaaccttaaaccgtgcatctatggcggctattattaatcccgatgatatgagtgccattgagcaggccttaaagttgaaggatgaaacaggctgtcaagttacagcccttaccatggggccacctcctgcggagggaatgttgcgggaaattatcgcaatgggcgcagatgatggcgttttaatttccgccagagagttcggtggttccgataccttcgcaacctctcaaattatatcggcggcgatacataaattagggctttccaatgaggatatgattttttgcggtaggcaggcaattgatggagatacagcacaggtaggaccgcaaattgcagaaaaattaagcattcctcaggtgacttatggggcagggattaaaaaaagcggagatttggttttggtaaagcgcatgctggaagatggatatatgatgatagaggtggaaacaccctgcttgattacttgcattcaggataaggctgtaaaaccacgctatatgactttgaatggaattatggaatgctatagcaagcctcttttggtattagattatgaagcccttaaggatgaacccctaatcgaattggatacgatcggtctgaaaggttctcctacaaatatatttaaatcctttacgccgccacaaaagggtgtaggcgttatgcttcaaggaacagacaaagaaaaagttgaagatttggtggacaaattgatgcagaagcatgtcatttaa

SEQ ID NO: 174 - DNA, 1562 bp, Artificial Sequence
  Synthesized Fdh1
ttcaactaaaaattgaactatttaaacactatgatttccttcaattatattaaaatcaatttcatatttccttacttctttttgctttattatacatcaataactcaattaactcattgattatttgaaaaaaaaaaacatttattaacttaactccccgattatatattatattattgactttacaaaatgaagatcgttttagtcttatatgatgctggtaagcacgctgctgatgaagaaaaattatatggttgtactgaaaataaattaggtattgctaattggttaaaagatcaaggtcatgaactaattactacttctgataaagaaggtgaaacaagtgaattggataaacatatcccagatgctgatattatcatcaccactcctttccatcctgcttatatcactaaggaaagacttgacaaggctaagaacttaaaattagtcgttgtcgctggtgttggttctgatcacattgatttagattatattaatcaaacaggtaagaaaatctcagtcttggaagttacaggttctaatgttgtctctgttgctgaacacgttgtcatgaccatgcttgtcttggttagaaatttcgttccagcacatgaacaaattattaaccacgattgggaggttgctgctatcgctaaggatgcttacgatatcgaaggtaaaactattgctaccattggtgctggtagaattggttacagagtcttggaaagattactcccttttaatccaaaagaattattatactacgattatcaagctttaccaaaagaagctgaagaaaaagttggtgctagaagagttgaaaatattgaagaattagttgctcaagctgatatcgttacagttaatgctccattacacgcaggtacaaaaggtttaattaataaggaattattatctaaatttaaaaaaggtgcttggttagtcaataccgcaagaggtgctatttgtgttgctgaagatgttgcagcagctttagaatctggtcaattaagaggttacggtggtgatgtttggttcccacaaccagctccaaaggatcacccatggagagatatgagaaataaatatggtgctggtaatgccatgactcctcactactctggtactactttagatgctcaaacaagatacgctgaaggtactaaaaatatcttggaatcattctttactggtaaatttgattacagaccacaagatattatcttattaaatggtgaatacgttactaaagcttacggtaaacacgataagaaataaattttcttaacttgaaaactataattgctataacaattcttcaatttctctttttcttcctttttttgaagaatttttaacaatcaaaattttgactctttgatttcccgcaatctctgagctcagcatactcattattattttattattattattattattacttttattattattatatttttycttctttaacgatatcgtttgtgttttatcttttatgatttaaattttatacgaatttatgaatacaacaaaatatttaagtttacacaatg

SEQ ID NO: 175 - DNA, 1131 bp, Artificial Sequence
  Synthesized Fdh1 (1)
atgtcgaagggaaaggttttgctggttctttacgaaggtggtaagcatgctgaagagcaggaaaagttattggggtgtattgaaaatgaacttggtatcagaaatttcattgaagaacagggatacgagttggttactaccattgacaaggaccctgagccaacctcaacggtagacagggagttgaaagacgctgaaattgtcattactacgccctttttccccgcctacatctcgagaaacaggattgcagaagctcctaacctgaagctctgtgtaaccgctggcgtcggttcagaccatgtcgatttagaagctgcaaatgaacggaaaatcacggtcaccgaagttactggttctaacgtcgtttctgtcgcagagcacgttatggccacaattttggttttgataagaaactataatggtggtcatcaacaagcaattaatggtgagtgggatattgccggcgtggctaaaaatgagtatgatctggaagacaaaataatttcaacggtaggtgccggtagaattggatatagggttctggaaagattggtcgcatttaatccgaagaagttactgtactacgactaccaggaactacctgcggaagcaatcaatagattgaacgaggccagcaagcttttcaatggcagaggtgatattgttcagagagtagagaaattggaggatatggttgctcagtcagatgttgttaccatcaactgtccattgcacaaggactcaaggggtttattcaataaaaagcttatttcccacatgaaagatggtgcatacttggtgaataccgctagaggtgctatttgtgtcgcagaagatgttgccgaggcagtcaagtctggtaaattggctggctatggtggtgatgtctgggataagcaaccagcaccaaaagaccatccctggaggactatggacaataaggaccacgtgggaaacgcaatgactgttcatatcagtggcacatctctggatgctcaaaagaggtacgctcagggagtaaagaacatcctaaatagttacttttccaaaaagtttgattaccgtccacaggatattattgtgcagaatggttcttatgccaccagagcttatggacagaagaaataa

SEQ ID NO: 176 - DNA, 438 bp, Artificial Sequence
  Synthesized Fdh2
atgtcgaagggaaaggttttgctggttctttatgaaggtggtaagcatgctgaagagcaggaaaagttattggggtgtattgaaaatgaacttggtatcagaaatttcattgaagaacagggatacgagttggttactaccattgacaaggaccctgagccaacctcaacggtagacagggagttgaaagacgctgaaattgtcattactacgccctttttccccgcctacatctcgagaaacaggattgcagaagctcctaacctgaagctctgtgtaaccgctggcgtcggttcagaccatgtcgatttagaagctgcaaatgaacggaaaatcacggtcaccgaagttactggttctaacgtcgtttctgtcgcagagcacgttatggccacaattttggttttgataagaaactataatggtggtcatcaataa

SEQ ID NO: 177 - DNA, 867 bp, Artificial Sequence
  Synthesized fdsC
atgatgcgctgcatgcagtcaccggaggtgcatccggccgcggccggagacgccgagccgcccactcacagcaccttcgccgtcagccgctggcgccgcggcgagctgatgctgagccccgatgaagtggccgaggaagtgccggtcgcgctggtgtacaacggcatctcgcacgcggtgatgctggcgacgccggccgacctggaggacttcgcactcggcttcagcctgagcgaaggcatcgttacccgtgccagcgacgtctatgacatcgagatcgacacgcgcgagcacggcatcgccgtgcagctggagatcgcatcggaagccttcatgcggctcaaggaccgccgccgctcgctggccgggcgcaccggctgcgggctgtgcggcaccgaatcgctggaacaggtgatgcgcctgccggcaccggtgcgcagcgatgccagcttccataccgacgtgatccaggccgcgttcgtgcaactgcaactgcggcaggaactgcagcaacacacgggtgcgacgcacgctgccgcatggctgcgtgccgatggccatgtatcactggtgcgtgaagacgtgggccgccacaacgcgctggacaagctggcgggcgcgctcgccagcagcggcgaggacatctccagcggcgcggtgctggtgaccagccgcgccagctatgaaatggtgctgaagaccgccgccatcggcgccggcgtgctcgccgcagtgtccgcaccgacggcgctggccgtgcggcttgccgaacaagccagcatcaccctggccggcttcgtgcgcgccggcgcgcacgtggtctatgcccatccccaacgcctgcagcacgaagcgagcctggcatga

SEQ ID NO: 178 - DNA, 2880 bp, Artificial Sequence
  Synthesized fdsA
atgaacgcccgcaacgagatcgatttcggcacgcccgccagcccatccaccgaactggtcaccctggaggtcgatggcgtcagcgtcaccgtgcccgccggcacctcggtgatgcgcgccgcgatggaagcgcagatcgccgtccccaagctgtgcgccaccgacagcctcgaagccttcggctcgtgccggctgtgcctggtcgagatcgaagggcgccgcggctatccggcatcgtgcaccacgccggtcgaagccggcatgaaggtcaagacccagagcgacaagctggccgacctgcgccgcggcgtgatggagctgtatatctccgaccacccgctcgattgcctgacctgcccgaccaacggcaactgcgagctgcaggacatggccggcgtggtcggcctgcgtgaagtgcgctacaacgacggcggcccggaagctgcgccgatcgcgacccacacgcagatgaagaaggacgaatccaatccttacttcacctacgacccctccaagtgcatcgtctgcaaccgctgcgtgcgcgcctgcgaggaaacgcagggcaccttcgccctgaccatcagcggccgcggcttcgattcccgcgtctcgcccggaaccagccagtcgttcatggaatcggactgcgtctcgtgcggcgcctgcgtgcaggcgtgcccgaccgcgacgctgaccgagacctcggtgatcaagttcggccagccctcgcacagcaccgtgaccacctgtgcctattgcggcgtgggctgttcgttcaaggccgagatgaagggcaatgaagtggtgcgcatggtgccgtacaaggacggcaaggccaatgaaggccacgcctgcgtcaagggccgctttgcctggggctacgccacgcacaaggaccgcatcctcaagccgatgatccgcgccaagatcaccgatccgtggcgcgaggtgtcgtgggaagaggcgatcgactatgccgcgtcgcagttcaagcgtatccaggccgagcacggcaaggactccatcggcggcatcgtgtcgtcgcgctgcaccaatgaagagggctacctggtgcagaagctggtgcgcgcagccttcggcaacaacaacgtcgacacctgcgcgcgcgtgtgccattcgccgaccggctacggcctgaagcagaccctgggcgaatcggccggcacgcagaccttcaagtcggtggagaaggccgacgtgatcatggtgatcggtgccaacccgaccgacggccacccggtctttgcgtcgcgcatgaagaagcgcctgcgcgccggcgccaggctgatcgtggtcgatccgcgccgcatcgacctggtcgactccccgcatatccgtgccgactatcacctgcaactgcgcccgggcaccaacgtggcgctggtgacctcgctggcccacgtgatcgtcaccgaaggcctgctcaacgaagctttcatcgccgagcgctgcgaggaccgcgccttccagcaatggcgcgatttcgtctcgctgccggagaactcgccggaggcgatggaaagcgtgaccggcattccggcggaacagctgcgcggtgccgcacgcctgtatgccaccggcggcaacgctgcgatctactacggcctgggcgtgaccgagcatgcgcaaggctcaaccaccgtgatgggcattgccaacctcgccatggccaccggcaatatcggccgcgaaggcgtgggtgtgaacccgctgcgcgggcagaacaatgtgcagggctcgtgcgacatcggttcgttcccgcatgagctgccgggctatcgccacgtgtcggactcgaccacgcgcggtctgttcgaagccgcgtggaatgtcgagatcagccccgagccgggcctgcgcatccccaatatgtttgaagccgcgctggccggcagcttcaagggcctctactgccagggcgaggacattgtccagtccgacccgaacacgcagcacgtgtccgaggcgctgtcatcgatggaatgcatcgtggtgcaggacatcttcctgaacgagaccgccaagtacgcgcacgtgttcctgccgggctcgtccttcctggaaaaggacggcaccttcaccaacgccgagcgccgcatctcgcgcgtgcgcaaggtgatgccgcccaaggcgcgctatgccgactgggaagccaccatcctgctggccaatgcgctgggctacccgatggactacaagcatccgtcggagatcatggacgagatcgcgcgcctgacgccgaccttcgccggtgtcagctacaagcgcctggaccagctcggcagcatccagtggccgtgcaacgccgacgcgccggaaggcacgccgaccatgcatatcgacaccttcgtgcgcggcaagggcaagttcatcatcaccaagtacgtgcccaccaccgagaagatcacgcgcgccttcccgctgatcctgaccaccggccgcatcctgtcgcaatacaacgtcggcgcgcagacgcgccgtaccgacaacgtctactggcatgccgaggaccggctcgagatccatccgcacgatgccgaggagcgcggcatcaaggacggcgactgggtcggggtgcagagccgtgccggcgacacggtgctgcgcgcgatcgtcagcgagcgcatgcagccgggcgtggtctacaccaccttccacttcccggaatccggcgccaacgtgatcaccaccgacaactccgactgggccaccaactgcccggagtacaaggtgaccgcggtgcaggtgctgccggtggcgcagccgtcggcgtggcagcgggagtaccaggagttcaacgcccagcagctgcaactgctggaagccgccagcgccgacccggcgcaggccgtacgctga

SEQ ID NO: 179 - DNA, 1563 bp, Artificial Sequence
  Synthesized fdsB
atgatcacgatcaccaccatcttcgtgccgcgcgattccaccgcgctggcactgggcgccgacgacgtcgcccgcgccatcgcgcgtgaagccgcggcgcgcaacgagcacgtgcgcattgtgcgcaatggctcgcgcggcatgttctggctggagccgctggtcgaggtgcagaccggagccggccgcgtggcctatggcccggtcagcgccgcagacgtgccggggctgttcgacgccggcttgctgcaaggcggcgagcacgcgctgtcgcagggcgtcaccgaagagatccccttcctgaagcagcaggagcgcctgaccttcgcccgcgtcggcatcaccgatccgctgtcgctggacgactaccgcgcgcatgagggctttgccggcctggagcgcgcgctggcgatgcagcccgccgagatcgtgcaggaggtcaccgactccggcctgcgcggccgcggcggcgcggcgttcccgaccggcatcaagtggaagaccgtgctgggcgcgcagtccgcggtcaagtacatcgtctgcaatgccgacgagggcgactcgggcacgttctccgatcgcatggtgatggaagacgacccgttcatgctgatcgaaggcatgaccattgccgcgcttgcggtgggtgcggagcagggctacatctactgccgttccgaatacccgcacgcgattgccgtgctggaaagcgcgattggtatcgccaacgccgccggctggctcggcgacgacatccgcggcagcggcaagcgcttccacctcgaagtgcgcaagggcgccggcgcctatgtctgcggcgaggaaaccgcgctgctggaaagcctggaaggacggcgcggcgtggtgcgcgccaagccgccgctgccggcgctgcaggggctgttcggcaagcccacggtgatcaacaacgtgatctcgctggccaccgtgccggtgatcctggcgcgcggcgcgcagtactaccgcgactacggcatgggccgttcgcgcggcacgctgccgttccagcttgccggcaacatcaagcagggcggactggtggaaaaggcgttcggcgtgacgctgcgcgagctgctggtcgactacggcggcggcacgcgcagcggccgcgccatccgcgcggtgcaggtgggcgggccgctgggcgcctacctgcccgagtcgcgcttcgacgtgccgctggactatgaagcctatgccgcgttcggcggcgtggtcggccacggcggcatcgtggtgttcgatgaaaccgtcgacatggcaaagcaggcccgctacgcgatggagttctgcgcgatcgaatcgtgcggcaagtgcaccccgtgccggatcggctcgacccgcggcgtcgaagtgatggaccgcatcatcgccggcgagcagccggtcaagcacgtcgccctggtgcgcgacctgtgcgacaccatgctcaacggctcgctgtgcgcgatgggcggcatgaccccgtacccggtgctgtccgcgctgaatgaattccccgaggacttcggcctcgcctccaacccagccaaggccgcctga

SEQ ID NO: 180 - DNA, 225 bp, Artificial Sequence
  Synthesized fdsD
atgaagatcgacaacctcatcaccatggccaaccagatcggcagcttcttcgaggccatgccggatcgggaagaggccgtctctgatattgcagggcatatcaagcggttttgggagccgcgcatgcgcaaggccttgctggggcatgtggatgccgaggcagggagcgggctgctggacatcgtgcgcgaggcgctggggcggcatcgggcgatgctggagtag

SEQ ID NO: 181 - DNA, 531 bp, Artificial Sequence
  Synthesized fdsG
atgccagaaatttccccccacgcaccggcatccgccgatgccacgcgcatcgccgccatcgtggccgcgcgccaggacataccgggcgccttgctgccgatcctgcatgagatccaggacacacagggctatatccccgacgccgccgtgcccgtcattgcccgcgcgctgaacctgtcgcgcgccgaggtgcacggcgtgatcaccttctaccaccatttccgccagcagccggccgggcgccacgtggtgcaggtctgccgcgccgaagcctgccagtcggtcggcgccgaagcgctggccgagcatgcgcagcgcgcacttggctgtggctttcatgaaaccaccgcggacgggcaggtgacgctggagccggtttattgcctgggccagtgcgcctgcggccccgccgtgatggtcggcgagcagctgcacggctatgtcgatgccaggcgcttcgacgcgctggtgcgctcgctgcgcgagtcgtccgcggaaaagaccacggaagccgcggaggcacaggcatga

SEQ ID NO: 182 - DNA, 1095 bp, Artificial Sequence
  Synthesized fdsR
atgattcgcatctcgatccacccgcacctgcagatccgggacgacgccagccccggtggcgaggccctggacgtgtcccgcctggtggccctgctcggccatatcgaggaatccggcagcatcagccactcggcgcaggcggtatcgctgtcctaccgctacgcctggggcatcctgcgcgatgccgaggcgctgttcggcggcccgctgatcgacaagacccgcgggcgcggcagcgcgctgacgccgctggcgcagcagttggtgtgggccagcaagcggatcggcgcgcggctgtcgccgacgctggacagcctggcgtccgagctggagatcgagttgaagaagctgatggaccagcccgaagccacggcgcggctgcatgccagccacggcttcgcggtggcggcgctgcgcgacttcctcgacgagcagcaggtgcggcacgacctgaagtactgcggcagcgtcgaggccgtggcggcactggccgaaggcgcctgcgatatcgccggcttccatgtgccggtgggcgagttcgagcacggcatgtggcggcatttcaccacctggctcaagccggacacccactgcctggtgcacctggcggtgcgcagccagggactgttcgtgcggccggacaacccgcttggcatccacacgctggaagacctgacccggcgcgaggtgcgcttcgtcaaccgccaggtgggctcgggcacgcgcctgctgctggacctgatgctggccgcgcgcggcatcgacacggcccgcatcgagggctacagcaacggtgaattcacccacgccgcggtggccgcgtatatcggcagcggcatggccgacgtgggctttggcgtggaaaccgcggcgcggcgcttcgggctggcgttcgtgccggtgatcaaggagcgctacttctttgcgatcgagcgcgccaagctgcgcagcgcggcactggccggcgcggtggacgcgcttaccagcgaagccttccgccagcgcgtcaatgcactgcccggctacgacggcacgctgaccggcaccgtgctgacgctggaagaagcgttcccggattacgctgaggcgcgctag

(19) United States
(12) Patent Application Publication
(10) Pub. No.: US 2015/0152440 A1
(43) Pub. Date: Jun. 4, 2015

(54) MODIFIED MICROORGANISMS AND METHODS OF CO-PRODUCING BUTADIENE WITH 1-PROPANOL AND/OR 1,2-PROPANEDIOL

(71) Applicant: Braskem S.A., Camacari, BA (BR)

(72) Inventors: Mateus Schreiner Garcez Lopes, Camacari (BR); Avram Michael Slovic, Camacari (BR); Paulo Luiz de Andrade Coutinho, Camacari (BR); Antonio Luiz Ribeiro De Castro Morschbacker, Camacari (BR)

(73) Assignee: Braskem S.A., Camacari, BA (BR)

(21) Appl. No.: 14/409,292

(22) PCT Filed: Jun. 18, 2013
(86) PCT No.: PCT/US2013/046330

§ 371 Date: Dec. 18, 2014

Related U.S. Application Data
(60) Provisional application No. 61/661,153, filed on Jun. 18, 2012.

(51) Int. Cl. C12P 5/02; C12P 7/04; C12N 15/63; C12P 7/18

(52) U.S. Cl. CPC C12P 5/026; C12P 7/18; C12P 7/04; C12N 15/63

(57) ABSTRACT

The present disclosure generally relates to microorganisms that comprise one or more polynucleotides coding for enzymes in a pathway that catalyzes a conversion of a fermentable carbon source to butadiene and/or one or more polynucleotides coding for enzymes in a pathway that catalyzes a conversion of a fermentable carbon source to 1-propanol and/or 1,2-propanadiol. Also provided are methods of using the microorganisms to produce butadiene and co-products such 1-propanol and/or 1,2-propanediol.

### BACKGROUND

Butadiene (1,3-butadiene, CH₂═CH—CH═CH₂, CAS 106-99-0) is a linear, 4-carbon molecule with 2 conjugated double bonds typically manufactured (along with other 4-carbon molecules) by steam cracking petroleum-based hydrocarbons. This process involves harsh conditions and high temperatures (≧850° C.). Other methods of butadiene production involve toxic and/or expensive catalysts, highly flammable and/or gaseous carbon sources, and high temperatures. Globally, several million tons of butadiene-containing polymers are produced annually. Butadiene can be polymerized to form polybutadiene, or reacted with hydrogen cyanide (prussic acid) in the presence of a nickel catalyst to form adiponitrile, a precursor to nylon. More commonly, however, butadiene is polymerized with other olefins to form copolymers such as acrylonitrile-butadiene-styrene (ABS), acrylonitrile-butadiene (ABR), or styrene-butadiene (SBR) copolymers.

1-propanol (n-propanol, CH₃CH₂CH₂OH, CAS 71-23-8) is a primary alcohol typically manufactured by catalytic hydrogenation of propionaldehyde, which is generally synthesized in large scale from ethylene in an energy-intensive multi-step industrial process. This process involves use of toxic chemicals such as carbon monoxide and hydrogen at high pressure (e.g., 10-100 ATM) and high temperature (up to 200° C.). Globally, more than 140,000 tonnes (154,000 tons) of 1-propanol are produced annually. 1-propanol can be used as an intermediate for further organic reactions or as a building block for polymers such as propylene. Propylene is a chemical compound that is widely used to synthesize a wide range of petrochemical products. For instance, this olefin is the raw material used for the production of polypropylene, its copolymers and other chemicals such as acrylonitrile, acrylic acid, epichloridrine and acetone. Propylene is typically obtained in large quantity scales as a byproduct of catalytical or thermal oil cracking, or as a co-product of ethylene production from natural gas. (Propylene, Jamie G. Lacson, CEH Marketing Research Report-2004, Chemical Economics Handbook-SRI International). Propylene is polymerized to produce thermoplastics resins for innumerous applications such as rigid or flexible packaging materials, blow molding and injection molding.

1,2-propanediol (propylene glycol, HO—CH2-CHOH—CH3, CAS 57-55-6) is an organic compound with formula C₃H₈O₂. Industrially, propylene glycol is produced from propylene oxide. Propylene glycol may be manufactured using either a non-catalytic high-temperature process at 200° C. (392° F.) to 220° C. (428° F.), or a catalytic method, which proceeds at 150° C. (302° F.) to 180° C. (356° F.) in the presence of ion exchange resin or a small amount of sulfuric acid or alkali. Propylene glycol can be used as a solvent, nontoxic antifreeze and to produce polyesteres compounds

Given the world-wide demand for butadiene, 1-propanol, and 1,2-propaneiol, and products derived therefrom, there exits a need in the art for improved methods for their production which overcome their current production drawbacks including the use of toxic and/or expensive catalysts, and highly flammable and/or gaseous carbon sources.

### SUMMARY

The present disclosure generally relates to microorganisms (e.g., non-naturally occurring microorganisms) that comprise one or more polynucleotides coding for enzymes in a pathway that catalyze a conversion of a carbon source to butadiene and 1-propanol and/or 1,2-propanediol and the use of such microorganisms for the production of butadiene and/or propylene. The methods of the present disclosure are advantageous over prior methods in that they reduce (including eliminate) the need for toxic and expensive catalysts, and can be performed anaerobically thereby reducing (or eliminating) the risk of fire or explosion.

The present disclosure also provides methods of co-producing butadiene and 1-propanol and/or 1,2-propanediol from a fermentable carbon source, comprising: providing a fermentable carbon source; contacting the fermentable carbon source with a microorganism comprising one or more polynucleotides coding for enzymes in a pathway that catalyzes a conversion of the fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol in a fermentation media; and expressing the one or more polynucleotides coding for the enzymes in the pathway that catalyzes a conversion of the fermentable carbon source to one or more intermediates in a pathway for the co-production of butadiene and 1-propanol and/or 1,2-propanediol and one or more polynucleotides coding for enzymes in a pathway that catalyze a conversion of the one or more intermediates to butadiene and 1-propanol and/or 1,2-propanediol in the microorganism to produce butadiene and 1-propanol and/or 1,2-propanediol.

In some embodiments of each or any of the above or below-mentioned embodiments, the enzymes that catalyze a conversion of the fermentable carbon source to one or more intermediates in the pathway for the co-production of butadiene and 1-propanol and/or 1,2-propanediol are set forth in any one of FIGS. 1-4.

In some embodiments of each or any of the above or below-mentioned embodiments, the enzymes that catalyze a conversion of the one or more intermediates to butadiene and 1-propanol and/or 1,2-propanediol are set forth in any one of FIGS. 1-4.

In some embodiments of each or any of the above or below-mentioned embodiments, the one or more intermediates in the pathway for the production of butadiene are selected from the group consisting of: crotonyl alcohol, 5-hydroxy-3-ketovaleryl-CoA, 3-ketopent-4-enoyl-CoA and 3,5-ketovaleryl-CoA.

In some embodiments of each or any of the above or below-mentioned embodiments, the one or more intermediates in the pathway for the production of 1-propanol and/or 1,2-propanediol are selected from the group consisting of: methylglyoxal and lactate.

In some embodiments of each or any of the above or below-mentioned embodiments, butadiene and 1-propanol and/or 1,2-propanediol are produced.

In some embodiments of each or any of the above or below-mentioned embodiments, butadiene and 1-propanol are produced.

In some embodiments of each or any of the above or below-mentioned embodiments, butadiene and 1,2-propanediol are produced.

In some embodiments of each or any of the above or below-mentioned embodiments, butadiene is produced via a crotonyl-alcohol intermediate and 1-propanol and/or 1,2-propanediol is produced via a methylglyoxal and a R/S lactate intermediate.

In some embodiments of each or any of the above or below-mentioned embodiments, butadiene is produced via a 5-hydroxy-3-ketovaleryl-CoA intermediate and 1-propanol and/or 1,2-propanediol is produced via a methylglyoxal and a R/S lactate intermediate.

In some embodiments of each or any of the above or below-mentioned embodiments, butadiene is produced via a 3-ketopent-4-enoyl-CoA intermediate and 1-propanol and/or 1,2-propanediol is produced via a methylglyoxal and a R/S lactate intermediate.

In some embodiments of each or any of the above or below-mentioned embodiments, butadiene is produced via a 3,5-ketovaleryl-CoA intermediate and 1-propanol and/or 1,2-propanediol is produced via a methylglyoxal and a R/S lactate intermediate.

In some embodiments of each or any of the above or below-mentioned embodiments, the microorganism is an archea, bacteria, or eukaryote.

In some embodiments of each or any of the above or below-mentioned embodiments, the bacteria is selected from the genera consisting of: Propionibacterium, Propionispira, Clostridium, Bacillus, Escherichia, Pelobacter, or Lactobacillus.

In some embodiments of each or any of the above or below-mentioned embodiments, the eukaryote is a yeast, filamentous fungi, protozoa, or algae.

In some embodiments of each or any of the above or below-mentioned embodiments, the yeast is Saccharomyces cerevisiae or Pichia pastoris.

In some embodiments of each or any of the above or below-mentioned embodiments, the carbon source is sugarcane juice, sugarcane molasses, hydrolyzed starch, hydrolyzed lignocellulosic materials, glucose, sucrose, fructose, lactate, lactose, xylose, pyruvate, or glycerol in any form or mixture thereof.

In some embodiments of each or any of the above or below-mentioned embodiments, the carbon source is a monosaccharide, oligosaccharide, or polysaccharide.

In some embodiments of each or any of the above or below-mentioned embodiments, the produced butadiene and 1-propanol and/or 1,2-propanediol are secreted by the microorganism into the fermentation media.

In some embodiments of each or any of the above or below-mentioned embodiments the methods may further comprise recovering the produced butadiene and 1-propanol and/or 1,2-propanediol from the fermentation media.

In some embodiments of each or any of the above or below-mentioned embodiments, the microorganism has been genetically modified to express the one or more polynucleotides coding for enzymes in a pathway that catalyzes a conversion of the fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol.

In some embodiments of each or any of the above or below-mentioned embodiments, the conversion of the fermentable carbon source to butadiene and 1-propanol and/or 1,2-propanediol is anaerobic.

The present disclosure also provides microorganisms comprising one or more polynucleotides coding for enzymes in a pathway that catalyzes a conversion of a fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol.

In some embodiments of each or any of the above or below-mentioned embodiments, the enzymes that catalyze a conversion of the fermentable carbon source to one or more intermediates in the pathway for the co-production of butadiene and 1-propanol and/or 1,2-propanediol are set forth in any one of FIGS. 1-4.

In some embodiments of each or any of the above or below-mentioned embodiments, the enzymes that catalyze a conversion of the one or more intermediates to butadiene and 1-propanol and/or 1,2-propanediol are set forth in any one of FIGS. 1-4.

In some embodiments of each or any of the above or below-mentioned embodiments, butadiene is produced via a crotonyl alcohol intermediate and 1-propanol and/or 1,2-propanediol is produced via a methylglyoxal and a R/S lactate intermediate.

In some embodiments of each or any of the above or below-mentioned embodiments, butadiene is produced via a 5-hydroxy-3-ketovaleryl-CoA intermediate and 1-propanol and/or 1,2-propanediol is produced via a methylglyoxal and a R/S lactate intermediate.

In some embodiments of each or any of the above or below-mentioned embodiments, butadiene is produced via a 3-ketopent-4-enoyl-CoA intermediate and 1-propanol and/or 1,2-propanediol is produced via a methylglyoxal and a R/S lactate intermediate.

In some embodiments of each or any of the above or below-mentioned embodiments, butadiene is produced via a 3,5-ketovaleryl-CoA intermediate and 1-propanol and/or 1,2-propanediol is produced via a methylglyoxal and a R/S lactate intermediate.

In some embodiments of each or any of the above or below-mentioned embodiments, the microorganism is an archea, bacteria, or eukaryote.

In some embodiments of each or any of the above or below-mentioned embodiments, the bacteria is selected from the genera consisting of: Propionibacterium, Propionispira, Clostridium, Bacillus, Escherichia, Pelobacter, or Lactobacillus.

In some embodiments of each or any of the above or below-mentioned embodiments, the eukaryote is a yeast, filamentous fungi, protozoa, or algae.

In some embodiments of each or any of the above or below-mentioned embodiments, the yeast is Saccharomyces cerevisiae or Pichia pastoris. In some embodiments of each or any of the above or below-mentioned embodiments, the microorganism has been genetically modified to express the one or more polynucleotides coding for enzymes in a pathway that catalyzes a conversion of the fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol.

These and other embodiments of the present disclosure will be disclosed in further detail herein below.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 depicts an exemplary pathway for the co-production of butadiene via crotonyl alcohol and 1-propanol or 1,2-propanediol via a methylglyoxal and/or a lactate intermediate.

FIG. 2 depicts an exemplary pathway for the co-production of butadiene via 5-hydroxy-3-ketovaleryl-CoA and 1-propanol or 1,2-propanediol via a methylglyoxal and/or a lactate intermediate.

FIG. 3 depicts an exemplary pathway for the co-production of butadiene via 3-ketopent-4-enoyl-CoA and 1-propanol or 1,2-propanediol via a methylglyoxal and/or a lactate intermediate.

FIG. 4 depicts an exemplary pathway for the co-production of butadiene via 3,5-ketovaleryl-CoA and 1-propanol or 1,2-propanediol via a methylglyoxal and/or a lactate intermediate.

FIG. 5 depicts a block flow diagram for the co-production of butadiene and 1-propanol from a sugar.

FIG. 6 depicts a block flow diagram for the co-production of butadiene and 1,2-propanediol from a sugar.

FIG. 7 depicts a block flow diagram for the co-production of butadiene, 1,2-propanediol and 1-propanol from a sugar.

### DETAILED DESCRIPTION

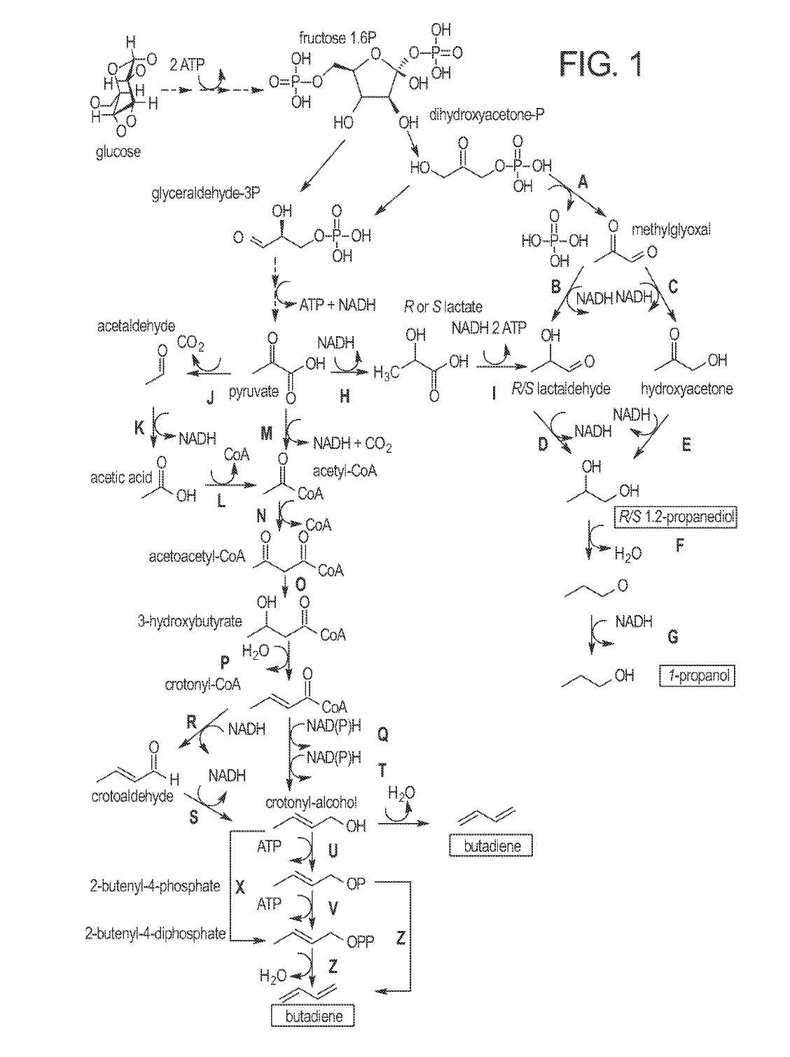

The present disclosure generally relates to microorganisms (e.g., non-naturally occurring microorganisms) that comprise a genetically modified pathway and uses of the microorganisms for the conversion of a fermentable carbon source to butadiene and 1-propanol and/or 1,2-propanediol (see, FIGS. 1-4). Such microorganisms may comprise one or more polynucleotides coding for enzymes that catalyze a conversion of a fermentable carbon source to butadiene and one or more polynucleotides coding for enzymes that catalyze a conversion of a fermentable carbon source to 1-propanol and/or 1,2-propanediol. In some embodiments, butadiene is further converted to polybutadiene or one or more of another butadiene-containing polymer. In some embodiments, 1-propanol is further converted to propylene. In some embodiments, 1,2-propanediol is further converted to polyurethane.

This disclosure provides, in part, the discovery of novel anaerobic enzymatic pathways including, for example, novel combinations of enzymatic pathways, for the production of butadiene and 1-propanol and/or 1,2-propanediol from a carbon source (e.g., a fermentable carbon source). In an embodiment butadiene and 1-propanol are produced. In another embodiment, butadiene and 1,2-propanediol are produced. In yet another embodiment, butadiene, 1-propanol, and 1,2-propanediol are produced.

The methods provided herein provide end-results similar those of sterilization without the high capital expenditure and continuing higher management costs required to establish and maintain sterility throughout a production process. In this regard, most industrial-scale butadiene and 1-propanol production processes are operated in the presence of measurable numbers of bacterial contaminants. It is believed that bacterial contamination of a butadiene and 1-propanol production processes causes a reduction in product yield and an inhibition of yeast growth (see, Chang et al., 1995, J. Microbiol. Biotechnol. 5:309-314; Ngang et al., 1990, Appl. Microbiol. Biotechnol. 33:490-493). Such drawbacks of prior methods are avoided by the presently disclosed methods as the toxic nature of the produced 1-propanol and/or 1,2-propanediol reduces contaminants in the production process.

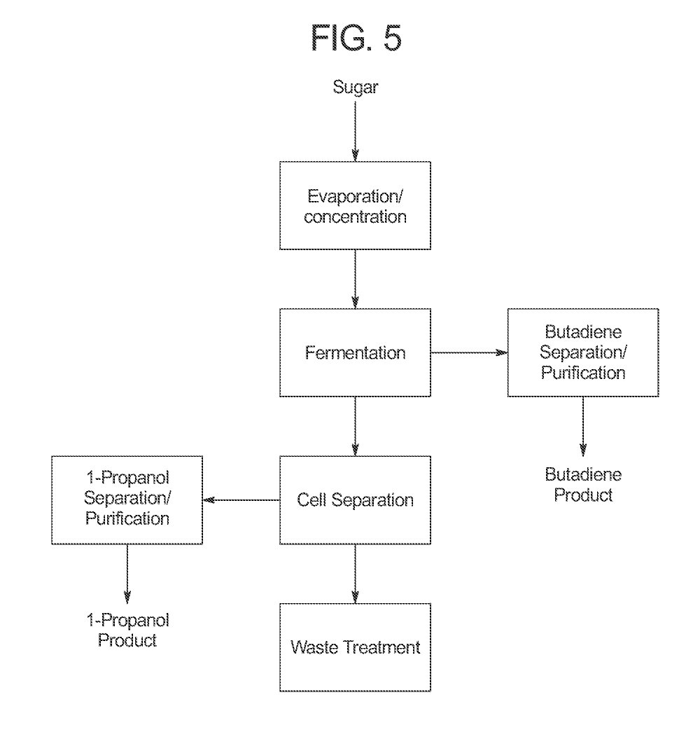

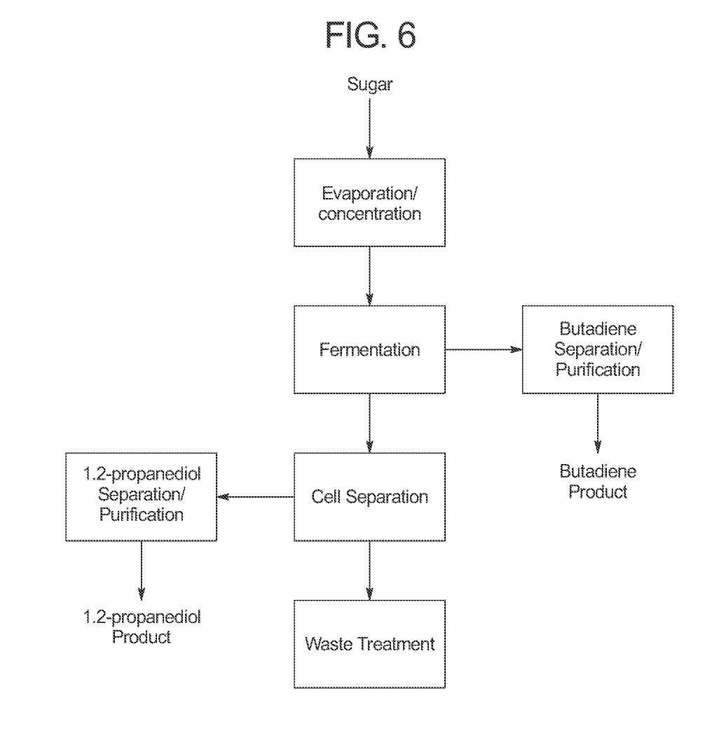

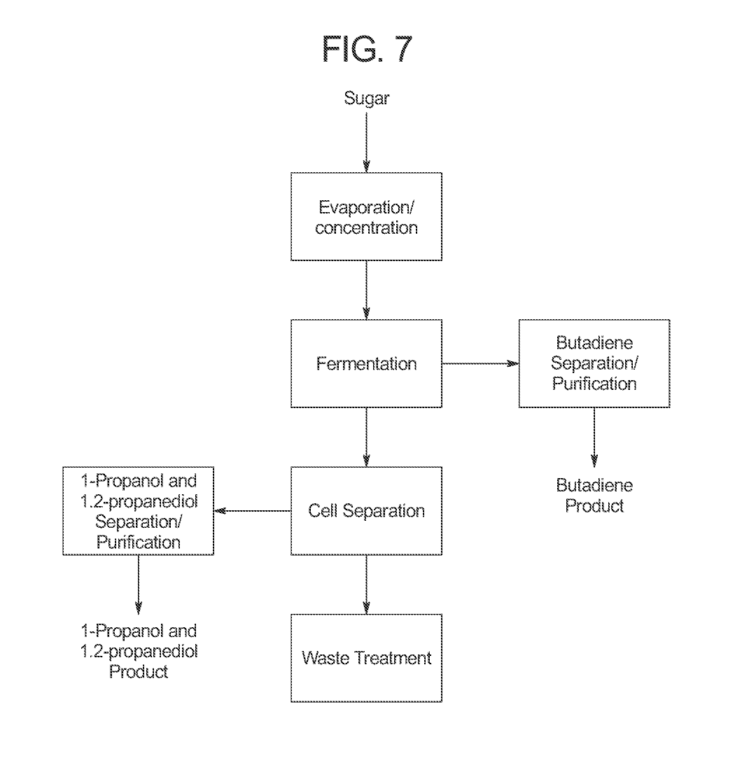

Further, the methods of the present disclosure avoid the separation drawbacks commonly associated with co-production of two or more products since the products are physically separated in the off-reactor streams: a gaseous butadiene upper stream and liquid 1-propanol and/or 1,2-propanediol stream (see, FIGS. 5-7).

Additionally, the enzymatic pathways disclosed herein are advantageous over prior known enzymatic pathways for the production of butadiene and 1-propanol and/or 1,2-propanediol in that the enzymatic pathways disclosed herein are anaerobic. While it is possible to use aerobic processes to produce butadiene and 1-propanol and/or 1,2-propanediol, anaerobic processes are preferred due risk incurred when olefins (which are by nature are explosive) are mixed with oxygen during the fermentation process. Moreover, the supplementation of oxygen and nitrogen in a fermenter requires an additional investment for aerobic process and another additional investment for the purification from the nitrogen from the butadiene. The presence of oxygen can also catalyze the polymerization of butadiene and can promote the growth of aerobic contaminants in the fermentor broth. Additionally, aerobic fermentation processes for the production of butadiene present several drawbacks at industrial scale (where it is technically challenging to maintain aseptic conditions) such as the fact that: (i) greater biomass is obtained reducing overall yields on carbon; (ii) the presence and oxygen favors the growth of contaminants (Weusthuis et al., 2011, Trends in Biotechnology, 2011, Vol. 29, No. 4, 153-158) and (iii) the mixture of oxygen and gaseous compounds such as butadiene poses serious risks of explosion, (iv) the oxygen can catalyze the unwanted reaction of polymerization of the olefin and, finally, (v) higher costs of fermentation and purification in aerobic conditions. Each of the drawbacks associated with aerobic fermentation including, for example, the risk of an explosion during the manufacture of butadiene, and dilution by oxygen and nitrogen are overcome by the anaerobic fermentation methods provided herein.

The present disclosure provides a microorganism comprising one or more polynucleotides coding for enzymes in a pathway that catalyzes a conversion of a fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol, wherein the one or more intermediates in the pathway for the production of butadiene are selected from the group consisting of: crotonyl alcohol, 5-hydroxy-3-ketovaleryl-CoA, 3-ketopent-4-enoyl-CoA and 3,5-ketovaleryl-CoA, wherein the one or more intermediates in the pathway for the production of 1-propanol and/or 1,2-propanediol are selected from the group consisting of: methylglyoxal and lactate.

The present disclosure also provides a method of co-producing butadiene and 1-propanol and/or 1,2-propanediol from a fermentable carbon source, the method comprising: providing a fermentable carbon source; contacting the fermentable carbon source with a microorganism comprising one or more polynucleotides coding for enzymes in a pathway that catalyzes a conversion of the fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol in a fermentation media; and expressing the one or more polynucleotides coding for the enzymes in the pathway that catalyzes a conversion of the fermentable carbon source to one or more intermediates in a pathway for the co-production of butadiene and 1-propanol and/or 1,2-propanediol and one or more polynucleotides coding for enzymes in a pathway that catalyze a conversion of the one or more intermediates to butadiene and 1-propanol and/or 1,2-propanediol in the microorganism to produce butadiene and 1-propanol and/or 1,2-propanediol, wherein the one or more intermediates in the pathway for the production of butadiene are selected from the group consisting of: crotonyl alcohol, 5-hydroxy-3-ketovaleryl-CoA, 3-ketopent-4-enoyl-CoA and 3,5-ketovaleryl-CoA, wherein the one or more intermediates in the pathway for the production of 1-propanol and/or 1,2-propanediol are selected from the group consisting of: methylglyoxal and lactate, and wherein the co-production method is anaerobic.

It will be understood that the steps involved in any and all of the methods described herein may be performed in any order and are not to be limited or restricted to the order in which they are particularly recited. For example, the present disclosure provides methods of co-producing butadiene and 1-propanol and/or 1,2-propanediol from a fermentable carbon source, comprising: providing a fermentable carbon source; contacting the fermentable carbon source with a microorganism comprising one or more polynucleotides coding for enzymes in a pathway that catalyzes a conversion of the fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol in a fermentation media; and expressing the one or more polynucleotides coding for the enzymes in the pathway that catalyzes a conversion of the fermentable carbon source to one or more intermediates in a pathway for the co-production of butadiene and 1-propanol and/or 1,2-propanediol and one or more polynucleotides coding for enzymes in a pathway that catalyze a conversion of the one or more intermediates to butadiene and 1-propanol and/or 1,2-propanediol in the microorganism to produce butadiene and 1-propanol and/or 1,2-propanediol. As such, expression of the one or more polynucleotides coding for the enzymes in the pathway that catalyzes a conversion of the fermentable carbon source to one or more intermediates in a pathway for the co-production of butadiene and 1-propanol and/or 1,2-propanediol and one or more polynucleotides coding for enzymes in a pathway that catalyze a conversion of the one or more intermediates to butadiene and 1-propanol and/or 1,2-propanediol in the microorganism to produce butadiene and 1-propanol and/or 1,2-propanediol may be preformed prior to or after contacting the fermentable carbon source with a microorganism comprising one or more polynucleotides coding for enzymes in a pathway that catalyzes a conversion of the fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol in a fermentation media.

In some embodiments, the ratio of grams of the produced 1-propanol and/or butadiene to grams of the fermentable carbon source is 0.01-0.84. As used herein, the term “biological activity” or “functional activity,” when referring to a protein, polypeptide or peptide, may mean that the protein, polypeptide or peptide exhibits a functionality or property that is useful as relating to some biological process, pathway or reaction. Biological or functional activity can refer to, for example, an ability to interact or associate with (e.g., bind to) another polypeptide or molecule, or it can refer to an ability to catalyze or regulate the interaction of other proteins or molecules (e.g., enzymatic reactions).

As used herein, the term “culturing” may refer to growing a population of cells, e.g., microbial cells, under suitable conditions for growth, in a liquid or on solid medium.

As used herein, the term “derived from” may encompass the terms originated from, obtained from, obtainable from, isolated from, and created from, and generally indicates that one specified material finds its origin in another specified material or has features that can be described with reference to the another specified material.

As used herein, the term “an expression vector” may refer to a DNA construct containing a polynucleotide or nucleic acid sequence encoding a polypeptide or protein, such as a DNA coding sequence (e.g., gene sequence) that is operably linked to one or more suitable control sequence(s) capable of affecting expression of the coding sequence in a host. Such control sequences include a promoter to affect transcription, an optional operator sequence to control such transcription, a sequence encoding suitable mRNA ribosome binding sites, and sequences which control termination of transcription and translation. The vector may be a plasmid, a phage particle, or simply a potential genomic insert. Once transformed into a suitable host, the vector may replicate and function independently of the host genome (e.g., independent vector or plasmid), or may, in some instances, integrate into the genome itself (e.g., integrated vector). The plasmid is the most commonly used form of expression vector. However, the disclosure is intended to include such other forms of expression vectors that serve equivalent functions and which are, or become, known in the art.

As used herein, the term “expression” may refer to the process by which a polypeptide is produced based on a nucleic acid sequence encoding the polypeptides (e.g., a gene). The process includes both transcription and translation.

As used herein, the term “gene” may refer to a DNA segment that is involved in producing a polypeptide or protein (e.g., fusion protein) and includes regions preceding and following the coding regions as well as intervening sequences (introns) between individual coding segments (exons).

As used herein, the term “heterologous,” with reference to a nucleic acid, polynucleotide, protein or peptide, may refer to a nucleic acid, polynucleotide, protein or peptide that does not naturally occur in a specified cell, e.g., a host cell. It is intended that the term encompass proteins that are encoded by naturally occurring genes, mutated genes, and/or synthetic genes. In contrast, the term homologous, with reference to a nucleic acid, polynucleotide, protein or peptide, refers to a nucleic acid, polynucleotide, protein or peptide that occurs naturally in the cell.

As used herein, the term a “host cell” may refer to a cell or cell line, including a cell such as a microorganism which a recombinant expression vector may be transfected for expression of a polypeptide or protein (e.g., fusion protein). Host cells include progeny of a single host cell, and the progeny may not necessarily be completely identical (in morphology or in total genomic DNA complement) to the original parent cell due to natural, accidental, or deliberate mutation. A host cell may include cells transfected or transformed in vivo with an expression vector.

As used herein, the term “introduced,” in the context of inserting a nucleic acid sequence or a polynucleotide sequence into a cell, may include transfection, transformation, or transduction and refers to the incorporation of a nucleic acid sequence or polynucleotide sequence into a eukaryotic or prokaryotic cell wherein the nucleic acid sequence or polynucleotide sequence may be incorporated into the genome of the cell (e.g., chromosome, plasmid, plastid, or mitochondrial DNA), converted into an autonomous replicon, or transiently expressed.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. A non-naturally occurring microbial organism of the disclosure can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely. Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

As used herein, “butadiene” is intended to mean a hydrocarbon with the molecular formula C4H6, a general formula of CH₂═CH—CH═CH₂ and a molecular mass of 54.09 g/mol. Butadiene is also known in the art as 1,3-butadiene, but-1,3-diene, biethylene, erythrene, divinyl, and vinylethylene.

As used herein, the term “operably linked” may refer to a juxtaposition or arrangement of specified elements that allows them to perform in concert to bring about an effect. For example, a promoter may be operably linked to a coding sequence if it controls the transcription of the coding sequence.

As used herein, the term “a promoter” may refer to a regulatory sequence that is involved in binding RNA polymerase to initiate transcription of a gene. A promoter may be an inducible promoter or a constitutive promoter. An inducible promoter is a promoter that is active under environmental or developmental regulatory conditions.

As used herein, the term “a polynucleotide” or “nucleic acid sequence” may refer to a polymeric form of nucleotides of any length and any three-dimensional structure and single or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Such polynucleotides or nucleic acid sequences may encode amino acids (e.g., polypeptides or proteins such as fusion proteins). Because the genetic code is degenerate, more than one codon may be used to encode a particular amino acid, and the present disclosure encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms polynucleotide, nucleic acid, and oligonucleotide are used herein interchangeably. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (thioate), P(S)S (dithioate), (O)NR₂ (amidate), P(O)R, P(O)OR′, COCH₂ (formacetal), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, the term “1-propanol” is intended to mean a hydrocarbon with the molecular formula C3H8O, a general formula CH₃CH₂CH₂OH, and a molecular mass of 60.10 g/mol. 1-propanol is also known in the art as propan-1-ol, 1-propyl alcohol, n-propyl alcohol, n-propanol, or simply propanol.

As used herein, the term “1,2-propanediol” is intended to mean a hydrocarbon with the molecular formula C3H8O2, a general formula HO—CH2-CHOH—CH3, and a molecular mass of 76.09 g/mol. 1,2-propanediol is also known in the art as propylene glycol or propane-1,2-diol.

As used herein, the term a “protein” or “polypeptide” may refer to a composition comprised of amino acids and recognized as a protein by those of skill in the art. The conventional one-letter or three-letter code for amino acid residues is used herein. The terms protein and polypeptide are used interchangeably herein to refer to polymers of amino acids of any length, including those comprising linked (e.g., fused) peptides/polypeptides (e.g., fusion proteins). The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, related proteins, polypeptides or peptides may encompass variant proteins, polypeptides or peptides. Variant proteins, polypeptides or peptides differ from a parent protein, polypeptide or peptide and/or from one another by a small number of amino acid residues. In some embodiments, the number of different amino acid residues is any of about 1, 2, 3, 4, 5, 10, 20, 25, 30, 35, 40, 45, or 50. In some embodiments, variants differ by about 1 to about 10 amino acids. Alternatively or additionally, variants may have a specified degree of sequence identity with a reference protein or nucleic acid, e.g., as determined using a sequence alignment tool, such as BLAST, ALIGN, and CLUSTAL (see, infra). For example, variant proteins or nucleic acid may have at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence.

As used herein, the term “recovered,” “isolated,” “purified,” and “separated” may refer to a material (e.g., a protein, peptide, nucleic acid, polynucleotide or cell) that is removed from at least one component with which it is naturally associated. For example, these terms may refer to a material which is substantially or essentially free from components which normally accompany it as found in its native state, such as, for example, an intact biological system.

As used herein, the term “recombinant” may refer to nucleic acid sequences or polynucleotides, polypeptides or proteins, and cells based thereon, that have been manipulated by man such that they are not the same as nucleic acids, polypeptides, and cells as found in nature. Recombinant may also refer to genetic material (e.g., nucleic acid sequences or polynucleotides, the polypeptides or proteins they encode, and vectors and cells comprising such nucleic acid sequences or polynucleotides) that has been modified to alter its sequence or expression characteristics, such as by mutating the coding sequence to produce an altered polypeptide, fusing the coding sequence to that of another coding sequence or gene, placing a gene under the control of a different promoter, expressing a gene in a heterologous organism, expressing a gene at decreased or elevated levels, expressing a gene conditionally or constitutively in manners different from its natural expression profile, and the like.

As used herein, the term “selective marker” or “selectable marker” may refer to a gene capable of expression in a host cell that allows for ease of selection of those hosts containing an introduced nucleic acid sequence, polynucleotide or vector. Examples of selectable markers include but are not limited to antimicrobial substances (e.g., hygromycin, bleomycin, or chloramphenicol) and/or genes that confer a metabolic advantage, such as a nutritional advantage, on the host cell.

As used herein, the term “substantially similar” and “substantially identical” in the context of at least two nucleic acids, polynucleotides, proteins or polypeptides may mean that a nucleic acid, polynucleotide, protein or polypeptide comprises a sequence that has at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference (e.g., wild-type) nucleic acid, polynucleotide, protein or polypeptide. Sequence identity may be determined using known programs such as BLAST, ALIGN, and CLUSTAL using standard parameters. (See, e.g., Altshul et al. (1990) J. Mol. Biol. 215:403-410; Henikoff et al. (1989) Proc. Natl. Acad. Sci. 89:10915; Karin et al. (1993) Proc. Natl. Acad. Sci. 90:5873; and Higgins et al. (1988) Gene 73:237). Software for performing BLAST analyses is publicly available through the National Center for Biotechnology Information. Also, databases may be searched using FASTA (Person et al. (1988) Proc. Natl. Acad. Sci. 85:2444-2448.) In some embodiments, substantially identical polypeptides differ only by one or more conservative amino acid substitutions. In some embodiments, substantially identical polypeptides are immunologically cross-reactive. In some embodiments, substantially identical nucleic acid molecules hybridize to each other under stringent conditions (e.g., within a range of medium to high stringency).

As used herein, the term “transfection” or “transformation” may refer to the insertion of an exogenous nucleic acid or polynucleotide into a host cell. The exogenous nucleic acid or polynucleotide may be maintained as a non-integrated vector, for example, a plasmid, or alternatively, may be integrated into the host cell genome. The term transfecting or transfection is intended to encompass all conventional techniques for introducing nucleic acid or polynucleotide into host cells. Examples of transfection techniques include, but are not limited to, calcium phosphate precipitation, DEAE-dextran-mediated transfection, lipofection, electroporation, and microinjection.

As used herein, the term “transformed,” “stably transformed,” and “transgenic” may refer to a cell that has a non-native (e.g., heterologous) nucleic acid sequence or polynucleotide sequence integrated into its genome or as an episomal plasmid that is maintained through multiple generations.

As used herein, the term “vector” may refer to a polynucleotide sequence designed to introduce nucleic acids into one or more cell types. Vectors include cloning vectors, expression vectors, shuttle vectors, plasmids, phage particles, single and double stranded cassettes and the like.

As used herein, the term “wild-type,” “native,” or “naturally-occurring” proteins may refer to those proteins found in nature. The terms wild-type sequence refers to an amino acid or nucleic acid sequence that is found in nature or naturally occurring. In some embodiments, a wild-type sequence is the starting point of a protein engineering project, for example, production of variant proteins.

Unless defined otherwise herein, all technical and scientific terms used herein have the same meaning as commonly understood by one of ordinary skill in the art to which this disclosure belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this disclosure.

Numeric ranges provided herein are inclusive of the numbers defining the range.

Unless otherwise indicated, nucleic acids sequences are written left to right in 5′ to 3′ orientation; amino acid sequences are written left to right in amino to carboxy orientation, respectively.

While the present disclosure is capable of being embodied in various forms, the description below of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

### Modification of Microorganism

A microorganism may be modified (e.g., genetically engineered) by any method known in the art to comprise and/or express one or more polynucleotides coding for enzymes in a pathway that catalyze a conversion of a fermentable carbon source to one or more intermediates in a pathway for the co-production of butadiene and 1-propanol and/or 1,2-propanediol. Such enzymes may include any of those enzymes as are forth in any one of FIGS. 1-4. For example, the microorganism may be modified to comprise one or more polynucleotides coding for enzymes that catalyze a conversion of crotonyl alcohol, 5-hydroxy-3-ketovaleryl-CoA, 3-ketopent-4-enoyl-CoA, or 3,5-ketovaleryl-CoA to butadiene. Additionally, for example, the microorganism may be modified to comprise one or more polynucleotides coding for enzymes that catalyze a conversion of methylglyoxal and/or lactate to 1-propanol and/or 1,2-propanediol.

A modified microorganism as provided herein may comprise one or more polynucleotides coding for enzymes in a pathway that catalyze a conversion of crotonyl-alcohol to butadiene and one or more polynucleotides coding for enzymes in a pathway that catalyze a conversion of methylglyoxal and/or lactate to 1-propanol and/or 1,2-propanediol. In some embodiments, the one or more polynucleotides include one or more polynucleotides coding for enzymes in a pathway that catalyze a conversion of fructose to dihydroxyacetone-phosphate, one or more polynucleotides coding for enzymes in a pathway that catalyze a conversion of dihydroxyacetone-phosphate to methylglyoxal (e.g., methylglyoxal synthase), one or more polynucleotides coding for enzymes in a pathway that catalyze a conversion of methylglyoxal to R/S lactaldehyde (e.g., methylglyoxal reductase), one or more polynucleotides coding for enzymes in a pathway that catalyze a conversion of methylglyoxal to hydroxyacetone (e.g., methylglyoxal oxidoreductase), one or more polynucleotides coding for enzymes in a pathway that catalyze a conversion of R/S lactaldehyde to R/S 1,2-propanediol (e.g., lactaldehyde reductase), one or more polynucleotides coding for enzymes in a pathway that catalyze a conversion of hydroxyacetone to R/S 1,2-propanediol (e.g., 1,2-propanediol dehydrogenase), one or more polynucleotides coding for enzymes in a pathway that catalyze a conversion of R/S 1,2-propanediol to propanal (e.g., 1,2-propanediol dehydratase), one or more polynucleotides coding for enzymes in a pathway that catalyze a conversion of propanal to 1-propanol (e.g., 1-propanol dehydrogenase), one or more polynucleotides coding for enzymes in a pathway that catalyze a conversion of glucose to fructose, one or more polynucleotides coding for enzymes in a pathway that catalyze a conversion of fructose to glyceraldehyde-3P (glyceraldehyde-3 phosphate), one or more polynucleotides coding for enzymes in a pathway that catalyze a conversion of glyceraldehyde-3P to pyruvate, one or more polynucleotides coding for enzymes in a pathway that catalyze a conversion of pyruvate to R/S lactate (e.g., lactate dehydrogenase), one or more polynucleotides coding for enzymes in a pathway that catalyze a conversion of R/S lactate to R/S lactaldehyde (e.g., carboxylic acid reductase and phosphopantetheinyl transferase), one or more polynucleotides coding for enzymes in a pathway that catalyze a conversion of pyruvate to acetaldehyde (e.g., pyruvate decarboxylase), one or more polynucleotides coding for enzymes in a pathway that catalyze a conversion of acetaldehyde to acetic acid (e.g., acetaldehyde dehydrogenase), one or more polynucleotides coding for enzymes in a pathway that catalyze a conversion of acetic acid to acetyl-CoA (e.g., acetyl-CoA synthase), one or more polynucleotides coding for enzymes in a pathway that catalyze a conversion of pyruvate to acetyl-CoA (e.g., pyruvate dehydrogenase), one or more polynucleotides coding for enzymes in a pathway that catalyze a conversion of acetyl-CoA and acetyl-CoA to acetoacetyl-CoA (e.g., acetoacetyl-CoA thiolase), one or more polynucleotides coding for enzymes in a pathway that catalyze a conversion of acetoacetyl-CoA to 3-hydroxybutyryl-CoA (e.g., 3-hydroxybutyryl-CoA dehydrogenase), one or more polynucleotides coding for enzymes in a pathway that catalyze a conversion of 3-hydroxybutyryl-CoA to crotonyl-CoA (e.g., crotonase), one or more polynucleotides coding for enzymes in a pathway that catalyze a conversion of crotonyl-CoA to crotonyl alcohol (e.g., crotonyl-CoA reductase (bifuncional)), one or more polynucleotides coding for enzymes in a pathway that catalyze a conversion of crotonyl-CoA to crotonaldehyde (e.g., crotonaldehyde dehydrogenase), one or more polynucleotides coding for enzymes in a pathway that catalyze a conversion of crotonaldehyde to crotonyl alcohol (e.g., alcohol dehydrogenase), one or more polynucleotides coding for enzymes in a pathway that catalyze a conversion of crotonyl alcohol to butadiene (e.g., crotonyl alcohol dehydratase), one or more polynucleotides coding for enzymes in a pathway that catalyze a conversion of crotonyl alcohol to 2-butenyl-4-phosphate (e.g., crotonyl alcohol kinase), one or more polynucleotides coding for enzymes in a pathway that catalyze a conversion of 2-butenyl-4-phosphate to 2-butenyl-4-diphosphate (e.g., 2-butenyl-4-phosphate kinase), and/or one or more polynucleotides coding for enzymes in a pathway that catalyze a conversion of 2-butenyl-4-diphosphate to butadiene (e.g., butadiene kinase).

Exemplary enzymes which convert crotonyl-alcohol to butadiene and methylglyoxal and lactate to 1-propanol are presented in Table 1 below, as well as, the substrates that they act upon and product that they produce. The enzyme number represented in Table 1 correlates with the enzyme numbering used in FIG. 1 which schematically represents the enzymatic conversion of a fermentable carbon source to butadiene and 1-propanol and/or 1,2-propanediol through a crotonyl-alcohol intermediate, and methylglyoxal and lactate intermediates, respectively. Additionally, Table 1 indicates a gene identifier (GI) number(s) that corresponds to an exemplary amino acid sequence(s) for the listed enzyme.

TABLE 1
Co-production of butadiene via a crotonyl-alcohol intermediate
and 1-propanol via methylglyoxal and lactate intermediates

| Enzyme | | E.C. | |
|---|---|---|---|
| No. | Enzyme Name | number | Mediated Conversion |
| A. | Methylglyoxal | 4.2.3.3 | Dihydroxyacetone phosphate → |
| | synthase | | Methylglyoxal |
| B. | methylglyoxal | 1.1.1.78 | Methylglyoxal → R/S |
| | reductase | | lactaldehyde |
| C. | methylglyoxal | 1.1.1.— | Methylglyoxal → |
| | oxidoreductase | 1.1.1.202 | hydroxyacetone |
| D. | lactaldehyde | 1.1.1.77 | R/S Lactaldehyde → R/S 1,2 |
| | reductase | | propanediol |
| E. | 1,2-propanediol | 1.1.1.— | Hydroxyacetone → R/S 1,2 |
| | dehydrogenase | 1.1.1.16 | propanediol |
| | | 1.1.1.21 | |
| | | 1.1.1.202 | |
| F. | 1,2 propanediol | 4.2.1.28 | R/S 1,2 propanediol → propanal |
| | dehydratase | | |
| G. | 1-propanol | 1.1.1.— | Propanal → 1-propanol |
| | dehydrogenase | | |
| H. | Lactate | 1.1.1.27 | Pyruvate → R/S Lactate |
| | dehydrogenase | | |
| I. | Carboxylic acid | 1.2.99.6 | R/S Lactate → R/S Lactaldehyde |
| | reductase | 2.7.8.7 | Apo-Car → Holo-Car |
| | Phosphopantetheinyl | | |
| | transferase | | |
| J. | Pyruvate | 4.1.1.1 | Pyruvate → Acetaldehyde |
| | decarboxylase | | |
| K. | acetaldehyde | 1.2.1.3 | Acetaldehyde → Acetic acid |
| | dehydrogenase | | |
| L. | acetyl-Coa synthase | 6.2.1.1 | Acetic acid → Acetyl-CoA |
| M. | Pyruvate | 1.2.4.1 | Pyruvate → Acetyl-CoA |
| | dehydrogenase | 2.3.1.12 | |
| | | 1.8.1.4 | |
| N. | acetoacetyl-CoA | 2.3.1.9 | Acetyl-CoA → Acetoacetyl-CoA |
| | thiolase | | |
| O. | 3-hydroxybutyryl-CoA | 1.1.1.35; | Acetoacetyl-CoA → |
| | dehydrogenase | 1.1.1.157 | 3-hydroxybutyryl-CoA |
| P. | Crotonase | 4.2.1.17 | 3-hydroxybutyryl-CoA → |
| | | | Crotonyl-CoA |
| Q. | Crotonyl-CoA | 1.1.1 | Crotonyl-CoA → crotonyl alcohol |
| | reductase | 1.1.1.34 | |
| | (Bifuncional) | 1.1.1.88 | |
| R. | Crotonaldehyde | 1.2.1. | Crotonyl-CoA → crotonaldehyde |
| | dehydrogenase | | |
| S. | Alcohol | 1.1.1 | Crotonaldehyde → crotonyl |
| | dehydrogenase | | alcohol |
| T. | Crotonyl alcohol | 4.2.1 | Crotonyl alcohol → butadiene |
| | dehydratase | 4.2.1.127 | |
| U. | crotonyl alcohol | 2.7.1.36 | Crotonyl alcohol → |
| | kinase | | 2-butenyl-4-phosphate |
| V. | 2-butenyl-4-phosphate | 2.7.4.2 | 2-butenyl-4-phosphate → |
| | kinase | | 2-butenyl-4-diphosphate |
| X. | crotonyl alcohol | 2.7.1.33 | Crotonyl alcohol → |
| | diphosphokinase | | 2-butenyl-4-diphosphate |
| Z. | butadiene synthase | 4.2.3.27 | 2-butenyl-4-diphosphate → |
| | | | butadiene |

A modified microorganism as provided herein may comprise one or more polynucleotides coding for enzymes in a pathway that catalyze a conversion of 5-hydroxy-3-ketovaleryl-CoA to butadiene and one or more polynucleotides coding for enzymes in a pathway that catalyze a conversion of methylglyoxal and/or lactate to 1-propanol and/or 1,2-propanediol. In some embodiments, the one or more polynucleotides include one or more polynucleotides coding for enzymes in a pathway that catalyze a conversion of fructose to dihydroxyacetone-phosphate, one or more polynucleotides coding for enzymes in a pathway that catalyze a conversion of dihydroxyacetone-phosphate to methylglyoxal (e.g., methylglyoxal synthase), one or more polynucleotides coding for enzymes in a pathway that catalyze a conversion of methylglyoxal to R/S lactaldehyde (e.g., methylglyoxal reductase), one or more polynucleotides coding for enzymes in a pathway that catalyze a conversion of methylglyoxal to hydroxyacetone (e.g., methylglyoxal oxidoreductase), one or more polynucleotides coding for enzymes in a pathway that catalyze a conversion of R/S lactaldehyde to R/S 1,2-propanediol (e.g., lactaldehyde reductase), one or more polynucleotides coding for enzymes in a pathway that catalyze a conversion of hydroxyacetone to R/S 1,2-propanediol (e.g., 1,2-propanediol dehydrogenase), one or more polynucleotides coding for enzymes in a pathway that catalyze a conversion of R/S 1,2-propanediol to propanal (e.g., 1,2-propanediol dehydratase), one or more polynucleotides coding for enzymes in a pathway that catalyze a conversion of propanal to 1-propanol (e.g., 1-propanol dehydrogenase), one or more polynucleotides coding for enzymes in a pathway that catalyze a conversion of glucose to fructose, one or more polynucleotides coding for enzymes in a pathway that catalyze a conversion of fructose to glyceraldehyde-3P, one or more polynucleotides coding for enzymes in a pathway that catalyze a conversion of glyceraldehyde-3P to pyruvate, one or more polynucleotides coding for enzymes in a pathway that catalyze a conversion of pyruvate to R/S lactate (e.g., lactate dehydrogenase), one or more polynucleotides coding for enzymes in a pathway that catalyze a conversion of R/S lactate to R/S lactaldehyde (e.g., carboxylic acid reductase), one or more polynucleotides coding for enzymes in a pathway that catalyze a conversion of pyruvate to acetaldehyde (e.g., pyruvate decarboxylase), one or more polynucleotides coding for enzymes in a pathway that catalyze a conversion of acetaldehyde to acetic acid (e.g., acetaldehyde dehydrogenase), one or more polynucleotides coding for enzymes in a pathway that catalyze a conversion of acetic acid to acetyl-CoA (e.g., acetyl-CoA synthase), one or more polynucleotides coding for enzymes in a pathway that catalyze a conversion of pyruvate to acetyl-CoA (e.g., pyruvate dehydrogenase), one or more polynucleotides coding for enzymes in a pathway that catalyze a conversion of R/S lactate to lactoyl-CoA (e.g., lactoyl-CoA transferase, or synthase), one or more polynucleotides coding for enzymes in a pathway that catalyze a conversion of lactoyl-CoA to acryloyl-CoA (e.g., lactoyl-CoA dehydratase), one or more polynucleotides coding for enzymes in a pathway that catalyze a conversion of acryloyl-CoA to 3-hydroxypropionyl-CoA (e.g., acryloyl-CoA hydratase), one or more polynucleotides coding for enzymes in a pathway that catalyze a conversion of acetyl-CoA and 3-hydroxypropionyl-CoA to 5-hydroxy-3-ketovaleryl-CoA (e.g., a 5-hydroxy-3-ketovaleryl-CoA thiolase), one or more polynucleotides coding for enzymes in a pathway that catalyze a conversion of 5-hydroxy-3-ketovaleryl-CoA to R/S 3,5-dihydroxy-valeryl-CoA (e.g., 5-hydroxy-3-ketovaleryl-CoA dehydrogenase), one or more polynucleotides coding for enzymes in a pathway that catalyze a conversion of R/S 3,5-dihydroxy-valeryl-CoA to R/S 3-hydroxy-4-pentenoyl-CoA (e.g., 3,5-hydroxyvaleryl-CoA dehydratase), one or more polynucleotides coding for enzymes in a pathway that catalyze a conversion of R/S 3-hydroxy-4-pentenoyl-CoA to 3-hydroxy-4-pentenoic acid (e.g., 3-hydroxy-4-pentenoyl-CoA hydrolase, transferase or synthase) and/or one or more polynucleotides coding for enzymes in a pathway that catalyze a conversion of 3-hydroxy-4-pentenoic acid to butadiene (e.g., 3-hydroxy-4-pentenoic acid decarboxylase).

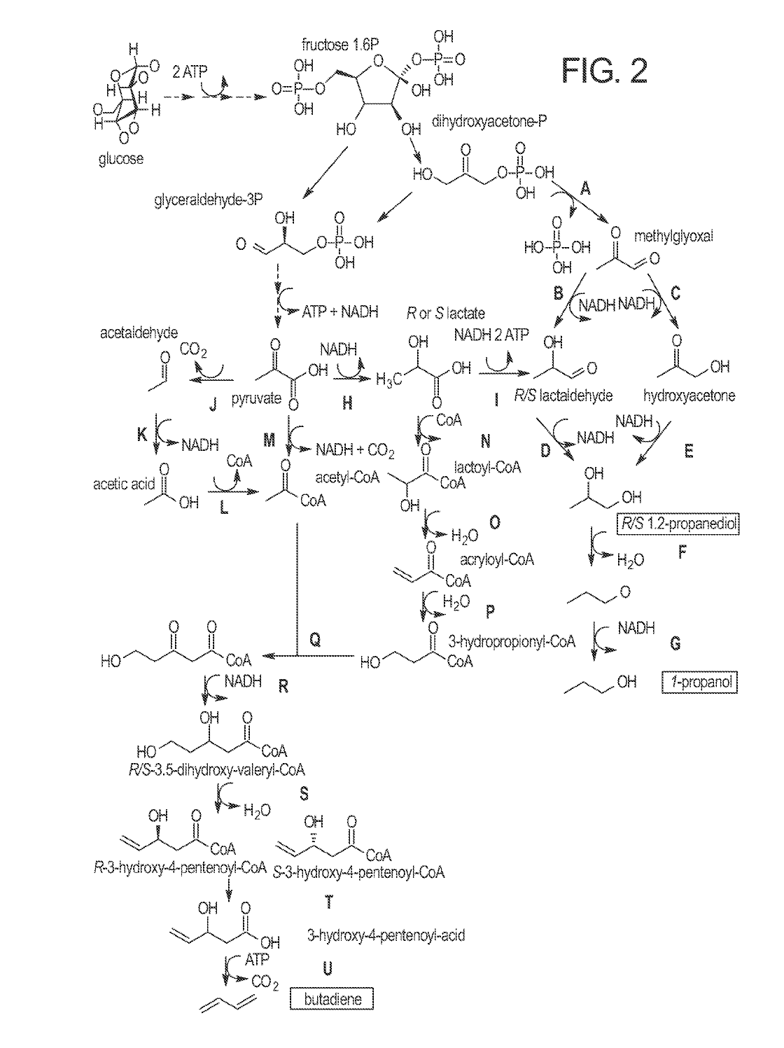

Exemplary enzymes which convert 5-hydroxy-3-ketovaleryl-CoA to butadiene and methylglyoxal and lactate to 1-propanol and/or 1,2-propanediol are presented in Table 2 below, as well as, the substrates that they act upon and product that they produce. The enzyme number represented in Table 2 correlates with the enzyme numbering used in FIG. 2 which schematically represents the enzymatic conversion of a fermentable carbon source to butadiene and 1-propanol and/or 1,2-propanediol through a 5-hydroxy-3-ketovaleryl-CoA intermediate, and a methylglyoxal and lactate intermediates, respectively. Additionally, Table 2 indicates a gene identifier (GI) number(s) that corresponds to an exemplary amino acid sequence(s) for the listed enzyme.

TABLE 2
Co-production of butadiene via a 5-hydroxy-3-ketovaleryl-CoA intermediate and
1-propanol and/or 1,2-propanediol via methylglyoxal and lactate intermediates

| Enzyme | | E.C. | |
|---|---|---|---|
| No. | Enzyme Name | number | Mediated Conversion |
| A. | Methylglyoxal | 4.2.3.3 | Dihydroxyacetone phosphate → |
| | synthase | | Methylglyoxal |
| B. | methylglyoxal | 1.1.1.78 | Methylglyoxal → R/S |
| | reductase | | lactaldehyde |
| C. | methylglyoxal | 1.1.1.— | Methylglyoxal → hydroxyacetone |
| | oxidoreductase | 1.1.1.202 | |
| D. | lactaldehyde | 1.1.1.77 | R/S Lactaldehyde → R/S 1,2 |
| | reductase | | propanediol |
| E. | 1,2-propanediol | 1.1.1.— | Hydroxyacetone → R/S 1,2 |
| | dehydrogenase | 1.1.1.16 | propanediol |
| | | 1.1.1.21 | |
| | | 1.1.1.202 | |
| F. | 1,2 propanediol | 4.2.1.28 | R/S 1,2 propanediol → propanal |
| | dehydratase | | |
| G. | 1-propanol | 1.1.1.— | Propanal → 1-propanol |
| | dehydrogenase | | |
| H. | Lactate | 1.1.1.27 | Pyruvate → Lactate |
| | dehydrogenase | | |
| I. | Carboxylic acid | 1.2.99.6 | Lactate → Lactaldehyde |
| | reductase | 2.7.8.7 | Apo-Car → Holo-Car |
| | Phosphopantetheinyl | | |
| | transferase | | |
| J. | Pyruvate | 4.1.1.1 | Pyruvate → Acetaldehyde |
| | decarboxylase | | |
| K. | acetaldehyde | 1.2.1.3 | Acetaldehyde → Acetic acid |
| | dehydrogenase | | |
| L. | acetyl-Coa synthase | 6.2.1.1 | Acetic acid → Acetyl-CoA |
| M. | Pyruvate | 1.2.4.1 | Pyruvate → Acetyl-CoA |
| | dehydrogenase | 2.3.1.12 | |
| | | 1.8.1.4 | |
| N. | lactoyl-CoA | 2.8.3.1 | Lactate → Lactoyl-CoA |
| | transferase or | 2.3.3 | |
| | synthase | | |
| O. | lactoyl-CoA | 4.2.1.54 | Lactoyl-CoA → Acryloyl-CoA |
| | dehydratase | | |
| P. | acryloyl-CoA | 4.2.1.17 | Acryloyl-CoA → |
| | hydratase | | 3-hydroxypropyonyl-CoA |
| Q. | 5-hydroxy-3- | 2.3.1.16 | Acetyl-CoA + |
| | ketovaleryl-CoA | | 3-hydroxypropionyl-CoA → |
| | thiolase | | 5-hydroxy-3-ketovaleryl-CoA |
| R. | 5-hydroxy-3- | 1.1.1.35 | 5-hydroxy-3-Ketovaleryl-CoA → |
| | Ketovaleryl-CoA | 1.1.1.36 | R/S 3,5-dihydroxy-valeryl-CoA |
| | dehydrogenase | | |
| S. | 3,5-hydroxyvaleryl- | 4.2.1 | R/S 3,5-dihydroxy-valeryl-CoA → |
| | CoA dehydratase | 4.2.1.17 | 3-hydroxy-4-pentenoyl-CoA |
| | | 4.2.1.54 | |
| T. | 3-hydroxy-4- | 3.1.2, | 3-hydroxy-4-pentenoyl-CoA → |
| | pentenoyl-CoA | 2.8.3 or | 3-hydroxy-4-pentenoic acid |
| | hydrolase, transferase | 2.3.3 | |
| | or synthase | | |
| U. | 3-hydroxy-4- | 4.1.1.33 | 3-hydroxy-4-pentenoic acid → |
| | pentenoic acid | | butadiene |
| | decarboxylase | | |

A modified microorganism as provided herein may comprise one or more polynucleotides coding for enzymes in a pathway that catalyze a conversion of 3-keto-pent-4-enoyl-CoA to butadiene and one or more polynucleotides coding for enzymes in a pathway that catalyze a conversion of methylglyoxal and/or lactate to 1-propanol and/or 1,2-propanediol. In some embodiments, the one or more polynucleotides include one or more polynucleotides coding for enzymes in a pathway that catalyze a conversion of fructose to dihydroxyacetone-phosphate, one or more polynucleotides coding for enzymes in a pathway that catalyze a conversion of dihydroxyacetone-phosphate to methylglyoxal (e.g., methylglyoxal synthase), one or more polynucleotides coding for enzymes in a pathway that catalyze a conversion of methylglyoxal to R/S lactaldehyde (e.g., methylglyoxal reductase), one or more polynucleotides coding for enzymes in a pathway that catalyze a conversion of methylglyoxal to hydroxyacetone (e.g., methylglyoxal oxidoreductase), one or more polynucleotides coding for enzymes in a pathway that catalyze a conversion of R/S lactaldehyde to R/S 1,2-propanediol (e.g., lactaldehyde reductase), one or more polynucleotides coding for enzymes in a pathway that catalyze a conversion of hydroxyacetone to R/S 1,2-propanediol (e.g., 1,2-propanediol dehydrogenase), one or more polynucleotides coding for enzymes in a pathway that catalyze a conversion of R/S 1,2-propanediol to propanal (e.g., 1,2-propanediol dehydratase), one or more polynucleotides coding for enzymes in a pathway that catalyze a conversion of propanal to 1-propanol (e.g., 1-propanol dehydrogenase), one or more polynucleotides coding for enzymes in a pathway that catalyze a conversion of glucose to fructose, one or more polynucleotides coding for enzymes in a pathway that catalyze a conversion of fructose to glyceraldehyde-3P, one or more polynucleotides coding for enzymes in a pathway that catalyze a conversion of glyceraldehyde-3P to pyruvate, one or more polynucleotides coding for enzymes in a pathway that catalyze a conversion of pyruvate to R/S lactate (e.g., lactate dehydrogenase), one or more polynucleotides coding for enzymes in a pathway that catalyze a conversion of R/S lactate to R/S lactaldehyde (e.g., carboxylic acid reductase and phosphopantetheinyl transferase), one or more polynucleotides coding for enzymes in a pathway that catalyze a conversion of pyruvate to acetaldehyde (e.g., pyruvate decarboxylase), one or more polynucleotides coding for enzymes in a pathway that catalyze a conversion of acetaldehyde to acetic acid (e.g., acetaldehyde dehydrogenase), one or more polynucleotides coding for enzymes in a pathway that catalyze a conversion of acetic acid to acetyl-CoA (e.g., acetyl-CoA synthase), one or more polynucleotides coding for enzymes in a pathway that catalyze a conversion of pyruvate to acetyl-CoA (e.g., pyruvate dehydrogenase), one or more polynucleotides coding for enzymes in a pathway that catalyze a conversion of R/S lactate to lactoyl-CoA (e.g., lactoyl-CoA transferase, or synthase), one or more polynucleotides coding for enzymes in a pathway that catalyze a conversion of lactoyl-CoA to acryloyl-CoA (e.g., lactoyl-CoA dehydratase), one or more polynucleotides coding for enzymes in a pathway that catalyze a conversion of acryloyl-CoA and acetyl-CoA to 3-keto-4-pentenoyl-CoA (e.g., 3-keto-4-pentenoyl-CoA thiolase), one or more polynucleotides coding for enzymes in a pathway that catalyze a conversion of 3-keto-4-pentenoyl-CoA to R/S 3-hydroxy-4-pentenoyl-CoA (e.g., 3-keto-4-pentenoyl-CoA dehydrogenase), one or more polynucleotides coding for enzymes in a pathway that catalyze a conversion of R/S 3-hydroxy-4-pentenoyl-CoA to 3-hydroxy-4-pentenoic acid (e.g., hydroxy-4-pentenoyl-CoA transferase, hydrolase, or synthase), and/or one or more polynucleotides coding for enzymes in a pathway that catalyze a conversion of 3-hydroxy-4-pentenoic acid to butadiene (e.g., 3-hydroxy-4-pentenoic acid decarboxylase).

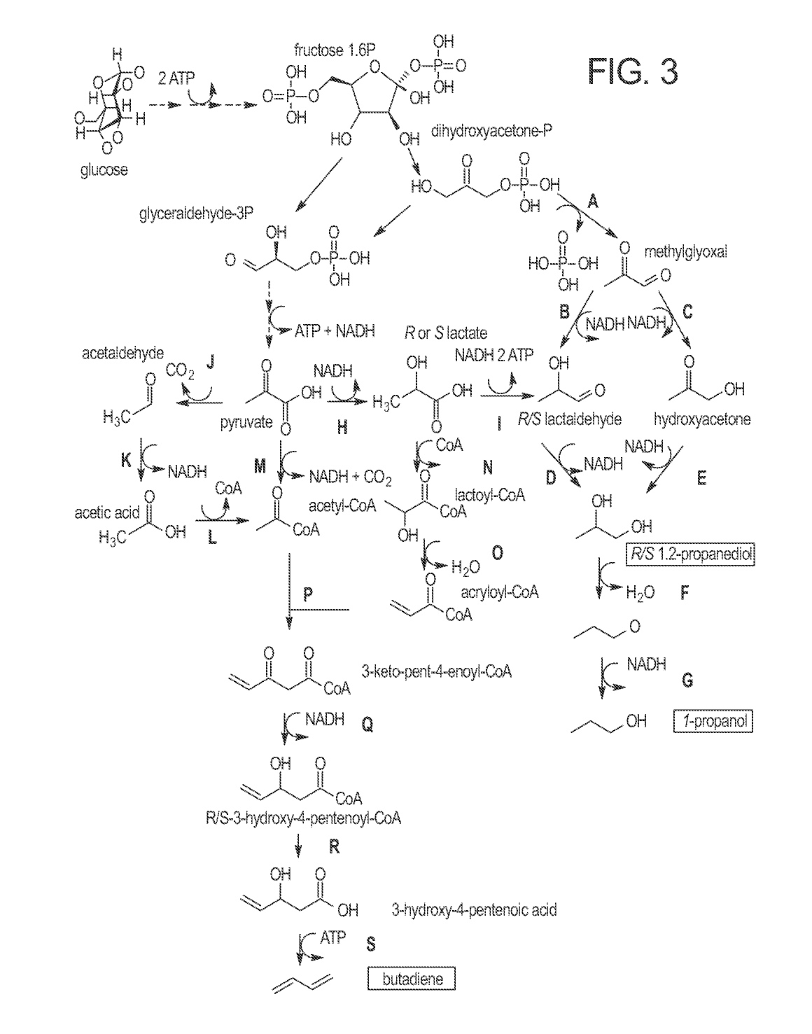

Exemplary enzymes which convert 3-keto-4-pentenoyl-CoA to butadiene and methylglyoxal and lactate to 1-propanol and/or propanediol are presented in Table 3 below, as well as, the substrates that they act upon and product that they produce. The enzyme number represented in Table 3 correlates with the enzyme numbering used in FIG. 3 which schematically represents the enzymatic conversion of a fermentable carbon source to butadiene through a 3-keto-4-pentenoyl-CoA intermediate, and 1-propanol and/or 1,2-propanediol through a methylglyoxal and lactate intermediate, respectively. Additionally, Table 3 indicates a gene identifier (GI) number(s) that corresponds to an exemplary amino acid sequence(s) for the listed enzyme.

TABLE 3
Co-production of butadiene via a 3-keto-4-pentenoyl-CoA intermediate and 1-
propanol and/or 1,2-propanediol via methylglyoxal and lactate intermediates

| Enzyme | | E.C. | |
|---|---|---|---|
| No. | Enzyme Name | number | Mediated Conversion |
| A. | Methylglyoxal | 4.2.3.3 | Dihydroxyacetone phosphate → |
| | synthase | | Methylglyoxal |
| B. | methylglyoxal | 1.1.1.78 | Methylglyoxal → R/S lactaldehyde |
| | reductase | | |
| C. | methylglyoxal | 1.1.1.— | Methylglyoxal → hydroxyacetone |
| | oxidoreductase | 1.1.1.202 | |
| D. | lactaldehyde | 1.1.1.77 | R/S Lactaldehyde → R/S 1,2 |
| | reductase | | propanediol |
| E. | 1,2-propanediol | 1.1.1.— | Hydroxyacetone → R/S 1,2 |
| | dehydrogenase | 1.1.1.16 | propanediol |
| | | 1.1.1.21 | |
| | | 1.1.1.202 | |
| F. | 1,2 propanediol | 4.2.1.28 | R/S 1,2 propanediol → propanal |
| | dehydratase | | |
| G. | 1-propanol | 1.1.1.— | Propanal → 1-propanol |
| | dehydrogenase | | |
| H. | Lactate | 1.1.1.27 | Pyruvate → Lactate |
| | dehydrogenase | | |
| I. | Carboxylic acid | 1.2.99.6 | Lactate → Lactaldehyde |
| | reductase | 2.7.8.7 | Apo-Car → Holo-Car |
| | Phosphopantetheinyl | | |
| | transferase | | |
| J. | Pyruvate | 4.1.1.1 | Pyruvate → Acetaldehyde |
| | decarboxylase | | |
| K. | acetaldehyde | 1.2.1.3 | Acetaldehyde → Acetic acid |
| | dehydrogenase | | |
| L. | acetyl-Coa synthase | 6.2.1.1 | Acetic acid → Acetyl-CoA |
| M. | Pyruvate | 1.2.4.1 | Pyruvate → Acetyl-CoA |
| | dehydrogenase | 2.3.1.12 | |
| | | 1.8.1.4 | |
| N. | lactoyl-CoA | 2.8.3.1 | Lactate → Lactoyl-CoA |
| | transferase | 2.3.3 | |
| | or synthase | | |
| O. | lactoyl-CoA | 4.2.1.54 | Lactoyl-CoA → |
| | dehydratase | | Acryloyl-CoA |
| P. | 3-keto-4-pentenoyl- | 2.3.1.9 | Acryloyl-CoA + Acetyl-CoA → |
| | CoA thiolase | 2.3.1.16 | 3-keto-4-pentenoyl-CoA |
| Q. | 3-keto-4-pentenoyl-CoA | 1.1.1.35 | 3-keto-4-pentenoyl-CoA → |
| | dehydrogenase | 1.1.1.36 | R/S 3-hydroxy-4-pentenoyl-CoA |
| R. | 3-hydroxy-4- | 2.8.3 | R/S 3-hydroxy-4-pentenoyl-CoA → |
| | pentenoyl-CoA | 3.1.2 | 3-hydroxy-4-pentenoic acid |
| | transferase | 6.2.1 | |
| | or hydrolase or | | |
| | synthase | | |
| S. | 3-hydroxy-4- | 4.1.1.33 | 3-hydroxy-4-pentenoic acid → |
| | pentenoic acid | | butadiene |
| | decarboxylase | | |

A modified microorganism as provided herein may comprise one or more polynucleotides coding for enzymes in a pathway that catalyze a conversion of 3,5-ketovaleryl-CoA to butadiene and one or more polynucleotides coding for enzymes in a pathway that catalyze a conversion of methylglyoxal and/or lactate to 1-propanol and/or 1,2-propanediol. In some embodiments, the one or more polynucleotides include one or more polynucleotides coding for enzymes in a pathway that catalyze a conversion of fructose to dihydroxyacetone-phosphate, one or more polynucleotides coding for enzymes in a pathway that catalyze a conversion of dihydroxyacetone-phosphate to methylglyoxal (e.g., methylglyoxal synthase), one or more polynucleotides coding for enzymes in a pathway that catalyze a conversion of methylglyoxal to R/S lactaldehyde (e.g., methylglyoxal reductase), one or more polynucleotides coding for enzymes in a pathway that catalyze a conversion of methylglyoxal to hydroxyacetone (e.g., methylglyoxal oxidoreductase), one or more polynucleotides coding for enzymes in a pathway that catalyze a conversion of R/S lactaldehyde to R/S 1,2-propanediol (e.g., lactaldehyde reductase), one or more polynucleotides coding for enzymes in a pathway that catalyze a conversion of hydroxyacetone to R/S 1,2-propanediol (e.g., 1,2-propanediol dehydrogenase), one or more polynucleotides coding for enzymes in a pathway that catalyze a conversion of R/S 1,2-propanediol to propanal (e.g., 1,2-propanediol dehydratase), one or more polynucleotides coding for enzymes in a pathway that catalyze a conversion of propanal to 1-propanol (e.g., 1-propanol dehydrogenase), one or more polynucleotides coding for enzymes in a pathway that catalyze a conversion of glucose to fructose, one or more polynucleotides coding for enzymes in a pathway that catalyze a conversion of fructose to glyceraldehyde-3P, one or more polynucleotides coding for enzymes in a pathway that catalyze a conversion of glyceraldehyde-3P to pyruvate, one or more polynucleotides coding for enzymes in a pathway that catalyze a conversion of pyruvate to R/S lactate (e.g., lactate dehydrogenase), one or more polynucleotides coding for enzymes in a pathway that catalyze a conversion of R/S lactate to R/S lactaldehyde (e.g., carboxylic acid reductase and phosphopantetheinyl transferase), one or more polynucleotides coding for enzymes in a pathway that catalyze a conversion of pyruvate to acetaldehyde (e.g., pyruvate decarboxylase), one or more polynucleotides coding for enzymes in a pathway that catalyze a conversion of acetaldehyde to acetic acid (e.g., acetaldehyde dehydrogenase), one or more polynucleotides coding for enzymes in a pathway that catalyze a conversion of acetic acid to acetyl-CoA (e.g., acetyl-CoA synthase), one or more polynucleotides coding for enzymes in a pathway that catalyze a conversion of pyruvate to acetyl-CoA (e.g., pyruvate dehydrogenase), one or more polynucleotides coding for enzymes in a pathway that catalyze a conversion of CO₂ to formate (e.g., a formate dehydrogenase), one or more polynucleotides coding for enzymes in a pathway that catalyze a conversion of pyruvate and CoA to acetyl-CoA and formate (e.g., an Acetyl-CoA:formate C-acetyltransferase), one or more polynucleotides coding for enzymes in a pathway that catalyze a conversion of formate to formyl-CoA (e.g., a formyl-CoA transferase, or formyl-CoA synthase), one or more polynucleotides coding for enzymes in a pathway that catalyze a conversion of acetoacetyl-CoA and formyl-CoA to 3,5-ketovaleryl-CoA (e.g., 3,5-ketovaleryl-CoA thiolase), one or more polynucleotides coding for enzymes in a pathway that catalyze a conversion of 3,5-ketovaleryl-CoA to 5-hydroxy-3-ketovaleryl-CoA (e.g., a 5-hydroxy-3-ketovaleryl-CoA dehydrogenase), one or more polynucleotides coding for enzymes in a pathway that catalyze a conversion of 3,5-ketovaleryl-CoA to 5-hydroxy-3-ketovaleryl-CoA (e.g., a 5-hydroxy-3-ketovaleryl-CoA dehydrogenase), one or more polynucleotides coding for enzymes in a pathway that catalyze a conversion of 5-hydroxy-3-ketovaleryl-CoA or R/S 3-hydroxy-5-ketovaleryl-CoA to R/S 3,5-hydroxyvaleryl-CoA (e.g., a 3,5-hydroxyvaleryl-CoA dehydrogenase), one or more polynucleotides coding for enzymes in a pathway that catalyze a conversion of R/S 3,5-hydroxyvaleryl-CoA to R/S 3-hydroxy-4-pentenoyl-CoA (e.g., a 3,5-hydroxyvaleryl-CoA dehydratase), one or more polynucleotides coding for enzymes in a pathway that catalyze a conversion of R/S 3-hydroxy-4-pentenoyl-CoA to 3-hydroxy-4-pentenoic acid (e.g., a 3-hydroxy-4-pentenoyl-CoA hydrolase, transferase or synthase), and/or one or more polynucleotides coding for enzymes in a pathway that catalyze a conversion of 3-hydroxy-4-pentenoic acid to butadiene (e.g., a 3-hydroxy-4-pentenoic acid decarboxylase).

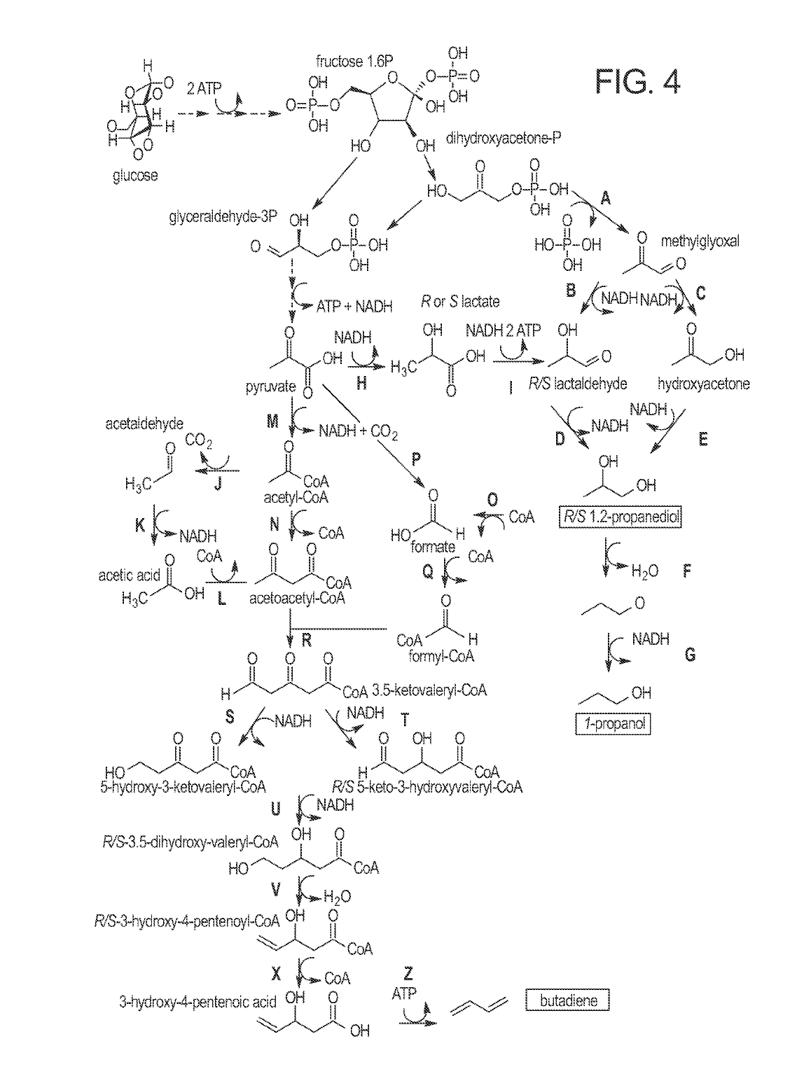

Exemplary enzymes which convert 3,5-ketovaleryl-CoA butadiene and 1-propanol and methylglyoxal and lactate to 1-propanol and/or 1,2-propanediol are presented in Table 4 below, as well as, the substrates that they act upon and product that they produce. The enzyme number represented in Table 4 correlates with the enzyme numbering used in FIG. 4 which schematically represents the enzymatic conversion of a fermentable carbon source to butadiene and 1-propanol and/or 1,2-propanediol through a 3,5-ketovaleryl-CoA intermediate, and a methylglyoxal and lactate intermediate, respectively. Additionally, Table 4 indicates a gene identifier (GI) number(s) that corresponds to an exemplary amino acid sequence(s) for the listed enzyme.

TABLE 4
Co-production of butadiene via a 3,5-ketovaleryl-CoA intermediate and 1-
propanol and/or 1,2-propanediol via methylglyoxal and lactate intermediates

| Enzyme | | E.C. | |
|---|---|---|---|
| No. | Enzyme Name | number | Mediated Conversion |
| A. | Methylglyoxal | 4.2.3.3 | Dihydroxyacetone phosphate → |
| | synthase | | Methylglyoxal |
| B. | methylglyoxal | 1.1.1.78 | Methylglyoxal → R/S lactaldehyde |
| | reductase | | |
| C. | methylglyoxal | 1.1.1.— | Methylglyoxal → hydroxyacetone |
| | oxidoreductase | 1.1.1.202 | |
| D. | lactaldehyde | 1.1.1.77 | R or S Lactaldehyde → R/S 1,2 |
| | reductase | | propanediol |
| E. | 1,2-propanediol | 1.1.1.— | Hydroxyacetone → R/S 1,2 |
| | dehydrogenase | 1.1.1.16 | propanediol |
| | | 1.1.1.21 | |
| | | 1.1.1.202 | |
| F. | 1,2 propanediol | 4.2.1.28 | R/S 1,2 propanediol → propanal |
| | dehydratase | | |
| G. | 1-propanol | 1.1.1.— | Propanal → 1-propanol |
| | dehydrogenase | | |
| H. | Lactate | 1.1.1.27 | Pyruvate → Lactate |
| | dehydrogenase | | |
| I. | Carboxylic acid | 1.2.99.6 | Lactate → Lactaldehyde |
| | reductase | 2.7.8.7 | Apo-Car → Holo-Car |
| | Phosphopantetheinyl | | |
| | transferase | | |
| J. | Pyruvate | 4.1.1.1 | Pyruvate → Acetaldehyde |
| | decarboxylase | | |
| K. | acetaldehyde | 1.2.1.3 | Acetaldehyde → Acetic acid |
| | dehydrogenase | | |
| L. | acetyl-Coa synthase | 6.2.1.1 | Acetic acid → Acetyl-CoA |
| M. | Pyruvate | 1.2.4.1 | Pyruvate → Acetyl-CoA |
| | dehydrogenase | 2.3.1.12 | |
| | | 1.8.1.4 | |
| N. | acetoacetyl-CoA | 2.3.1.9 | Acetyl-CoA → Acetoacetyl-CoA |
| | thiolase | | |
| O. | Formate | 1.2.1.2 | CO₂ → Formate |
| | dehydrogenase | | |
| P. | Acetyl-CoA: formate | 2.3.1.54 | Pyruvate + CoA → Acetyl-CoA + |
| | C-acetyltransferase | | Formate |
| Q. | formyl-CoA | 2.8.3.16 | Formate → formyl-CoA |
| | transferase | 6.2.1 | |
| | formyl-CoA synthase | | |
| R. | 3,5-ketovaleryl-CoA | 2.3.1.16 | Acetoacetyl-CoA + formyl-CoA → |
| | thiolase | | 3,5-ketovaleryl-CoA |
| S. | 5-hydroxy-3- | 1.1.1.35 | 3,5-ketovaleryl-CoA → |
| | Ketovaleryl-CoA | 1.1.1.36 | 5-hydroxy-3-Ketovaleryl-CoA |
| | dehydrogenase | | |
| T. | 3-hydroxy-5- | 1.1.1.35 | 3,5-ketovaleryl-CoA → R/S |
| | Ketovaleryl-CoA | 1.1.1.36 | 3-hydroxy-5-Ketovaleryl-CoA |
| | dehydrogenase | | |
| U. | 3,5-hydroxyvaleryl- | 1.1.1.35 | 5-hydroxy-3-Ketovaleryl-CoA or |
| | CoA dehydrogenase | 1.1.1.36 | R/S 3-hydroxy-5-Ketovaleryl-CoA → |
| | | | R/S 3,5-hydroxyvaleryl-CoA |
| V. | 3,5-hydroxyvaleryl- | 4.2.1.17 | R/S 3,5-hydroxyvaleryl-CoA → R/S |
| | CoA dehydratase | 4.2.1.54 | 3-hydroxy-4-pentenoyl-CoA |
| X. | 3-hydroxy-4- | 3.1.2, | R/S 3-hydroxy-4-pentenoyl-CoA → |
| | pentenoyl-CoA | 2.8.3 or | 3-hydroxy-4-pentenoic acid |
| | hydrolase, transferase | 2.3.3 | |
| | or synthase | | |
| Z. | 3-hydroxy-4- | 4.1.1.33 | 3-hydroxy-4-pentenoic acid → |
| | pentenoic acid | | butadiene |
| | decarboxylase | | |

The microorganism may be an archea, bacteria, or eukaryote. In some embodiments, the bacteria is a Propionibacterium, Propionispira, Clostridium, Bacillus, Escherichia, Pelobacter, or Lactobacillus including, for example, Pelobacter propionicus, Clostridium propionicum, Clostridium acetobutylicum, Lactobacillus, Propionibacterium acidipropionici or Propionibacterium freudenreichii. In some embodiments, the eukaryote is a yeast, filamentous fungi, protozoa, or algae. In some embodiments, the yeast is Saccharomyces cerevisiae or Pichia pastoris.

In some embodiments, the microorganism is additionally modified to comprise one or more tolerance mechanisms including, for example, tolerance to a produced biofuel (i.e., butadiene, 1-propanol, and/or 1,2-propanediol), and/or organic solvents. A microorganism modified to comprise such a tolerance mechanism may provide a means to increase titers of fermentations and/or may control contamination in an industrial scale process.

In some embodiments, the disclosure contemplates the modification (e.g., engineering) of one or more of the enzymes provided herein. Such modification may be performed to redesign the substrate specificity of the enzyme and/or to modify (e.g., reduce) its activity against others substrates in order to increase its selectivity for a given substrate. Additionally or alternatively, one or more enzymes as provided herein may be engineered to alter (e.g., enhance including, for example, increase its catalytic activity or its substrate specificity) one or more of its properties.

In some embodiments, sequence alignment and comparative modeling of proteins may be used to alter one or more of the enzymes disclosed herein. Homology modeling or comparative modeling refers to building an atomic-resolution model of the desired protein from its primary amino acid sequence and an experimental three-dimensional structure of a similar protein. This model may allow for the enzyme substrate binding site to be defined, and the identification of specific amino acid positions that may be replaced to other natural amino acid in order to redesign its substrate specificity.

Variants or sequences having substantial identity or homology with the polynucleotides encoding enzymes as disclosed herein may be utilized in the practice of the disclosure. Such sequences can be referred to as variants or modified sequences. That is, a polynucleotide sequence may be modified yet still retain the ability to encode a polypeptide exhibiting the desired activity. Such variants or modified sequences are thus equivalents in the sense that they retain their intended function. Generally, the variant or modified sequence may comprise at least about 40%-60%, preferably about 60%-80%, more preferably about 80%-90%, and even more preferably about 90%-95% sequence identity with the native sequence.

In some embodiments, a microorganism may be modified to express including, for example, overexpress, one or more enzymes as provided herein. The microorganism may be modified by genetic engineering techniques (i.e., recombinant technology), classical microbiological techniques, or a combination of such techniques and can also include naturally occurring genetic variants to produce a genetically modified microorganism. Some of such techniques are generally disclosed, for example, in Sambrook et al., 1989, Molecular Cloning: A Laboratory Manual, Cold Spring Harbor Labs Press.

A genetically modified microorganism may include a microorganism in which a polynucleotide has been inserted, deleted or modified (i.e., mutated; e.g., by insertion, deletion, substitution, and/or inversion of nucleotides), in such a manner that such modifications provide the desired effect of expression (e.g., over-expression) of one or more enzymes as provided herein within the microorganism. Genetic modifications which result in an increase in gene expression or function can be referred to as amplification, overproduction, overexpression, activation, enhancement, addition, or up-regulation of a gene. Addition of cloned genes to increase gene expression can include maintaining the cloned gene(s) on replicating plasmids or integrating the cloned gene(s) into the genome of the production organism. Furthermore, increasing the expression of desired cloned genes can include operatively linking the cloned gene(s) to native or heterologous transcriptional control elements.

Where desired, the expression of one or more of the enzymes provided herein are under the control of a regulatory sequence that controls directly or indirectly the expression of the enzyme in a time-dependent fashion during a fermentation reaction.

In some embodiments, a microorganism is transformed or transfected with a genetic vehicle such as, an expression vector comprising an exogenous polynucleotide sequence coding for the enzymes provided herein.

Polynucleotide constructs prepared for introduction into a prokaryotic or eukaryotic host may typically, but not always, comprise a replication system (i.e. vector) recognized by the host, including the intended polynucleotide fragment encoding the desired polypeptide, and may preferably, but not necessarily, also include transcription and translational initiation regulatory sequences operably linked to the polypeptide-encoding segment. Expression systems (expression vectors) may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, mRNA stabilizing sequences, nucleotide sequences homologous to host chromosomal DNA, and/or a multiple cloning site. Signal peptides may also be included where appropriate, preferably from secreted polypeptides of the same or related species, which allow the protein to cross and/or lodge in cell membranes or be secreted from the cell.

The vectors can be constructed using standard methods (see, e.g., Sambrook et al., Molecular Biology: A Laboratory Manual, Cold Spring Harbor, N.Y. 1989; and Ausubel, et al., Current Protocols in Molecular Biology, Greene Publishing, Co. N.Y, 1995).

The manipulation of polynucleotides of the present disclosure including polynucleotides coding for one or more of the enzymes disclosed herein is typically carried out in recombinant vectors. Numerous vectors are publicly available, including bacterial plasmids, bacteriophage, artificial chromosomes, episomal vectors and gene expression vectors, which can all be employed. A vector of use according to the disclosure may be selected to accommodate a protein coding sequence of a desired size. A suitable host cell is transformed with the vector after in vitro cloning manipulations. Host cells may be prokaryotic, such as any of a number of bacterial strains, or may be eukaryotic, such as yeast or other fungal cells, insect or amphibian cells, or mammalian cells including, for example, rodent, simian or human cells. Each vector contains various functional components, which generally include a cloning site, an origin of replication and at least one selectable marker gene. If given vector is an expression vector, it additionally possesses one or more of the following: enhancer element, promoter, transcription termination and signal sequences, each positioned in the vicinity of the cloning site, such that they are operatively linked to the gene encoding a polypeptide repertoire member according to the disclosure.

Vectors, including cloning and expression vectors, may contain nucleic acid sequences that enable the vector to replicate in one or more selected host cells. For example, the sequence may be one that enables the vector to replicate independently of the host chromosomal DNA and may include origins of replication or autonomously replicating sequences. Such sequences are well known for a variety of bacteria, yeast and viruses. For example, the origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

A cloning or expression vector may contain a selection gene also referred to as a selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate, hygromycin, thiostrepton, apramycin or tetracycline, complement auxotrophic deficiencies, or supply critical nutrients not available in the growth media.

The replication of vectors may be performed in E. coli (e.g., strain TB1 or TG1, DH5α, DH10α, JM110). An E. coli-selectable marker, for example, the β-lactamase gene that confers resistance to the antibiotic ampicillin, may be of use. These selectable markers can be obtained from E. coli plasmids, such as pBR322 or a pUC plasmid such as pUC18 or pUC19, or pUC119.

Expression vectors may contain a promoter that is recognized by the host organism. The promoter may be operably linked to a coding sequence of interest. Such a promoter may be inducible or constitutive. Polynucleotides are operably linked when the polynucleotides are in a relationship permitting them to function in their intended manner.

Promoters suitable for use with prokaryotic hosts may include, for example, the α-lactamase and lactose promoter systems, alkaline phosphatase, the tryptophan (trp) promoter system, the erythromycin promoter, apramycin promoter, hygromycin promoter, methylenomycin promoter and hybrid promoters such as the tac promoter. Moreover, host constitutive or inducible promoters may be used. Promoters for use in bacterial systems will also generally contain a Shine-Dalgarno sequence operably linked to the coding sequence.

Viral promoters obtained from the genomes of viruses include promoters from polyoma virus, fowlpox virus, adenovirus (e.g., Adenovirus 2 or 5), herpes simplex virus (thymidine kinase promoter), bovine papilloma virus, avian sarcoma virus, cytomegalovirus, a retrovirus (e.g., MoMLV, or RSV LTR), Hepatitis-B virus, Myeloproliferative sarcoma virus promoter (MPSV), VISNA, and Simian Virus 40 (SV40). Heterologous mammalian promoters include, e.g., the actin promoter, immunoglobulin promoter, heat-shock protein promoters.

The early and late promoters of the SV40 virus are conveniently obtained as a restriction fragment that also contains the SV40 viral origin of replication (see, e.g., Fiers et al., Nature, 273:113 (1978); Mulligan and Berg, Science, 209:1422-1427 (1980); and Pavlakis et al., Proc. Natl. Acad. Sci. USA, 78:7398-7402 (1981)). The immediate early promoter of the human cytomegalovirus (CMV) is conveniently obtained as a Hind III E restriction fragment (see, e.g., Greenaway et al., Gene, 18:355-360 (1982)). A broad host range promoter, such as the SV40 early promoter or the Rous sarcoma virus LTR, is suitable for use in the present expression vectors.

Generally, a strong promoter may be employed to provide for high level transcription and expression of the desired product. Among the eukaryotic promoters that have been identified as strong promoters for high-level expression are the SV40 early promoter, adenovirus major late promoter, mouse metallothionein-I promoter, Rous sarcoma virus long terminal repeat, and human cytomegalovirus immediate early promoter (CMV or CMV IE). In an embodiment, the promoter is a SV40 or a CMV early promoter.

The promoters employed may be constitutive or regulatable, e.g., inducible. Exemplary inducible promoters include jun, fos and metallothionein and heat shock promoters. One or both promoters of the transcription units can be an inducible promoter. In an embodiment, the GFP is expressed from a constitutive promoter while an inducible promoter drives transcription of the gene coding for one or more enzymes as disclosed herein and/or the amplifiable selectable marker.

The transcriptional regulatory region in higher eukaryotes may comprise an enhancer sequence. Many enhancer sequences from mammalian genes are known e.g., from globin, elastase, albumin, α-fetoprotein and insulin genes. A suitable enhancer is an enhancer from a eukaryotic cell virus. Examples include the SV40 enhancer on the late side of the replication origin (bp 100-270), the enhancer of the cytomegalovirus immediate early promoter (Boshart et al. Cell 41:521 (1985)), the polyoma enhancer on the late side of the replication origin, and adenovirus enhancers (see also, e.g., Yaniv, Nature, 297:17-18 (1982) on enhancing elements for activation of eukaryotic promoters). The enhancer sequences may be introduced into the vector at a position 5′ or 3′ to the gene of interest, but is preferably located at a site 5′ to the promoter.

Yeast and mammalian expression vectors may contain prokaryotic sequences that facilitate the propagation of the vector in bacteria. Therefore, the vector may have other components such as an origin of replication (e.g., a nucleic acid sequence that enables the vector to replicate in one or more selected host cells), antibiotic resistance genes for selection in bacteria, and/or an amber stop codon which can permit translation to read through the codon. Additional eukaryotic selectable gene(s) may be incorporated. Generally, in cloning vectors the origin of replication is one that enables the vector to replicate independently of the host chromosomal DNA, and includes origins of replication or autonomously replicating sequences. Such sequences are well known, e.g., the ColE1 origin of replication in bacteria. Various viral origins (e.g., SV40, polyoma, adenovirus, VSV or BPV) are useful for cloning vectors in mammalian cells. Generally, a eukaryotic replicon is not needed for expression in mammalian cells unless extrachromosomal (episomal) replication is intended (e.g., the SV40 origin may typically be used only because it contains the early promoter).

To facilitate insertion and expression of different genes coding for the enzymes as disclosed herein from the constructs and expression vectors, the constructs may be designed with at least one cloning site for insertion of any gene coding for any enzyme disclosed herein. The cloning site may be a multiple cloning site, e.g., containing multiple restriction sites.

The plasmids may be propagated in bacterial host cells to prepare DNA stocks for subcloning steps or for introduction into eukaryotic host cells. Transfection of eukaryotic host cells can be any performed by any method well known in the art. Transfection methods include lipofection, electroporation, calcium phosphate co-precipitation, rubidium chloride or polycation mediated transfection, protoplast fusion and microinjection. Preferably, the transfection is a stable transfection. The transfection method that provides optimal transfection frequency and expression of the construct in the particular host cell line and type, is favored. Suitable methods can be determined by routine procedures. For stable transfectants, the constructs are integrated so as to be stably maintained within the host chromosome.

Vectors may be introduced to selected host cells by any of a number of suitable methods known to those skilled in the art. For example, vector constructs may be introduced to appropriate cells by any of a number of transformation methods for plasmid vectors. For example, standard calcium-chloride-mediated bacterial transformation is still commonly used to introduce naked DNA to bacteria (see, e.g., Sambrook et al., 1989, Molecular Cloning, A Laboratory Manual, Cold Spring Harbor Laboratory Press, Cold Spring Harbor, N.Y.), but electroporation and conjugation may also be used (see, e.g., Ausubel et al., 1988, Current Protocols in Molecular Biology, John Wiley & Sons, Inc., NY, N.Y.).

For the introduction of vector constructs to yeast or other fungal cells, chemical transformation methods may be used (e.g., Rose et al., 1990, Methods in Yeast Genetics, Cold Spring Harbor Laboratory Press, Cold Spring Harbor, N.Y.). Transformed cells may be isolated on selective media appropriate to the selectable marker used. Alternatively, or in addition, plates or filters lifted from plates may be scanned for GFP fluorescence to identify transformed clones.

For the introduction of vectors comprising differentially expressed sequences to mammalian cells, the method used may depend upon the form of the vector. Plasmid vectors may be introduced by any of a number of transfection methods, including, for example, lipid-mediated transfection (“lipofection”), DEAE-dextran-mediated transfection, electroporation or calcium phosphate precipitation (see, e.g., Ausubel et al., 1988, Current Protocols in Molecular Biology, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

The host cell may be capable of expressing the construct encoding the desired protein, processing the protein and transporting a secreted protein to the cell surface for secretion. Processing includes co- and post-translational modification such as leader peptide cleavage, GPI attachment, glycosylation, ubiquitination, and disulfide bond formation. Immortalized host cell cultures amenable to transfection and in vitro cell culture and of the kind typically employed in genetic engineering are preferred. Examples of useful mammalian host cell lines are monkey kidney CV1 line transformed by SV40 (COS-7, ATCC CRL 1651); human embryonic kidney line (293 or 293 derivatives adapted for growth in suspension culture, Graham et al., J. Gen Virol., 36:59 (1977); baby hamster kidney cells (BHK, ATCC CCL 10); DHFR—Chinese hamster ovary cells (ATCC CRL-9096); dp12.CHO cells, a derivative of CHO/DHFR-(EP 307,247 published 15 Mar. 1989); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); PEER human acute lymphoblastic cell line (Ravid et al. Int. J. Cancer 25:705-710 (1980)); MRC 5 cells; FS4 cells; human hepatoma line (Hep G2), human HT1080 cells, KB cells, JW-2 cells, Detroit 6 cells, NIH-3T3 cells, hybridoma and myeloma cells. Embryonic cells used for generating transgenic animals are also suitable (e.g., zygotes and embryonic stem cells).

Suitable host cells for cloning or expressing polynucleotides (e.g., DNA) in vectors may include, for example, prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), E. coli JM110 (ATCC 47,013) and E. coli W3110 (ATCC 27,325) are suitable.

In addition to prokaryotes, eukaryotic microbes such as filamentous fungi or yeast may be suitable cloning or expression hosts for vectors comprising polynucleotides coding for one or more enzymes. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

When the enzyme is glycosylated, suitable host cells for expression may be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori (silk moth) have been identified. A variety of viral strains for transfection are publicly available, e.g., the L-1 variant of Autographa californica NPV and the Bm-5 strain of Bombyx mori NPV, and such viruses may be used as the virus herein according to the present disclosure, particularly for transfection of Spodoptera frugiperda cells.

Plant cell cultures of cotton, corn, potato, soybean, petunia, tomato, tobacco, lemna, and other plant cells can also be utilized as host cells.

Examples of useful mammalian host cells are Chinese hamster ovary cells, including CHOK1 cells (ATCC CCL61), DXB-11, DG-44, and Chinese hamster ovary cells/-DHFR (CHO, Urlaub et al., Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, (Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y Acad. Sci. 383: 44-68 (1982)); MRC 5 cells; FS4 cells; and a human hepatoma line (Hep G2).

Host cells are transformed or transfected with the above-described expression or cloning vectors for production of one or more enzymes as disclosed herein or with polynucleotides coding for one or more enzymes as disclosed herein and cultured in conventional nutrient media modified as appropriate for inducing promoters, selecting transformants, or amplifying the genes encoding the desired sequences.

Host cells containing desired nucleic acid sequences coding for the disclosed enzymes may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44, (1979); Barnes et al., Anal. Biochem. 102: 255 (1980); U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adeNOSine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Polynucleotides and Encoded Enzymes

Any known polynucleotide (e.g., gene) that codes for an enzyme or variant thereof that is capable of catalyzing an enzymatic conversion including, for example, an enzyme as set forth in any one of Tables 1-4 or FIGS. 1-4, is contemplated for use by the present disclosure. Such polynucleotides may be modified (e.g., genetically engineered) to modulate (e.g., increase or decrease) the substrate specificity of an encoded enzyme, or the polynucleotides may be modified to change the substrate specificity of the encoded enzyme (e.g., a polynucleotide that codes for an enzyme with specificity for a substrate may be modified such that the enzyme has specificity for an alternative substrate). Preferred microorganisms may comprise polynucleotides coding for one or more of the enzymes as set forth in any one of Tables 1-4 and FIG. 1-4.

In some embodiments, the microorganism may comprise an oxidoreductase as set forth in EC 1.1.1 including, for example, any one of SEQ ID NOS: 134-166 (Table 5). In some embodiments, the microorganism may comprise a dehydrogenase as set forth in EC 1.2.1 including, for example, any one of SEQ ID NOS: 20-25 (Table 5). In some embodiments, the microorganism may comprise an oxidoreductase as set forth in EC 1.2.1 including, for example, any one of SEQ ID NOS: 14-19 (Table 5). In some embodiments, the microorganism may comprise a transferase as set forth in EC 2.8.3 including, for example, any one of SEQ ID NOS: 29-57 (Table 5). In some embodiments, the microorganism may comprise a synthase as set forth in EC 2.3.3 including, for example, any one of SEQ ID NOS: 1-4 (Table 5). In some embodiments, the microorganism may comprise a hydrolase as set forth in EC 3.1.2 including, for example, any one of SEQ ID NOS: 58-62 (Table 5). In some embodiments, the microorganism may comprise a CoA synthetase as set forth in EC 6.2.1 including, for example, any one of SEQ ID NOS: 63-67 (Table 5). In some embodiments, the microorganism may comprise a ketothiolase as set forth in EC 2.3.1 including, for example, any one of SEQ ID NOS: 91-111 (Table 5). In some embodiments, the microorganism may comprise a dehydratase as set forth in EC 4.2.1 including, for example, any one of SEQ ID NOS: 68-88 (Table 5). In some embodiments, the microorganism may comprise a phosphate-lyase as set forth in EC 4.2.3 including, for example, any one of SEQ ID NOS: 26-28 (Table 5). In some embodiments, the microorganism may comprise a decarboxylase as set forth in EC 4.1.1 including, for example, any one of SEQ ID NOS: 112-133 (Table 5). In some embodiments, the microorganism may comprise a phosphotransferase as set forth in EC 2.7.1 or 2.7.4 including, for example, any one of SEQ ID NOS: 9-13 or 5-8, respectively (Table 5).

Enzymes for catalyzing the conversions in FIGS. 1-4 are categorized in Table 5 by Enzyme Commission (EC) number, function, and the step in FIGS. 1-4 in which they catalyze a conversion.

TABLE 5
EC number for employed enzymes

| Label | Function | Figure (Number) and Step (Letter) |
|---|---|---|
| 1.1.1 | Oxidoreductase | 1B, 1C, 1D, 1E, 1G, 1H, 1I, 1O, 1Q, 1S |
| | | 2B, 2C, 2D, 2E, 2G, 2H, 2I, 2R, |
| | | 3B, 3C, 3D, 3E, 3G, 3H, 3I, 3Q |
| | | 4B, 4C, 4D, 4E, 4G, 4H, 4I, 4S, 4T, 4U |
| 1.2.1 | Dehydrogenase | 1K, 1R |
| | | 2K |
| | | 3K |
| | | 4K, 4O, |
| 1.2.4 | Oxidoreductase | 1M |
| | | 2M |
| | | 3M |
| | | 4M |
| 2.8.3 | Transferase | 2N, 2T |
| | | 3R |
| | | 4Q, 4X |
| 2.3.3 | Synthase | 2T |
| | | 4X |
| 3.1.2 | Hydrolase | 2T |
| | | 3R |
| | | 4X |
| 6.2.1 | CoA Synthetase | 1L |
| | | 2L, 2N |
| | | 3N, 3R |
| | | 4L, 4Q |
| 2.3.1 | Ketothiolase | 1N, |
| | | 2Q |
| | | 3P |
| | | 4N, 4P, 4R |
| 4.2.1 | Dehydratase | 1F, 1P, 1T |
| | | 2F, 2O, 2P, 2S |
| | | 3F, 3O, |
| | | 4F, 4V |
| 4.2.3 | phosphate-lyase | 1A, 1Z, |
| | | 2A, |
| | | 3A, |
| | | 4A |
| 4.1.1 | Decarboxylase | 1J |
| | | 2J, 2U |
| | | 3J, 3R |
| | | 4J, 4Z |
| 2.7.1 | Phosphotransferase | 1U, 1X |
| 2.7.4 | Phosphotransferase | 1V |

Step T of FIG. 2 and step X of FIG. 4 can be catalyzed by synthases in EC 2.3.3 including, for example, a synthase that converts acyl groups into alkyl groups upon transfer. Any known polynucleotide coding for a synthase enzyme including, for example, those polynucleotides set forth in Table 6 below, are contemplated for use by the present disclosure.

TABLE 6
Exemplary genes coding for enzymes in EC 2.3.3

| | Gene ID (GI) | | SEQ |
|---|---|---|---|
| | or Accession | | ID |
| Gene | Number (AN) | Organism | NO: |
| menD | 12665319 | Escherichia coli | 1 |
| leuA | 947465 | Escherichia coli | 2 |
| ECBD_4023 | 8156527 | Escherichia coli | 3 |
| Lbuc_0961 | 10525118 | Lactobacillus buchneri | 4 |

Step V of FIG. 1 can be catalyzed by phosphotransferases in EC 2.7.4 including, for example, a phosphotransferase that transfers phosphorus-containing groups with a phosphorus group as an acceptor. Any known polynucleotide coding for a phosphotransferase enzyme including, for example, those polynucleotides set forth in Table 7 below, are contemplated for use by the present disclosure.

TABLE 7
Exemplary genes coding for enzymes in EC 2.7.4

| | Gene ID (GI) | | SEQ |
|---|---|---|---|
| | or Accession | | ID |
| Gene | Number (AN) | Organism | NO: |
| pmvk | 8570979 | Lactobacillus johnsonii | 5 |
| mvaK2 | 1120565 | Streptococcus pneumoniae | 6 |
| mvaK2 | 8149124 | Streptococcus suis | 7 |
| mvaK2 | 10868289 | Lactobacillus reuteri | 8 |

Steps U and X of FIG. 1 can be catalyzed by phosphotransferases in EC 2.7.1 including, for example, a phosphotransferase that transfer phosphorus-containing groups with an alcohol group as an acceptor. Any known polynucleotide coding for a phosphotransferase enzyme including, for example, those polynucleotides set forth in Table 8 below, are contemplated for use by the present disclosure.

TABLE 8
Exemplary genes coding for enzymes in EC 2.7.1

| | Gene ID (GI) | | SEQ |
|---|---|---|---|
| | or Accession | | ID |
| Gene | Number (AN) | Organism | NO: |
| Erg12 | 855248 | Saccharomyces cerevisiae | 9 |
| Erg12 | 2542818 | Schizosaccharomyces pombe | 10 |
| SFUL_6422 | 15406199 | Streptomyces fulvissimus | 11 |
| Nmlp_1448 | 14650715 | Natronomonas moolapensis | 12 |
| aroK | 12930303 | Escherichia coli K12 | 13 |

Steps M of FIGS. 1, 2, 3 and 4 can be catalyzed by oxidoreductases in EC 1.2.4 including, for example, an oxidoreductase acting on the aldehyde or oxo group of donors with a disulfide as acceptor. Any known polynucleotide coding for an oxidoreductase enzyme including, for example, those polynucleotides set forth in Table 9 below, are contemplated for use by the present disclosure.

TABLE 9
Exemplary genes coding for enzymes in EC 1.2.4

| | Gene ID (GI) | | SEQ |
|---|---|---|---|
| | or Accession | | ID |
| Gene | Number (AN) | Organism | NO: |
| pdhA | 1200253 | Enterococcus faecalis | 14 |
| pdhB | 1200254 | Enterococcus faecalis | 15 |
| aceF | 1200255 | Enterococcus faecalis | 16 |
| PDA1 | 856925 | Saccharomyces cerevisiae | 17 |
| PDB1 | 852522 | Saccharomyces cerevisiae | 18 |
| PDX1 | 853107 | Saccharomyces cerevisiae | 19 |

Steps K of FIGS. 1, 2, 3 and 4, step R in FIG. 1, and step O in FIG. 4 can be catalyzed by dehydrogenases in EC 1.2.1 including, for example, a dehydrogenase acting on the aldehyde or oxo group of donors with NAD(+) or NADP(+) as acceptor. Any known polynucleotide coding for a dehydrogenase enzyme including, for example, those polynucleotides set forth in Table 10 below, are contemplated for use by the present disclosure.

TABLE 10
Exemplary genes coding for enzymes in EC 1.2.1

| | Gene ID (GI) | | SEQ |
|---|---|---|---|
| | or Accession | | ID |
| Gene | Number (AN) | Organism | NO: |
| Ald6p | 856044 | Saccharomyces cerevisiae | 20 |
| adhE | 251817349 | Streptococcus suis | 21 |
| FdsA | 882378 | Burkholderia rhizoxinica | 22 |
| FdsB | 882378 | Burkholderia rhizoxinica | 23 |
| FdsG | 882378 | Burkholderia rhizoxinica | 24 |
| FdsD | 882378 | Burkholderia rhizoxinica | 25 |

Steps A of FIGS. 1, 2, 3 and 4 and step Z in FIG. 1 can be catalyzed by phosphate-lyases in EC 4.2.3 including, for example, a phosphate-lyase that can catalyze carbon-oxygen breakdown by acting on phosphate groups. Any known polynucleotide coding for a phosphate-lyase enzyme including, for example, those polynucleotides set forth in Table 11 below, are contemplated for use by the present disclosure.

TABLE 11
Exemplary genes coding for enzymes in EC 4.2.3

| | Gene ID (GI) | | SEQ |
|---|---|---|---|
| | or Accession | | ID |
| Gene | Number (AN) | Organism | NO: |
| mgsA | 16079305 | Bacillus subtilis | 26 |
| ispS | 13539551 | Populus alba | 27 |
| IspS | 118200117 | Populus nigra | 28 |

Steps N and T of FIG. 2, step R in FIG. 3, and steps Q and X in FIG. 4 can be catalyzed by CoA transferases in EC 2.8.3 including, for example, a CoA transferase that catalyzes the reversible transfer of a CoA moiety from one molecule to another. Any known polynucleotide coding for a CoA transferase enzyme including, for example, those polynucleotides set forth in Table 12 below, are contemplated for use by the present disclosure.

TABLE 12
Exemplary genes coding for enzymes in EC 2.8.3

| | Gene ID (GI) | | SEQ |
|---|---|---|---|
| | or Accession | | ID |
| Gene | Number (AN) | Organism | NO: |
| atoA | 2492994 | Escherichia coli K12 | 29 |
| atoD | 2492990 | Escherichia coli K12 | 30 |
| actA | 62391407 | Corynebacterium glutamicum | 31 |
| | | ATCC 13032 | |
| Cg0592 | 62289399 | Corynebacterium glutamicum | 32 |
| | | ATCC 13032 | |
| ctfA | 15004866 | Clostridium acetobutylicum | 33 |
| ctfB | 15004867 | Clostridium acetobutylicum | 34 |
| Ach1 | 60396828 | Roseburia sp. A2-183 | 35 |
| Pct | 7242549 | Clostridium propionicum | 36 |
| Cbei_4543 | 150019354 | Clostridium beijerinchii | 37 |
| pcaI | 50084858 | Acinetobacter sp. ADP1 | 38 |
| PcaJ | 141776 | Acinetobacter sp. ADP1 | 39 |
| pcaI | 24985644 | Pseudomonas putida | 40 |
| pcaJ | 141776 | Pseudomonas putida | 41 |
| ScoA | 16080950 | Bacillus subtilis | 42 |
| ScoB | 16080949 | Bacillus subtilis | 43 |
| Cat1 | 729048 | Clostridium kluyveri | 44 |
| Cat2 | 172046066 | Clostridium kluyveri | 45 |
| Cat3 | 146349050 | Clostridium kluyveri | 46 |
| gctA | 559392 | Acidaminococcus fermentans | 47 |
| gctB | 559393 | Acidaminococcus fermentans | 48 |
| frc | 12931869 | Escherichia coli | 49 |
| BBta_3113 | 5149017 | Bradyrhizobium sp. | 50 |
| RPA1945 | 2688995 | Rhodopseudomonas palustris | 51 |
| SDY_2572 | 3797090 | Shigella dysenteriae | 52 |
| RPB_3427 | 3911229 | Rhodopseudomonas palustris | 53 |
| frc | 8191935 | Methylobacterium extorquens | 54 |
| H16_B1711 | 4455693 | Ralstonia eutropha H16 | 55 |
| Bxe_B2760 | 4006524 | Burkholderia xenovorans | 56 |
| cat | 9283801 | Propionibacterium freudenreichii | 57 |

Alternatively, step T of FIG. 1, step R of FIG. 2, and step X of FIG. 3 can be catalyzed by hydrolases in EC 3.1.2 including, for example, a hydrolase with broad substrate ranges and that are suitable for hydrolyzing 2-petentenoyl-CoA; 2,4-pentenoyl-CoA, 3-hydroxypentenoyl-CoA and other compounds to their correspondent acids. Any known polynucleotide coding for a hydrolase enzyme including, for example, those polynucleotides set forth in Table 13 below, are contemplated for use by the present disclosure.

TABLE 13
Exemplary genes coding for enzymes in EC 3.1.2

| | Gene ID (GI) | | SEQ |
|---|---|---|---|
| | or Accession | | ID |
| Gene | Number (AN) | Organism | NO: |
| Orf1 | 23664428 | Azoarcus evansii | 58 |
| COG0824 | 46200680 | Magnetospirillum | 59 |
| | | magnetotacticum | |
| Jann_0674 | 89052491 | Jannaschia sp. CCS1 | 60 |
| SSE37_24444 | 126729407 | Sagittula stellata | 61 |
| entH | 1786813 | Escherichia coli | 62 |

Alternatively, step L of FIG. 1, steps L and N of FIG. 2, steps N and R of FIG. 3, and steps L and Q of FIG. 4 can be catalyzed by CoA synthetases in EC 6.2.1 including, for example, a CoA synthetase with a broad substrate range. Any known polynucleotide coding for a CoA synthetase enzyme including, for example, those polynucleotides set forth in Table 14 below, are contemplated for use by the present disclosure.

TABLE 14
Exemplary genes coding for enzymes in EC 6.2.1

| | Gene ID (GI) | | SEQ |
|---|---|---|---|
| | or Accession | | ID |
| Gene | Number (AN) | Organism | NO: |
| acs | 8434601 | Acetobacter pasteurianus | 63 |
| Avin_10660 | 7760010 | Azotobacter vinelandii | 64 |
| acs | 8657923 | Dehalococcoides sp. | 65 |
| Acs2 | 850846 | Saccharomyces cerevisiae | 66 |
| Acs1 | 851245 | Saccharomyces cerevisiae | 67 |

Steps F, P and T in FIG. 1, steps F, O, P and S in FIG. 2, steps F, O, and H in FIG. 3, and steps F and V in FIG. 4 involve the addition or removal of water from a given substrate. Such conversions can be catalyzed by hydratase or dehydratase enzymes in EC 4.2.1. Any known polynucleotide coding for a hydratase enzyme including, for example, those polynucleotides set forth in Table 15 below, are contemplated for use by the present disclosure.

TABLE 15
Exemplary genes coding for enzymes in EC 4.2.1

| | Gene ID (GI) | | SEQ |
|---|---|---|---|
| | or Accession | | ID |
| Gene | Number (AN) | Organism | NO: |
| mhpD | 87081722 | Escherichia coli | 68 |
| ctmF | 1263188 | Pseudomonas putida | 69 |
| todG | 1263188 | Pseudomonas putida | 70 |
| hpaH | 7150958100 | Klebsiella pneumoniae | 71 |
| hpaH | 8178258 | Escherichia coli | 72 |
| cnbE | 6386628 | Comamonas testosteroni | 73 |
| leuD | 2122345 | Methanocaldococcus jannaschii | 74 |
| dmdA | 9884634 | Eubacterium limosum | 75 |
| dmdB | 9884633 | Eubacterium limosum | 76 |
| Olhyd_maccj | 7390838 | Macrococcus caseolyticus | 77 |
| ech | 1047000 | Pseudomonas putida | 78 |
| crt | 1118895 | Clostridium acetobutylicum | 79 |
| phaB | 1046931 | Pseudomonas putida | 80 |
| fadA | 12934462 | Escherechia coli | 81 |
| fadB | 12934454 | Escherechia coli | 82 |
| fadI | 12933009 | Escherechia coli | 83 |
| fadJ | 12931539 | Escherechia coli | 84 |
| fadR | 12931108 | Escherechia coli | 85 |
| ldi | 302064203 | Castellaniella defragrans | 86 |
| dhaB2 | 5341465 | Klebsiella pneumoniae | 87 |
| dhaB1 | 5341416 | Klebsiella pneumoniae | 88 |

A dehydratase/isomerase is engineered to accept crotonyl-alcohol as a substrate, thus representing a suitable candidate for step T in FIG. 1. The linalool dehydratase-isomerase from Castellaniella defragrans strain 65Phen catalyzes the stereospecific hydration of beta-myrcene to (3S)-linalool and the isomerization of (3S)-linalool to geraniol. This enzyme also catalyzes the reverse reactions, i.e., the isomerization of geraniol to linalool and the dehydration of linalool to myrcene. In this direction, the formation of myrcene from geraniol may be seen as a detoxification process for the monoterpene alcohol. The enzymes has been overexpressed in E. coli and is well-characterized biochemically. Other dehydratase-isomerases include, for example, 4-hydroxybutyryl-CoA dehydratase/vinylacetyl-CoA-Delta-isomerase. Any known polynucleotide may be engineered including, for example, those polynucleotides set forth in Table 16 below, are contemplated for use by the present disclosure.

TABLE 16
Exemplary genes coding for linalool dehydratase-isomerases

| | Gene ID (GI) | | SEQ |
|---|---|---|---|
| | or Accession | | ID |
| Gene | Number (AN) | Organism | NO: |
| ldi | 302064203 | Castellaniella defragrans | 89 |
| abdD | 1453964 | Sulfolobus solfataricus | 90 |

Step N of FIG. 1, step Q of FIG. 2, step P of FIG. 3, and steps N, P, and R of FIG. 4 may be catalyzed by ketothiolases in EC 2.3.1. Any known polynucleotide coding for a hydratase enzyme including, for example, those polynucleotides set forth in Table 17 below, are contemplated for use by the present disclosure.

TABLE 17
Exemplary genes coding for enzymes in EC 2.3.1

| | Gene ID (GI) | | SEQ |
|---|---|---|---|
| | or Accession | | ID |
| Gene | Number (AN) | Organism | NO: |
| paaJ | 12934018 | Escherichia coli | 91 |
| phaD | 1046928 | Pseudomonas putida | 92 |
| pcaF | 10441755 | Pseudomonas putida | 93 |
| pcaF | 11639550 | Acinetobacter calcoaceticus | 94 |
| fadA | 4490319 | Aeromonas hydrophila | 95 |
| AtoB | 4997503 | Aeromonas salmonicida | 96 |
| pcaF | 4383639 | Pseudomonas aeroginosa | 97 |
| bktB | 428815 | Ralstonia eutropha | 98 |
| pimB | 2692199 | Rhodopseudomonas palustris | 99 |
| syn_02642 | 3882984 | Syntrophus aciditrophicus | 100 |
| phaA | 10921806 | Cupriavidus necator | 101 |
| atoB | 12934272 | Escherichia coli | 102 |
| thlA | 1119056 | Clostridium acetobutylicum | 103 |
| thlB | 1116083 | Clostridium acetobutylicum | 104 |
| ERG10 | 856079 | Saccahromyces cerevisiae | 105 |
| pflB | 12931841 | Escherichia coli | 106 |
| pflA | 12930359 | Escherichia coli | 107 |
| pfl | 15671982 | Lactococcus lactis | 108 |
| pfl | 3168596 | Streptococcus equinus | 109 |
| act | 14141682 | Streptococcus equinus | 110 |
| Clo1313_1716 | 12421448 | Clostridium thermocellum | 111 |

Step J of FIG. 1, step J and U of FIG. 2, step J and R of FIG. 3, and step J and Z of FIG. 4 can be catalyzed by decarboxylases in EC 4.1.1. Exemplary enzymes for step J of FIG. 1 include, for example, sorbic acid decarboxylase and aconitate decarboxylase (EC 4.1.1.16). An exemplary enzyme for step T of FIG. 2 and step S of FIG. 3 and, step Z of FIG. 4 includes, for example, diphosphomevalonate decarboxylase (EC 4.1.1.33). Any known polynucleotide coding for a decarboxylase enzyme including, for example, those polynucleotides set forth in Table 18 below, are contemplated for use by the present disclosure.

TABLE 18
Exemplary genes coding for enzymes in EC 4.1.1

| | Gene ID (GI) | | SEQ |
|---|---|---|---|
| | or Accession | | ID |
| Gene | Number (AN) | Organism | NO: |
| OleT_{JE} | HQ709266. | Jeotgalicoccus sp; ATCC8456 | 112 |
| PadA1 | 145235767 | Aspergillus niger | 113 |
| ohbA1 | 145235771 | Aspergillus niger | 114 |
| sdrA | 145235769 | Aspergillus niger | 115 |
| padA1 | 169786362 | Aspergillis oryzae | 116 |
| ohbA1 | 169768360 | Aspergillis oryzae | 117 |
| sdrA | 169768362 | Aspergillis oryzae | 118 |
| Mvd | 2845318 | Picrophilus torridus | 119 |
| mvd | 2845209 | Picrophilus torridus | 120 |
| mvd | 855779 | Saccharomyces cerevisiae | 121 |
| mvd | 162312575 | Schizosaccharomyces pombe | 122 |
| mvd | 257051090 | Halorhabdus utahensis | 123 |
| mvd | 8741675 | Haloterrigena turkmenica | 124 |
| mvd | 9132821 | Leuconostoc kimchii | 125 |
| dvd | 1447408 | Halobacterium salinarum | 126 |
| dfd | 121708954 | Aspergillus clavatus | 127 |
| | 4593483 | Neosartorya fischeri | 128 |
| mvaD | 11027973 | Streptococcus pseudopneumoniae | 129 |
| mvaD | 8433456 | Lactobacillus rhamnosus | 130 |
| mvaD | 12158799 | Borrelia afzelii | 131 |
| Pdc2 | 851654 | Saccharomyces cerevisae | 132 |
| pdc-1 | 12759328 | Escherichia coli | 133 |

Steps B, C, D, E, G, H and I of FIGS. 1, 2, 3 and 4, steps O, Q and S in FIG. 1, step R of FIG. 3, and steps S, T and U of FIG. 4 can be catalyzed by oxidoreductases in EC 1.1.1 including, for example, an oxidoreductase that can reduce a ketone to an alcohol. Any known polynucleotide coding for a hydratase enzyme including, for example, those polynucleotides set forth in Table 19 below, are contemplated for use by the present disclosure.

TABLE 19
Exemplary genes coding for enzymes in EC 1.1.1

| | Gene ID (GI) | | SEQ |
|---|---|---|---|
| | or Accession | | ID |
| Gene | Number (AN) | Organism | NO: |
| mdh | 6059112 | Escherichia coli | 134 |
| idhA | 5591397 | Escherichia coli | 135 |
| idh | 113866693 | Ralstonia eutropha | 136 |
| adh | 60592974 | Clostridium beijerinckii | 137 |
| Adh | 113443 | Thermoanaerobacter brockii | 138 |
| Sadh | 21615552 | Rhodococcus ruber | 139 |
| adhA | 3288810 | Pyrococcus furiosus | 140 |
| adhE | 12930611 | Escherichia coli | 141 |
| adhE2 | 12958626 | Clostridium acetobutylicum | 142 |
| adhE | 55818563 | Leuconostoc mesenteroides | 143 |
| HMG1 | 854900 | Saccharomyces cerevisiae | 144 |
| CtCNB1_3119 | 8560791 | Comamonas testosteroni | 145 |
| DKAM_0720 | 7170894 | Desulfurococcus kamchatkensis | 146 |
| mvaA | 1004602 | Staphylococcus aureus | 147 |
| LJ1608 | 2742117 | Lactobacillus johnsonii | 148 |
| acr1 | 2879608 | Acinetobacter sp. ADP1 | 149 |
| acr1 | 1684885 | Acinetobacter baylyi | 150 |
| sucD | 5394466 | Clostridium kluyveri | 151 |
| sucD | 2551522 | Porphyromonas gingivalis | 152 |
| bld | 31075383 | Clostridium | 153 |
| | | saccharoperbutylacetonicum | |
| Cbei_3832 | 5294993 | Clostridium beijerinckii | 154 |
| yeaE | 251785229 | Escherichia coli | 155 |
| dhaT | 206570135 | Klebsiella pneumoniae | 156 |
| fucO | 291280824 | Escherichia coli | 157 |
| Cbei_2421 | 5293624 | Clostridium beijerinckii | 158 |
| Lreu_0194 | 148543431 | Lactobacillus reuteri | 159 |
| car | 40796035; | Nocardia sp. | 160 |
| | 114848891; | | |
| | 13728088 | | |
| acpS | 114848891, | Nocardia sp. | 161 |
| | 13728088 | | |
| crt | 300869639 | Brachyspira pilosicoli | 162 |
| fadb | 13857931 | Salmonella enterica | 163 |
| tpa | 281809 | Bos taurus | 164 |
| fadb | 56412276 | Salmonella enterica | 165 |
| fadb | 4010435 | Burkholderia xenovorans LB400 | 166 |

Other reactions involve dehydrogenation of CH—CH group of donors with NAD+ or NADP+ as an electron acceptor, family EC 1.2.1. Numerous dehydrogenases have characterized and show to dehydrogenate structurally similar substrates to crotonyl—CoA (Step R of FIG. 1), CO₂ (Step O in FIG. 4) and acetaldehyde (Step K in FIG. 1, FIG. 2 and FIG. 3). Any known polynucleotide coding for a dehydrogenase enzyme including, for example, those polynucleotides set forth in Table 20 below, are contemplated for use by the present disclosure.

TABLE 20
Exemplary genes coding for dehydrogenases

| | Gene ID (GI) | | SEQ |
|---|---|---|---|
| | or Accession | | ID |
| Gene | Number (AN) | Organism | NO: |
| Msed_1426 | 5104797 | Metallosphaera sedula | 167 |
| ST0480 | 1458422 | Sulfolobus tokodaii | 168 |
| Mcup_0809 | 10493000 | Metallosphaera cuprina | 169 |
| RBRH_02090 | 9986550 | Streptomyces clavuligerus | 170 |
| RSP_1434 | 3718801 | Rhodobacter sphaeroides | 171 |
| acrA | JN244654.1 | Clostridium propionicum | 172 |
| acrB | JN244655 | Clostridium propionicum | 173 |
| Fdh1 | 2276464 | Candida boidinii | 174 |
| Fdh1 | 854570 | Saccharomyces cerevisiae | 175 |
| Fdh2 | 1370568 | Saccharomyces cerevisiae | 176 |
| fdsC | 4248880 | Cupriavidus necator | 177 |
| fdsA | 4248878 | Cupriavidus necator | 178 |
| fdsB | 4248879 | Cupriavidus necator | 179 |
| fdsD | 4248881 | Cupriavidus necator | 180 |
| fdsG | 4248882 | Cupriavidus necator | 181 |
| fdsR | 4248883 | Cupriavidus necator | 182 |

Methods for the Co-Production of Butadiene and 1-Propanol and/or 1,2-Propandiol

Butadiene and 1-propanol and/or 1,2-propanediol may be produced by contacting any of the genetically modified microorganisms provided herein with a fermentable carbon source. Such methods may preferably comprise contacting a fermentable carbon source with a microorganism comprising one or more polynucleotides coding for enzymes in a pathway that catalyzes a conversion of the fermentable carbon source to any of the intermediates provided in FIGS. 1-4 (tables 1-4) and one or more polynucleotides coding for enzymes in a pathway that catalyze a conversion of the one or more intermediates provided in FIGS. 1-4 (tables 1-4) to butadiene and 1-propanol and/or 1,2-propanediol in a fermentation media; and expressing the one or more polynucleotides coding for the enzymes in the pathway that catalyzes a conversion of the fermentable carbon source to the one or more intermediates provided in FIGS. 1-4 (tables 1-4) and one or more polynucleotides coding for enzymes in a pathway that catalyze a conversion of the one or more intermediates provided in FIGS. 1-4 (tables 1-4) to butadiene and 1-propanol and/or 1,2-propanediol.

The metabolic pathways that lead to the production of industrially important compounds involve oxidation-reduction (redox) reactions. For example, during fermentation, glucose is oxidized in a series of enzymatic reactions into smaller molecules with the concomitant release of energy. The electrons released are transferred from one reaction to another through universal electron carriers, such Nicotinamide Adenine Dinucleotide (NAD) and Nicotinamide Adenine Dinucleotide Phosphate (NAD(P)), which act as cofactors for oxidoreductase enzymes. In microbial catabolism, glucose is oxidized by enzymes using the oxidized form of the cofactors (NAD(P)+ and/or NAD+) as cofactor thus generating reducing equivalents in the form of the reduced cofactor (NAD(P)H and NADH). In order for fermentation to continue, redox-balanced metabolism is required, i.e., the cofactors must be regenerated by the reduction of microbial cell metabolic compounds.

Microorganism-catalyzed fermentation for the production of natural products is a widely known application of biocatalysis. Industrial microorganisms can affect multistep conversions of renewable feedstocks to high value chemical products in a single reactor. Products of microorganism-catalyzed fermentation processes range from chemicals such as ethanol, lactic acid, amino acids and vitamins, to high value small molecule pharmaceuticals, protein pharmaceuticals, and industrial enzymes. In many of these processes, the biocatalysts are whole-cell microorganisms, including microorganisms that have been genetically modified to express heterologous genes.

Some key parameters for efficient microorganism-catalyzed fermentation processes include the ability to grow microorganisms to a greater cell density, increased yield of desired products, increased amount of volumetric productivity, removal of unwanted co-metabolites, improved utilization of inexpensive carbon and nitrogen sources, adaptation to varying fermenter conditions, increased production of a primary metabolite, increased production of a secondary metabolite, increased tolerance to acidic conditions, increased tolerance to basic conditions, increased tolerance to organic solvents, increased tolerance to high salt conditions and increased tolerance to high or low temperatures. Inefficiencies in any of these parameters can result in high manufacturing costs, inability to capture or maintain market share, and/or failure to bring fermented end-products to market.

The methods and compositions of the present disclosure can be adapted to conventional fermentation bioreactors (e.g., batch, fed-batch, cell recycle, and continuous fermentation).

In some embodiments, a microorganism (e.g., a genetically modified microorganism) as provided herein is cultivated in liquid fermentation media (i.e., a submerged culture) which leads to excretion of the fermented product(s) into the fermentation media. In one embodiment, the fermented end product(s) can be isolated from the fermentation media using any suitable method known in the art.

In some embodiments, formation of the fermented product occurs during an initial, fast growth period of the microorganism. In one embodiment, formation of the fermented product occurs during a second period in which the culture is maintained in a slow-growing or non-growing state. In one embodiment, formation of the fermented product occurs during more than one growth period of the microorganism. In such embodiments, the amount of fermented product formed per unit of time is generally a function of the metabolic activity of the microorganism, the physiological culture conditions (e.g., pH, temperature, medium composition), and the amount of microorganisms present in the fermentation process.

In some embodiments, the fermentation product is recovered from the periplasm or culture medium as a secreted metabolite. In one embodiment, the fermentation product is extracted from the microorganism, for example when the microorganism lacks a secretory signal corresponding to the fermentation product. In one embodiment, the microorganisms are ruptured and the culture medium or lysate is centrifuged to remove particulate cell debris. The membrane and soluble protein fractions may then be separated if necessary. The fermentation product of interest may then be purified from the remaining supernatant solution or suspension by, for example, distillation, fractionation, chromatography, precipitation, filtration, and the like.

The methods of the present disclosure are preferably preformed under anaerobic conditions. Both the degree of reduction of a product as well as the ATP requirement of its synthesis determines whether a production process is able to proceed aerobically or anaerobically. To produce butadiene and 1-propanol and/or 1,2-propanediol via anaerobic microbial conversion, or at least by using a process with reduced oxygen consumption, redox imbalances should be avoided. Several types of metabolic conversion steps involve redox reactions including some of the conversions as set forth in FIGS. 1-4. Such redox reactions involve electron transfer mediated by the participation of redox cofactors such as NADH, NADPH and ferredoxin. Since the amounts of redox cofactors in the cell are limited to permit the continuation of metabolic processes, the cofactors have to be regenerated. In order to avoid such redox imbalances, alternative ways of cofactor regeneration may be engineered, and in some cases additional sources of ATP generation may be provided. Alternatively, oxidation and reduction processes may be separated spatially in bioelectrochemical systems (Rabaey and. Rozendal, 2010, Nature reviews, Microbiology, vol 8: 706-716).

In some embodiment, redox imbalances may be avoided by using substrates (e.g., fermentable carbon sources) that are more oxidized or more reduced. for example, if the utilization of a substrate results in a deficit or surplus of electrons, a requirement for oxygen can be circumvented by using substrates that are more reduced or oxidized, respectively. For example, glycerol which is a major byproduct of biodiesel production is more reduced than sugars, and is therefore more suitable for the synthesis of compounds whose production from sugar results in cofactor oxidation, such as succinic acid. In some embodiments, if the conversion of a substrate to a product results in an electron deficit, co-substrates can be added that function as electron donors (Babel 2009, Eng. Life Sci. 9,285-290). An important criterion for the anaerobic use of co-substrates is that their redox potential is higher than that of NADH (Geertman et al., 2006, FEMS Yeast Res. 6, 1193-1203). If the conversion of substrate to produce results in an electron surplus, co-substrates can be added that function as electron acceptors.

Methods for the Production of Polybutadiene and Other Compounds from Butadiene

Butadiene is gaseous at room temperature or in fermentative conditions (20-45° C.), and their production from a fermentation process results in a gas that could accumulate in the headspace of a fermentation tank, and be siphoned and concentrated. Butadiene may be purified from fermentation of gases, including gaseous alcohol, CO₂ and other compound by solvent extraction, cryogenic processes, distillation, fractionation, chromatography, precipitation, filtration, and the like.

Butadiene produced via any of the processes or methods disclosed herein may be converted to polybutadiene. Alternatively, butadiene produced via methods disclosed herein may be polymerized with other olefins to form copolymers such as acrylonitrile-butadiene-styrene (ABS), acrylonitrile-butadiene (ABR), or styrene-butadiene (SBR) copolymers, BR butyl rubber (RB), poly butadiene rubber (PBR), nitrile rubber and polychloroprene (Neoprene). Those synthetic rubbers or plastic elastomers applications include productions of tires, plastic materials, sole, shoe hills, technical goods, home appliance, neoprene, paper coatings, gloves, gaskets and seals.

Methods for the Production of Polypropylene from 1-Propanol

1-propanol produced from a fermentation described herein may be separated from one or more microorganisms by centrifugation. Additionally, 1-propanol could be purified from broth using distillation, membranes or adsorption columns. After distillation, 1-propanol could dehydrated to propylene. Propylene is a chemical compound that is widely used to synthesize a wide range of petrochemical products. For example, it is the raw material used for the production of polypropylene, their copolymers and other chemicals such as acrylonitrile, acrylic acid, epichloridrine and acetone. In an embodiment, propylene may be polymerized to produce thermoplastics resins for innumerous applications such as rigid or flexible packaging materials, blow molding and injection molding.

Methods for the Production of Polyurethanes from 1,2-Propanediol

1,2-propanediol produced from a fermentation described herein may be separated from one or more microorganisms by centrifugation. Additionally, 1-propanol could be purified from broth using distillation, membranes or adsorption columns. In some embodiments, the purified 1,2-propanediol could be used to produce polyurethane. Polyurethane may be produced via a reaction between a diisocyanate (aromatic and aliphatic types are available) and a polyol, typically a polypropylene glycol or polyester polyol, in the presence of catalysts and materials for controlling the cell structure, (surfactants) in the case of foams. It will be understood that polyurethane can be made in a variety of densities and hardness by varying the type of monomer(s) used and adding other substances to modify their characteristics, notably density, or enhance their performance by any method known in the art. In an embodiment, other additives can be used to improve the fire performance, stability in difficult chemical environments and other properties of the polyurethane products.

### EXAMPLES

### Example 1

### Modification of Microorganism for Co-Production of Butadiene and 1-Propanol and/or 1,2-Propanediol

A microorganism such as a bacterium is genetically modified to co-produce butadiene and 1-propanol and/or 1,2-propanediol from a fermentable carbon source including, for example, glucose.

In an exemplary method, a microorganism may be genetically engineered by any methods known in the art to comprise: i.) one or more polynucleotides coding for enzymes in a pathway that catalyze a conversion of the fermentable carbon source to crotonyl alcohol, 5-hydroxy-3-ketovaleryl-CoA, 3-ketopent-4-enoyl-CoA, or 3,5-ketovaleryl-CoA and one or more polynucleotides coding for enzymes in a pathway that catalyze a conversion of crotonyl alcohol, 5-hydroxy-3-ketovaleryl-CoA, 3-ketopent-4-enoyl-CoA, or 3,5-ketovaleryl-CoA to butadiene. Additionally, the microorganism is modified to comprise one or more polynucleotides coding for enzymes that catalyze a conversion of the fermentable carbon source to methylglyoxal and lactate and one or more polynucleotides coding for enzymes in a pathway that catalyze a conversion of methylglyoxal and lactate to 1-propanol and/or 1,2-propanediol.

Alternatively, a microorganism that lacks one or more enzymes (e.g., one or more functional enzymes that are catalytically active) for the conversion of a fermentable carbon source to butadiene may be genetically modified to comprise one or more polynucleotides coding for enzymes (e.g., functional enzymes including, for example any enzyme disclosed herein) in a pathway that the microorganism lacks to catalyze a conversion of the fermentable carbon source to butadiene and 1-propanol and/or 1,2-propanediol.

### Example 2

### Fermentation of Glucose by Genetically Modified Microorganism to Produce 1-Propanol and/or Butadiene

A genetically modified microorganism, as produced in Example 1 above, is used to ferment a carbon source, to produce butadiene and 1-propanol and/or 1,2-propanediol.

In an exemplary method, a previously-sterilized culture medium comprising a fermentable carbon source (e.g., 9 g/L glucose, 1 g/L KH₂PO₄, 2 g/L (NH₄)₂HPO₄, 5 mg/L FeSO₄.7H₂O, 10 mg/L MgSO₄.7H₂O, 2.5 mg/L MnSO₄.H₂O, 10 mg/L CaCl₂.6H₂O, 10 mg/L CoCl₂.6H₂O, and 10 g/L yeast extract) is charged in a bioreactor. One or more modified microorganisms as produced in Example 1 are then added to the bioreactor.

During fermentation, anaerobic conditions are maintained by, for example, sparging nitrogen through the culture medium. A suitable temperature for fermentation (e.g., about 30° C.) is maintained using any method known in the art. A near physiological pH (e.g., about 6.5) is maintained by, for example, automatic addition of sodium hydroxide. The bioreactor is agitated at, for example, about 50 rpm. Fermentation is allowed to run to completion. During or after fermentation, butadiene and 1-propanol and/or 1,2-propanediol may be removed (e.g., separated from the fermentable carbon source) from the bioreactor.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A method of co-producing butadiene and 1-propanol and/or 1,2-propanediol from a fermentable carbon source, the method comprising:
providing a fermentable carbon source;
contacting the fermentable carbon source with a microorganism comprising one or more polynucleotides coding for enzymes in a pathway that catalyzes a conversion of the fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol in a fermentation media; and
expressing the one or more polynucleotides coding for the enzymes in the pathway that catalyzes a conversion of the fermentable carbon source to one or more intermediates in a pathway for the co-production of butadiene and 1-propanol and/or 1,2-propanediol and one or more polynucleotides coding for enzymes in a pathway that catalyze a conversion of the one or more intermediates to butadiene and 1-propanol and/or 1,2-propanediol in the microorganism to produce butadiene and 1-propanol and/or 1,2-propanediol,
wherein the one or more intermediates in the pathway for the production of butadiene are selected from the group consisting of: crotonyl alcohol, 5-hydroxy-3-ketovaleryl-CoA, 3-ketopent-4-enoyl-CoA and 3,5-ketovaleryl-CoA, wherein the one or more intermediates in the pathway for the production of 1-propanol and/or 1,2-propanediol are selected from the group consisting of: methylglyoxal and lactate, and wherein the co-production method is anaerobic.

2. The method of claim 1, wherein butadiene and 1-propanol and/or 1,2-propanediol are produced.

3. The method of claim 1, wherein butadiene and 1-propanol are produced.

4. The method of claim 1, wherein butadiene and 1,2-propanediol are produced.

5. The method of claim 1, wherein butadiene is produced via a crotonyl-alcohol intermediate and 1-propanol and/or 1,2-propanediol is produced via a methylglyoxal and a R/S lactate intermediate.

6. The method of claim 1, wherein butadiene is produced via a 5-hydroxy-3-ketovaleryl-CoA intermediate and 1-propanol and/or 1,2-propanediol is produced via a methylglyoxal and a R/S lactate intermediate.

7. The method of claim 1, wherein butadiene is produced via a 3-ketopent-4-enoyl-CoA intermediate and 1-propanol and/or 1,2-propanediol is produced via a methylglyoxal and a R/S lactate intermediate.

8. The method of claim 1, wherein butadiene is produced via a 3,5-ketovaleryl-CoA intermediate and 1-propanol and/or 1,2-propanediol is produced via a methylglyoxal and a R/S lactate intermediate.

9. The method of claim 1, wherein the microorganism is an archea, bacteria, or eukaryote.

10. The method of claim 9, wherein the bacteria is selected from the genera consisting of: Propionibacterium, Propionispira, Clostridium, Bacillus, Escherichia, Pelobacter, or Lactobacillus.

11. The method of claim 10, wherein the eukaryote is a yeast, filamentous fungi, protozoa, or algae.

12. The method of claim 11, wherein the yeast is Saccharomyces cerevisiae or Pichia pastoris.

13. The method of claim 1, wherein the carbon source is sugarcane juice, sugarcane molasses, hydrolyzed starch, hydrolyzed lignocellulosic materials, glucose, sucrose, fructose, lactate, lactose, xylose, pyruvate, or glycerol in any form or mixture thereof.

14. The method of claim 1, wherein the carbon source is a monosaccharide, oligosaccharide, or polysaccharide.

15. The method of claim 1, wherein the produced butadiene and 1-propanol and/or 1,2-propanediol are secreted by the microorganism into the fermentation media.

16. The method of claim 15 further comprising recovering the produced butadiene and 1-propanol and/or 1,2-propanediol from the fermentation media.

17. The method of claim 1, wherein the microorganism has been genetically modified to express the one or more polynucleotides coding for enzymes in a pathway that catalyzes a conversion of the fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol.

18. A microorganism comprising one or more polynucleotides coding for enzymes in a pathway that catalyzes a conversion of a fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol
wherein the one or more intermediates in the pathway for the production of butadiene are selected from the group consisting of: crotonyl alcohol, 5-hydroxy-3-ketovaleryl-CoA, 3-ketopent-4-enoyl-CoA and 3,5-ketovaleryl-CoA, wherein the one or more intermediates in the pathway for the production of 1-propanol and/or 1,2-propanediol are selected from the group consisting of: methylglyoxal and lactate.

19. The microorganism of claim 18, wherein the microorganism is an archea, bacteria, or eukaryote.

20. The microorganism of claim 19, wherein the bacteria is selected from the genera consisting of: Propionibacterium, Propionispira, Clostridium, Bacillus, Escherichia, Pelobacter, or Lactobacillus.

21. The microorganism of claim 19, wherein the eukaryote is a yeast, filamentous fungi, protozoa, or algae.

22. The microorganism of claim 21, wherein the yeast is Saccharomyces cerevisiae or Pichia pastoris.

23. The microorganism of claim 18, wherein the microorganism has been genetically modified to express the one or more polynucleotides coding for enzymes in a pathway that catalyzes a conversion of the fermentable carbon source to one or more intermediates in a pathway for the co-production of butadiene and 1-propanol and/or 1,2-propanediol, and one or more polynucleotides coding for enzymes in a pathway that catalyze a conversion of the one or more intermediates to butadiene and 1-propanol and/or 1,2-propanediol.
